(19) United States
(12) Patent Application Publication
(10) Pub. No.: US 2020/0368162 A1
(43) Pub. Date: Nov. 26, 2020

(54) Nucleic Acid-Based Therapy of Muscular Dystrophies

(71) Applicant: ModernaTX, Inc., Cambridge, MA (US)

(72) Inventor: Paolo Martini, Boston, MA (US)

(21) Appl. No.: 16/487,734

(22) PCT Filed: Feb. 24, 2018
(86) PCT No.: PCT/US2018/019597

§ 371 Date: Aug. 21, 2019

Related U.S. Application Data
(60) Provisional application No. 62/463,548, filed on Feb. 24, 2017.

(51) Int. Cl. A61K 9/127; C07K 14/47; A61K 9/51; A61K 31/221

(52) U.S. Cl. CPC A61K 9/1271; C07K 14/4708; A61K 9/0019; A61K 9/5123; A61K 31/221; A61K 9/5138

(57) ABSTRACT

The invention related to polynucleotides comprising an open reading frame of linked nucleosides encoding therapeutic proteins or variant therapeutic proteins, isoforms thereof, functional fragments thereof, and fusion proteins comprising therapeutic proteins. In some embodiments, the open reading frame is sequence-optimized. The invention also relates to methods of treating muscular dystrophies.

### RELATED APPLICATIONS

This application claims the benefit under 35 U.S.C. § 119(e) of U.S. provisional application No. 62/463,548, filed Feb. 24, 2017, which is incorporated by reference herein in its entirety.

### BACKGROUND

Muscle dystrophy (MD) is a group of genetic diseases that result in progressive weakness and loss of muscle mass. There are nine major categories of muscular dystrophy, and over 30 specific types of disease, each of which vary in terms of the muscles affected, progression of disease, and onset of disease. The most prevalent, Duchenne muscular dystrophy (DMD), accounts for nearly half of the patients with muscular dystrophy.

Duchenne muscular dystrophy (DMD) is a muscle wasting disease caused by mutations in the DMD gene, which encodes dystrophin, in all types of muscle (i.e., skeletal, cardiac, and smooth) and in neurons. The mutations are generally X-linked recessive; however, de novo mutations are also possible. The DMD gene contains 79 exons distributed over 2.3 million basepairs (bp) on the X chromosome; however, only approximately 14,000 bp (<1%) are used for translation into protein. The remaining 99.5% of the gene is spliced out of the 2.3 million bp initial heteronuclear RNA transcript, resulting in the mature 14,000 bp mRNA that contains all the information necessary for dystrophin protein production. Dystrophin is expressed at the sarcolemma of skeletal muscle, where it maintains the strength, flexibility, and stability of the muscle fiber. Further, the protein forms a critical link between the cytoskeleton and the dystrophin-associated complex at the sarcolemma. Mutations in the DMD gene impact the integrity of the muscle fiber's cell membrane, leading to muscle loss and marked dystrophin deficiency in muscle. DMD affects approximately one in 3,500 males at birth, and affected individuals generally live into their early 30s.

There is presently no cure for DMD or any of the other muscular dystrophies.

### SUMMARY OF THE INVENTION

In certain aspects, the invention relates to compositions and delivery formulations comprising a polynucleotide, e.g., a ribonucleic acid (RNA), e.g., a messenger RNA (mRNA), encoding a therapeutic protein and methods for treating muscular dystrophy in a subject in need thereof by administering the same.

Aspects of the invention relate to an RNA polynucleotide comprising an open reading frame (ORF) encoding a therapeutic polypeptide formulated in a cationic lipid nanoparticle, wherein the cationic lipid nanoparticle has a molar ratio of about 20-60% cationic lipid:about 5-25% non-cationic lipid: about 25-55% sterol; and about 0.5-15% PEG-modified lipid. Some aspects of the invention relate to an RNA polynucleotide comprising an open reading frame (ORF) encoding a therapeutic variant polypeptide formulated in a cationic lipid nanoparticle. In some embodiments, the therapeutic protein is a muscle therapeutic protein.

Other aspects of the invention relate to an RNA polynucleotide comprising an open reading frame (ORF) encoding a therapeutic polypeptide formulated in a cationic lipid nanoparticle, wherein the RNA polynucleotide in the cationic lipid nanoparticle has a therapeutic index of greater than 10% of the therapeutic index of the RNA polynucleotide alone.

In some embodiments, the therapeutic polypeptide is a therapeutic variant polypeptide. In some embodiments, at least 30%-50% of the mRNA is on the surface of the cationic lipid nanoparticle. In other embodiments, the cationic lipid nanoparticle has a mean diameter of 50-200 nm.

In some embodiments, the cationic lipid nanoparticle has a 5:1 to 18:1 weight ratio of total lipid to RNA polynucleotide. In some embodiments, the composition is a unit dosage form having a dosage of 25-200 micrograms of the RNA polynucleotide. In some embodiments, the cationic lipid is a lipid selected from compound 1-20. In some embodiments, the open reading frame is codon optimized.

In other embodiments, the RNA comprises at least one chemical modification. In some embodiments, the chemical modification is selected from pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine and 2′-O-methyl uridine.

In some embodiments, the RNA polynucleotide formulated in the cationic lipid nanoparticle has a therapeutic index of greater than 60% of the therapeutic index of the RNA polynucleotide alone. In some embodiments, the RNA polynucleotide formulated in the cationic lipid nanoparticle has a therapeutic index of greater than 10% of the therapeutic index of the RNA polynucleotide alone.

In other embodiments, the cationic lipid is a lipid of Formula (I):

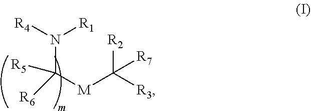

or a salt or isomer thereof, as defined herein.

In some embodiments, a subset of compounds of Formula (I) includes those of Formula (IA):

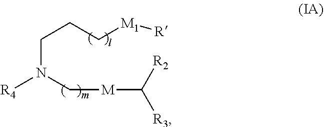

or a salt or isomer thereof, as defined herein.

In some embodiments, the nanoparticle has a polydispersity value of less than 0.4. In some embodiments, the nanoparticle has a net neutral charge at a neutral pH.

In some embodiments, 80% of the uracil in the open reading frame have a chemical modification. In some embodiments, 100% of the uracil in the open reading frame have a chemical modification. In some embodiments, the chemical modification is in the 5-position of the uracil. In some embodiments, the chemical modification is N1-methylpseudouridine. In other embodiments, the uracil and thymine content of the RNA polynucleotide is 100-150% greater than that of wild-type therapeutic polynucleotides.

Aspects of the invention relate to a method of increasing the therapeutic index of an RNA polynucleotide comprising an open reading frame (ORF) encoding a therapeutic polypeptide, the method comprising associating the RNA polynucleotide with a cationic lipid to produce a composition, thereby increasing the therapeutic index of the RNA polynucleotide in the composition relative to the therapeutic index of the RNA polynucleotide alone.

In some embodiments, the therapeutic index of the RNA polynucleotide in the composition is greater than 10:1. In other embodiments, the therapeutic index of the RNA polynucleotide in the composition is greater than 50:1.

Further aspects of the invention relate to a method for treating a subject comprising administering to a subject in need thereof the composition produced in an effective amount to treat the subject.

Aspects of the invention relate to a method of treating muscular dystrophy in a subject in need thereof, comprising administering to the subject a therapeutically effective amount of an RNA polynucleotide comprising an open reading frame (ORF) encoding a therapeutic polypeptide wherein administration of the RNA polynucleotide results in an increase in the subject's deficient protein to a physiological level.

In some embodiments, the method of treating muscular dystrophy involves a single administration of the RNA polynucleotide. In some embodiments, the method of treating muscular dystrophy further comprises administering a weekly dose. In other embodiments, the RNA polynucleotide is formulated in a cationic lipid nanoparticle.

In some embodiments, the RNA polynucleotide is in a composition as previously described. In some embodiments, upon administration to the subject the dosage form exhibits a pharmacokinetic (PK) profile comprising: a) a Tₘₐₓ at about 30 to about 240 minutes after administration; and b) a plasma drug (therapeutic polypeptide produced by RNA polynucleotide) concentration plateau of at least 50% Cₘₐₓ for a duration of about 90 to about 240 minutes.

In some embodiments, upon administration to the subject at least a 25% increase in therapeutic protein level relative to baseline levels is achieved. In other embodiments, upon administration to the subject at least a 50% increase in therapeutic protein level relative to baseline levels is achieved.

In some embodiments, upon administration to the subject at least a 60% increase in therapeutic protein level relative to baseline levels is achieved. In other embodiments, the therapeutic protein level increase is achieved for up to 3 days. In other embodiments, the therapeutic protein level increase is achieved for up to 5 days.

In some embodiments, therapeutic protein level increase is achieved for up to 7 days. In some embodiments, therapeutic protein level increase is achieved within 1 hour of dosing the subject. In other embodiments, therapeutic protein level increase is achieved within 3 hours of dosing the subject.

In some embodiments, the RNA polynucleotide is administered 1 per week for 3 weeks to 1 year. In some embodiments, the RNA polynucleotide is administered to the subject by intravenous administration. In some embodiments, the RNA polynucleotide is administered to the subject by subcutaneous administration.

In some embodiments, the RNA polynucleotide is present in a dosage of between 25 and 100 micrograms. In other embodiments, the method comprises administering to the subject a single dosage of between 0.001 mg/kg and 0.005 mg/kg of the RNA polynucleotide.

The present disclosure further provides a method of expressing the therapeutic polypeptide in a human subject in need thereof comprising administering to the subject an effective amount of a pharmaceutical composition or a polynucleotide, e.g. an mRNA, described herein, wherein the pharmaceutical composition or polynucleotide is suitable for administrating as a single dose or as a plurality of single unit doses to the subject. The drug may be administered in a clinical setting, e.g., hospital or clinical site, in an IV infusion over a few hours. For instance, it may be administered as a bolus IV injection, or as a procedure carried out in a day for a patient in the clinic/hospital. The single dose may be followed up by subsequent treatments, at a certain frequency, every week, two weeks, three weeks, four weeks, 5 weeks, 6 weeks, 7 weeks, 8 weeks, every month, two months, three months, four months, five months, six months, or every year.

Aspects of the invention relate to a method of treating muscular dystrophy in a subject in need thereof, comprising administering to the subject an RNA polynucleotide comprising an open reading frame (ORF) encoding a therapeutic polypeptide and a standard of care therapy for muscular dystrophy wherein the combined administration of the RNA polynucleotide and standard of care therapy results in an increase in the subject's therapeutic protein levels to a physiological level.

The present disclosure provides a polynucleotide comprising an open reading frame (ORF) encoding a therapeutic polypeptide, wherein the uracil or thymine content of the ORF is between 100% and about 150% of the theoretical minimum uracil or thymine content of a nucleotide sequence encoding the therapeutic polypeptide (% U_{TM} or % T_{TM}, respectively). In some embodiments, the ORF further comprises at least one low-frequency codon.

In some embodiments, the ORF has at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, or 100% sequence identity to a sequence selected from the sequences in Table 5. In some embodiments, the therapeutic polypeptide comprises an amino acid sequence at least about 95%, at least about 96%, at least about 97%, at least about 98%, at least about 99%, or about 100% identical to the polypeptide sequence of the wild type therapeutic protein (Table 5), and wherein the therapeutic polypeptide has therapeutic activity. In some embodiments, the therapeutic polypeptide is a variant, derivative, or mutant having a therapeutic activity. In some embodiments, the polynucleotide sequence further comprises a nucleotide sequence encoding a transit peptide.

In some embodiments, the polynucleotide further comprises a miRNA binding site. In some embodiments, the miRNA binding site comprises one or more nucleotide sequences selected from TABLE 4. In some embodiments, the miRNA binding site binds to miR-142. In some embodiments, the miRNA binding site binds to miR-142-3p or miR-142-5p. In some embodiments, the miR142 comprises SEQ ID NO: 44.

In some embodiments, the polynucleotide further comprises a 5′ UTR. In some embodiments, the 5′ UTR comprises a nucleic acid sequence at least 90%, at least about 95%, at least about 96%, at least about 97%, at least about 98%, at least about 99%, or about 100% identical to a sequence selected from the group consisting of SEQ ID NO: 1-25, or any combination thereof. In some embodiments, the polynucleotide further comprises a 3′ UTR. In some embodiments, the 3′ UTR comprises a nucleic acid sequence at least about 90%, at least about 95%, at least about 96%, at least about 97%, at least about 98%, at least about 99%, or about 100% identical to a sequence selected from the group consisting of SEQ ID NO: 26-43, or any combination thereof. In some embodiments, the miRNA binding site is located within the 3′ UTR.

In some embodiments, the polynucleotide further comprises a 5′ terminal cap. In some embodiments, the 5′ terminal cap comprises a Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof. In some embodiments, the polynucleotide further comprises a poly-A region. In some embodiments, the poly-A region is at least about 10, at least about 20, at least about 30, at least about 40, at least about 50, at least about 60, at least about 70, at least about 80, or at least about 90 nucleotides in length. In some embodiments, the poly-A region has about 10 to about 200, about 20 to about 180, about 50 to about 160, about 70 to about 140, about 80 to about 120 nucleotides in length.

In some embodiments, upon administration to a subject, the polynucleotide has: (i) a longer plasma half-life; (ii) increased expression of a therapeutic polypeptide encoded by the ORF; (iii) a lower frequency of arrested translation resulting in an expression fragment; (iv) greater structural stability; or (v) any combination thereof, relative to a corresponding polynucleotide comprising the wild type therapeutic polynucleotide.

In some embodiments, the polynucleotide comprises: (i) a 5′-terminal cap; (ii) a 5′-UTR; (iii) an ORF encoding a therapeutic polypeptide; (iv) a 3′-UTR; and (v) a poly-A region. In some embodiments, the 3′-UTR comprises a miRNA binding site.

The present disclosure also provides a method of producing the polynucleotide described herein, the method comprising modifying an ORF encoding a therapeutic polypeptide by substituting at least one uracil nucleobase with an adenine, guanine, or cytosine nucleobase, or by substituting at least one adenine, guanine, or cytosine nucleobase with a uracil nucleobase, wherein all the substitutions are synonymous substitutions. In some embodiments, the method further comprises replacing at least about 90%, at least about 95%, at least about 99%, or about 100% of uracils with 5-methoxyuracils.

In certain embodiments, a subset of compounds of Formula (I) includes those of Formula (IIa), (IIb), (IIc), or (IIe):

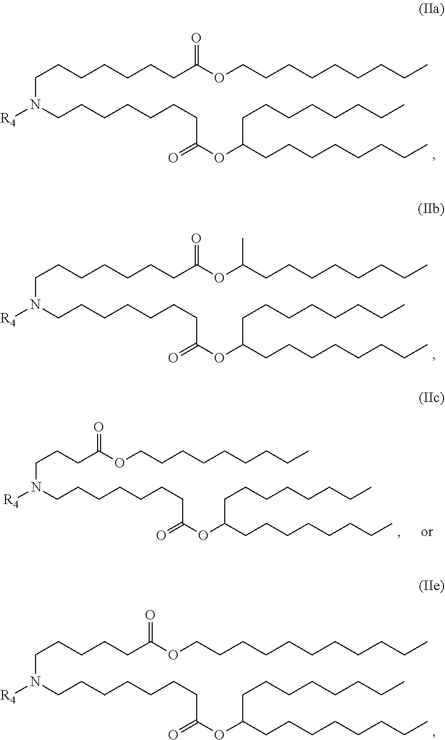

or a salt or isomer thereof, wherein R₄ is as described herein.

In some embodiments, R₄ is as described herein.

In some embodiments, the compound is of the Formula (IId),

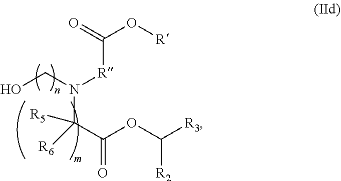

or a salt or stereoisomer thereof,

wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined in

In some embodiments, R₂ is C₈ alkyl. In some embodiments, R₃ is C₅ alkyl, C₆ alkyl, C₇ alkyl, C₈ alkyl, or C₉ alkyl. In some embodiments, m is 5, 7, or 9. In some embodiments, each R₅ is H. In some embodiments, each R₆ is H.

In another aspect, the disclosure features a nanoparticle composition including a lipid component comprising a compound as described herein (e.g., a compound according to Formula (I), (IA), (II), (IIa), (IIb), (IIc), (IId) or (IIe)).

In yet another aspect, the disclosure features a pharmaceutical composition comprising a nanoparticle composition according to the preceding aspects and a pharmaceutically acceptable carrier. For example, the pharmaceutical composition is refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition is a solution that is refrigerated for storage and/or shipment at, for example, about −20° C., −30° C., −40° C., −50° C., −60° C., −70° C., or −80° C.

In another aspect, the disclosure provides a method of delivering a therapeutic and/or prophylactic (e.g., an mRNA) to a cell (e.g., a mammalian cell). This method includes the step of administering to a subject (e.g., a mammal, such as a human) a nanoparticle composition including (i) a lipid component including a phospholipid (such as a polyunsaturated lipid), a PEG lipid, a structural lipid, and a compound of Formula (I), (IA), (II), (IIa), (IIb), (IIc), (IId) or (IIe) and (ii) a therapeutic and/or prophylactic, in which administering involves contacting the cell with the nanoparticle composition, whereby the therapeutic and/or prophylactic is delivered to the cell.

In another aspect, the disclosure provides a method of producing a polypeptide of interest in a cell (e.g., a mammalian cell). The method includes the step of contacting the cell with a nanoparticle composition including (i) a lipid component including a phospholipid (such as a polyunsaturated lipid), a PEG lipid, a structural lipid, and a compound of Formula (I), (IA), (II), (IIa), (IIb), (IIc), (IId) or (IIe) and (ii) an mRNA encoding the polypeptide of interest, whereby the mRNA is capable of being translated in the cell to produce the polypeptide.

In another aspect, the disclosure provides a method of treating a disease or disorder in a mammal (e.g., a human) in need thereof. The method includes the step of administering to the mammal a therapeutically effective amount of a nanoparticle composition including (i) a lipid component including a phospholipid (such as a polyunsaturated lipid), a PEG lipid, a structural lipid, and a compound of Formula (I), (IA), (II), (IIa), (IIb), (IIc), (IId) or (IIe) and (ii) a therapeutic and/or prophylactic (e.g., an mRNA). In some embodiments, the disease or disorder is characterized by dysfunctional or aberrant protein or polypeptide activity.

In another aspect, the disclosure provides a method of delivering (e.g., specifically delivering) a therapeutic and/or prophylactic to a mammalian organ (e.g., a liver, spleen, lung, or femur). This method includes the step of administering to a subject (e.g., a mammal) a nanoparticle composition including (i) a lipid component including a phospholipid, a PEG lipid, a structural lipid, and a compound of Formula (I), (IA), (II), (IIa), (IIb), (IIc), (IId) or (IIe) and (ii) a therapeutic and/or prophylactic (e.g., an mRNA), in which administering involves contacting the cell with the nanoparticle composition, whereby the therapeutic and/or prophylactic is delivered to the target organ (e.g., a liver, spleen, lung, or femur).

In another aspect, the disclosure features a method for the enhanced delivery of a therapeutic and/or prophylactic (e.g., an mRNA) to a target tissue (e.g., a liver, spleen, lung, muscle, or femur). This method includes administering to a subject (e.g., a mammal) a nanoparticle composition, the composition including (i) a lipid component including a compound of Formula (I), (IA), (II), (IIa), (IIb), (IIc), (IId) or (IIe), a phospholipid, a structural lipid, and a PEG lipid; and (ii) a therapeutic and/or prophylactic, the administering including contacting the target tissue with the nanoparticle composition, whereby the therapeutic and/or prophylactic is delivered to the target tissue

In some embodiments, the composition disclosed herein is a nanoparticle composition. In some embodiments, the delivery agent further comprises a phospholipid. In some embodiments, the phospholipid is selected from the group consisting of
1,2-dilinoleoyl-sn-glycero-3-phosphocholine (DLPC),
1,2-dioleoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanolamine (DOPE),
1,2-diphytanoyl-sn-glycero-3-phosphoethanolamine (ME 16:0 PE),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), sphingomyelin, and any mixtures thereof.

In some embodiments, the delivery agent further comprises a structural lipid. In some embodiments, the structural lipid is selected from the group consisting of cholesterol, fecosterol, sitosterol, ergosterol, campesterol, stigmasterol, brassicasterol, tomatidine, ursolic acid, alpha-tocopherol, and any mixtures thereof.

In some embodiments, the delivery agent further comprises a PEG lipid. In some embodiments, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and any mixtures thereof.

In some embodiments, the delivery agent further comprises an ionizable lipid selected from the group consisting of
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),

heptatriaconta-6,9,28,31-tetraen-19-yl 4-(dimethylamino)butanoate (DLin-MC3-DMA),
2,2-dilinoleyl-4-(2-dimethylaminoethyl)[1,3]-dioxolane (DLin-KC2-DMA),
1,2-dioleyloxy-N,N-dimethylaminopropane (DODMA),
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

In some embodiments, the delivery agent further comprises a phospholipid, a structural lipid, a PEG lipid, or any combination thereof.

In some embodiments, the composition is formulated for in vivo delivery. In some embodiments, the composition is formulated for intramuscular, subcutaneous, or intradermal delivery.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF THE DRAWINGS/FIGURES

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIGS. 1A-1C show zebrafish embryo microinjection sites. FIG. 1A shows 1-cell stage embryos with injection needles targeting the large yolk. From the 1-cell to at least the 4-cell stage, cytoplasmic streaming will carry injected mRNAs from the yolk into the blastomeres on top of the yolk. FIG. 1B shows that, in 24 hours post-fertilization (hpf) embryos, injection needles can target the hindbrain ventricle, the caudal vein, and trunk skeletal muscle. FIG. 1C shows that, in 48 hpf embryos, injection needles can target the hindbrain ventricle, the caudal vein, and trunk skeletal muscle. In FIGS. 1B and 1C, anterior is to the left.

FIGS. 2A-2C show live GFP expression in 24 hpf embryos following 1-cell-stage injections. FIG. 2A depicts control, non-injected embryos, which show no GFP expression and some auto-fluorescence from the yolk. FIG. 2B shows embryos injected with naked gfp mRNA, which demonstrate very robust GFP expression throughout the embryo. FIG. 2C depicts embryos injected with packaged gfp mRNA, showing broad GFP expression.

FIGS. 3A-3E show live GFP expression in 48 hpf embryos following 24 hpf-stage injections. FIG. 3A depicts control, non-injected embryos, which show no GFP expression, with some auto-fluorescence from the yolk and along the edge of the head. FIGS. 3B and 3D depict embryos injected with naked gfp mRNA, which show little or no GFP expression. FIG. 3C illustrates embryos injected with packaged gfp mRNA into the hindbrain ventricle, which show GFP expression in the forebrain (arrow), in the midbrain/hindbrain, and in the pharyngeal region (arrowhead), likely in cells derived from hindbrain neural crest. FIG. 3E depicts embryos injected with packaged gfp mRNA into trunk muscle, which show GFP expression in myotomes around the injection site (arrow) and also show GFP in the spinal cord broadly along the body axis, centered around the injection site. Embryos injected in the caudal vein are not shown.

FIGS. 4A-4E show live GFP expression in 72 hpf embryos following 48 hpf-stage injections. FIG. 4A depicts control, non-injected embryos, which show no GFP expression and some auto-fluorescence from the yolk. FIG. 4B depicts embryos injected with packaged gfp mRNA into the hindbrain ventricle, which show GFP expression in the forebrain (arrow), in the midbrain/hindbrain, and in the spinal cord (arrowhead). FIG. 4C illustrates embryos injected with packaged gfp mRNA into trunk muscle, which show GFP expression in myotomes around the injection site (arrow) and also show GFP in the spinal cord broadly along the body axis (arrowhead), centered around the injection site. FIGS. 4D and 4E show that embryos injected with naked or packaged gfp mRNA into the caudal vein can exhibit strong GFP expression in the yolk cell, possibly as a result of the injection nicking the yolk. Occasionally, other cell types labeled from the caudal vein packaged gfp mRNA injections, including myotomes (arrow, FIG. 4E) were observed.

FIGS. 5A-5F show confocal images of anti-GFP expression in 72 hpf embryos following 48 hpf-stage injections. FIGS. 5A-5C show a dorsal view of the head, anterior to left. FIGS. 5A-5B show control non-injected embryo and naked gfp mRNA-injected embryos exhibit some auto-fluorescence from blood cells (arrowhead, FIG. 5A). FIG. 5C illustrates an embryo injected with packaged gfp mRNA into the hindbrain ventricle, which shows GFP expression in clusters of forebrain, midbrain, and hindbrain neurons (arrows). FIGS. 5D-5F show a lateral view of the trunk, anterior to left. FIGS. 5D-5E show control non-injected embryo and naked gfp mRNA-injected embryo, respectively, which exhibit some auto-fluorescence from blood cells in vasculature and yolk (arrowheads, FIG. 5D). FIG. 5F shows an embryo injected with packaged gfp mRNA into trunk muscle, which exhibits GFP expression in myotomes around the injection site (white arrows), in the spinal cord broadly along the body axis (top arrow), and in neural crest cells that populate myotome boundaries (rightmost arrow).

### DETAILED DESCRIPTION

Gene therapy-based clinical trials for DMD and other muscular dystrophies using the “replacement” approach have been hindered due to the size of the mutated genes. For example, the DMD gene is over 11 kb in size. Consequently, partially functional, intact, truncated DMD copies are delivered to the cell. However, these approaches have had problems.

It has been discovered that proteins which are therapeutic to muscle tissue can be delivered in vivo in the form of a therapeutic RNA. Using a unique in vivo model of zebrafish it was demonstrated that relevant levels of proteins were delivered to tissue using a mRNA in a cationic lipid carrier.

Thus, the invention, in some aspects, is a composition of an RNA polynucleotide comprising an open reading frame (ORF) encoding a therapeutic polypeptide which may be formulated in a cationic lipid nanoparticle. The therapeutic protein may be a wild type therapeutic or a variant polypeptide. The compositions of the invention have several advantages over prior art methods for managing muscular dystrophies, including prior art therapeutic formulations such as protein or nucleic acid therapeutic formulations.

While there are more than 30 types of muscular dystrophy (MD), there are nine major forms. Each is caused by genetic or de novo mutations in specific gene(s) and is described further below.

Duchenne muscular dystrophy (DMD) is a muscle wasting disease caused by mutations in the DMD gene, which encodes dystrophin (Hoffman et al., 1997). The current standard of care is corticosteroid treatment, which delays the progression of skeletal muscle and cardiac dysfunction but also has serious side effects (Bushby et al., 2010; Goemans and Buyse, 2014; Kinnett et al., 2015). Therapies being pursued include dystrophin replacement through AAV vector delivery and CRISPR-Cas9 repair of dystrophin mutations (Guiraud et al., 2015; Robinson-Hamm and Gersbach, 2016). Through the use of DMD animal models, in particular the GRMD dog, the mdx mouse, and the zebrafish dmd null mutant strain, promising mRNA therapeutic targets, such as jag1, have been identified (Kawahara et al., 2014; Kornegay et al., 2014; Vieira et al., 2015). However, a major hurdle for advancing these different therapies involves developing approaches for delivering therapeutic reagents to muscle.

Becker muscular dystrophy also results from a dystrophin deficiency, and patients exhibit milder symptoms than those with DMD. Congenital muscular dystrophy is predominantly caused by a defected in merosin, a protein that surrounds muscle fibers. Consequently, the patient may experience symptoms associated with the central nervous system in addition to muscle weakening. Emery-Dreifuss muscular dystrophy predominantly affects boys, and is caused by mutations in the EMD and LMNA genes, which code for nuclear envelope components. Facioscapulohumeral muscular dystrophy, the third most common genetic skeletal muscle disease, is caused by contraction of the D4Z4 repeat in the 4q35 subtelomeric region of chromosome 4, in addition to a “toxic gain of function” of the DUX4 gene. Limb-girdle muscular dystrophy encompasses several types, each of which are caused by different gene mutations, including mutations in the LMNA, CAV3, CAPN3, DYSF, SGCA, SGCB, SGCC, SGCD, TTN, AND ANOS genes. Distal muscular dystrophy, which affects muscles of the forearms, hands, lower legs, and feet, is caused by defects in the protein dysferlin. Oculopharyngeal muscular dystrophy, which has a later onset, is caused by mutations in the PABPN1 gene, which has an abnormally extended polyalanine tract, which causes PABPN1 protein to accumulate within muscle cells.

A, “muscle therapeutic protein or polypeptide”, “therapeutic protein” or “therapeutic polypeptide,” refers to a protein that promotes or supports muscle maintenance or development. These proteins include any protein that alleviates one or more symptoms of a muscular disease or dystrophy. Therapeutic proteins or polypeptides include, for instance proteins which can have a systemic effect as well as those which have a local effect in one or more tissues, such as muscle tissue. For example, Notch signaling proteins, such as JAG1 (a systemic protein) may be administered to patients with DMD to increase dystrophin levels. Further, truncated forms of dystrophin (mini- and micro-dystrophin, Harper et al., 2002) may be used. The truncated forms include those sequences with or without central ‘hinge’ regions as well as those with fewer specrtin-like repeats. In some embodiments, the micro- or mini-dystrophin includes but is not limited to, exon 17 to exon 48, Δ17-48, ΔH2-R19, ΔH2-H3, ΔR2-R21, ΔR2-R21+H3, ΔR4-R23, ΔR9-R16 constructs. In early clinical trials, a T-cell specific response to dystrophin as well as AAV was seen in patients administered mini-dystrophin via an AAV vector (rAAV2.5.CMV.Δ3990) (Mendell et al., 2010). The present disclosure avoids such immune responses through administration using lipid nanoparticles. JAG1 (jagged1) has also been identified as a therapeutic target (Vieira et al., 2015). Other therapeutic proteins useful to counter the effects of different muscular dystrophies are also within the scope of the present disclosure. For example, dystrophin, utrophin, follistatin, follistatin 3, Wnt inhibitory factor-I, Wnt5, midkine (neurite growth-promoting factor 2, NEGF2), merosin (laminin α2), emerin, lamins A and C, KAI/CD82, α-dystroglycan, β-dystroglycan, integrin α7, β-dystroglycan, sarcospan, α-sarcoglycan, β-sarcoglycan, δ-sarcoglycan, γ-sarcoglycan, neuronal nitric oxide synthase (nNOS), mitsugumin 53 (MG53), O-mannosyltransferase (POMT) enzyme complex, fukutin, fukutin-related protein, dolichol-phosphate-mannose (DPM) synthase, anoctamin 5, dolichol kinase, like-acetylglucosaminyltransferase, beta-1,4-glucuronyltransferase 1, beta-1,3-N-acetylgalactosaminyltransferase 2, nebulin, isoprenoid synthase domain containing (ISPD), transmembrane protein 5, GDP-mannose pyrophosphorylase B, caveolin-3, titin, telethonin, nebulin, and dysferlin may be encoded and administered as described herein.

The RNA polynucleotides useful in the invention include RNA encoding for one or more therapeutic proteins.

In some embodiments the RNA polynucleotide formulated in a cationic lipid nanoparticle has a therapeutic index of greater than 10% of the therapeutic index of the RNA polynucleotide alone. In other embodiments the RNA polynucleotide formulated in the cationic lipid nanoparticle has a therapeutic index of greater than 20%, 30%, 40%, 50%, 60%, 70%, 80%, or 90% of the therapeutic index of the RNA polynucleotide alone. The therapeutic index (TI) (also referred to as therapeutic ratio) is a comparison of the amount of a therapeutic agent that causes the therapeutic effect to the amount that causes toxicity.

The invention involves methods for increasing therapeutic proteins such as dystrophin. In some embodiments the composition is in a dosage form that exhibits a pharmacokinetic (PK) profile comprising: a) a Tmax at about 30 to about 240 minutes after administration; and b) a plasma drug concentration plateau of at least 50% Cmax for a duration of about 90 to about 240 minutes. In other embodiments at least a 25%, 30%, 35%, 40%, 45%, 50%, 55%, 60%, 65%, 70%, 75%, 80%, 85%, 90, 95%, or 99% increase in therapeutic protein level relative to baseline levels is achieved.

Another advantage of the methods of the invention is that the therapeutic protein level increase is achieved rapidly following dosing of the subject. For instance therapeutic or maximal therapeutic levels may be achieved within 1, 2, 3, or 4 hours of dosing the subject. The term Cmax refers to the maximum (or peak) serum concentration that a drug achieves in a specified compartment or test area of the body after the drug has been administrated and before the administration of a second dose. Tmax refers to the time after administration of a drug when the maximum plasma concentration is reached; when the rate of absorption equals the rate of elimination.

The coding sequence (CDS) for wild type dystrophin canonical mRNA sequence is described at the NCBI Reference Sequence database (RefSeq) under accession number AH003182 (“Homo sapiens dystrophin (DMD) gene, complete cds”). The wild type dystrophin canonical protein sequence is described at the RefSeq database under accession number AAA53189 (“dystrophin [Homo sapiens]”). It is noted that the specific nucleic acid sequences encoding the reference protein sequence in the Ref Seq sequences are the coding sequence (CDS) as indicated in the respective RefSeq database entry.

In certain aspects, the invention provides a polynucleotide (e.g., a ribonucleic acid (RNA), e.g., a messenger RNA (mRNA)) comprising a nucleotide sequence (e.g., an open reading frame (ORF)) encoding a therapeutic polypeptide. In some embodiments, the therapeutic polypeptide of the invention is a wild type therapeutic or variant therapeutic protein. In some embodiments, the therapeutic polypeptide of the invention is a variant, a peptide or a polypeptide containing a substitution, and insertion and/or an addition, a deletion and/or a covalent modification with respect to a wild-type therapeutic protein sequence. In some embodiments, sequence tags or amino acids, can be added to the sequences encoded by the polynucleotides of the invention (e.g., at the N-terminal or C-terminal ends), e.g., for localization. In some embodiments, amino acid residues located at the carboxy, amino terminal, or internal regions of a polypeptide of the invention can optionally be deleted providing for fragments.

In some embodiments, the polynucleotide (e.g., a RNA, e.g., an mRNA) comprising a nucleotide sequence (e.g., an ORF) of the invention encodes a substitutional variant of a wild type therapeutic protein sequence, which can comprise one, two, three or more than three substitutions. In some embodiments, the substitutional variant can comprise one or more conservative amino acids substitutions. In other embodiments, the variant is an insertional variant. In other embodiments, the variant is a deletional variant.

As recognized by those skilled in the art, wild type or variant therapeutic protein fragments, functional protein domains, variants, and homologous proteins (orthologs) are also considered to be within the scope of the therapeutic polypeptides of the invention.

Certain compositions and methods presented in this disclosure refer to the protein or polynucleotide sequences of wild type or variant therapeutic protein. A person skilled in the art will understand that such disclosures are equally applicable to any other isoforms of therapeutic proteins known in the art.

In certain aspects, the invention provides polynucleotides (e.g., a RNA, e.g., an mRNA) that comprise a nucleotide sequence (e.g., an ORF) encoding one or more therapeutic polypeptides. In some embodiments, the encoded therapeutic polypeptide of the invention can be selected from:

a full length therapeutic polypeptide (e.g., having the same or essentially the same length as the wild type therapeutic polypeptide);

a variant such as a functional fragment of any of wild type therapeutic proteins described herein (e.g., a truncated (e.g., deletion of carboxy, amino terminal, or internal regions) sequence shorter than one of wild type therapeutic proteins; but still retaining the functional activity of the protein);

a variant such as a full length or truncated wild type therapeutic proteins in which one or more amino acids have been replaced, e.g., variants that retain all or most of the therapeutic activity of the polypeptide with respect to a reference isoform (e.g., any natural or artificial variant known in the art); or

a fusion protein comprising (i) a full length wild type therapeutic protein, variant therapeutic protein, a functional fragment or a variant thereof, and (ii) a heterologous protein.

In certain embodiments, the encoded therapeutic polypeptide is a mammalian therapeutic polypeptide, such as a human therapeutic polypeptide, a functional fragment or a variant thereof.

In some embodiments, the polynucleotide (e.g., a RNA, e.g., an mRNA) of the invention increases therapeutic protein expression levels in cells when introduced in those cells, e.g., by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, compared to therapeutic protein expression levels in the cells prior to the administration of the polynucleotide of the invention. Therapeutic protein expression levels can be measured according to methods know in the art. In some embodiments, the polynucleotide is introduced to the cells in vitro. In some embodiments, the polynucleotide is introduced to the cells in vivo.

In some embodiments, the polynucleotide (e.g., a RNA, e.g., an mRNA) of the invention comprises a codon optimized nucleic acid sequence, wherein the open reading frame (ORF) of the codon optimized nucleic sequence is derived from a therapeutic protein sequence. For example, for polynucleotides of invention comprising a sequence optimized ORF encoding a specific therapeutic protein, the corresponding wild type sequence is the native therapeutic protein. Similarly, for a sequence optimized mRNA encoding a functional fragment of a therapeutic protein, the corresponding wild type sequence is the corresponding fragment from the wild-type therapeutic protein.

In some embodiments, the polynucleotides (e.g., a RNA, e.g., an mRNA) of the invention comprise a nucleotide sequence encoding wild type therapeutic protein having the full length sequence of wild type human therapeutic protein (i.e., including the initiator methionine). In mature wild type therapeutic protein, the initiator methionine can be removed to yield a “mature therapeutic protein” comprising amino acid residues of 2 to the remaining amino acids of the translated product. The teachings of the present disclosure directed to the full sequence of human therapeutic protein are also applicable to the mature form of human therapeutic protein lacking the initiator methionine. Thus, in some embodiments, the polynucleotides (e.g., a RNA, e.g., an mRNA) of the invention comprise a nucleotide sequence encoding wild type therapeutic protein having the mature sequence of wild type human therapeutic protein (i.e., lacking the initiator methionine). In some embodiments, the polynucleotide (e.g., a RNA, e.g., an mRNA) of the invention comprising a nucleotide sequence encoding wild type therapeutic protein having the full length or mature sequence of human wild type therapeutic protein is sequence optimized.

In some embodiments, the polynucleotides (e.g., a RNA, e.g., an mRNA) of the invention comprise a nucleotide sequence (e.g., an ORF) encoding a mutant therapeutic polypeptide. In some embodiments, the polynucleotides of the invention comprise an ORF encoding a therapeutic polypeptide that comprises at least one point mutation in the therapeutic protein sequence and retains therapeutic protein activity. In some embodiments, the mutant therapeutic polypeptide has a therapeutic activity which is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the therapeutic activity of the corresponding wild-type therapeutic protein (i.e., the same wild type therapeutic protein but without the mutation(s)). In some embodiments, the polynucleotide (e.g., a RNA, e.g., an mRNA) of the invention comprising an ORF encoding a mutant therapeutic polypeptide is sequence optimized.

In some embodiments, the polynucleotide (e.g., a RNA, e.g., an mRNA) of the invention comprises a nucleotide sequence (e.g., an ORF) that encodes a therapeutic polypeptide with mutations that do not alter therapeutic protein activity. Such mutant therapeutic polypeptides can be referred to as function-neutral. In some embodiments, the polynucleotide comprises an ORF that encodes a mutant therapeutic polypeptide comprising one or more function-neutral point mutations.

In some embodiments, the mutant therapeutic polypeptide has higher therapeutic protein activity than the corresponding wild-type therapeutic protein. In some embodiments, the mutant therapeutic polypeptide has a therapeutic activity that is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the activity of the corresponding wild-type therapeutic protein (i.e., the same wild type therapeutic protein but without the mutation(s)).

In some embodiments, the polynucleotides (e.g., a RNA, e.g., an mRNA) of the invention comprise a nucleotide sequence (e.g., an ORF) encoding a functional therapeutic protein fragment, e.g., where one or more fragments correspond to a polypeptide subsequence of a wild type therapeutic polypeptide and retain therapeutic protein activity. In some embodiments, the therapeutic protein fragment has activity which is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the therapeutic protein activity of the corresponding full length therapeutic protein. In some embodiments, the polynucleotides (e.g., a RNA, e.g., an mRNA) of the invention comprising an ORF encoding a functional therapeutic protein fragment is sequence optimized.

In some embodiments, the polynucleotide (e.g., a RNA, e.g., an mRNA) of the invention comprises a nucleotide sequence (e.g., an ORF) encoding a therapeutic protein fragment that has higher therapeutic protein activity than the corresponding full length therapeutic protein. Thus, in some embodiments the therapeutic protein fragment has therapeutic activity which is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% higher than the therapeutic activity of the corresponding full length therapeutic protein.

In some embodiments, the polynucleotide (e.g., a RNA, e.g., an mRNA) of the invention comprises a nucleotide sequence (e.g., an ORF) encoding a therapeutic protein fragment that is at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25% shorter than wild-type therapeutic protein.

In some embodiments, the polynucleotide (e.g., a RNA, e.g., an mRNA) of the invention comprises from about 1,200 to about 100,000 nucleotides (e.g., from 1,200 to 1,500, from 1,200 to 1,600, from 1,200 to 1,700, from 1,200 to 1,800, from 1,200 to 1,900, from 1,200 to 2,000, from 1,300 to 1,500, from 1,300 to 1,600, from 1,300 to 1,700, from 1,300 to 1,800, from 1,300 to 1,900, from 1,300 to 2,000, from 1,425 to 1,500, from 1,425 to 1,600, from 1,425 to 1,700, from 1,425 to 1,800, from 1,425 to 1,900, from 1,425 to 2,000, from 1,425 to 3,000, from 1,425 to 5,000, from 1,425 to 7,000, from 1,425 to 10,000, from 1,425 to 25,000, from 1,425 to 50,000, from 1,425 to 70,000, or from 1,425 to 100,000).

In some embodiments, the polynucleotide of the invention (e.g., a RNA, e.g., an mRNA) comprises a nucleotide sequence (e.g., an ORF) encoding a therapeutic polypeptide (e.g., the wild-type sequence, functional fragment, or variant thereof), wherein the length of the nucleotide sequence (e.g., an ORF) is at least 500 nucleotides in length (e.g., at least or greater than about 500, 600, 700, 80, 900, 1,000, 1,100, 1,200, 1,300, 1,400, 1,425, 1450, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the polynucleotide of the invention (e.g., a RNA, e.g., an mRNA) comprises a nucleotide sequence (e.g., an ORF) encoding a therapeutic polypeptide (e.g., the wild-type sequence, functional fragment, or variant thereof) further comprises at least one nucleic acid sequence that is noncoding, e.g., a miRNA binding site.

In some embodiments, the polynucleotide of the invention comprising a nucleotide sequence (e.g., an ORF) encoding a therapeutic polypeptide (e.g., the wild-type sequence, functional fragment, or variant thereof) is RNA. In some embodiments, the polynucleotide of the invention is, or functions as, a messenger RNA (mRNA). In some embodiments, the mRNA comprises a nucleotide sequence (e.g., an ORF) that encodes at least one therapeutic polypeptide, and is capable of being translated to produce the encoded therapeutic polypeptide in vitro, in vivo, in situ or ex vivo.

In some embodiments, the polynucleotide of the present disclosure (e.g., a RNA, e.g., an mRNA) comprises a sequence-optimized nucleotide sequence (e.g., an ORF) encoding a therapeutic polypeptide (e.g., the wild-type sequence, functional fragment, or variant thereof), wherein the polynucleotide comprises 1-methylpseudouridines. In some embodiments, the polynucleotide further comprises a 5′ UTR disclosed herein and a 3′UTR disclosed herein. In some embodiments, the polynucleotide disclosed herein is formulated with a delivery agent, e.g., a lipid nanoparticle comprised of an ionizable lipid of compound 18 or 25, a neutral lipid, a structural lipid and a PEG lipid. In some embodiments the delivery agent is an LNP comprised of:

an ionizable cationic lipid of

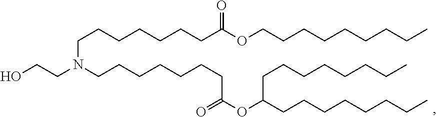 [SMILES: CCCCCCCCCOC(=O)CCCCCCCN(CCO)CCCCCCCC(=O)OC(CCCCCCCC)CCCCCCCC]

and a PEG lipid comprising Formula VI, or

an ionizable cationic lipid of

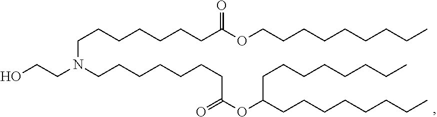 [SMILES: CCCCCCCCCOC(=O)CCCCCCCN(CCO)CCCCCCCC(=O)OC(CCCCCCCC)CCCCCCCC]

and an alternative lipid comprising oleic acid, or

an ionizable cationic lipid of

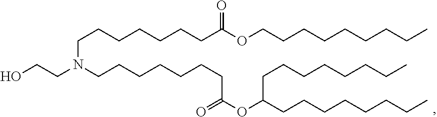 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

### Signal Sequences

The polynucleotides (e.g., a RNA, e.g., an mRNA) of the invention can also comprise nucleotide sequences that encode additional features that facilitate trafficking of the encoded polypeptides to therapeutically relevant sites. One such feature that aids in protein trafficking is the signal sequence, or targeting sequence. The peptides encoded by these signal sequences are known by a variety of names, including targeting peptides, transit peptides, and signal peptides. In some embodiments, the polynucleotide (e.g., a RNA, e.g., an mRNA) comprises a nucleotide sequence (e.g., an ORF) that encodes a signal peptide operably linked a nucleotide sequence that encodes a therapeutic polypeptide described herein.

In some embodiments, the “signal sequence” or “signal peptide” is a polynucleotide or polypeptide, respectively, which is from about 30-210, e.g., about 45-80 or 15-60 nucleotides (3-70 amino acids) in length that, optionally, is incorporated at the 5′ (or N-terminus) of the coding region or the polypeptide, respectively. Addition of these sequences results in trafficking the encoded polypeptide to a desired site, such as the endoplasmic reticulum or the mitochondria through one or more targeting pathways. Some signal peptides are cleaved from the protein, for example by a signal peptidase after the proteins are transported to the desired site.

In some embodiments, the polynucleotide of the invention comprises a nucleotide sequence encoding a wild type therapeutic polypeptide, wherein the nucleotide sequence further comprises a 5′ nucleic acid sequence encoding a native signal peptide. In another embodiment, the polynucleotide of the invention comprises a nucleotide sequence encoding a wild type therapeutic polypeptide, wherein the nucleotide sequence lacks the nucleic acid sequence encoding a native signal peptide.

In some embodiments, the polynucleotide of the invention comprises a nucleotide sequence encoding a therapeutic polypeptide, wherein the nucleotide sequence further comprises a 5′ nucleic acid sequence encoding a heterologous signal peptide.

### Fusion Proteins

In some embodiments, the polynucleotide of the invention (e.g., a RNA, e.g., an mRNA) can comprise more than one nucleic acid sequence (e.g., an ORF) encoding a polypeptide of interest. In some embodiments, polynucleotides of the invention comprise a single ORF encoding a therapeutic polypeptide, a functional fragment, or a variant thereof. However, in some embodiments, the polynucleotide of the invention can comprise more than one ORF, for example, a first ORF encoding a therapeutic polypeptide (a first polypeptide of interest), a functional fragment, or a variant thereof, and a second ORF expressing a second polypeptide of interest. In some embodiments, two or more polypeptides of interest can be genetically fused, i.e., two or more polypeptides can be encoded by the same ORF. In some embodiments, the polynucleotide can comprise a nucleic acid sequence encoding a linker (e.g., a G₄S peptide linker or another linker known in the art) between two or more polypeptides of interest.

In some embodiments, a polynucleotide of the invention (e.g., a RNA, e.g., an mRNA) can comprise two, three, four, or more ORFs, each expressing a polypeptide of interest.

In some embodiments, the polynucleotide of the invention (e.g., a RNA, e.g., an mRNA) can comprise a first nucleic acid sequence (e.g., a first ORF) encoding a therapeutic polypeptide and a second nucleic acid sequence (e.g., a second ORF) encoding a second polypeptide of interest.

### Sequence Optimization of Nucleotide Sequence Encoding a Therapeutic Polypeptide

In some embodiments, the polynucleotide (e.g., a RNA, e.g., an mRNA) of the invention is sequence optimized. In some embodiments, the polynucleotide (e.g., a RNA, e.g., an mRNA) of the invention comprises a nucleotide sequence (e.g., an ORF) encoding a therapeutic polypeptide, optionally, a nucleotide sequence (e.g, an ORF) encoding another polypeptide of interest, a 5′-UTR, a 3′-UTR, the 5′ UTR or 3′ UTR optionally comprising at least one microRNA binding site, optionally a nucleotide sequence encoding a linker, a polyA tail, or any combination thereof), in which the ORF(s) that are sequence optimized.

A sequence-optimized nucleotide sequence, e.g., a codon-optimized mRNA sequence encoding a therapeutic polypeptide, is a sequence comprising at least one synonymous nucleobase substitution with respect to a reference sequence (e.g., a wild type nucleotide sequence encoding a therapeutic polypeptide).

A sequence-optimized nucleotide sequence can be partially or completely different in sequence from the reference sequence. For example, a reference sequence encoding polyserine uniformly encoded by TCT codons can be sequence-optimized by having 100% of its nucleobases substituted (for each codon, T in position 1 replaced by A, C in position 2 replaced by G, and T in position 3 replaced by C) to yield a sequence encoding polyserine which would be uniformly encoded by AGC codons. The percentage of sequence identity obtained from a global pairwise alignment between the reference polyserine nucleic acid sequence and the sequence-optimized polyserine nucleic acid sequence would be 0%. However, the protein products from both sequences would be 100% identical.

Some sequence optimization (also sometimes referred to codon optimization) methods are known in the art and can be useful to achieve one or more desired results. These results can include, e.g., matching codon frequencies in certain tissue targets and/or host organisms to ensure proper folding; biasing G/C content to increase mRNA stability or reduce secondary structures; minimizing tandem repeat codons or base runs that can impair gene construction or expression; customizing transcriptional and translational control regions; inserting or removing protein trafficking sequences; removing/adding post translation modification sites in an encoded protein (e.g., glycosylation sites); adding, removing or shuffling protein domains; inserting or deleting restriction sites; modifying ribosome binding sites and mRNA degradation sites; adjusting translational rates to allow the various domains of the protein to fold properly; and/or reducing or eliminating problem secondary structures within the polynucleotide. Sequence optimization tools, algorithms and services are known in the art, non-limiting examples include services from GeneArt (Life Technologies), DNA2.0 (Menlo Park Calif.) and/or proprietary methods.

In some embodiments, a polynucleotide (e.g., a RNA, e.g., an mRNA) of the invention comprises a sequence-optimized nucleotide sequence (e.g., an ORF) encoding a therapeutic polypeptide, a functional fragment, or a variant thereof, wherein the therapeutic polypeptide, functional fragment, or a variant thereof encoded by the sequence-optimized nucleotide sequence has improved properties (e.g., compared to a therapeutic polypeptide, functional fragment, or a variant thereof encoded by a reference nucleotide sequence that is not sequence optimized), e.g., improved properties related to expression efficacy after administration in vivo. Such properties include, but are not limited to, improving nucleic acid stability (e.g., mRNA stability), increasing translation efficacy in the target tissue, reducing the number of truncated proteins expressed, improving the folding or prevent misfolding of the expressed proteins, reducing toxicity of the expressed products, reducing cell death caused by the expressed products, increasing and/or decreasing protein aggregation.

In some embodiments, the sequence-optimized nucleotide sequence sequence (e.g., an ORF) is codon optimized for expression in human subjects, having structural and/or chemical features that avoid one or more of the problems in the art, for example, features which are useful for optimizing formulation and delivery of nucleic acid-based therapeutics while retaining structural and functional integrity; overcoming a threshold of expression; improving expression rates; half-life and/or protein concentrations; optimizing protein localization; and avoiding deleterious bio-responses such as the immune response and/or degradation pathways.

In some embodiments, the polynucleotides of the invention comprise a nucleotide sequence (e.g., a nucleotide sequence (e.g, an ORF) encoding a therapeutic polypeptide, a nucleotide sequence (e.g, an ORF) encoding another polypeptide of interest, a 5′-UTR, a 3′-UTR, a microRNA, a nucleic acid sequence encoding a linker, or any combination thereof) that is sequence-optimized according to a method comprising: (i) substituting at least one codon in a reference nucleotide sequence (e.g., an ORF encoding a therapeutic polypeptide) with an alternative codon to increase or decrease uridine content to generate a uridine-modified sequence; (ii) substituting at least one codon in a reference nucleotide sequence (e.g., an ORF encoding a therapeutic polypeptide) with an alternative codon having a higher codon frequency in the synonymous codon set; (iii) substituting at least one codon in a reference nucleotide sequence (e.g., an ORF encoding a therapeutic polypeptide) with an alternative codon to increase G/C content; or (iv) a combination thereof.

In some embodiments, the sequence-optimized nucleotide sequence (e.g., an ORF encoding a therapeutic polypeptide) has at least one improved property with respect to the reference nucleotide sequence.

In some embodiments, the sequence optimization method is multiparametric and comprises one, two, three, four, or more methods disclosed herein and/or other optimization methods known in the art.

Features, which can be considered beneficial in some embodiments of the invention, can be encoded by or within regions of the polynucleotide and such regions can be upstream (5′) to, downstream (3′) to, or within the region that encodes the therapeutic polypeptide. These regions can be incorporated into the polynucleotide before and/or after sequence-optimization of the protein encoding region or open reading frame (ORF). Examples of such features include, but are not limited to, untranslated regions (UTRs), microRNA sequences, Kozak sequences, oligo(dT) sequences, poly-A tail, and detectable tags and can include multiple cloning sites that can have XbaI recognition.

In some embodiments, the polynucleotide of the invention comprises a 5′ UTR. a 3′ UTR and/or a miRNA. In some embodiments, the polynucleotide comprises two or more 5′ UTRs and/or 3′ UTRs, which can be the same or different sequences. In some embodiments, the polynucleotide comprises two or more miRNA, which can be the same or different sequences. Any portion of the 5′ UTR, 3′ UTR, and/or miRNA, including none, can be sequence-optimized and can independently contain one or more different structural or chemical modifications, before and/or after sequence optimization.

In some embodiments, after optimization, the polynucleotide is reconstituted and transformed into a vector such as, but not limited to, plasmids, viruses, cosmids, and artificial chromosomes. For example, the optimized polynucleotide can be reconstituted and transformed into chemically competent E. coli, yeast, neurospora, maize, drosophila, etc. where high copy plasmid-like or chromosome structures occur by methods described herein.

### Sequence-Optimized Nucleotide Sequences Encoding Therapeutic Polypeptides

In some embodiments, the polynucleotide of the invention comprises a sequence-optimized nucleotide sequence encoding a therapeutic polypeptide disclosed herein. In some embodiments, the polynucleotide of the invention comprises an open reading frame (ORF) encoding a therapeutic polypeptide, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence (e.g., encoding a therapeutic polypeptide, a functional fragment, or a variant thereof) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the invention is greater than the uracil or thymine content in the reference wild-type sequence and still maintain beneficial effects, e.g., increased expression and/or reduced Toll-Like Receptor (TLR) response when compared to the reference wild-type sequence. Methods for optimizing codon usage are known in the art.

### Modified Nucleotide Sequences Encoding Therapeutic Polypeptides

In some embodiments, the polynucleotide (e.g., a RNA, e.g., an mRNA) of the invention comprises a chemically modified nucleobase, for example, a chemically modified uracil, e.g., pseudouracil, 1-methylpseuodouracil, 5-methoxyuracil, or the like. In some embodiments, the mRNA is a uracil-modified sequence comprising an ORF encoding a Factor VIII polypeptide, wherein the mRNA comprises a chemically modified nucleobase, for example, a chemically modified uracil, e.g., pseudouracil, 1-methylpseuodouracil, or 5-methoxyuracil. The invention includes modified polynucleotides comprising a polynucleotide described herein (e.g., a polynucleotide comprising a nucleotide sequence encoding a therapeutic polypeptide). The modified polynucleotides can be chemically modified and/or structurally modified. When the polynucleotides of the present invention are chemically and/or structurally modified the polynucleotides can be referred to as “modified polynucleotides.”

Polynucleotides of the present disclosure comprise, in some embodiments, at least one nucleic acid (e.g., RNA) having an open reading frame encoding at least one therapeutic protein, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

In some embodiments, the mRNA is a uracil-modified sequence comprising an ORF encoding a therapeutic polypeptide, wherein the mRNA comprises a chemically modified nucleobase, e.g., 5-methoxyuracil. In certain aspects of the invention, when the 5-methoxyuracil base is connected to a ribose sugar, as it is in polynucleotides, the resulting modified nucleoside or nucleotide is referred to as 5-methoxyuridine. In some embodiments, uracil in the polynucleotide is at least about 25%, at least about 30%, at least about 40%, at least about 50%, at least about 60%, at least about 70%, at least about 80%, at least 90%, at least 95%, at least 99%, or about 100% modified uracil. In one embodiment, uracil in the polynucleotide is at least 95% modified uracil. In another embodiment, uracil in the polynucleotide is 100% modified uracil.

In embodiments where uracil in the polynucleotide is at least 95% 5-methoxyuracil, overall uracil content can be adjusted such that an mRNA provides suitable protein expression levels while inducing little to no immune response. In some embodiments, the uracil content of the ORF is between about 105% and about 145%, about 105% and about 140%, about 110% and about 140%, about 110% and about 145%, about 115% and about 135%, about 105% and about 135%, about 110% and about 135%, about 115% and about 145%, or about 115% and about 140% of the theoretical minimum uracil content in the corresponding wild-type ORF (% Utm). In other embodiments, the uracil content of the ORF is between about 117% and about 134% or between 118% and 132% of the % UTM. In some embodiments, the uracil content of the ORF encoding a therapeutic polypeptide is about 115%, about 120%, about 125%, about 130%, about 135%, about 140%, about 145%, or about 150% of the % Utm. In this context, the term “uracil” can refer to 5-methoxyuracil and/or naturally occurring uracil.

In some embodiments, the uracil content in the ORF of the mRNA encoding a therapeutic polypeptide of the invention is less than about 50%, about 40%, about 30%, about 20%, about 15%, or about 12% of the total nucleobase content in the ORF. In some embodiments, the uracil content in the ORF is between about 12% and about 25% of the total nucleobase content in the ORF. In other embodiments, the uracil content in the ORF is between about 15% and about 17% of the total nuclebase content in the ORF. In one embodiment, the uracil content in the ORF of the mRNA encoding a therapeutic polypeptide is less than about 20% of the total nucleobase content in the open reading frame. In this context, the term “uracil” can refer to 5-methoxyuracil and/or naturally occurring uracil.

In further embodiments, the ORF of the mRNA encoding a therapeutic polypeptide of the invention comprises 5-methoxyuracil and has an adjusted uracil content containing less uracil pairs (UU) and/or uracil triplets (UUU) and/or uracil quadruplets (UUUU) than the corresponding wild-type nucleotide sequence encoding the therapeutic polypeptide. In some embodiments, the ORF of the mRNA encoding a therapeutic polypeptide of the invention contains no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the therapeutic polypeptide. In a particular embodiment, the ORF of the mRNA encoding the therapeutic polypeptide of the invention contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the the ORF of the mRNA encoding the therapeutic polypeptide contains no non-phenylalanine uracil pairs and/or triplets.

In further embodiments, the ORF of the mRNA encoding a therapeutic polypeptide of the invention comprises modified uracil and has an adjusted uracil content containing less uracil-rich clusters than the corresponding wild-type nucleotide sequence encoding the therapeutic polypeptide. In some embodiments, the ORF of the mRNA encoding the therapeutic polypeptide of the invention contains uracil-rich clusters that are shorter in length than corresponding uracil-rich clusters in the corresponding wild-type nucleotide sequence encoding the therapeutic polypeptide.

In further embodiments, alternative lower frequency codons are employed. At least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the therapeutic polypeptide-encoding ORF of the modified uracil-comprising mRNA are substituted with alternative codons, each alternative codon having a codon frequency lower than the codon frequency of the substituted codon in the synonymous codon set. The ORF also has adjusted uracil content, as described above. In some embodiments, at least one codon in the ORF of the mRNA encoding the therapeutic polypeptide is substituted with an alternative codon having a codon frequency lower than the codon frequency of the substituted codon in the synonymous codon set.

In some embodiments, the adjusted uracil content, of the therapeutic polypeptide-encoding ORF of the modified uracil-comprising mRNA exhibits expression levels of the therapeutic protein when administered to a mammalian cell that are higher than expression levels of the therapeutic protein from the corresponding wild-type mRNA. In some embodiments, the mammalian cell is a mouse cell, a rat cell, or a rabbit cell. In other embodiments, the mammalian cell is a monkey cell or a human cell. In some embodiments, the human cell is a HeLa cell, a BJ fibroblast cell, or a peripheral blood mononuclear cell (PBMC). In some embodiments, a therapeutic protein is expressed when the mRNA is administered to a mammalian cell in vivo. In some embodiments, the mRNA is administered to mice, rabbits, rats, monkeys, or humans. In one embodiment, mice are null mice. In some embodiments, the mRNA is administered to mice in an amount of about 0.01 mg/kg, about 0.05 mg/kg, about 0.1 mg/kg, 0.2 mg/kg or about 0.5 mg/kg. In some embodiments, the mRNA is administered intravenously or intramuscularly. In other embodiments, the therapeutic polypeptide is expressed when the mRNA is administered to a mammalian cell in vitro. In some embodiments, the expression is increased by at least about 2-fold, at least about 5-fold, at least about 10-fold, at least about 50-fold, at least about 500-fold, at least about 1500-fold, or at least about 3000-fold. In other embodiments, the expression is increased by at least about 10%, about 20%, about 30%, about 40%, about 50%, 60%, about 70%, about 80%, about 90%, or about 100%.

In some embodiments, adjusted uracil content, a therapeutic polypeptide-encoding ORF of the 5-methoxyuracil-comprising mRNA exhibits increased stability. In some embodiments, the mRNA exhibits increased stability in a cell relative to the stability of a corresponding wild-type mRNA under the same conditions. In some embodiments, the mRNA exhibits increased stability including resistance to nucleases, thermal stability, and/or increased stabilization of secondary structure. In some embodiments, increased stability exhibited by the mRNA is measured by determining the half-life of the mRNA (e.g., in a plasma, cell, or tissue sample) and/or determining the area under the curve (AUC) of the protein expression by the mRNA over time (e.g., in vitro or in vivo). An mRNA is identified as having increased stability if the half-life and/or the AUC is greater than the half-life and/or the AUC of a corresponding wild-type mRNA under the same conditions.

In some embodiments, the mRNA of the present invention induces a detectably lower immune response (e.g., innate or acquired) relative to the immune response induced by a corresponding wild-type mRNA under the same conditions. In other embodiments, the mRNA of the present disclosure induces a detectably lower immune response (e.g., innate or acquired) relative to the immune response induced by an mRNA that encodes for a therapeutic polypeptide but does not comprise 5-methoxyuracil under the same conditions, or relative to the immune response induced by an mRNA that encodes for a therapeutic polypeptide and that comprises 5-methoxyuracil but that does not have adjusted uracil content under the same conditions. The innate immune response can be manifested by increased expression of pro-inflammatory cytokines, activation of intracellular PRRs (RIG-I, MDA5, etc), cell death, and/or termination or reduction in protein translation. In some embodiments, a reduction in the innate immune response can be measured by expression or activity level of Type 1 interferons (e.g., IFN-α, IFN-β, IFN-κ, IFN-δ, IFN-ε, IFN-τ, IFN-ω, and IFN-ξ) or the expression of interferon-regulated genes such as the toll-like receptors (e.g., TLR7 and TLR8), and/or by decreased cell death following one or more administrations of the mRNA of the invention into a cell.

In some embodiments, the expression of Type-1 interferons by a mammalian cell in response to the mRNA of the present disclosure is reduced by at least 10%, 20%, 30%, 40%, 50%, 60%, 70%, 80%, 90%, 95%, 99%, 99.9%, or greater than 99.9% relative to a corresponding wild-type mRNA, to an mRNA that encodes a therapeutic polypeptide but does not comprise modified uracil, or to an mRNA that encodes a therapeutic polypeptide and that comprises modified uracil but that does not have adjusted uracil content. In some embodiments, the interferon is IFN-β. In some embodiments, cell death frequency cased by administration of mRNA of the present disclosure to a mammalian cell is 10%, 25%, 50%, 75%, 85%, 90%, 95%, or over 95% less than the cell death frequency observed with a corresponding wild-type mRNA, an mRNA that encodes for a therapeutic polypeptide but does not comprise modified uracil, or an mRNA that encodes for a therapeutic polypeptide and that comprises modified uracil but that does not have adjusted uracil content. In some embodiments, the mammalian cell is a BJ fibroblast cell. In other embodiments, the mammalian cell is a splenocyte. In some embodiments, the mammalian cell is that of a mouse or a rat. In other embodiments, the mammalian cell is that of a human. In one embodiment, the mRNA of the present disclosure does not substantially induce an innate immune response of a mammalian cell into which the mRNA is introduced.

In some embodiments, the polynucleotide is an mRNA that comprises an ORF that encodes a therapeutic polypeptide, wherein uracil in the mRNA is at least about 95% modified uracil, wherein the uracil content of the ORF is between about 115% and about 135% of the theoretical minimum uracil content in the corresponding wild-type ORF, and wherein the uracil content in the ORF encoding the therapeutic polypeptide is less than about 23% of the total nucleobase content in the ORF. In some embodiments, the ORF that encodes the therapeutic polypeptide is further modified to decrease G/C content of the ORF (absolute or relative) by at least about 40%, as compared to the corresponding wild-type ORF. In yet other embodiments, the ORF encoding the therapeutic polypeptide contains less than 20 non-phenylalanine uracil pairs and/or triplets. In some embodiments, at least one codon in the ORF of the mRNA encoding the therapeutic polypeptide is further substituted with an alternative codon having a codon frequency lower than the codon frequency of the substituted codon in the synonymous codon set. In some embodiments, the expression of the therapeutic polypeptide encoded by an mRNA comprising an ORF wherein uracil in the mRNA is at least about 95% modified uracil, and wherein the uracil content of the ORF is between about 115% and about 135% of the theoretical minimum uracil content in the corresponding wild-type ORF, is increased by at least about 10-fold when compared to expression of the therapeutic polypeptide from the corresponding wild-type mRNA. In some embodiments, the mRNA comprises an open ORF wherein uracil in the mRNA is at least about 95% modified uracil, and wherein the uracil content of the ORF is between about 115% and about 135% of the theoretical minimum uracil content in the corresponding wild-type ORF, and wherein the mRNA does not substantially induce an innate immune response of a mammalian cell into which the mRNA is introduced.

In certain embodiments, the chemical modification is at nucleobases in the polynucleotides (e.g., RNA polynucleotide, such as mRNA polynucleotide). In some embodiments, modified nucleobases in the polynucleotide (e.g., RNA polynucleotide, such as mRNA polynucleotide) are selected from the group consisting of 1-methyl-pseudouridine (m1ψ), 1-ethyl-pseudouridine (e1ψ), 5-methoxy-uridine (mo5U), 5-methyl-cytidine (m5C), pseudouridine (ψ), α-thio-guanosine and α-thio-adenosine. In some embodiments, the polynucleotide includes a combination of at least two (e.g., 2, 3, 4 or more) of the aforementioned modified nucleobases.

In some embodiments, the polynucleotide (e.g., RNA polynucleotide, such as mRNA polynucleotide) comprises pseudouridine (ψ) and 5-methyl-cytidine (m5C). In some embodiments, the polynucleotide (e.g., RNA polynucleotide, such as mRNA polynucleotide) comprises 1-methyl-pseudouridine (m1ψ). In some embodiments, the polynucleotide (e.g., RNA polynucleotide, such as mRNA polynucleotide) comprises 1-ethyl-pseudouridine (e1ψ). In some embodiments, the polynucleotide (e.g., RNA polynucleotide, such as mRNA polynucleotide) comprises 1-methyl-pseudouridine (m1ψ) and 5-methyl-cytidine (m5C). In some embodiments, the polynucleotide (e.g., RNA polynucleotide, such as mRNA polynucleotide) comprises 1-ethyl-pseudouridine (e1ψ) and 5-methyl-cytidine (m5C). In some embodiments, the polynucleotide (e.g., RNA polynucleotide, such as mRNA polynucleotide) comprises 2-thiouridine (s2U). In some embodiments, the polynucleotide (e.g., RNA polynucleotide, such as mRNA polynucleotide) comprises 2-thiouridine and 5-methyl-cytidine (m5C). In some embodiments, the polynucleotide (e.g., RNA polynucleotide, such as mRNA polynucleotide) comprises methoxy-uridine (mo5U). In some embodiments, the polynucleotide (e.g., RNA polynucleotide, such as mRNA polynucleotide) comprises 5-methoxy-uridine (mo5U) and 5-methyl-cytidine (m5C). In some embodiments, the polynucleotide (e.g., RNA polynucleotide, such as mRNA polynucleotide) comprises 2′-O-methyl uridine. In some embodiments, the polynucleotide (e.g., RNA polynucleotide, such as mRNA polynucleotide) comprises 2′-O-methyl uridine and 5-methyl-cytidine (m5C). In some embodiments, the polynucleotide (e.g., RNA polynucleotide, such as mRNA polynucleotide) comprises N6-methyl-adenosine (m6A). In some embodiments, the polynucleotide (e.g., RNA polynucleotide, such as mRNA polynucleotide) comprises N6-methyl-adenosine (m6A) and 5-methyl-cytidine (m5C).

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some embodiments, the chemically modified nucleosides in the open reading frame are selected from the group consisting of uridine, adenine, cytosine, guanine, and any combination thereof.

In some embodiments, the modified nucleobase is a modified cytosine. Examples of nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine.

In some embodiments, a modified nucleobase is a modified uridine. Example nucleobases and nucleosides having a modified uridine include 5-cyano uridine or 4′-thio uridine.

In some embodiments, a modified nucleobase is a modified adenine. Example nucleobases and nucleosides having a modified adenine include 7-deaza-adenine, 1-methyl-adenosine (m1A), 2-methyl-adenine (m2A), N6-methyl-adenine (m6A), and 2,6-Diaminopurine.

In some embodiments, a modified nucleobase is a modified guanine. Example nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some embodiments, the nucleobase modified nucleotides in the polynucleotide (e.g., RNA polynucleotide, such as mRNA polynucleotide) are 5-methoxyuridine.

In some embodiments, the polynucleotide (e.g., RNA polynucleotide, such as mRNA polynucleotide) includes a combination of at least two (e.g., 2, 3, 4 or more) of modified nucleobases.

In some embodiments, the polynucleotide (e.g., RNA polynucleotide, such as mRNA polynucleotide) comprises 5-methoxyuridine (5mo5U) and 5-methyl-cytidine (m5C).

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 5-methoxyuridine, meaning that substantially all uridine residues in the mRNA sequence are replaced with 5-methoxyuridine. Similarly, a polynucleotide can be uniformly modified for any type of nucleoside residue present in the sequence by replacement with a modified residue such as any of those set forth above.

In some embodiments, the modified nucleobase is a modified cytosine.

In some embodiments, a modified nucleobase is a modified uracil. Example nucleobases and nucleosides having a modified uracil include 5-methoxyuracil.

In some embodiments, a modified nucleobase is a modified adenine.

In some embodiments, a modified nucleobase is a modified guanine.

In some embodiments, the polynucleotides can include any useful linker between the nucleosides. Such linkers, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

The modified nucleosides and nucleotides (e.g., building block molecules), which can be incorporated into a polynucleotide (e.g., RNA or mRNA, as described herein), can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are taught International Patent Publication Nos. WO2013052523 and WO2014093924, the contents of each of which are incorporated herein by reference in their entireties.

The polynucleotides of the invention (e.g., a polynucleotide comprising a nucleotide sequence encoding a therapeutic polypeptide or a functional fragment or variant thereof) can include a combination of modifications to the sugar, the nucleobase, and/or the internucleoside linkage. These combinations can include any one or more modifications described herein.

### Untranslated Regions (UTRs)

Untranslated regions (UTRs) are nucleic acid sections of a polynucleotide before a start codon (5′UTR) and after a stop codon (3′UTR) that are not translated. In some embodiments, a polynucleotide (e.g., a ribonucleic acid (RNA), e.g., a messenger RNA (mRNA)) of the invention comprising an open reading frame (ORF) encoding a therapeutic polypeptide further comprises UTR (e.g., a 5′UTR or functional fragment thereof, a 3′UTR or functional fragment thereof, or a combination thereof).

A UTR can be homologous or heterologous to the coding region in a polynucleotide. In some embodiments, the UTR is homologous to the ORF encoding the therapeutic polypeptide. In some embodiments, the UTR is heterologous to the ORF encoding the therapeutic polypeptide. In some embodiments, the polynucleotide comprises two or more 5′UTRs or functional fragments thereof, each of which have the same or different nucleotide sequences. In some embodiments, the polynucleotide comprises two or more 3′UTRs or functional fragments thereof, each of which have the same or different nucleotide sequences.

In some embodiments, the 5′UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′UTR or 3′UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTRs also have been known to form secondary structures that are involved in elongation factor binding.

By engineering the features typically found in abundantly expressed genes of specific target organs, one can enhance the stability and protein production of a polynucleotide. For example, introduction of 5′UTR of liver-expressed mRNA, such as albumin, serum amyloid A, Apolipoprotein AB/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′UTR and the 3′UTR can be heterologous. In some embodiments, the 5′UTR can be derived from a different species than the 3′UTR. In some embodiments, the 3′UTR can be derived from a different species than the 5′UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or (3-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

Other exemplary 5′ and 3′ UTRs include, but are not limited to, those described in Karikó et al., Mol. Ther. 2008 16(11):1833-1840; Karikó et al., Mol. Ther. 2012 20(5):948-953; Karikó et al., Nucleic Acids Res. 2011 39(21):e142; Strong et al., Gene Therapy 1997 4:624-627; Hansson et al., J. Biol. Chem. 2015 290(9):5661-5672; Yu et al., Vaccine 2007 25(10):1701-1711; Cafri et al., Mol. Ther. 2015 23(8):1391-1400; Andries et al., Mol. Pharm. 2012 9(8):2136-2145; Crowley et al., Gene Ther. 2015 Jun. 30, doi:10.1038/gt.2015.68; Ramunas et al., FASEB J. 2015 29(5):1930-1939; Wang et al., Curr. Gene Ther. 2015 15(4):428-435; Holtkamp et al., Blood 2006 108(13):4009-4017; Kormann et al., Nat. Biotechnol. 2011 29(2):154-157; Poleganov et al., Hum. Gen. Ther. 2015 26(11):751-766; Warren et al., Cell Stem Cell 2010 7(5):618-630; Mandal and Rossi, Nat. Protoc. 2013 8(3):568-582; Holcik and Liebhaber, PNAS 1997 94(6):2410-2414; Ferizi et al., Lab Chip. 2015 15(17):3561-3571; Thess et al., Mol. Ther. 2015 23(9):1456-1464; Boros et al., PLoS One 2015 10(6):e0131141; Boros et al., J. Photochem. Photobiol. B. 2013 129:93-99; Andries et al., J. Control. Release 2015 217:337-344; Zinckgraf et al., Vaccine 2003 21(15):1640-9; Garneau et al., J. Virol. 2008 82(2):880-892; Holden and Harris, Virology 2004 329(1):119-133; Chiu et al., J. Virol. 2005 79(13):8303-8315; Wang et al., EMBO J. 1997 16(13):4107-4116; Al-Zoghaibi et al., Gene 2007 391(1-2):130-9; Vivinus et al., Eur. J. Biochem. 2001 268(7):1908-1917; Gan and Rhoads, J. Biol. Chem. 1996 271(2):623-626; Boado et al., J. Neurochem. 1996 67(4):1335-1343; Knirsch and Clerch, Biochem. Biophys. Res. Commun. 2000 272(1):164-168; Chung et al., Biochemistry 1998 37(46):16298-16306; Izquierdo and Cuevza, Biochem. J. 2000 346 Pt 3:849-855; Dwyer et al., J. Neurochem. 1996 66(2):449-458; Black et al., Mol. Cell. Biol. 1997 17(5):2756-2763; Izquierdo and Cuevza, Mol. Cell. Biol. 1997 17(9):5255-5268; U.S. Pat. Nos. 8,278,036; 8,748,089; 8,835,108; 9,012,219; US2010/0129877; US2011/0065103; US2011/0086904; US2012/0195936; US2014/020675; US2013/0195967; US2014/029490; US2014/0206753; WO2007/036366; WO2011/015347; WO2012/072096; WO2013/143555; WO2014/071963; WO2013/185067; WO2013/182623; WO2014/089486; WO2013/185069; WO2014/144196; WO2014/152659; 2014/152673; WO2014/152940; WO2014/152774; WO2014/153052; WO2014/152966, WO2014/152513; WO2015/101414; WO2015/101415; WO2015/062738; and WO2015/024667; the contents of each of which are incorporated herein by reference in their entirety.

In some embodiments, the 5′UTR is selected from the group consisting of a β-globin 5′UTR; a 5′UTR containing a strong Kozak translational initiation signal; a cytochrome b-245 α polypeptide (CYBA) 5′UTR; a hydroxysteroid (17-β) dehydrogenase (HSD17B4) 5′UTR; a Tobacco etch virus (TEV) 5′UTR; a Venezuelen equine encephalitis virus (TEEV) 5′UTR; a 5′ proximal open reading frame of rubella virus (RV) RNA encoding nonstructural proteins; a Dengue virus (DEN) 5′UTR; a heat shock protein 70 (Hsp70) 5′UTR; a eIF4G 5′UTR; a GLUT1 5′UTR; functional fragments thereof and any combination thereof.

In some embodiments, the 3′UTR is selected from the group consisting of a β-globin 3′UTR; a CYBA 3′UTR; an albumin 3′UTR; a growth hormone (GH) 3′UTR; a VEEV 3′UTR; a hepatitis B virus (HBV) 3′UTR; α-globin 3′UTR; a DEN 3′UTR; a PAV barley yellow dwarf virus (BYDV-PAV) 3′UTR; an elongation factor 1 α1 (EEF1A1) 3′UTR; a manganese superoxide dismutase (MnSOD) 3′UTR; a β subunit of mitochondrial H(+)-ATP synthase (β-mRNA) 3′UTR; a GLUT1 3′UTR; a MEF2A 3′UTR; a β-F1-ATPase 3′UTR; functional fragments thereof and combinations thereof.

Other exemplary UTRs include, but are not limited to, one or more of the UTRs, including any combination of UTRs, disclosed in WO2014/164253, the contents of which are incorporated herein by reference in their entirety. Shown in Table 21 of U.S. Provisional Application No. 61/775,509 and in Table 22 of U.S. Provisional Application No. 61/829,372, the contents of each are incorporated herein by reference in their entirety, is a listing start and stop sites for 5′UTRs and 3′UTRs. Below, each 5′UTR (5′-UTR-005 to 5′-UTR 68511) is identified by its start and stop site relative to its native or wild-type (homologous) transcript (ENST; the identifier used in the ENSEMBL database).

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, and sequences available at www.addgene.org/Derrick_Rossi/, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′UTR or 3′UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the invention comprise a 5′UTR and/or a 3′UTR selected from any of the UTRs disclosed herein. In some embodiments, the 5′UTR comprises:

5′UTR-001 (Upstream UTR)
(SEQ ID NO. 1)
(GGGAAATAAGAGAGAAAAGAAGAGTAAGAAGAAATATAAGAGCCACC);
5′UTR-002 (Upstream UTR)
(SEQ ID NO. 2)
(GGGAGATCAGAGAGAAAAGAAGAGTAAGAAGAAATATAAGAGCCACC);
5′UTR-003 (Upstream UTR)
(SEQ ID NO. 3)
(GGAATAAAAGTCTCAACACAACATATACAAAACAAACGAATCTCAAGCA
ATCAAGCATTCTACTTCTATTGCAGCAATTTAAATCATTTCTTTTAAAGC
AAAAGCAATTTTCTGAAAATTTTCACCATTTACGAACGATAGCAAC);
5′UTR-004 (Upstream UTR)
(SEQ ID NO. 4)
(GGGAGACAAGCUUGGCAUUCCGGUACUGUUGGUAAAGCCACC);
5′UTR-005 (Upstream UTR)
(SEQ ID NO. 5)
(GGGAGATCAGAGAGAAAAGAAGAGTAAGAAGAAATATAAGAGCCACC);
UTR 5′UTR-006 (Upstream UTR)
(SEQ ID NO. 6)
(GGAATAAAAGTCTCAACACAACATATACAAAACAAACGAATCTCAAGCA
ATCAAGCATTCTACTTCTATTGCAGCAATTTAAATCATTTCTTTTAAAGC
AAAAGCAATTTTCTGAAAATTTTCACCATTTACGAACGATAGCAAC);
5′UTR-007 (Upstream UTR)
(SEQ ID NO. 7)
(GGGAGACAAGCUUGGCAUUCCGGUACUGUUGGUAAAGCCACC);
5′UTR-008 (Upstream UTR)
(SEQ ID NO. 8)
(GGGAATTAACAGAGAAAAGAAGAGTAAGAAGAAATATAAGAGCCACC);
5′UTR-009 (Upstream UTR)
(SEQ ID NO. 9)
(GGGAAATTAGACAGAAAAGAAGAGTAAGAAGAAATATAAGAGCCACC);
UTR 5′UTR-010, Upstream
(SEQ ID NO. 10)
(GGGAAATAAGAGAGTAAAGAACAGTAAGAAGAAATATAAGAGCCACC);
5′UTR-011 (Upstream UTR)
(SEQ ID NO. 11)
(GGGAAAAAAGAGAGAAAAGAAGACTAAGAAGAAATATAAGAGCCACC);
5′UTR-012 (Upstream UTR)
(SEQ ID NO. 12)
(GGGAAATAAGAGAGAAAAGAAGAGTAAGAAGATATATAAGAGCCACC);
5′UTR-013 (Upstream UTR)
(SEQ ID NO. 13)
(GGGAAATAAGAGACAAAACAAGAGTAAGAAGAAATATAAGAGCCACC);
5′UTR-014 (Upstream UTR)
(SEQ ID NO. 14)
(GGGAAATTAGAGAGTAAAGAACAGTAAGTAGAATTAAAAGAGCCACC);
5′UTR-15 (Upstream UTR)
(SEQ ID NO. 15)
(GGGAAATAAGAGAGAATAGAAGAGTAAGAAGAAATATAAGAGCCACC);
5′UTR-016 (Upstream UTR)
(SEQ ID NO. 16)
(GGGAAATAAGAGAGAAAAGAAGAGTAAGAAGAAAATTAAGAGCCACC);
5′UTR-017 (Upstream UTR)
(SEQ ID NO. 17)
(GGGAAATAAGAGAGAAAAGAAGAGTAAGAAGAAATTTAAGAGCCACC);
5′UTR-018 (Upstream UTR)
(SEQ ID NO. 18)
(TCAAGCTTTTGGACCCTCGTACAGAAGCTAATACGACTCACTATAGGGA
AATAAGAGAGAAAAGAAGAGTAAGAAGAAATATAAGAGCCACC);
142-3p 5′UTR-001 (Upstream UTR including
miR142-3p)
(SEQ ID NO. 19)
(TGATAATAGTCCATAAAGTAGGAAACACTACAGCTGGAGCCTCGGTGGC
CATGCTTCTTGCCCCTTGGGCCTCCCCCCAGCCCCTCCTCCCCTTCCTGC
ACCCGTACCCCCGTGGTCTTTGAATAAAGTCTGAGTGGGCGGC);
142-3p 5′UTR-002 (Upstream UTR including
miR142-3p)
(SEQ ID NO. 20)
(TGATAATAGGCTGGAGCCTCGGTGGCTCCATAAAGTAGGAAACACTACA
CATGCTTCTTGCCCCTTGGGCCTCCCCCCAGCCCCTCCTCCCCTTCCTGC
ACCCGTACCCCCGTGGTCTTTGAATAAAGTCTGAGTGGGCGGC);
142-3p 5′UTR-003 (Upstream UTR including
miR142-3p)
(SEQ ID NO. 21)
(TGATAATAGGCTGGAGCCTCGGTGGCCATGCTTCTTGCCCCTTCCATAA
AGTAGGAAACACTACATGGGCCTCCCCCCAGCCCCTCCTCCCCTTCCTGC
ACCCGTACCCCCGTGGTCTTTGAATAAAGTCTGAGTGGGCGGC);
142-3p 5′UTR-004 (Upstream UTR including
miR142-3p)
(SEQ ID NO. 22)
(TGATAATAGGCTGGAGCCTCGGTGGCCATGCTTCTTGCCCCTTGGGCCT
CCCCCCAGTCCATAAAGTAGGAAACACTACACCCCTCCTCCCCTTCCTGC
ACCCGTACCCCCGTGGTCTTTGAATAAAGTCTGAGTGGGCGGC);
142-3p 5′UTR-005 (Upstream UTR including
miR142-3p)
(SEQ ID NO. 23)
(TGATAATAGGCTGGAGCCTCGGTGGCCATGCTTCTTGCCCCTTGGGCCT
CCCCCCAGCCCCTCCTCCCCTTCTCCATAAAGTAGGAAACACTACACTGC
ACCCGTACCCCCGTGGTCTTTGAATAAAGTCTGAGTGGGCGGC);
142-3p 5′UTR-006 (Upstream UTR including
miR142-3p)
(SEQ ID NO. 24)
(TGATAATAGGCTGGAGCCTCGGTGGCCATGCTTCTTGCCCCTTGGGCCT
CCCCCCAGCCCCTCCTCCCCTTCCTGCACCCGTACCCCCTCCATAAAGTA
GGAAACACTACAGTGGTCTTTGAATAAAGTCTGAGTGGGCGGC);
or
142-3p 5′UTR-007 (Upstream UTR including
miR142-3p)
(SEQ ID NO. 25)
(TGATAATAGGCTGGAGCCTCGGTGGCCATGCTTCTTGCCCCTTGGGCCT
CCCCCCAGCCCCTCCTCCCCTTCCTGCACCCGTACCCCCGTGGTCTTTGA
ATAAAGTTCCATAAAGTAGGAAACACTACACTGAGTGGGCGGC).
In some embodiments, the 3′UTR comprises:
3′UTR-001 (Creatine Kinase UTR)
(SEQ ID NO. 26)
(GCGCCTGCCCACCTGCCACCGACTGCTGGAACCCAGCCAGTGGGAGGGC
CTGGCCCACCAGAGTCCTGCTCCCTCACTCCTCGCCCCGCCCCCTGTCCC
AGAGTCCCACCTGGGGGCTCTCTCCACCCTTCTCAGAGTTCCAGTTTCAA
CCAGAGTTCCAACCAATGGGCTCCATCCTCTGGATTCTGGCCAATGAAAT
ATCTCCCTGGCAGGGTCCTCTTCTTTTCCCAGAGCTCCACCCCAACCAGG
AGCTCTAGTTAATGGAGAGCTCCCAGCACACTCGGAGCTTGTGCTTTGTC
TCCACGCAAAGCGATAAATAAAAGCATTGGTGGCCTTTGGTCTTTGAATA
AAGCCTGAGTAGGAAGTCTAGA);
3′UTR-002 (Myoglobin UTR)
(SEQ ID NO. 27)
(GCCCCTGCCGCTCCCACCCCCACCCATCTGGGCCCCGGGTTCAAGAGAG
AGCGGGGTCTGATCTCGTGTAGCCATATAGAGTTTGCTTCTGAGTGTCTG
CTTTGTTTAGTAGAGGTGGGCAGGAGGAGCTGAGGGGCTGGGGCTGGGGT
GTTGAAGTTGGCTTTGCATGCCCAGCGATGCGCCTCCCTGTGGGATGTCA
TCACCCTGGGAACCGGGAGTGGCCCTTGGCTCACTGTGTTCTGCATGGTT
TGGATCTGAATTAATTGTCCTTTCTTCTAAATCCCAACCGAACTTCTTCC
AACCTCCAAACTGGCTGTAACCCCAAATCCAAGCCATTAACTACACCTGA
CAGTAGCAATTGTCTGATTAATCACTGGCCCCTTGAAGACAGCAGAATGT
CCCTTTGCAATGAGGAGGAGATCTGGGCTGGGCGGGCCAGCTGGGGAAGC
ATTTGACTATCTGGAACTTGTGTGTGCCTCCTCAGGTATGGCAGTGACTC
ACCTGGTTTTAATAAAACAACCTGCAACATCTCATGGTCTTTGAATAAAG
CCTGAGTAGGAAGTCTAGA);
3′UTR-003 (α-actinUTR)
(SEQ ID NO. 28)
(ACACACTCCACCTCCAGCACGCGACTTCTCAGGACGACGAATCTTCTCA
ATGGGGGGGCGGCTGAGCTCCAGCCACCCCGCAGTCACTTTCTTTGTAAC
AACTTCCGTTGCTGCCATCGTAAACTGACACAGTGTTTATAACGTGTACA
TACATTAACTTATTACCTCATTTTGTTATTTTTCGAAACAAAGCCCTGTG
GAAGAAAATGGAAAACTTGAAGAAGCATTAAAGTCATTCTGTTAAGCTGC
GTAAATGGTCTTTGAATAAAGCCTGAGTAGGAAGTCTAGA);
3′UTR-004 (Albumin UTR)
(SEQ ID NO. 29)
(CATCACATTTAAAAGCATCTCAGCCTACCATGAGAATAAGAGAAAGAAA
ATGAAGATCAAAAGCTTATTCATCTGTTTTTCTTTTTCGTTGGTGTAAAG
CCAACACCCTGTCTAAAAAACATAAATTTCTTTAATCATTTTGCCTCTTT
TCTCTGTGCTTCAATTAATAAAAAATGGAAAGAATCTAATAGAGTGGTAC
AGCACTGTTATTTTTCAAAGATGTGTTGCTATCCTGAAAATTCTGTAGGT
TCTGTGGAAGTTCCAGTGTTCTCTCTTATTCCACTTCGGTAGAGGATTTC
TAGTTTCTTGTGGGCTAATTAAATAAATCATTAATACTCTTCTAATGGTC
TTTGAATAAAGCCTGAGTAGGAAGTCTAGA);
3′UTR-005 (α-globin UTR)
(SEQ ID NO. 30)
(GCTGCCTTCTGCGGGGCTTGCCTTCTGGCCATGCCCTTCTTCTCTCCCT
TGCACCTGTACCTCTTGGTCTTTGAATAAAGCCTGAGTAGGAAGGCGGCC
GCTCGAGCATGCATCTAGA);
3′UTR-006 (G-CSF UTR)
(SEQ ID NO. 31)
(GCCAAGCCCTCCCCATCCCATGTATTTATCTCTATTTAATATTTATGTC
TATTTAAGCCTCATATTTAAAGACAGGGAAGAGCAGAACGGAGCCCCAGG
CCTCTGTGTCCTTCCCTGCATTTCTGAGTTTCATTCTCCTGCCTGTAGCA
GTGAGAAAAAGCTCCTGTCCTCCCATCCCCTGGACTGGGAGGTAGATAGG
TAAATACCAAGTATTTATTACTATGACTGCTCCCCAGCCCTGGCTCTGCA
ATGGGCACTGGGATGAGCCGCTGTGAGCCCCTGGTCCTGAGGGTCCCCAC
CTGGGACCCTTGAGAGTATCAGGTCTCCCACGTGGGAGACAAGAAATCCC
TGTTTAATATTTAAACAGCAGTGTTCCCCATCTGGGTCCTTGCACCCCTC
ACTCTGGCCTCAGCCGACTGCACAGCGGCCCCTGCATCCCCTTGGCTGTG
AGGCCCCTGGACAAGCAGAGGTGGCCAGAGCTGGGAGGCATGGCCCTGGG
GTCCCACGAATTTGCTGGGGAATCTCGTTTTTCTTCTTAAGACTTTTGGG
ACATGGTTTGACTCCCGAACATCACCGACGCGTCTCCTGTTTTTCTGGGT
GGCCTCGGGACACCTGCCCTGCCCCCACGAGGGTCAGGACTGTGACTCTT
TTTAGGGCCAGGCAGGTGCCTGGACATTTGCCTTGCTGGACGGGGACTGG
GGATGTGGGAGGGAGCAGACAGGAGGAATCATGTCAGGCCTGTGTGTGAA
AGGAAGCTCCACTGTCACCCTCCACCTCTTCACCCCCCACTCACCAGTGT
CCCCTCCACTGTCACATTGTAACTGAACTTCAGGATAATAAAGTGTTTGC
CTCCATGGTCTTTGAATAAAGCCTGAGTAGGAAGGCGGCCGCTCGAGCAT
GCATCTAGA);
3′UTR-007(Col1a2; collagen, type I,
alpha 2 UTR)
(SEQ ID NO. 32)
(ACTCAATCTAAATTAAAAAAGAAAGAAATTTGAAAAAACTTTCTCTTTG
CCATTTCTTCTTCTTCTTTTTTAACTGAAAGCTGAATCCTTCCATTTCTT
CTGCACATCTACTTGCTTAAATTGTGGGCAAAAGAGAAAAAGAAGGATTG
ATCAGAGCATTGTGCAATACAGTTTCATTAACTCCTTCCCCCGCTCCCCC
AAAAATTTGAATTTTTTTTTCAACACTCTTACACCTGTTATGGAAAATGT
CAACCTTTGTAAGAAAACCAAAATAAAAATTGAAAAATAAAAACCATAAA
CATTTGCACCACTTGTGGCTTTTGAATATCTTCCACAGAGGGAAGTTTAA
AACCCAAACTTCCAAAGGTTTAAACTACCTCAAAACACTTTCCCATGAGT
GTGATCCACATTGTTAGGTGCTGACCTAGACAGAGATGAACTGAGGTCCT
TGTTTTGTTTTGTTCATAATACAAAGGTGCTAATTAATAGTATTTCAGAT
ACTTGAAGAATGTTGATGGTGCTAGAAGAATTTGAGAAGAAATACTCCTG
TATTGAGTTGTATCGTGTGGTGTATTTTTTAAAAAATTTGATTTAGCATT
CATATTTTCCATCTTATTCCCAATTAAAAGTATGCAGATTATTTGCCCAA
ATCTTCTTCAGATTCAGCATTTGTTCTTTGCCAGTCTCATTTTCATCTTC
TTCCATGGTTCCACAGAAGCTTTGTTTCTTGGGCAAGCAGAAAAATTAAA
TTGTACCTATTTTGTATATGTGAGATGTTTAAATAAATTGTGAAAAAAAT
GAAATAAAGCATGTTTGGTTTTCCAAAAGAACATAT);
3′UTR-008 (Col6a2; collagen, type VI,
alpha 2 UTR)
(SEQ ID NO. 33)
(CGCCGCCGCCCGGGCCCCGCAGTCGAGGGTCGTGAGCCCACCCCGTCCA
TGGTGCTAAGCGGGCCCGGGTCCCACACGGCCAGCACCGCTGCTCACTCG
GACGACGCCCTGGGCCTGCACCTCTCCAGCTCCTCCCACGGGGTCCCCGT
AGCCCCGGCCCCCGCCCAGCCCCAGGTCTCCCCAGGCCCTCCGCAGGCTG
CCCGGCCTCCCTCCCCCTGCAGCCATCCCAAGGCTCCTGACCTACCTGGC
CCCTGAGCTCTGGAGCAAGCCCTGACCCAATAAAGGCTTTGAACCCAT);
3′UTR-009 (RPN1; ribophorin I UTR)
(SEQ ID NO. 34)
(GGGGCTAGAGCCCTCTCCGCACAGCGTGGAGACGGGGCAAGGAGGGGGG
TTATTAGGATTGGTGGTTTTGTTTTGCTTTGTTTAAAGCCGTGGGAAAAT
GGCACAACTTTACCTCTGTGGGAGATGCAACACTGAGAGCCAAGGGGTGG
GAGTTGGGATAATTTTTATATAAAAGAAGTTTTTCCACTTTGAATTGCTA
AAAGTGGCATTTTTCCTATGTGCAGTCACTCCTCTCATTTCTAAAATAGG
GACGTGGCCAGGCACGGTGGCTCATGCCTGTAATCCCAGCACTTTGGGAG
GCCGAGGCAGGCGGCTCACGAGGTCAGGAGATCGAGACTATCCTGGCTAA
CACGGTAAAACCCTGTCTCTACTAAAAGTACAAAAAATTAGCTGGGCGTG
GTGGTGGGCACCTGTAGTCCCAGCTACTCGGGAGGCTGAGGCAGGAGAAA
GGCATGAATCCAAGAGGCAGAGCTTGCAGTGAGCTGAGATCACGCCATTG
CACTCCAGCCTGGGCAACAGTGTTAAGACTCTGTCTCAAATATAAATAAA
TAAATAAATAAATAAATAAATAAATAAAAATAAAGCGAGATGTTGCCCTC
AAA);
3′UTR-010 (LRP1; low density lipoprotein
receptor-related protein 1 UTR)
(SEQ ID NO. 35)
(GGCCCTGCCCCGTCGGACTGCCCCCAGAAAGCCTCCTGCCCCCTGCCAG
TGAAGTCCTTCAGTGAGCCCCTCCCCAGCCAGCCCTTCCCTGGCCCCGCC
GGATGTATAAATGTAAAAATGAAGGAATTACATTTTATATGTGAGCGAGC
AAGCCGGCAAGCGAGCACAGTATTATTTCTCCATCCCCTCCCTGCCTGCT
CCTTGGCACCCCCATGCTGCCTTCAGGGAGACAGGCAGGGAGGGCTTGGG
GCTGCACCTCCTACCCTCCCACCAGAACGCACCCCACTGGGAGAGCTGGT
GGTGCAGCCTTCCCCTCCCTGTATAAGACACTTTGCCAAGGCTCTCCCCT
CTCGCCCCATCCCTGCTTGCCCGCTCCCACAGCTTCCTGAGGGCTAATTC
TGGGAAGGGAGAGTTCTTTGCTGCCCCTGTCTGGAAGACGTGGCTCTGGG
TGAGGTAGGCGGGAAAGGATGGAGTGTTTTAGTTCTTGGGGGAGGCCACC
CCAAACCCCAGCCCCAACTCCAGGGGCACCTATGAGATGGCCATGCTCAA
CCCCCCTCCCAGACAGGCCCTCCCTGTCTCCAGGGCCCCCACCGAGGTTC
CCAGGGCTGGAGACTTCCTCTGGTAAACATTCCTCCAGCCTCCCCTCCCC
TGGGGACGCCAAGGAGGTGGGCCACACCCAGGAAGGGAAAGCGGGCAGCC
CCGTTTTGGGGACGTGAACGTTTTAATAATTTTTGCTGAATTCCTTTACA
ACTAAATAACACAGATATTGTTATAAATAAAATTGT);
3′UTR-011 (Nnt1; cardiotrophin-like
cytokine factor 1 UTR)
(SEQ ID NO. 36)
(ATATTAAGGATCAAGCTGTTAGCTAATAATGCCACCTCTGCAGTTTTGG
GAACAGGCAAATAAAGTATCAGTATACATGGTGATGTACATCTGTAGCAA
AGCTCTTGGAGAAAATGAAGACTGAAGAAAGCAAAGCAAAAACTGTATAG
AGAGATTTTTCAAAAGCAGTAATCCCTCAATTTTAAAAAAGGATTGAAAA
TTCTAAATGTCTTTCTGTGCATATTTTTTGTGTTAGGAATCAAAAGTATT
TTATAAAAGGAGAAAGAACAGCCTCATTTTAGATGTAGTCCTGTTGGATT
TTTTATGCCTCCTCAGTAACCAGAAATGTTTTAAAAAACTAAGTGTTTAG
GATTTCAAGACAACATTATACATGGCTCTGAAATATCTGACACAATGTAA
ACATTGCAGGCACCTGCATTTTATGTTTTTTTTTTCAACAAATGTGACTA
ATTTGAAACTTTTATGAACTTCTGAGCTGTCCCCTTGCAATTCAACCGCA
GTTTGAATTAATCATATCAAATCAGTTTTAATTTTTTAAATTGTACTTCA
GAGTCTATATTTCAAGGGCACATTTTCTCACTACTATTTTAATACATTAA
AGGACTAAATAATCTTTCAGAGATGCTGGAAACAAATCATTTGCTTTATA
TGTTTCATTAGAATACCAATGAAACATACAACTTGAAAATTAGTAATAGT
ATTTTTGAAGATCCCATTTCTAATTGGAGATCTCTTTAATTTCGATCAAC
TTATAATGTGTAGTACTATATTAAGTGCACTTGAGTGGAATTCAACATTT
GACTAATAAAATGAGTTCATCATGTTGGCAAGTGATGTGGCAATTATCTC
TGGTGACAAAAGAGTAAAATCAAATATTTCTGCCTGTTACAAATATCAAG
GAAGACCTGCTACTATGAAATAGATGACATTAATCTGTCTTCACTGTTTA
TAATACGGATGGATTTTTTTTCAAATCAGTGTGTGTTTTGAGGTCTTATG
TAATTGATGACATTTGAGAGAAATGGTGGCTTTTTTTAGCTACCTCTTTG
TTCATTTAAGCACCAGTAAAGATCATGTCTTTTTATAGAAGTGTAGATTT
TCTTTGTGACTTTGCTATCGTGCCTAAAGCTCTAAATATAGGTGAATGTG
TGATGAATACTCAGATTATTTGTCTCTCTATATAATTAGTTTGGTACTAA
GTTTCTCAAAAAATTATTAACACATGAAAGACAATCTCTAAACCAGAAAA
AGAAGTAGTACAAATTTTGTTACTGTAATGCTCGCGTTTAGTGAGTTTAA
AACACACAGTATCTTTTGGTTTTATAATCAGTTTCTATTTTGCTGTGCCT
GAGATTAAGATCTGTGTATGTGTGTGTGTGTGTGTGTGCGTTTGTGTGTT
AAAGCAGAAAAGACTTTTTTAAAAGTTTTAAGTGATAAATGCAATTTGTT
AATTGATCTTAGATCACTAGTAAACTCAGGGCTGAATTATACCATGTATA
TTCTATTAGAAGAAAGTAAACACCATCTTTATTCCTGCCCTTTTTCTTCT
CTCAAAGTAGTTGTAGTTATATCTAGAAAGAAGCAATTTTGATTTCTTGA
AAAGGTAGTTCCTGCACTCAGTTTAAACTAAAAATAATCATACTTGGATT
TTATTTATTTTTGTCATAGTAAAAATTTTAATTTATATATATTTTTATTT
AGTATTATCTTATTCTTTGCTATTTGCCAATCCTTTGTCATCAATTGTGT
TAAATGAATTGAAAATTCATGCCCTGTTCATTTTATTTTACTTTATTGGT
TAGGATATTTAAAGGATTTTTGTATATATAATTTCTTAAATTAATATTCC
AAAAGGTTAGTGGACTTAGATTATAAATTATGGCAAAAATCTAAAAACAA
CAAAAATGATTTTTATACATTCTATTTCATTATTCCTCTTTTTCCAATAA
GTCATACAATTGGTAGATATGACTTATTTTATTTTTGTATTATTCACTAT
ATCTTTATGATATTTAAGTATAAATAATTAAAAAAATTTATTGTACCTTA
TAGTCTGTCACCAAAAAAAAAAAATTATCTGTAGGTAGTGAAATGCTAAT
GTTGATTTGTCTTTAAGGGCTTGTTAACTATCCTTTATTTTCTCATTTGT
CTTAAATTAGGAGTTTGTGTTTAAATTACTCATCTAAGCAAAAAATGTAT
ATAAATCCCATTACTGGGTATATACCCAAAGGATTATAAATCATGCTGCT
ATAAAGACACATGCACACGTATGTTTATTGCAGCACTATTCACAATAGCA
AAGACTTGGAACCAACCCAAATGTCCATCAATGATAGACTTGATTAAGAA
AATGTGCACATATACACCATGGAATACTATGCAGCCATAAAAAAGGATGA
GTTCATGTCCTTTGTAGGGACATGGATAAAGCTGGAAACCATCATTCTGA
GCAAACTATTGCAAGGACAGAAAACCAAACACTGCATGTTCTCACTCATA
GGTGGGAATTGAACAATGAGAACACTTGGACACAAGGTGGGGAACACCAC
ACACCAGGGCCTGTCATGGGGTGGGGGGAGTGGGGAGGGATAGCATTAGG
AGATATACCTAATGTAAATGATGAGTTAATGGGTGCAGCACACCAACATG
GCACATGTATACATATGTAGCAAACCTGCACGTTGTGCACATGTACCCTA
GAACTTAAAGTATAATTAAAAAAAAAAAGAAAACAGAAGCTATTTATAAA
GAAGTTATTTGCTGAAATAAATGTGATCTTTCCCATTAAAAAAATAAAGA
AATTTTGGGGTAAAAAAACACAATATATTGTATTCTTGAAAAATTCTAAG
AGAGTGGATGTGAAGTGTTCTCACCACAAAAGTGATAACTAATTGAGGTA
ATGCACATATTAATTAGAAAGATTTTGTCATTCCACAATGTATATATACT
TAAAAATATGTTATACACAATAAATACATACATTAAAAAATAAGTAAATG
TA);
3′UTR-012 (Col6a1; collagen, type VI,
alpha 1 UTR)
(SEQ ID NO. 37)
(CCCACCCTGCACGCCGGCACCAAACCCTGTCCTCCCACCCCTCCCCACT
CATCACTAAACAGAGTAAAATGTGATGCGAATTTTCCCGACCAACCTGAT
TCGCTAGATTTTTTTTAAGGAAAAGCTTGGAAAGCCAGGACACAACGCTG
CTGCCTGCTTTGTGCAGGGTCCTCCGGGGCTCAGCCCTGAGTTGGCATCA
CCTGCGCAGGGCCCTCTGGGGCTCAGCCCTGAGCTAGTGTCACCTGCACA
GGGCCCTCTGAGGCTCAGCCCTGAGCTGGCGTCACCTGTGCAGGGCCCTC
TGGGGCTCAGCCCTGAGCTGGCCTCACCTGGGTTCCCCACCCCGGGCTCT
CCTGCCCTGCCCTCCTGCCCGCCCTCCCTCCTGCCTGCGCAGCTCCTTCC
CTAGGCACCTCTGTGCTGCATCCCACCAGCCTGAGCAAGACGCCCTCTCG
GGGCCTGTGCCGCACTAGCCTCCCTCTCCTCTGTCCCCATAGCTGGTTTT
TCCCACCAATCCTCACCTAACAGTTACTTTACAATTAAACTCAAAGCAAG
CTCTTCTCCTCAGCTTGGGGCAGCCATTGGCCTCTGTCTCGTTTTGGGAA
ACCAAGGTCAGGAGGCCGTTGCAGACATAAATCTCGGCGACTCGGCCCCG
TCTCCTGAGGGTCCTGCTGGTGACCGGCCTGGACCTTGGCCCTACAGCCC
TGGAGGCCGCTGCTGACCAGCACTGACCCCGACCTCAGAGAGTACTCGCA
GGGGCGCTGGCTGCACTCAAGACCCTCGAGATTAACGGTGCTAACCCCGT
CTGCTCCTCCCTCCCGCAGAGACTGGGGCCTGGACTGGACATGAGAGCCC
CTTGGTGCCACAGAGGGCTGTGTCTTACTAGAAACAACGCAAACCTCTCC
TTCCTCAGAATAGTGATGTGTTCGACGTTTTATCAAAGGCCCCCTTTCTA
TGTTCATGTTAGTTTTGCTCCTTCTGTGTTTTTTTCTGAACCATATCCAT
GTTGCTGACTTTTCCAAATAAAGGTTTTCACTCCTCTC);
3′UTR-013 (Cair; calreticulin UTR)
(SEQ ID NO. 38)
(AGAGGCCTGCCTCCAGGGCTGGACTGAGGCCTGAGCGCTCCTGCCGCAG
AGCTGGCCGCGCCAAATAATGTCTCTGTGAGACTCGAGAACTTTCATTTT
TTTCCAGGCTGGTTCGGATTTGGGGTGGATTTTGGTTTTGTTCCCCTCCT
CCACTCTCCCCCACCCCCTCCCCGCCCTTTTTTTTTTTTTTTTTTAAACT
GGTATTTTATCTTTGATTCTCCTTCAGCCCTCACCCCTGGTTCTCATCTT
TCTTGATCAACATCTTTTCTTGCCTCTGTCCCCTTCTCTCATCTCTTAGC
TCCCCTCCAACCTGGGGGGCAGTGGTGTGGAGAAGCCACAGGCCTGAGAT
TTCATCTGCTCTCCTTCCTGGAGCCCAGAGGAGGGCAGCAGAAGGGGGTG
GTGTCTCCAACCCCCCAGCACTGAGGAAGAACGGGGCTCTTCTCATTTCA
CCCCTCCCTTTCTCCCCTGCCCCCAGGACTGGGCCACTTCTGGGTGGGGC
AGTGGGTCCCAGATTGGCTCACACTGAGAATGTAAGAACTACAAACAAAA
TTTCTATTAAATTAAATTTTGTGTCTCC);
3′UTR-014 (Col1a1; collagen, type I,
alpha 1 UTR)
(SEQ ID NO. 39)
(CTCCCTCCATCCCAACCTGGCTCCCTCCCACCCAACCAACTTTCCCCCC
AACCCGGAAACAGACAAGCAACCCAAACTGAACCCCCTCAAAAGCCAAAA
AATGGGAGACAATTTCACATGGACTTTGGAAAATATTTTTTTCCTTTGCA
TTCATCTCTCAAACTTAGTTTTTATCTTTGACCAACCGAACATGACCAAA
AACCAAAAGTGCATTCAACCTTACCAAAAAAAAAAAAAAAAAAAGAATAA
ATAAATAACTTTTTAAAAAAGGAAGCTTGGTCCACTTGCTTGAAGACCCA
TGCGGGGGTAAGTCCCTTTCTGCCCGTTGGGCTTATGAAACCCCAATGCT
GCCCTTTCTGCTCCTTTCTCCACACCCCCCTTGGGGCCTCCCCTCCACTC
CTTCCCAAATCTGTCTCCCCAGAAGACACAGGAAACAATGTATTGTCTGC
CCAGCAATCAAAGGCAATGCTCAAACACCCAAGTGGCCCCCACCCTCAGC
CCGCTCCTGCCCGCCCAGCACCCCCAGGCCCTGGGGGACCTGGGGTTCTC
AGACTGCCAAAGAAGCCTTGCCATCTGGCGCTCCCATGGCTCTTGCAACA
TCTCCCCTTCGTTTTTGAGGGGGTCATGCCGGGGGAGCCACCAGCCCCTC
ACTGGGTTCGGAGGAGAGTCAGGAAGGGCCACGACAAAGCAGAAACATCG
GATTTGGGGAACGCGTGTCAATCCCTTGTGCCGCAGGGCTGGGCGGGAGA
GACTGTTCTGTTCCTTGTGTAACTGTGTTGCTGAAAGACTACCTCGTTCT
TGTCTTGATGTGTCACCGGGGCAACTGCCTGGGGGCGGGGATGGGGGCAG
GGTGGAAGCGGCTCCCCATTTTATACCAAAGGTGCTACATCTATGTGATG
GGTGGGGTGGGGAGGGAATCACTGGTGCTATAGAAATTGAGATGCCCCCC
CAGGCCAGCAAATGTTCCTTTTTGTTCAAAGTCTATTTTTATTCCTTGAT
ATTTTTCTTTTTTTTTTTTTTTTTTTGTGGATGGGGACTTGTGAATTTTT
CTAAAGGTGCTATTTAACATGGGAGGAGAGCGTGTGCGGCTCCAGCCCAG
CCCGCTGCTCACTTTCCACCCTCTCTCCACCTGCCTCTGGCTTCTCAGGC
CTCTGCTCTCCGACCTCTCTCCTCTGAAACCCTCCTCCACAGCTGCAGCC
CATCCTCCCGGCTCCCTCCTAGTCTGTCCTGCGTCCTCTGTCCCCGGGTT
TCAGAGACAACTTCCCAAAGCACAAAGCAGTTTTTCCCCCTAGGGGTGGG
AGGAAGCAAAAGACTCTGTACCTATTTTGTATGTGTATAATAATTTGAGA
TGTTTTTAATTATTTTGATTGCTGGAATAAAGCATGTGGAAATGACCCAA
ACATAATCCGCAGTGGCCTCCTAATTTCCTTCTTTGGAGTTGGGGGAGGG
GTAGACATGGGGAAGGGGCTTTGGGGTGATGGGCTTGCCTTCCATTCCTG
CCCTTTCCCTCCCCACTATTCTCTTCTAGATCCCTCCATAACCCCACTCC
CCTTTCTCTCACCCTTCTTATACCGCAAACCTTTCTACTTCCTCTTTCAT
TTTCTATTCTTGCAATTTCCTTGCACCTTTTCCAAATCCTCTTCTCCCCT
GCAATACCATACAGGCAATCCACGTGCACAACACACACACACACTCTTCA
CATCTGGGGTTGTCCAAACCTCATACCCACTCCCCTTCAAGCCCATCCAC
TCTCCACCCCCTGGATGCCCTGCACTTGGTGGCGGTGGGATGCTCATGGA
TACTGGGAGGGTGAGGGGAGTGGAACCCGTGAGGAGGACCTGGGGGCCTC
TCCTTGAACTGACATGAAGGGTCATCTGGCCTCTGCTCCCTTCTCACCCA
CGCTGACCTCCTGCCGAAGGAGCAACGCAACAGGAGAGGGGTCTGCTGAG
CCTGGCGAGGGTCTGGGAGGGACCAGGAGGAAGGCGTGCTCCCTGCTCGC
TGTCCTGGCCCTGGGGGAGTGAGGGAGACAGACACCTGGGAGAGCTGTGG
GGAAGGCACTCGCACCGTGCTCTTGGGAAGGAAGGAGACCTGGCCCTGCT
CACCACGGACTGGGTGCCTCGACCTCCTGAATCCCCAGAACACAACCCCC
CTGGGCTGGGGTGGTCTGGGGAACCATCGTGCCCCCGCCTCCCGCCTACT
CCTTTTTAAGCTT);
3′UTR-015 (Plod1; procollagen-lysine,
2-oxoglutarate 5-dioxygenase 1 UTR)
(SEQ ID NO. 40)
(TTGGCCAGGCCTGACCCTCTTGGACCTTTCTTCTTTGCCGACAACCACT
GCCCAGCAGCCTCTGGGACCTCGGGGTCCCAGGGAACCCAGTCCAGCCTC
CTGGCTGTTGACTTCCCATTGCTCTTGGAGCCACCAATCAAAGAGATTCA
AAGAGATTCCTGCAGGCCAGAGGCGGAACACACCTTTATGGCTGGGGCTC
TCCGTGGTGTTCTGGACCCAGCCCCTGGAGACACCATTCACTTTTACTGC
TTTGTAGTGACTCGTGCTCTCCAACCTGTCTTCCTGAAAAACCAAGGCCC
CCTTCCCCCACCTCTTCCATGGGGTGAGACTTGAGCAGAACAGGGGCTTC
CCCAAGTTGCCCAGAAAGACTGTCTGGGTGAGAAGCCATGGCCAGAGCTT
CTCCCAGGCACAGGTGTTGCACCAGGGACTTCTGCTTCAAGTTTTGGGGT
AAAGACACCTGGATCAGACTCCAAGGGCTGCCCTGAGTCTGGGACTTCTG
CCTCCATGGCTGGTCATGAGAGCAAACCGTAGTCCCCTGGAGACAGCGAC
TCCAGAGAACCTCTTGGGAGACAGAAGAGGCATCTGTGCACAGCTCGATC
TTCTACTTGCCTGTGGGGAGGGGAGTGACAGGTCCACACACCACACTGGG
TCACCCTGTCCTGGATGCCTCTGAAGAGAGGGACAGACCGTCAGAAACTG
GAGAGTTTCTATTAAAGGTCATTTAAACCA);
3′UTR-016 (Nucb1; nucleobindin 1 UTR)
(SEQ ID NO. 41)
(TCCTCCGGGACCCCAGCCCTCAGGATTCCTGATGCTCCAAGGCGACTGA
TGGGCGCTGGATGAAGTGGCACAGTCAGCTTCCCTGGGGGCTGGTGTCAT
GTTGGGCTCCTGGGGCGGGGGCACGGCCTGGCATTTCACGCATTGCTGCC
ACCCCAGGTCCACCTGTCTCCACTTTCACAGCCTCCAAGTCTGTGGCTCT
TCCCTTCTGTCCTCCGAGGGGCTTGCCTTCTCTCGTGTCCAGTGAGGTGC
TCAGTGATCGGCTTAACTTAGAGAAGCCCGCCCCCTCCCCTTCTCCGTCT
GTCCCAAGAGGGTCTGCTCTGAGCCTGCGTTCCTAGGTGGCTCGGCCTCA
GCTGCCTGGGTTGTGGCCGCCCTAGCATCCTGTATGCCCACAGCTACTGG
AATCCCCGCTGCTGCTCCGGGCCAAGCTTCTGGTTGATTAATGAGGGCAT
GGGGTGGTCCCTCAAGACCTTCCCCTACCTTTTGTGGAACCAGTGATGCC
TCAAAGACAGTGTCCCCTCCACAGCTGGGTGCCAGGGGCAGGGGATCCTC
AGTATAGCCGGTGAACCCTGATACCAGGAGCCTGGGCCTCCCTGAACCCC
TGGCTTCCAGCCATCTCATCGCCAGCCTCCTCCTGGACCTCTTGGCCCCC
AGCCCCTTCCCCACACAGCCCCAGAAGGGTCCCAGAGCTGACCCCACTCC
AGGACCTAGGCCCAGCCCCTCAGCCTCATCTGGAGCCCCTGAAGACCAGT
CCCACCCACCTTTCTGGCCTCATCTGACACTGCTCCGCATCCTGCTGTGT
GTCCTGTTCCATGTTCCGGTTCCATCCAAATACACTTTCTGGAACAAA);
3′UTR-017 (α-globin)
(SEQ ID NO. 42)
(GCTGGAGCCTCGGTGGCCATGCTTCTTGCCCCTTGGGCCTCCCCCCAGC
CCCTCCTCCCCTTCCTGCACCCGTACCCCCGTGGTCTTTGAATAAAGTCT
GAGTGGGCGGC);
or
3′UTR-018
(SEQ ID NO. 43)
(TGATAATAGGCTGGAGCCTCGGTGGCCATGCTTCTTGCCCCTTGGGCCT
CCCCCCAGCCCCTCCTCCCCTTCCTGCACCCGTACCCCCGTGGTCTTTGA
ATAAAGTCTGAGTGGGCGGC).

In certain embodiments, the 5′UTR and/or 3′UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′UTR sequences comprising any of SEQ ID NOs: 1-25 and/or 3′UTR sequences comprises any of SEQ ID NOs: 26-43, and any combination thereof.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

It is also within the scope of the present invention to have patterned UTRs. As used herein “patterned UTRs” include a repeating or alternating pattern, such as ABABAB or AABBAABBAABB or ABCABCABC or variants thereof repeated once, twice, or more than 3 times. In these patterns, each letter, A, B, or C represent a different UTR nucleic acid sequence.

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide of the invention comprises 5′ and/or 3′ sequence associated with the 5′ and/or 3′ ends of rubella virus (RV) genomic RNA, respectively, or deletion derivatives thereof, including the 5′ proximal open reading frame of RV RNA encoding nonstructural proteins (e.g., see Pogue et al., J. Virol. 67(12):7106-7117, the contents of which are incorporated herein by reference in their entirety). Viral capsid sequences can also be used as a translational enhancer, e.g., the 5′ portion of a capsid sequence, (e.g., semliki forest virus and sindbis virus capsid RNAs as described in Sjöberg et al., Biotechnology (NY) 1994 12(11):1127-1131, and Frolov and Schlesinger J. Virol. 1996 70(2):1182-1190, the contents of each of which are incorporated herein by reference in their entirety). In some embodiments, the polynucleotide comprises an IRES instead of a 5′UTR sequence. In some embodiments, the polynucleotide comprises an ORF and a viral capsid sequence. In some embodiments, the polynucleotide comprises a synthetic 5′UTR in combination with a non-synthetic 3′UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′UTR comprises a TEE.

In some embodiments, a 5′UTR and/or 3′UTR comprising at least one TEE described herein can be incorporated in a monocistronic sequence such as, but not limited to, a vector system or a nucleic acid vector.

In some embodiments, a 5′UTR and/or 3′UTR of a polynucleotide of the invention comprises a TEE or portion thereof described herein. In some embodiments, the TEEs in the 3′UTR can be the same and/or different from the TEE located in the 5′UTR.

In some embodiments, a 5′UTR and/or 3′UTR of a polynucleotide of the invention can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In one embodiment, the 5′UTR of a polynucleotide of the invention can include 1-60, 1-55, 1-50, 1-45, 1-40, 1-35, 1-30, 1-25, 1-20, 1-15, 1-10, 9, 8, 7, 6, 5, 4, 3, 2, or 1 TEE sequences. The TEE sequences in the 5′UTR of the polynucleotide of the invention can be the same or different TEE sequences. A combination of different TEE sequences in the 5′UTR of the polynucleotide of the invention can include combinations in which more than one copy of any of the different TEE sequences are incorporated. The TEE sequences can be in a pattern such as ABABAB or AABBAABBAABB or ABCABCABC or variants thereof repeated one, two, three, or more than three times. In these patterns, each letter, A, B, or C represent a different TEE nucleotide sequence.

In some embodiments, the TEE can be identified by the methods described in US2007/0048776, US2011/0124100, WO2007/025008, WO2012/009644, the contents of each of which are incorporated herein by reference in their entirety.

In some embodiments, the 5′UTR and/or 3′UTR comprises a spacer to separate two TEE sequences. As a non-limiting example, the spacer can be a 15 nucleotide spacer and/or other spacers known in the art. As another non-limiting example, the 5′UTR and/or 3′UTR comprises a TEE sequence-spacer module repeated at least once, at least twice, at least 3 times, at least 4 times, at least 5 times, at least 6 times, at least 7 times, at least 8 times, at least 9 times, at least 10 times, or more than 10 times in the 5′UTR and/or 3′UTR, respectively. In some embodiments, the 5′UTR and/or 3′UTR comprises a TEE sequence-spacer module repeated 1, 2, 3, 4, 5, 6, 7, 8, 9, or 10 times.

In some embodiments, the spacer separating two TEE sequences can include other sequences known in the art that can regulate the translation of the polynucleotide of the invention, e.g., miR sequences described herein (e.g., miR binding sites and miR seeds). As a non-limiting example, each spacer used to separate two TEE sequences can include a different miR sequence or component of a miR sequence (e.g., miR seed sequence).

In some embodiments, a polynucleotide of the invention comprises a miR and/or TEE sequence. In some embodiments, the incorporation of a miR sequence and/or a TEE sequence into a polynucleotide of the invention can change the shape of the stem loop region, which can increase and/or decrease translation. See e.g., Kedde et al., Nature Cell Biology 2010 12(10):1014-20, herein incorporated by reference in its entirety).

MicroRNA (miRNA) Binding Sites

Polynucleotides of the invention can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences”.

In some embodiments, a polynucleotide (e.g., a ribonucleic acid (RNA), e.g., a messenger RNA (mRNA)) of the invention comprises an open reading frame (ORF) encoding a polypeptide of interest and further comprises one or more miRNA binding site(s). Inclusion or incorporation of miRNA binding site(s) provides for regulation of polynucleotides of the invention, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA.

As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the invention comprising an ORF encoding a polypeptide of interest and further comprises one or more miRNA binding site(s). In exemplary embodiments, a 5′UTR and/or 3′UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the invention, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with an miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the invention, the polynucleotide can be targeted for degradation or reduced translation, provided the miRNA in question is available. This can reduce off-target effects upon delivery of the polynucleotide. For example, if a polynucleotide of the invention is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the polynucleotide.

Conversely, miRNA binding sites can be removed from polynucleotide sequences in which they naturally occur in order to increase protein expression in specific tissues. For example, a binding site for a specific miRNA can be removed from a polynucleotide to improve protein expression in tissues or cells containing the miRNA.

In one embodiment, a polynucleotide of the invention can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a polynucleotide of the invention can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune-response to infection, inflammation, as well as unwanted immune response after gene therapy and tissue/organ transplantation. Immune cells specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a polynucleotide of the invention can selectively repress gene expression in antigen presenting cells through miR-142 mediated degradation, limiting antigen presentation in antigen presenting cells (e.g., dendritic cells) and thereby preventing antigen-mediated immune response after the delivery of the polynucleotide. The polynucleotide is then stably expressed in target tissues or cells without triggering cytotoxic elimination.

In one embodiment, binding sites for miRNAs that are known to be expressed in immune cells, in particular, antigen presenting cells, can be engineered into a polynucleotide of the invention to suppress the expression of the polynucleotide in antigen presenting cells through miRNA mediated RNA degradation, subduing the antigen-mediated immune response. Expression of the polynucleotide is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a polynucleotide of the invention.

To further drive the selective degradation and suppression in APCs and macrophage, a polynucleotide of the invention can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. MiRNA binding sites from any liver specific miRNA can be introduced to or removed from a polynucleotide of the invention to regulate expression of the polynucleotide in the liver. Liver specific miRNA binding sites can be engineered alone or further in combination with immune cell (e.g., APC) miRNA binding sites in a polynucleotide of the invention.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a polynucleotide of the invention to regulate expression of the polynucleotide in the lung. Lung specific miRNA binding sites can be engineered alone or further in combination with immune cell (e.g., APC) miRNA binding sites in a polynucleotide of the invention.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. mMiRNA binding sites from any heart specific microRNA can be introduced to or removed from a polynucleotide of the invention to regulate expression of the polynucleotide in the heart. Heart specific miRNA binding sites can be engineered alone or further in combination with immune cell (e.g., APC) miRNA binding sites in a polynucleotide of the invention.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657.

miRNA binding sites from any CNS specific miRNA can be introduced to or removed from a polynucleotide of the invention to regulate expression of the polynucleotide in the nervous system. Nervous system specific miRNA binding sites can be engineered alone or further in combination with immune cell (e.g., APC) miRNA binding sites in a polynucleotide of the invention.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. MiRNA binding sites from any pancreas specific miRNA can be introduced to or removed from a polynucleotide of the invention to regulate expression of the polynucleotide in the pancreas. Pancreas specific miRNA binding sites can be engineered alone or further in combination with immune cell (e.g. APC) miRNA binding sites in a polynucleotide of the invention.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a polynucleotide of the invention to regulate expression of the polynucleotide in the kidney. Kidney specific miRNA binding sites can be engineered alone or further in combination with immune cell (e.g., APC) miRNA binding sites in a polynucleotide of the invention.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. MiRNA binding sites from any muscle specific miRNA can be introduced to or removed from a polynucleotide of the invention to regulate expression of the polynucleotide in the muscle. Muscle specific miRNA binding sites can be engineered alone or further in combination with immune cell (e.g., APC) miRNA binding sites in a polynucleotide of the invention.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a polynucleotide of the invention to regulate expression of the polynucleotide in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a polynucleotide of the invention to regulate expression of the polynucleotide in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety). MiRNAs abundant in embryonic stem cells include, but are not limited to, let-7a-2-3p, let-a-3p, let-7a-5p, 1et7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-548l, miR-548m, miR-548n, miR-5480-3p, miR-5480-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941, miR-96-3p, miR-96-5p, miR-99b-3p and miR-99b-5p. Many predicted novel miRNAs are discovered by deep sequencing in human embryonic stem cells (e.g., Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by reference in its entirety).

In one embodiment, the binding sites of embryonic stem cell specific miRNAs can be included in or removed from the 3′UTR of a polynucleotide of the invention to modulate the development and/or differentiation of embryonic stem cells, to inhibit the senescence of stem cells in a degenerative condition (e.g. degenerative diseases), or to stimulate the senescence and apoptosis of stem cells in a disease condition (e.g. cancer stem cells).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in its entirety.)

As a non-limiting example, miRNA binding sites for miRNAs that are over-expressed in certain cancer and/or tumor cells can be removed from the 3′UTR of a polynucleotide of the invention, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the polynucleotides of the invention, miRNA binding sites that are involved in such processes can be removed or introduced, in order to tailor the expression of the polynucleotides to biologically relevant cell types or relevant biological processes. In this context, the polynucleotides of the invention are defined as auxotrophic polynucleotides.

In some embodiments, a polynucleotide of the invention comprises a miRNA binding site, wherein the miRNA binding site comprises one or more nucleotide sequences selected from TABLE 4, including one or more copies of any one or more of the miRNA binding site sequences. In some embodiments, a polynucleotide of the invention further comprises at least one, two, three, four, five, six, seven, eight, nine, ten, or more of the same or different miRNA binding sites selected from TABLE 4, including any combination thereof. In some embodiments, the miRNA binding site binds to miR-142 or is complementary to miR-142. In some embodiments, the miR-142 comprises SEQ ID NO: 44. In some embodiments, the miRNA binding site binds to miR-142-3p or miR-142-5p. In some embodiments, the miR-142-3p binding site comprises SEQ ID NO: 46. In some embodiments, the miR-142-5p binding site comprises SEQ ID NO: 48. In some embodiments, the miRNA binding site comprises a nucleotide sequence at least 80%, at least 85%, at least 90%, at least 95%, or 100% identical to SEQ ID NO: 46 or SEQ ID NO: 48.

TABLE 4
miR-142 and miR-142 binding sites

| SEQ | | |
|---|---|---|
| ID | | |
| NO. | Description | Sequence |
| 44 | miR-142 | GACAGUGCAGUCACCCAUAAAGUAGAAAGCACU |
| | | ACUAACAGCACUGGAGGGUGUAGUGUUUCCUAC |
| | | UUUAUGGAUGAGUGUACUGUG |
| 45 | miR-142-3p | UGUAGUGUUUCCUACUUUAUGGA |
| 46 | miR-142-3p | UCCAUAAAGUAGGAAACACUACA |
| | binding | |
| | site | |
| 47 | miR-142-5p | CAUAAAGUAGAAAGCACUACU |
| 48 | miR-142-5p | AGUAGUGCUUUCUACUUUAUG |
| | binding | |
| | site | |

In some embodiments, a miRNA binding site is inserted in the polynucleotide of the invention in any position of the polynucleotide (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide.

In some embodiments, a miRNA binding site is inserted in at least about 30 nucleotides downstream from the stop codon of an ORF in a polynucleotide of the invention comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the invention. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the invention.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The polynucleotides of the invention can further include this structured 5′UTR in order to enhance microRNA mediated gene regulation.

At least one miRNA binding site can be engineered into the 3′UTR of a polynucleotide of the invention. In this context, at least two, at least three, at least four, at least five, at least six, at least seven, at least eight, at least nine, at least ten, or more miRNA binding sites can be engineered into a 3′UTR of a polynucleotide of the invention. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the invention. In one embodiment, miRNA binding sites incorporated into a polynucleotide of the invention can be the same or can be different miRNA sites. A combination of different miRNA binding sites incorporated into a polynucleotide of the invention can include combinations in which more than one copy of any of the different miRNA sites are incorporated. In another embodiment, miRNA binding sites incorporated into a polynucleotide of the invention can target the same or different tissues in the body. As a non-limiting example, through the introduction of tissue-, cell-type-, or disease-specific miRNA binding sites in the 3′-UTR of a polynucleotide of the invention, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a polynucleotide of the invention. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a polynucleotide of the invention can be engineered to include more than one miRNA site expressed in different tissues or different cell types of a subject. As a non-limiting example, a polynucleotide of the invention can be engineered to include miR-192 and miR-122 to regulate expression of the polynucleotide in the liver and kidneys of a subject. In another embodiment, a polynucleotide of the invention can be engineered to include more than one miRNA site for the same tissue.

In some embodiments, the therapeutic window and or differential expression associated with the polypeptide encoded by a polynucleotide of the invention can be altered with a miRNA binding site. For example, a polynucleotide encoding a polypeptide that provides a death signal can be designed to be more highly expressed in cancer cells by virtue of the miRNA signature of those cells. Where a cancer cell expresses a lower level of a particular miRNA, the polynucleotide encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell. Multiple polynucleotides can be designed and administered having different signals based on the use of miRNA binding sites as described herein.

In some embodiments, the expression of a polynucleotide of the invention can be controlled by incorporating at least one sensor sequence in the polynucleotide and formulating the polynucleotide for administration. As a non-limiting example, a polynucleotide of the invention can be targeted to a tissue or cell by incorporating a miRNA binding site and formulating the polynucleotide in a lipid nanoparticle comprising a cationic lipid, including any of the lipids described herein.

A polynucleotide of the invention can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the invention can be designed for optimal protein expression in a tissue or cell, or in the context of a biological condition.

In some embodiments, a polynucleotide of the invention can be designed to incorporate miRNA binding sites that either have 100% identity to known miRNA seed sequences or have less than 100% identity to miRNA seed sequences. In some embodiments, a polynucleotide of the invention can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment, the 5′-UTR of a polynucleotide of the invention can comprise at least one miRNA sequence. The miRNA sequence can be, but is not limited to, a 19 or 22 nucleotide sequence and/or a miRNA sequence without the seed.

In one embodiment the miRNA sequence in the 5′UTR can be used to stabilize a polynucleotide of the invention described herein.

In another embodiment, a miRNA sequence in the 5′UTR of a polynucleotide of the invention can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A polynucleotide of the invention can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site.

In some embodiments, a polynucleotide of the invention can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the invention can be specific to the hematopoietic system. As another non-limiting example, a miRNA incorporated into a polynucleotide of the invention to dampen antigen presentation is miR-142-3p.

In some embodiments, a polynucleotide of the invention can include at least one miRNA in order to dampen expression of the encoded polypeptide in a tissue or cell of interest. As a non-limiting example, a polynucleotide of the invention can include at least one miR-122 binding site in order to dampen expression of an encoded polypeptide of interest in the liver. As another non-limiting example a polynucleotide of the invention can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the invention can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a polynucleotide of the invention more unstable in antigen presenting cells. Non-limiting examples of these miRNAs include mir-142-5p, mir-142-3p, mir-146a-5p, and mir-146-3p.

In one embodiment, a polynucleotide of the invention comprises at least one miRNA sequence in a region of the polynucleotide that can interact with a RNA binding protein.

In some embodiments, the polynucleotide of the invention (e.g., a RNA, e.g., an mRNA) comprising (i) a sequence-optimized nucleotide sequence (e.g., an ORF) encoding a wild type therapeutic polypeptide and (ii) a miRNA binding site (e.g., a miRNA binding site that binds to miR-142).

### 3′ UTR and the AU Rich Elements

In certain embodiments, a polynucleotide of the present invention (e.g., a polynucleotide comprising a nucleotide sequence encoding a therapeutic polypeptide of the invention) further comprises a 3′ UTR.

3′-UTR is the section of mRNA that immediately follows the translation termination codon and often contains regulatory regions that post-transcriptionally influence gene expression. Regulatory regions within the 3′-UTR can influence polyadenylation, translation efficiency, localization, and stability of the mRNA. In one embodiment, the 3′-UTR useful for the invention comprises a binding site for regulatory proteins or microRNAs. In some embodiments, the 3′-UTR has a silencer region, which binds to repressor proteins and inhibits the expression of the mRNA. In other embodiments, the 3′-UTR comprises an AU-rich element. Proteins bind AREs to affect the stability or decay rate of transcripts in a localized manner or affect translation initiation. In other embodiments, the 3′-UTR comprises the sequence AAUAAA that directs addition of several hundred adenine residues called the poly(A) tail to the end of the mRNA transcript.

Natural or wild type 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides of the invention. When engineering specific polynucleotides, one or more copies of an ARE can be introduced to make polynucleotides of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using polynucleotides of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

### Regions Having a 5′ Cap

The invention also includes a polynucleotide that comprises both a 5′ Cap and a polynucleotide of the present invention (e.g., a polynucleotide comprising a nucleotide sequence encoding a therapeutic polypeptide).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a therapeutic polypeptide) incorporate a cap moiety.

In some embodiments, polynucleotides of the present invention (e.g., a polynucleotide comprising a nucleotide sequence encoding a therapeutic polypeptide) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′O-Me-m7G(5)ppp(5′)G). The 3′-0 atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dicucleotide form of a cap analog known in the art and/or described herein. Non-limiting examples of a N7-(4-chlorophenoxyethyl) substituted dicucleotide form of a cap analog include a N7-(4-chlorophenoxyethyl)-G(5′)ppp(5′)G and a N7-(4-chlorophenoxyethyl)-m^{3′-O}G(5)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding a therapeutic polypeptide) can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% when a cap analog is linked to a chimeric polynucleotide in the course of an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly-A Tails

In some embodiments, the polynucleotides of the present disclosure (e.g., a polynucleotide comprising a nucleotide sequence encoding a therapeutic polypeptide) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long.

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Start Codon Region

The invention also includes a polynucleotide that comprises both a start codon region and the polynucleotide described herein (e.g., a polynucleotide comprising a nucleotide sequence encoding a therapeutic polypeptide). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide in order to increase the likelihood that translation will initiate on an alternative start codon. In some embodiments, a masking agent can be used to mask a first start codon or alternative start codon in order to increase the chance that translation will initiate on a start codon or alternative start codon downstream to the masked start codon or alternative start codon.

In some embodiments, a start codon or alternative start codon can be located within a perfect complement for a miR binding site. The perfect complement of a miR binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence in order to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

### Stop Codon Region

The invention also includes a polynucleotide that comprises both a stop codon region and the polynucleotide described herein (e.g., a polynucleotide comprising a nucleotide sequence encoding a therapeutic polypeptide). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

Polynucleotide Comprising an mRNA Encoding a Therapeutic Polypeptide

In certain embodiments, a polynucleotide of the present disclosure, for example a polynucleotide comprising an mRNA nucleotide sequence encoding a therapeutic polypeptide, comprises from 5′ to 3′ end:

(i) a 5′ cap provided above;

(ii) a 5′ UTR, such as the sequences provided above;

(iii) an open reading frame encoding a therapeutic polypeptide, e.g., a sequence optimized nucleic acid sequence encoding a therapeutic polypeptide disclosed herein;

(iv) at least one stop codon;

(v) a 3′ UTR, such as the sequences provided above; and

(vi) a poly-A tail provided above.

In some embodiments, the polynucleotide further comprises a miRNA binding site, e.g, a miRNA binding site that binds to miRNA-142. In some embodiments, the 5′UTR comprises the miRNA binding site.

In some embodiments, a polynucleotide of the present disclosure comprises a nucleotide sequence encoding a polypeptide sequence at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the protein sequence of a wild type therapeutic protein.

### Methods of Making Polynucleotides

The present disclosure also provides methods for making a polynucleotide of the invention (e.g., a polynucleotide comprising a nucleotide sequence encoding a therapeutic polypeptide) or a complement thereof.

In some aspects, a polynucleotide (e.g., a RNA, e.g., an mRNA) disclosed herein, and encoding a therapeutic polypeptide, can be constructed using in vitro transcription. In other aspects, a polynucleotide (e.g., a RNA, e.g., an mRNA) disclosed herein, and encoding a therapeutic polypeptide, can be constructed by chemical synthesis using an oligonucleotide synthesizer.

In other aspects, a polynucleotide (e.g., a RNA, e.g., an mRNA) disclosed herein, and encoding a therapeutic polypeptide is made by using a host cell. In certain aspects, a polynucleotide (e.g., a RNA, e.g., an mRNA) disclosed herein, and encoding a therapeutic polypeptide is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., a RNA, e.g., an mRNA) encoding a therapeutic polypeptide. The resultant polynucleotides, e.g., mRNAs, can then be examined for their ability to produce protein and/or produce a therapeutic outcome.

a. In Vitro Transcription/Enzymatic Synthesis

The polynucleotides of the present invention disclosed herein (e.g., a polynucleotide comprising a nucleotide sequence encoding a therapeutic polypeptide) can be transcribed using an in vitro transcription (IVT) system. The system typically comprises a transcription buffer, nucleotide triphosphates (NTPs), an RNase inhibitor and a polymerase. The NTPs can be selected from, but are not limited to, those described herein including natural and unnatural (modified) NTPs. The polymerase can be selected from, but is not limited to, T7 RNA polymerase, T3 RNA polymerase and mutant polymerases such as, but not limited to, polymerases able to incorporate polynucleotides disclosed herein. See U.S. Publ. No. US20130259923, which is herein incorporated by reference in its entirety.

Any number of RNA polymerases or variants can be used in the synthesis of the polynucleotides of the present invention. RNA polymerases can be modified by inserting or deleting amino acids of the RNA polymerase sequence. As a non-limiting example, the RNA polymerase can be modified to exhibit an increased ability to incorporate a 2′-modified nucleotide triphosphate compared to an unmodified RNA polymerase (see International Publication WO2008078180 and U.S. Pat. No. 8,101,385; herein incorporated by reference in their entireties).

Variants can be obtained by evolving an RNA polymerase, optimizing the RNA polymerase amino acid and/or nucleic acid sequence and/or by using other methods known in the art. As a non-limiting example, T7 RNA polymerase variants can be evolved using the continuous directed evolution system set out by Esvelt et al. (Nature 472:499-503 (2011); herein incorporated by reference in its entirety) where clones of T7 RNA polymerase can encode at least one mutation such as, but not limited to, lysine at position 93 substituted for threonine (K93T), I4M, A7T, E63V, V64D, A65E, D66Y, T76N, C125R, S128R, A136T, N165S, G175R, H176L, Y178H, F182L, L196F, G198V, D208Y, E222K, S228A, Q239R, T243N, G259D, M2671, G280C, H300R, D351A, A354S, E356D, L360P, A383V, Y385C, D388Y, S397R, M401T, N410S, K450R, P451T, G452V, E484A, H523L, H524N, G542V, E565K, K577E, K577M, N601S, S684Y, L699I, K713E, N748D, Q754R, E775K, A827V, D851N or L864F. As another non-limiting example, T7 RNA polymerase variants can encode at least mutation as described in U.S. Pub. Nos. 20100120024 and 20070117112; herein incorporated by reference in their entireties. Variants of RNA polymerase can also include, but are not limited to, substitutional variants, conservative amino acid substitution, insertional variants, deletional variants and/or covalent derivatives.

In one aspect, the polynucleotide can be designed to be recognized by the wild type or variant RNA polymerases. In doing so, the polynucleotide can be modified to contain sites or regions of sequence changes from the wild type or parent chimeric polynucleotide.

Polynucleotide or nucleic acid synthesis reactions can be carried out by enzymatic methods utilizing polymerases. Polymerases catalyze the creation of phosphodiester bonds between nucleotides in a polynucleotide or nucleic acid chain. Currently known DNA polymerases can be divided into different families based on amino acid sequence comparison and crystal structure analysis. DNA polymerase I (pol I) or A polymerase family, including the Klenow fragments of E. coli, Bacillus DNA polymerase I, Thermus aquaticus (Taq) DNA polymerases, and the T7 RNA and DNA polymerases, is among the best studied of these families. Another large family is DNA polymerase a (pol a) or B polymerase family, including all eukaryotic replicating DNA polymerases and polymerases from phages T4 and RB69. Although they employ similar catalytic mechanism, these families of polymerases differ in substrate specificity, substrate analog-incorporating efficiency, degree and rate for primer extension, mode of DNA synthesis, exonuclease activity, and sensitivity against inhibitors.

DNA polymerases are also selected based on the optimum reaction conditions they require, such as reaction temperature, pH, and template and primer concentrations. Sometimes a combination of more than one DNA polymerases is employed to achieve the desired DNA fragment size and synthesis efficiency. For example, Cheng et al. increase pH, add glycerol and dimethyl sulfoxide, decrease denaturation times, increase extension times, and utilize a secondary thermostable DNA polymerase that possesses a 3′ to 5′ exonuclease activity to effectively amplify long targets from cloned inserts and human genomic DNA. (Cheng et al., PNAS 91:5695-5699 (1994), the contents of which are incorporated herein by reference in their entirety). RNA polymerases from bacteriophage T3, T7, and SP6 have been widely used to prepare RNAs for biochemical and biophysical studies. RNA polymerases, capping enzymes, and poly-A polymerases are disclosed in the co-pending International Publication No. WO2014028429, the contents of which are incorporated herein by reference in their entirety.

In one aspect, the RNA polymerase which can be used in the synthesis of the polynucleotides of the present invention is a Syn5 RNA polymerase. (see Zhu et al. Nucleic Acids Research 2013, doi:10.1093/nar/gkt1193, which is herein incorporated by reference in its entirety). The Syn5 RNA polymerase was recently characterized from marine cyanophage Syn5 by Zhu et al. where they also identified the promoter sequence (see Zhu et al. Nucleic Acids Research 2013, the contents of which is herein incorporated by reference in its entirety). Zhu et al. found that Syn5 RNA polymerase catalyzed RNA synthesis over a wider range of temperatures and salinity as compared to T7 RNA polymerase. Additionally, the requirement for the initiating nucleotide at the promoter was found to be less stringent for Syn5 RNA polymerase as compared to the T7 RNA polymerase making Syn5 RNA polymerase promising for RNA synthesis.

In one aspect, a Syn5 RNA polymerase can be used in the synthesis of the polynucleotides described herein. As a non-limiting example, a Syn5 RNA polymerase can be used in the synthesis of the polynucleotide requiring a precise 3′-terminus.

In one aspect, a Syn5 promoter can be used in the synthesis of the polynucleotides. As a non-limiting example, the Syn5 promoter can be 5′-ATTGGGCACCCGTAAGGG-3′ (SEQ ID NO: 49) as described by Zhu et al. (Nucleic Acids Research 2013).

In one aspect, a Syn5 RNA polymerase can be used in the synthesis of polynucleotides comprising at least one chemical modification described herein and/or known in the art (see e.g., the incorporation of pseudo-UTP and 5Me-CTP described in Zhu et al. Nucleic Acids Research 2013).

In one aspect, the polynucleotides described herein can be synthesized using a Syn5 RNA polymerase which has been purified using modified and improved purification procedure described by Zhu et al. (Nucleic Acids Research 2013).

Various tools in genetic engineering are based on the enzymatic amplification of a target gene which acts as a template. For the study of sequences of individual genes or specific regions of interest and other research needs, it is necessary to generate multiple copies of a target gene from a small sample of polynucleotides or nucleic acids. Such methods can be applied in the manufacture of the polynucleotides of the invention.

Polymerase chain reaction (PCR) has wide applications in rapid amplification of a target gene, as well as genome mapping and sequencing. The key components for synthesizing DNA comprise target DNA molecules as a template, primers complementary to the ends of target DNA strands, deoxynucleoside triphosphates (dNTPs) as building blocks, and a DNA polymerase. As PCR progresses through denaturation, annealing and extension steps, the newly produced DNA molecules can act as a template for the next circle of replication, achieving exponentially amplification of the target DNA. PCR requires a cycle of heating and cooling for denaturation and annealing. Variations of the basic PCR include asymmetric PCR (Innis et al., PNAS 85: 9436-9440 (1988)), inverse PCR (Ochman et al., Genetics 120(3): 621-623, (1988)), reverse transcription PCR (RT-PCR) (Freeman et al., BioTechniques 26(1): 112-22, 124-5 (1999), the contents of which are incorporated herein by reference in their entirety and so on). In RT-PCR, a single stranded RNA is the desired target and is converted to a double stranded DNA first by reverse transcriptase.

A variety of isothermal in vitro nucleic acid amplification techniques have been developed as alternatives or complements of PCR. For example, strand displacement amplification (SDA) is based on the ability of a restriction enzyme to form a nick (Walker et al., PNAS 89: 392-396 (1992), which is incorporated herein by reference in its entirety)). A restriction enzyme recognition sequence is inserted into an annealed primer sequence. Primers are extended by a DNA polymerase and dNTPs to form a duplex. Only one strand of the duplex is cleaved by the restriction enzyme. Each single strand chain is then available as a template for subsequent synthesis. SDA does not require the complicated temperature control cycle of PCR.

Nucleic acid sequence-based amplification (NASBA), also called transcription mediated amplification (TMA), is also an isothermal amplification method that utilizes a combination of DNA polymerase, reverse transcriptase, RNAse H, and T7 RNA polymerase. (Compton, Nature 350:91-92 (1991)) the contents of which are incorporated herein by reference in their entirety. A target RNA is used as a template and a reverse transcriptase synthesizes its complementary DNA strand. RNAse H hydrolyzes the RNA template, making space for a DNA polymerase to synthesize a DNA strand complementary to the first DNA strand which is complementary to the RNA target, forming a DNA duplex. T7 RNA polymerase continuously generates complementary RNA strands of this DNA duplex. These RNA strands act as templates for new cycles of DNA synthesis, resulting in amplification of the target gene.

Rolling-circle amplification (RCA) amplifies a single stranded circular polynucleotide and involves numerous rounds of isothermal enzymatic synthesis where 029 DNA polymerase extends a primer by continuously progressing around the polynucleotide circle to replicate its sequence over and over again. Therefore, a linear copy of the circular template is achieved. A primer can then be annealed to this linear copy and its complementary chain can be synthesized. See Lizardi et al., Nature Genetics 19:225-232 (1998), the contents of which are incorporated herein by reference in their entirety. A single stranded circular DNA can also serve as a template for RNA synthesis in the presence of an RNA polymerase. (Daubendiek et al., JACS 117:7818-7819 (1995), the contents of which are incorporated herein by reference in their entirety). An inverse rapid amplification of cDNA ends (RACE) RCA is described by Polidoros et al. A messenger RNA (mRNA) is reverse transcribed into cDNA, followed by RNAse H treatment to separate the cDNA. The cDNA is then circularized by CircLigase into a circular DNA. The amplification of the resulting circular DNA is achieved with RCA. (Polidoros et al., BioTechniques 41:35-42 (2006), the contents of which are incorporated herein by reference in their entirety).

Any of the foregoing methods can be utilized in the manufacture of one or more regions of the polynucleotides of the present invention.

Assembling polynucleotides or nucleic acids by a ligase is also widely used. DNA or RNA ligases promote intermolecular ligation of the 5′ and 3′ ends of polynucleotide chains through the formation of a phosphodiester bond. Ligase chain reaction (LCR) is a promising diagnosing technique based on the principle that two adjacent polynucleotide probes hybridize to one strand of a target gene and couple to each other by a ligase. If a target gene is not present, or if there is a mismatch at the target gene, such as a single-nucleotide polymorphism (SNP), the probes cannot ligase. (Wiedmann et al., PCR Methods and Application, vol. 3 (4), s51-s64 (1994), the contents of which are incorporated herein by reference in their entirety). LCR can be combined with various amplification techniques to increase sensitivity of detection or to increase the amount of products if it is used in synthesizing polynucleotides and nucleic acids.

Several library preparation kits for nucleic acids are now commercially available. They include enzymes and buffers to convert a small amount of nucleic acid samples into an indexed library for downstream applications. For example, DNA fragments can be placed in a NEBNEXT® ULTRA™ DNA Library Prep Kit by NEWENGLAND BIOLABS® for end preparation, ligation, size selection, clean-up, PCR amplification and final clean-up.

Continued development is going on to improvement the amplification techniques. For example, U.S. Pat. No. 8,367,328 to Asada et al. the contents of which are incorporated herein by reference in their entirety, teaches utilizing a reaction enhancer to increase the efficiency of DNA synthesis reactions by DNA polymerases. The reaction enhancer comprises an acidic substance or cationic complexes of an acidic substance. U.S. Pat. No. 7,384,739 to Kitabayashi et al. the contents of which are incorporated herein by reference in their entirety, teaches a carboxylate ion-supplying substance that promotes enzymatic DNA synthesis, wherein the carboxylate ion-supplying substance is selected from oxalic acid, malonic acid, esters of oxalic acid, esters of malonic acid, salts of malonic acid, and esters of maleic acid. U.S. Pat. No. 7,378,262 to Sobek et al. the contents of which are incorporated herein by reference in their entirety, discloses an enzyme composition to increase fidelity of DNA amplifications. The composition comprises one enzyme with 3′ exonuclease activity but no polymerase activity and another enzyme that is a polymerase. Both of the enzymes are thermostable and are reversibly modified to be inactive at lower temperatures.

U.S. Pat. No. 7,550,264 to Getts et al. teaches multiple round of synthesis of sense RNA molecules are performed by attaching oligodeoxynucleotides tails onto the 3′ end of cDNA molecules and initiating RNA transcription using RNA polymerase, the contents of which are incorporated herein by reference in their entirety. U.S. Pat. Pub. No. 2013/0183718 to Rohayem teaches RNA synthesis by RNA-dependent RNA polymerases (RdRp) displaying an RNA polymerase activity on single-stranded DNA templates, the contents of which are incorporated herein by reference in their entirety. Oligonucleotides with non-standard nucleotides can be synthesized with enzymatic polymerization by contacting a template comprising non-standard nucleotides with a mixture of nucleotides that are complementary to the nucleotides of the template as disclosed in U.S. Pat. No. 6,617,106 to Benner, the contents of which are incorporated herein by reference in their entirety.

b. Chemical Synthesis

Standard methods can be applied to synthesize an isolated polynucleotide sequence encoding an isolated polypeptide of interest, such as a polynucleotide of the invention (e.g., a polynucleotide comprising a nucleotide sequence encoding a therapeutic polypeptide). For example, a single DNA or RNA oligomer containing a codon-optimized nucleotide sequence coding for the particular isolated polypeptide can be synthesized. In other aspects, several small oligonucleotides coding for portions of the desired polypeptide can be synthesized and then ligated. In some aspects, the individual oligonucleotides typically contain 5′ or 3′ overhangs for complementary assembly.

A polynucleotide disclosed herein (e.g., a RNA, e.g., an mRNA) can be chemically synthesized using chemical synthesis methods and potential nucleobase substitutions known in the art. See, for example, International Publication Nos. WO2014093924, WO2013052523; WO2013039857, WO2012135805, WO2013151671; U.S. Publ. No. US20130115272; or U.S. Pat. No. 8,999,380 or 8,710,200, all of which are herein incorporated by reference in their entireties.

c. Purification of Polynucleotides Encoding Therapeutic Polypeptide

Purification of the polynucleotides described herein (e.g., a polynucleotide comprising a nucleotide sequence encoding a therapeutic polypeptide) can include, but is not limited to, polynucleotide clean-up, quality assurance and quality control. Clean-up can be performed by methods known in the arts such as, but not limited to, AGENCOURT® beads (Beckman Coulter Genomics, Danvers, Mass.), poly-T beads, LNA′ oligo-T capture probes (EXIQON® Inc., Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC).

The term “purified” when used in relation to a polynucleotide such as a “purified polynucleotide” refers to one that is separated from at least one contaminant. As used herein, a “contaminant” is any substance that makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide of the invention (e.g., a polynucleotide comprising a nucleotide sequence encoding a therapeutic polypeptide) removes impurities that can reduce or remove an unwanted immune response, e.g., reducing cytokine activity.

### Preparation of High Purity RNA

In order to enhance the purity of synthetically produced RNA, modified in vitro transcription (IVT) processes which produce RNA preparations having vastly different properties from RNA produced using a traditional IVT process may be used. The RNA preparations produced according to these methods have properties that enable the production of qualitatively and quantitatively superior compositions. Even when coupled with extensive purification processes, RNA produced using traditional IVT methods is qualitatively and quantitatively distinct from the RNA preparations produced by the modified IVT processes. For instance, the purified RNA preparations are less immunogenic in comparison to RNA preparations made using traditional IVT. Additionally, increased protein expression levels with higher purity are produced from the purified RNA preparations.

Traditional IVT reactions are performed by incubating a DNA template with an RNA polymerase and equimolar quantities of nucleotide triphosphates, including GTP, ATP, CTP, and UTP in a transcription buffer. An RNA transcript having a 5′ terminal guanosine triphosphate is produced from this reaction. These reactions also result in the production of a number of impurities such as double stranded and single stranded RNAs which are immunostimulatory and may have an additive impact. The purity methods described herein prevent formation of reverse complements and thus prevent the innate immune recognition of both species. In some embodiments the modified IVT methods result in the production of RNA having significantly reduced T cell activity than an RNA preparation made using prior art methods with equimolar NTPs. The prior art attempts to remove these undesirable components using a series of subsequent purification steps. Such purification methods are undesirable because they involve additional time and resources and also result in the incorporation of residual organic solvents in the final product, which is undesirable for a pharmaceutical product. It is labor and capital intensive to scale up processes like reverse phase chromatography (RP): utilizing for instance explosion proof facilities, HPLC columns and purification systems rated for high pressure, high temperature, flammable solvents etc. The scale and throughput for large scale manufacture are limited by these factors. Subsequent purification is also required to remove alkylammonium ion pair utilized in RP process. In contrast the methods described herein even enhance currently utilized methods (eg RP). Lower impurity load leads to higher purification recovery of full length RNA devoid of cytokine inducing contaminants eg. higher quality of materials at the outset.

The modified IVT methods involve the manipulation of one or more of the reaction parameters in the IVT reaction to produce a RNA preparation of highly functional RNA without one or more of the undesirable contaminants produced using the prior art processes. One parameter in the IVT reaction that may be manipulated is the relative amount of a nucleotide or nucleotide analog in comparison to one or more other nucleotides or nucleotide analogs in the reaction mixture (e.g., disparate nucleotide amounts or concentration). For instance, the IVT reaction may include an excess of a nucleotides, e.g., nucleotide monophosphate, nucleotide diphosphate or nucleotide triphosphate and/or an excess of nucleotide analogs and/or nucleoside analogs. The methods produce a high yield product which is significantly more pure than products produced by traditional IVT methods.

Nucleotide analogs are compounds that have the general structure of a nucleotide or are structurally similar to a nucleotide or portion thereof. In particular, nucleotide analogs are nucleotides which contain, for example, an analogue of the nucleic acid portion, sugar portion and/or phosphate groups of the nucleotide. Nucleotides include, for instance, nucleotide monophosphates, nucleotide diphosphates, and nucleotide triphosphates. A nucleotide analog, as used herein is structurally similar to a nucleotide or portion thereof but does not have the typical nucleotide structure (nucleobase-ribose-phosphate). Nucleoside analogs are compounds that have the general structure of a nucleoside or are structurally similar to a nucleoside or portion thereof. In particular, nucleoside analogs are nucleosides which contain, for example, an analogue of the nucleic acid and/or sugar portion of the nucleoside.

The nucleotide analogs useful in the methods are structurally similar to nucleotides or portions thereof but, for example, are not polymerizable by T7. Nucleotide/nucleoside analogs as used herein (including C, T, A, U, G, dC, dT, dA, dU, or dG analogs) include for instance, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia, or ligase), a nucleotide labelled with a functional group to facilitate ligation/conjugation of cap or 5′ moiety (IRES), a nucleotide labelled with a 5′ PO4 to facilitate ligation of cap or 5′ moiety, or a nucleotide labelled with a functional group/protecting group that can be chemically or enzymatically cleavable. Antiviral nucleotide/nucleoside analogs include but are not limited to Ganciclovir, Entecavir, Telbivudine, Vidarabine and Cidofovir.

The IVT reaction typically includes the following: an RNA polymerase, e.g., a T7 RNA polymerase at a final concentration of, e.g., 1000-12000 U/mL, e.g., 7000 U/mL; the DNA template at a final concentration of, e.g., 10-70 nM, e.g., 40 nM; nucleotides (NTPs) at a final concentration of e.g., 0.5-10 mM, e.g., 7.5 mM each; magnesium at a final concentration of, e.g., 12-60 mM, e.g., magnesium acetate at 40 mM; a buffer such as, e.g., HEPES or Tris at a pH of, e.g., 7-8.5, e.g. 40 mM Tris HCl, pH 8. In some embodiments 5 mM dithiothreitol (DTT) and/or 1 mM spermidine may be included. In some embodiments, an RNase inhibitor is included in the IVT reaction to ensure no RNase induced degradation during the transcription reaction. For example, murine RNase inhibitor can be utilized at a final concentration of 1000 U/mL. In some embodiments a pyrophosphatase is included in the IVT reaction to cleave the inorganic pyrophosphate generated following each nucleotide incorporation into two units of inorganic phosphate. This ensures that magnesium remains in solution and does not precipitate as magnesium pyrophosphate. For example, an E. coli inorganic pyrophosphatase can be utilized at a final concentration of 1 U/mL.

Similar to traditional methods, the modified method may also be produced by forming a reaction mixture comprising a DNA template, and one or more NTPs such as ATP, CTP, UTP, GTP (or corresponding analog of aforementioned components) and a buffer. The reaction is then incubated under conditions such that the RNA is transcribed. However, the modified methods utilize the presence of an excess amount of one or more nucleotides and/or nucleotide analogs that can have significant impact on the end product. These methods involve a modification in the amount (e.g., molar amount or quantity) of nucleotides and/or nucleotide analogs in the reaction mixture. In some aspects, one or more nucleotides and/or one or more nucleotide analogs may be added in excess to the reaction mixture. An excess of nucleotides and/or nucleotide analogs is any amount greater than the amount of one or more of the other nucleotides such as NTPs in the reaction mixture. For instance, an excess of a nucleotide and/or nucleotide analog may be a greater amount than the amount of each or at least one of the other individual NTPs in the reaction mixture or may refer to an amount greater than equimolar amounts of the other NTPs.

In the embodiment when the nucleotide and/or nucleotide analog that is included in the reaction mixture is an NTP, the NTP may be present in a higher concentration than all three of the other NTPs included in the reaction mixture. The other three NTPs may be in an equimolar concentration to one another. Alternatively one or more of the three other NTPs may be in a different concentration than one or more of the other NTPs.

Thus, in some embodiments the IVT reaction may include an equimolar amount of nucleotide triphosphate relative to at least one of the other nucleotide triphosphates.

In some embodiments the RNA is produced by a process or is preparable by a process comprising

(a) forming a reaction mixture comprising a DNA template and NTPs including adenosine triphosphate (ATP), cytidine triphosphate (CTP), uridine triphosphate (UTP), guanosine triphosphate (GTP) and optionally guanosine diphosphate (GDP), and (eg. buffer containing T7 co-factor eg. magnesium).

(b) incubating the reaction mixture under conditions such that the RNA is transcribed,

wherein the concentration of at least one of GTP, CTP, ATP, and UTP is at least 2× greater than the concentration of any one or more of ATP, CTP or UTP or the reaction further comprises a nucleotide analog and wherein the concentration of the nucleotide analog is at least 2× greater than the concentration of any one or more of ATP, CTP or UTP.

In some embodiments the ratio of concentration of GTP to the concentration of any one ATP, CTP or UTP is at least 2:1, at least 3:1, at least 4:1, at least 5:1 or at least 6:1. The ratio of concentration of GTP to concentration of ATP, CTP and UTP is, in some embodiments 2:1, 4:1 and 4:1, respectively. In other embodiments the ratio of concentration of GTP to concentration of ATP, CTP and UTP is 3:1, 6:1 and 6:1, respectively. The reaction mixture may comprise GTP and GDP and wherein the ratio of concentration of GTP plus GDP to the concentration of any one of ATP, CTP or UTP is at least 2:1, at least 3:1, at least 4:1, at least 5:1 or at least 6:1 In some embodiments the ratio of concentration of GTP plus GDP to concentration of ATP, CTP and UTP is 3:1, 6:1 and 6:1, respectively.

In some embodiments the method involves incubating the reaction mixture under conditions such that the RNA is transcribed, wherein the effective concentration of phosphate in the reaction is at least 150 mM phosphate, at least 160 mM, at least 170 mM, at least 180 mM, at least 190 mM, at least 200 mM, at least 210 mM or at least 220 mM. The effective concentration of phosphate in the reaction may be 180 mM. The effective concentration of phosphate in the reaction in some embodiments is 195 mM. In other embodiments the effective concentration of phosphate in the reaction is 225 mM.

In other embodiments the RNA is produced by a process or is preparable by a process comprising wherein a buffer magnesium-containing buffer is used when forming the reaction mixture comprising a DNA template and ATP, CTP, UTP, GTP. In some embodiments the magnesium-containing buffer comprises Mg2+ and wherein the molar ratio of concentration of ATP plus CTP plus UTP pus GTP to concentration of Mg2+ is at least 1.0, at least 1.25, at least 1.5, at least 1.75, at least 1.85, at least 3 or higher. The molar ratio of concentration of ATP plus CTP plus UTP pus GTP to concentration of Mg2+ may be 1.5. The molar ratio of concentration of ATP plus CTP plus UTP pus GTP to concentration of Mg2+ in some embodiments is 1.88. The molar ratio of concentration of ATP plus CTP plus UTP pus GTP to concentration of Mg2+ in some embodiments is 3.

In some embodiments the composition is produced by a process which does not comprise an dsRNase (e.g., RNaseIII) treatment step. In other embodiments the composition is produced by a process which does not comprise a reverse phase (RP) chromatography purification step. In yet other embodiments the composition is produced by a process which does not comprise a high-performance liquid chromatography (HPLC) purification step.

In some embodiments the ratio of concentration of GTP to the concentration of any one ATP, CTP or UTP is at least 2:1, at least 3:1, at least 4:1, at least 5:1 or at least 6:1 to produce the RNA.

The purity of the products may be assessed using known analytical methods and assays. For instance, the amount of reverse complement transcription product or cytokine-inducing RNA contaminant may be determined by high-performance liquid chromatography (such as reverse-phase chromatography, size-exclusion chromatography), Bioanalyzer chip-based electrophoresis system, ELISA, flow cytometry, acrylamide gel, a reconstitution or surrogate type assay. The assays may be performed with or without nuclease treatment (P1, RNase III, RNase H etc.) of the RNA preparation. Electrophoretic/chromatographic/mass spec analysis of nuclease digestion products may also be performed.

In some embodiments the purified RNA preparations comprise contaminant transcripts that have a length less than a full length transcript, such as for instance at least 100, 200, 300, 400, 500, 600, 700, 800, or 900 nucleotides less than the full length. Contaminant transcripts can include reverse or forward transcription products (transcripts) that have a length less than a full length transcript, such as for instance at least 100, 200, 300, 400, 500, 600, 700, 800, or 900 nucleotides less than the full length. Exemplary forward transcripts include, for instance, abortive transcripts. In certain embodiments the composition comprises a tri-phosphate poly-U reverse complement of less than 30 nucleotides. In some embodiments the composition comprises a tri-phosphate poly-U reverse complement of any length hybridized to a full length transcript. In other embodiments the composition comprises a single stranded tri-phosphate forward transcript. In other embodiments the composition comprises a single stranded RNA having a terminal tri-phosphate-G. In other embodiments the composition comprises single or double stranded RNA of less than 12 nucleotides or base pairs (including forward or reverse complement transcripts). In any of these embodiments the composition may include less than 50%, 45%, 40%, 35%, 30%, 25%, 20%, 15%, 10%, 9%, 8%, 7%, 6%, 5%, 4%, 3%, 2%, 1%, or 0.5% of any one of or combination of these less than full length transcripts.

In some embodiments, the polynucleotide of the invention (e.g., a polynucleotide comprising a nucleotide sequence encoding a therapeutic polypeptide) is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)).

In some embodiments, the polynucleotide of the invention (e.g., a polynucleotide comprising a nucleotide sequence a therapeutic polypeptide) purified using column chromatography (e.g., strong anion exchange HPLC, weak anion exchange HPLC, reverse phase HPLC (RP-HPLC, hydrophobic interaction HPLC (HIC-HPLC), or (LCMS)) presents increased expression of the encoded therapeutic protein compared to the expression level obtained with the same polynucleotide of the present disclosure purified by a different purification method.

In some embodiments, a column chromatography (e.g., strong anion exchange HPLC, weak anion exchange HPLC, reverse phase HPLC (RP-HPLC), hydrophobic interaction HPLC (HIC-HPLC), or (LCMS)) purified polynucleotide comprises a nucleotide sequence encoding a therapeutic polypeptide comprising one or more of the point mutations known in the art.

In some embodiments, the use of RP-HPLC purified polynucleotide increases therapeutic protein expression levels in cells when introduced into those cells, e.g., by 10-100%, i.e., at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 90%, at least about 95%, or at least about 100% with respect to the expression levels of therapeutic protein in the cells before the RP-HPLC purified polynucleotide was introduced in the cells, or after a non-RP-HPLC purified polynucleotide was introduced in the cells.

In some embodiments, the use of RP-HPLC purified polynucleotide increases functional therapeutic protein expression levels in cells when introduced into those cells, e.g., by 10-100%, i.e., at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 90%, at least about 95%, or at least about 100% with respect to the functional expression levels of therapeutic protein in the cells before the RP-HPLC purified polynucleotide was introduced in the cells, or after a non-RP-HPLC purified polynucleotide was introduced in the cells.

In some embodiments, the use of RP-HPLC purified polynucleotide increases detectable therapeutic activity in cells when introduced into those cells, e.g., by 10-100%, i.e., at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 90%, at least about 95%, or at least about 100% with respect to the activity levels of functional therapeutic protein in the cells before the RP-HPLC purified polynucleotide was introduced in the cells, or after a non-RP-HPLC purified polynucleotide was introduced in the cells.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC. In another embodiment, the polynucleotide can be sequenced by methods including, but not limited to reverse-transcriptase-PCR.

d. Quantification of Expressed Polynucleotides Encoding Therapeutic Protein

In some embodiments, the polynucleotides of the present invention (e.g., a polynucleotide comprising a nucleotide sequence encoding a therapeutic polypeptide), their expression products, as well as degradation products and metabolites can be quantified according to methods known in the art.

In some embodiments, the polynucleotides of the present invention can be quantified in exosomes or when derived from one or more bodily fluid. As used herein “bodily fluids” include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes can be retrieved from an organ selected from the group consisting of lung, heart, pancreas, stomach, intestine, bladder, kidney, ovary, testis, skin, colon, breast, prostate, brain, esophagus, liver, and placenta.

In the exosome quantification method, a sample of not more than 2 mL is obtained from the subject and the exosomes isolated by size exclusion chromatography, density gradient centrifugation, differential centrifugation, nanomembrane ultrafiltration, immunoabsorbent capture, affinity purification, microfluidic separation, or combinations thereof. In the analysis, the level or concentration of a polynucleotide can be an expression level, presence, absence, truncation or alteration of the administered construct. It is advantageous to correlate the level with one or more clinical phenotypes or with an assay for a human disease biomarker.

The assay can be performed using construct specific probes, cytometry, qRT-PCR, real-time PCR, PCR, flow cytometry, electrophoresis, mass spectrometry, or combinations thereof while the exosomes can be isolated using immunohistochemical methods such as enzyme linked immunosorbent assay (ELISA) methods. Exosomes can also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of polynucleotides remaining or delivered. This is possible because the polynucleotides of the present invention differ from the endogenous forms due to the structural or chemical modifications.

In some embodiments, the polynucleotide can be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified polynucleotide can be analyzed in order to determine if the polynucleotide can be of proper size, check that no degradation of the polynucleotide has occurred. Degradation of the polynucleotide can be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Pharmaceutical Compositions and Formulations

The present invention provides pharmaceutical compositions and formulations that comprise any of the polynucleotides described above. In some embodiments, the composition or formulation further comprises a delivery agent.

In some embodiments, the composition or formulation can contain a polynucleotide comprising a sequence optimized nucleic acid sequence disclosed herein which encodes a therapeutic polypeptide. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes a therapeutic polypeptide. In some embodiments, the polynucleotide further comprises a miRNA binding site, e.g., a miRNA binding site that binds miR-142.

Pharmaceutical compositions or formulation can optionally comprise one or more additional active substances, e.g., therapeutically and/or prophylactically active substances. Pharmaceutical compositions or formulation of the present invention can be sterile and/or pyrogen-free. General considerations in the formulation and/or manufacture of pharmaceutical agents can be found, for example, in Remington: The Science and Practice of Pharmacy 21^{st} ed., Lippincott Williams & Wilkins, 2005 (incorporated herein by reference in its entirety). In some embodiments, compositions are administered to humans, human patients or subjects. For the purposes of the present disclosure, the phrase “active ingredient” generally refers to polynucleotides to be delivered as described herein.

Formulations and pharmaceutical compositions described herein can be prepared by any method known or hereafter developed in the art of pharmacology. In general, such preparatory methods include the step of associating the active ingredient with an excipient and/or one or more other accessory ingredients, and then, if necessary and/or desirable, dividing, shaping and/or packaging the product into a desired single- or multi-dose unit.

A pharmaceutical composition or formulation in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1 and 30%, between 5 and 80%, or at least 80% (w/w) active ingredient.

In some embodiments, the compositions and formulations described herein can contain at least one polynucleotide of the invention. As a non-limiting example, the composition or formulation can contain 1, 2, 3, 4 or 5 polynucleotides of the invention. In some embodiments, the compositions or formulations described herein can comprise more than one type of polynucleotide. In some embodiments, the composition or formulation can comprise a polynucleotide in linear and circular form. In another embodiment, the composition or formulation can comprise a circular polynucleotide and an IVT polynucleotide. In yet another embodiment, the composition or formulation can comprise an IVT polynucleotide, a chimeric polynucleotide and a circular polynucleotide.

Although the descriptions of pharmaceutical compositions and formulations provided herein are principally directed to pharmaceutical compositions and formulations that are suitable for administration to humans, it will be understood by the skilled artisan that such compositions are generally suitable for administration to any other animal, e.g., to non-human animals, e.g. non-human mammals. Modification of pharmaceutical compositions or formulations suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions or formulation is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

The present invention provides pharmaceutical formulations that comprise a polynucleotide described herein (e.g., a polynucleotide comprising a nucleotide sequence encoding a therapeutic polypeptide). The polynucleotides described herein can be formulated using one or more excipients to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In some embodiments, the pharmaceutical formulation further comprises a delivery agent, (e.g., a compound having the Formula (I), e.g., any of Compounds 1-20 or 25).

A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, Md., 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary granulating and/or dispersing agents include, but are not limited to, starches, pregelatinized starches, or microcrystalline starch, alginic acid, guar gum, agar, poly(vinyl-pyrrolidone), (providone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate (VEEGUM®), sodium lauryl sulfate, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN® 80], sorbitan monopalmitate [SPAN® 40], glyceryl monooleate, polyoxyethylene esters, polyethylene glycol fatty acid esters (e.g., CREMOPHOR®), polyoxyethylene ethers (e.g., polyoxyethylene lauryl ether [BRIJ® 30]), PLUORINC® F 68, POLOXAMER® 188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethyl cellulose, hydroxypropyl methylcellulose, etc., and combinations thereof.

Oxidation is a potential degradation pathway for mRNA, especially for liquid mRNA formulations. In order to prevent oxidation, antioxidants can be added to the formulations. Exemplary antioxidants include, but are not limited to, alpha tocopherol, ascorbic acid, acorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of polynucleotide solutions are maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc. and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition or formulation described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition or formulation described here can contain a bulking agent in lyophilized polynucleotide formulations to yield a “pharmaceutically elegant” cake, stabilize the lyophilized polynucleotides during long term (e.g., 36 month) storage. Exemplary bulking agents of the present invention can include, but are not limited to sucrose, trehalose, mannitol, glycine, lactose, raffinose, and combinations thereof.

In some embodiments, the pharmaceutical composition or formulation further comprises a delivery agent.

### Delivery Agents

a. Lipid Compound

The present disclosure provides pharmaceutical compositions with advantageous properties. The disclosure relates to novel lipids and lipid nanoparticle compositions including a novel lipid. The disclosure also provides methods of delivering a therapeutic and/or prophylactic to a mammalian cell, specifically delivering a therapeutic and/or prophylactic to a mammalian organ, producing a polypeptide of interest in a mammalian cell, and treating a disease or disorder in a mammal in need thereof. For example, a method of producing a polypeptide of interest in a cell involves contacting a nanoparticle composition comprising an mRNA with a mammalian cell, whereby the mRNA may be translated to produce the polypeptide of interest. A method of delivering a therapeutic and/or prophylactic to a mammalian cell or organ may involve administration of a nanoparticle composition including the therapeutic and/or prophylactic to a subject, in which the administration involves contacting the cell or organ with the composition, whereby the therapeutic and/or prophylactic is delivered to the cell or organ.

### Lipid Nanoparticles (LNPs)

In some embodiments, therapeutic RNA (e.g., mRNA) vaccines of the disclosure are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, an ionizable cationic lipid of the disclosure comprises a compound of Formula (I):

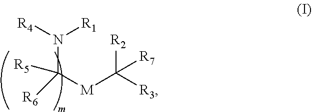

or a salt or isomer thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)—CHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)—N(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)—OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)—CHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)—CHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)—CHQR, —CHQR,

and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, a subset of compounds of Formula (I) includes those of Formula (IA):

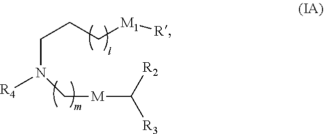

or a salt or isomer thereof, wherein l is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected

from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

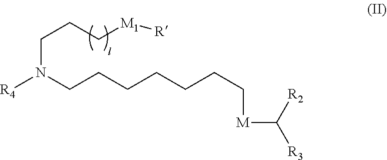

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

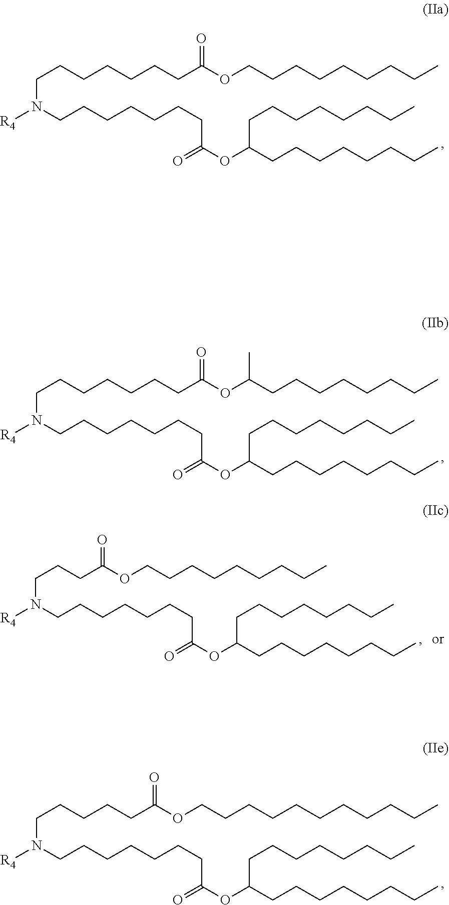

or a salt or isomer thereof, wherein R₄ is as described herein.

In some embodiments, a subset of compounds of Formula (I) includes those of Formula (IId):

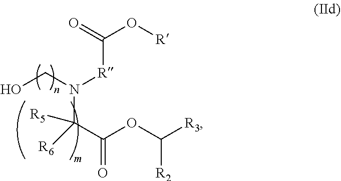

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

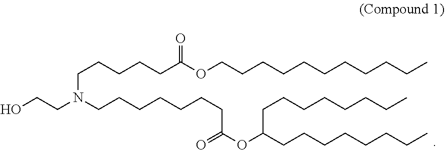 [SMILES: CCCCCCCCCCCOC(=O)CCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

In some embodiments, the compound of Formula (I) is selected from the group consisting of:

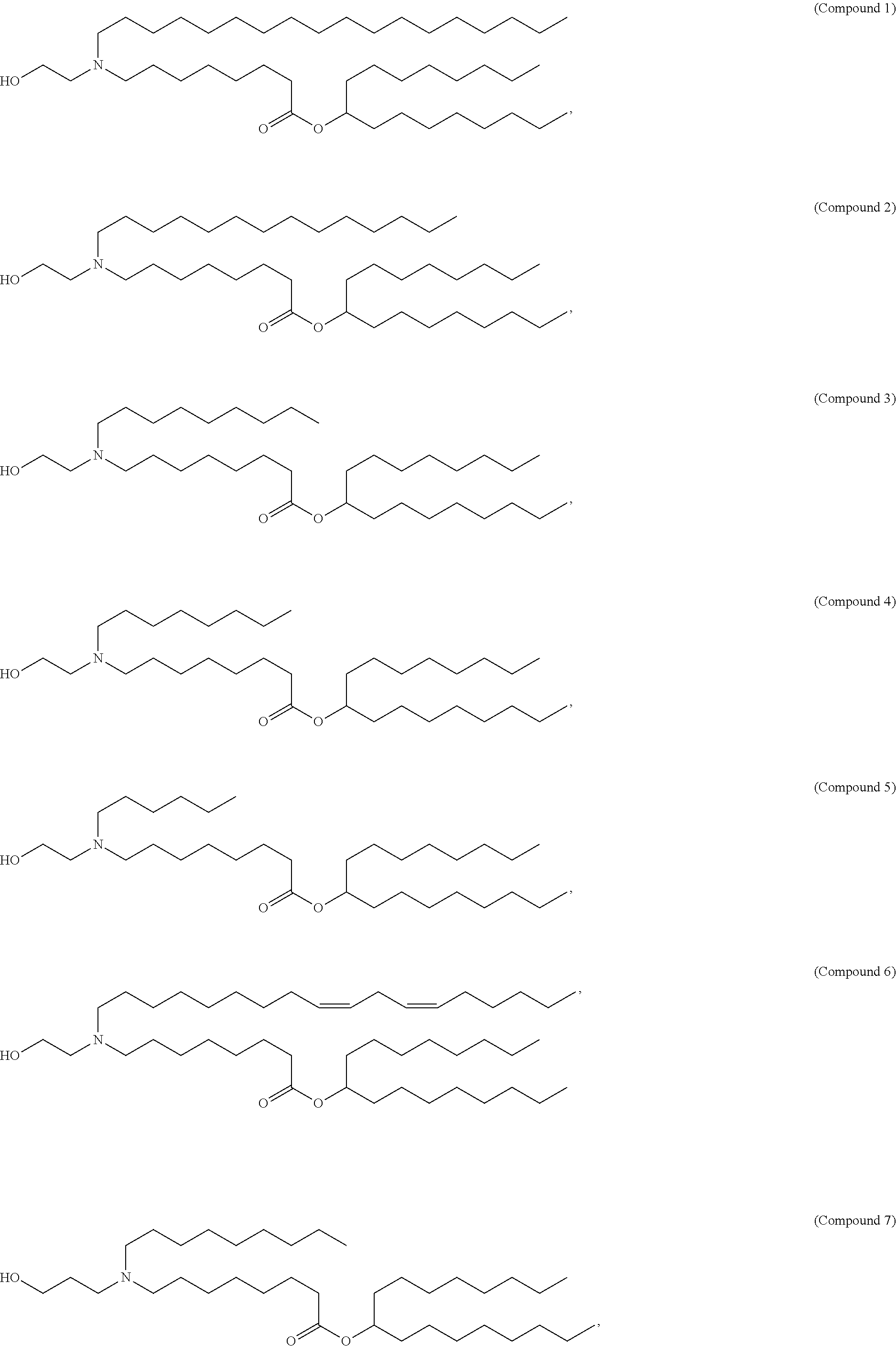

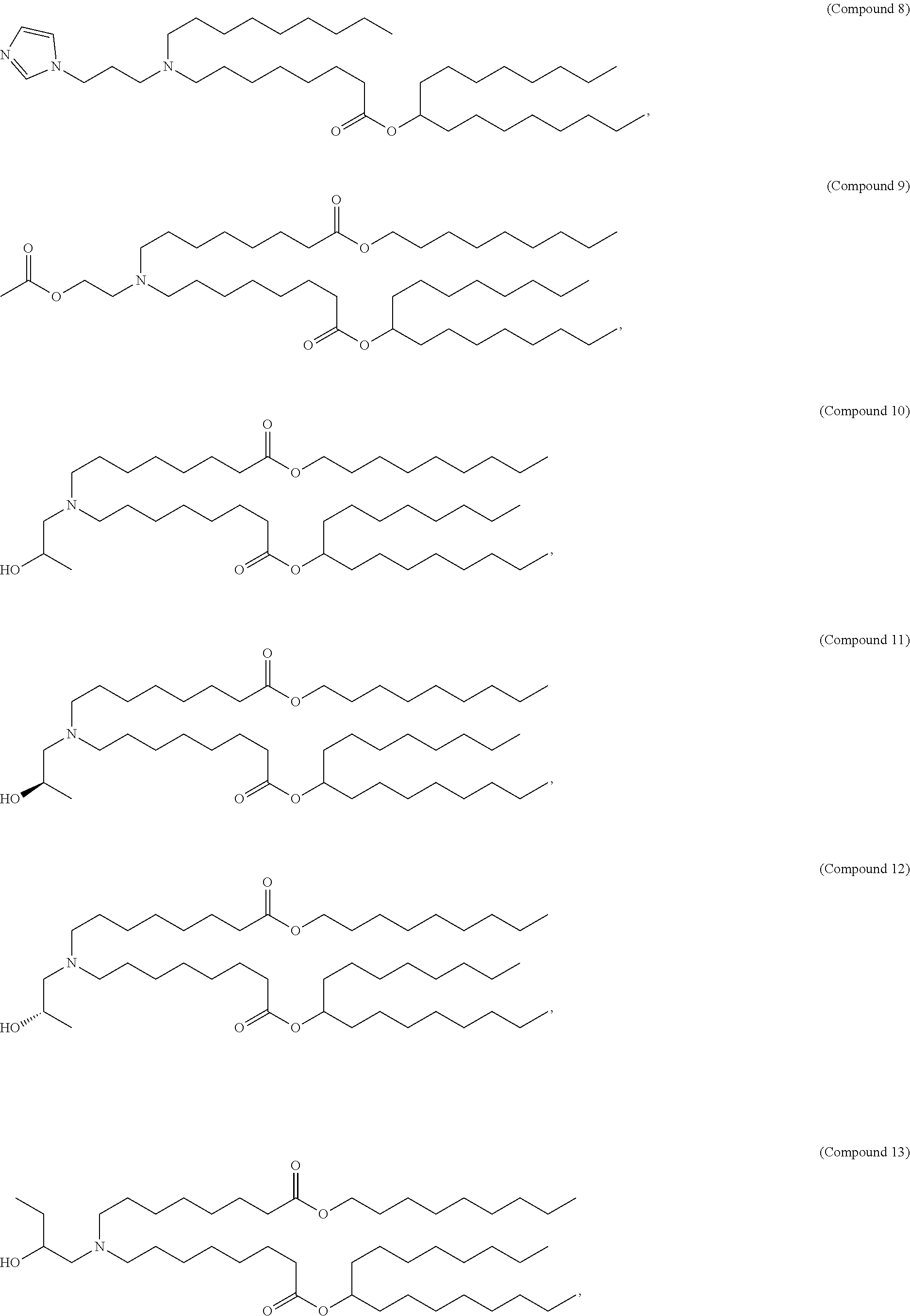

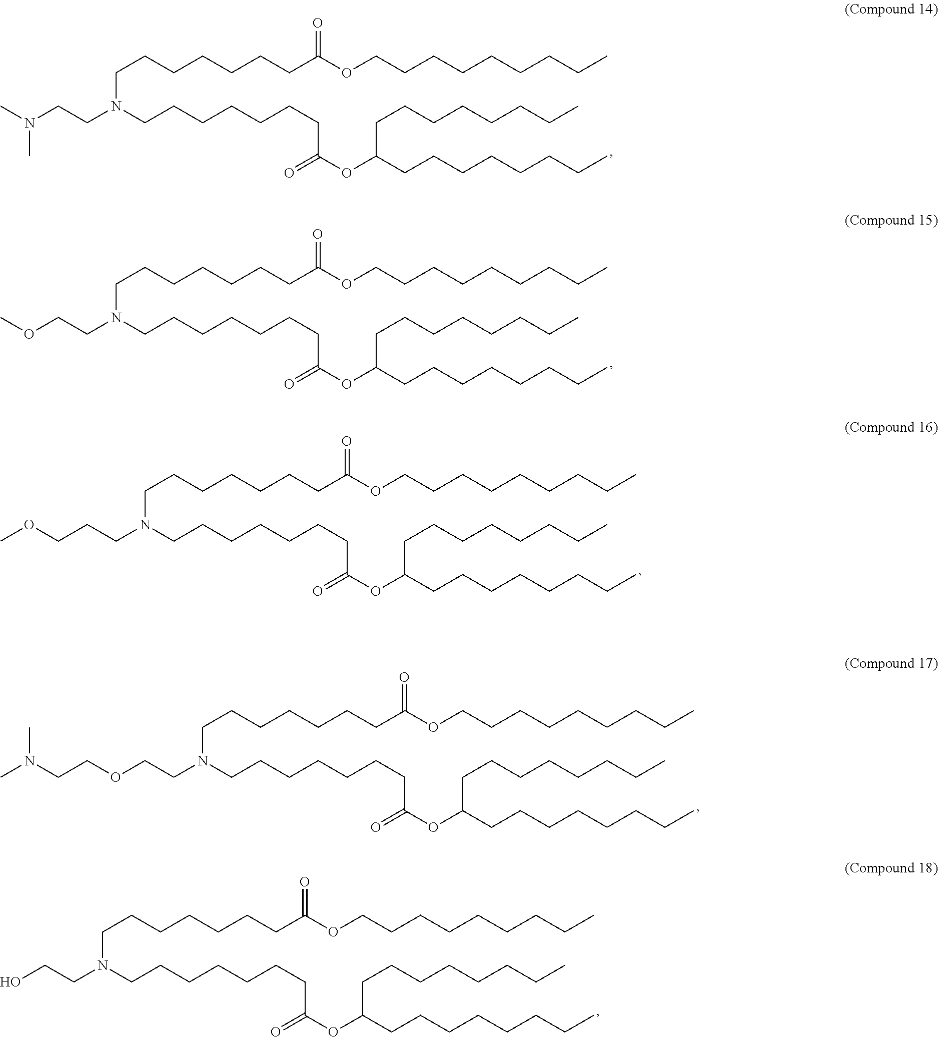

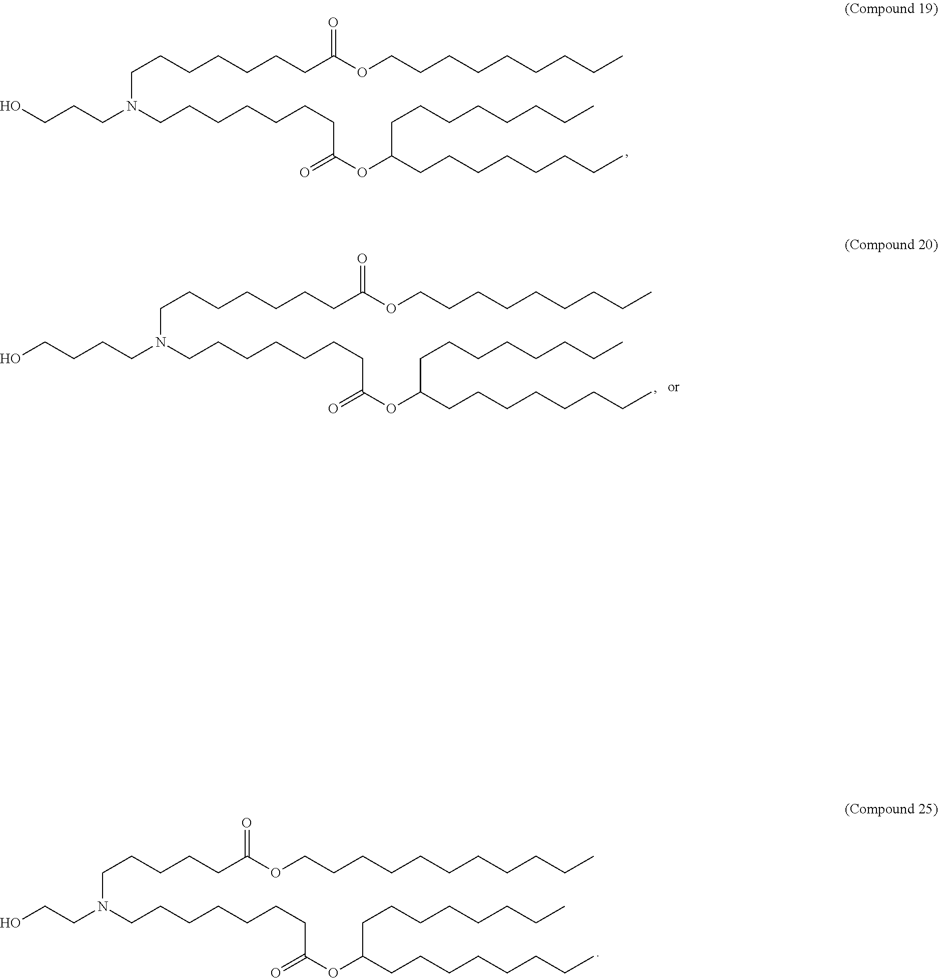 [SMILES: CCCCCCCCCOC(=O)CCCCCCCN(CCCO)CCCCCCCC(=O)OC(CCCCCCCC)CCCCCCCC ; CCCCCCCCCOC(=O)CCCCCCCN(CCCCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In another aspect, the present application provides a lipid composition (e.g., a lipid nanoparticle (LNP)) comprising: (1) a compound having the formula (I); (2) optionally a helper lipid (e.g. a phospholipid); (3) optionally a structural lipid (e.g. a sterol); (4) optionally a lipid conjugate (e.g. a PEG-lipid); and (5) optionally a quaternary amine compound. In exemplary embodiments, the lipid composition (e.g., LNP) further comprises a polynucleotide encoding a therapeutic polypeptide, e.g., a polynucleotide encapsulated therein.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. For example, C₁₈ alkenyl may include one or more double bonds. A C₁₈ alkenyl group including two double bonds may be a linoleyl group. Unless otherwise specified, an alkenyl group described herein refers to both unsubstituted and substituted alkenyl groups.

As used herein, the term “alkynyl” or “alkynyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C₁₈ alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2-dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R″″, in which each OR are alkoxy groups that can be the same or different and R′″ is an alkyl or alkenyl group), a phosphate (e.g., P(O)₄³⁻), a thiol (e.g., —SH), a sulfoxide (e.g., —S(O)R), a sulfinic acid (e.g., —S(O)OH), a sulfonic acid (e.g., —S(O)₂OH), a thial (e.g., —C(S)H), a sulfate (e.g., S(O)₄²⁻), a sulfonyl (e.g., —S(O)₂—), an amide (e.g., —C(O)NR₂, or —N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C₁₋₆ alkyl group may be further substituted with one, two, three, four, five, or six substituents as described herein.

About, Approximately: As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean+/−10% of the recited value. For instance, a nanoparticle composition including a lipid component having about 40% of a given compound may include 30-50% of the compound.

As used herein, the term “compound,” is meant to include all isomers and isotopes of the structure depicted. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver) compared to the level of delivery of a therapeutic and/or prophylactic by a control nanoparticle to a target tissue of interest (e.g., MC3, KC2, or DLinDMA). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. It will be understood that the enhanced delivery of a nanoparticle to a target tissue need not be determined in a subject being treated, it may be determined in a surrogate such as an animal model (e.g., a rat model). In certain embodiments, a nanoparticle composition including a compound according to Formula (I), (IA), (II), (IIa), (IIb), (IIc), (IId) or (IIe) has substantively the same level of delivery enhancement regardless of administration routes. For example, certain compounds disclosed herein exhibit similar delivery enhancement when they are used for delivering a therapeutic and/or prophylactic either intravenously or intramuscularly. In other embodiments, certain compounds disclosed herein (e.g., a compound of Formula (IA) or (II), such as Compound 18, 25, 30, 60, 108-112, or 122) exhibit a higher level of delivery enhancement when they are used for delivering a therapeutic and/or prophylactic intramuscularly than intravenously.

As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target tissue of interest (e.g., mammalian liver) compared to an off-target tissue (e.g., mammalian spleen). The level of delivery of a nanoparticle to a particular tissue may be measured by comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. For example, for renovascular targeting, a therapeutic and/or prophylactic is specifically provided to a mammalian kidney as compared to the liver and spleen if 1.5, 2-fold, 3-fold, 5-fold, 10-fold, 15 fold, or 20 fold more therapeutic and/or prophylactic per 1 g of tissue is delivered to a kidney compared to that delivered to the liver or spleen following systemic administration of the therapeutic and/or prophylactic. It will be understood that the ability of a nanoparticle to specifically deliver to a target tissue need not be determined in a subject being treated, it may be determined in a surrogate such as an animal model (e.g., a rat model).

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, “expression” of a nucleic acid sequence refers to translation of an mRNA into a polypeptide or protein and/or post-translational modification of a polypeptide or protein.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (−)) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

As used herein, a “lipid component” is that component of a nanoparticle composition that includes one or more lipids. For example, the lipid component may include one or more cationic/ionizable, PEGylated, structural, or other lipids, such as phospholipids.

As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. For example, two nucleosides of a cap analog may be linked at their 5′ positions by a triphosphate group or by a chain including two phosphate moieties and a boranophosphate moiety.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

As used herein, the “N:P ratio” is the molar ratio of ionizable (in the physiological pH range) nitrogen atoms in a lipid to phosphate groups in an RNA, e.g., in a nanoparticle composition including a lipid component and an RNA.

As used herein, a “nanoparticle composition” is a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less.

As used herein, “naturally occurring” means existing in nature without artificial aid.

As used herein, “patient” refers to a subject who may seek or be in need of treatment, requires treatment, is receiving treatment, will receive treatment, or a subject who is under care by a trained professional for a particular disease or condition.

As used herein, a “PEG lipid” or “PEGylated lipid” refers to a lipid comprising a polyethylene glycol component.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, the “polydispersity index” is a ratio that describes the homogeneity of the particle size distribution of a system. A small value, e.g., less than 0.3, indicates a narrow particle size distribution.

As used herein, the term “polypeptide” or “polypeptide of interest” refers to a polymer of amino acid residues typically joined by peptide bonds that can be produced naturally (e.g., isolated or purified) or synthetically.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

As used herein, a “single unit dose” is a dose of any therapeutic administered in one dose/at one time/single route/single point of contact, i.e., single administration event.

As used herein, a “split dose” is the division of single unit dose or total daily dose into two or more doses.

As used herein, a “total daily dose” is an amount given or prescribed in 24 hour period. It may be administered as a single unit dose.

As used herein, “size” or “mean size” in the context of nanoparticle compositions refers to the mean diameter of a nanoparticle composition.

As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient.

As used herein “target tissue” refers to any one or more tissue types of interest in which the delivery of a therapeutic and/or prophylactic would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

The term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.” Such agents include, but are not limited to, cytotoxins, radioactive ions, chemotherapeutic agents, small molecule drugs, proteins, and nucleic acids.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, “transfection” refers to the introduction of a species (e.g., an RNA) into a cell. Transfection may occur, for example, in vitro, ex vivo, or in vivo.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the “zeta potential” is the electrokinetic potential of a lipid, e.g., in a particle composition.

In one specific embodiment, the compound of formula (I) is Compound 18.

In some embodiments, the amount the compound of formula (I) ranges from about 1 mol % to 99 mol % in the lipid composition.

In one embodiment, the amount of compound of formula (I) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the compound of formula (I) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the compound of formula (I) is about 50 mol % in the lipid composition.

In addition to the compound of formula (I), the lipid composition of the pharmaceutical compositions disclosed herein can comprise additional components such as phospholipids, structural lipids, quaternary amine compounds, PEG-lipids, and any combination thereof.

b. Additional Components in the Lipid Composition

The disclosure also features nanoparticle compositions comprising a lipid component comprising a compound according to Formula (I), (IA), (II), (IIa), (IIb), (IIc), (IId) or (IIe) as described herein.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels.

Nanoparticle compositions comprise a lipid component including at least one compound according to Formula (I), (IA), (II), (IIa), (IIb), (IIc), (IId) or (IIe). For example, the lipid component of a nanoparticle composition may include one or more of Compounds 1-20 or 25. Nanoparticle compositions may also include a variety of other components. For example, the lipid component of a nanoparticle composition may include one or more other lipids in addition to a lipid according to Formula (I), (IA), (II), (IIa), (IIb), (IIc), (IId) or (IIe).

### (i) Cationic/Ionizable Lipids

A nanoparticle composition may include one or more cationic and/or ionizable lipids (e.g., lipids that may have a positive or partial positive charge at physiological pH) in addition to a lipid according to Formula (I), (IA), (II), (IIa), (IIb), (IIc), (IId) or (IIe). Cationic and/or ionizable lipids may be selected from the non-limiting group consisting of
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
heptatriaconta-6,9,28,31-tetraen-19-yl 4-(dimethylamino)butanoate (DLin-MC3-DMA),
2,2-dilinoleyl-4-(2-dimethylaminoethyl)[1,3]-dioxolane (DLin-KC2-DMA),
1,2-dioleyloxy-N,N-dimethylaminopropane (DODMA),
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, a cationic lipid may also be a lipid including a cyclic amine group.

### (ii) PEG Lipids

The lipid component of a nanoparticle composition may include one or more PEG or PEG-modified lipids. Such species may be alternately referred to as PEGylated lipids. A PEG lipid is a lipid modified with polyethylene glycol. A PEG lipid may be selected from the non-limiting group consisting of PEG-modified phosphatidylethanolamines, PEG-modified phosphatidic acids, PEG-modified ceramides, PEG-modified di alkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

(iii) Structural Lipids

The lipid component of a nanoparticle composition may include one or more structural lipids. Structural lipids can be selected from the group consisting of, but are not limited to, cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof.

### (iv) Phospholipids

The lipid composition of the pharmaceutical composition disclosed herein can comprise one or more phospholipids, for example, one or more saturated or (poly)unsaturated phospholipids or a combination thereof. In general, phospholipids comprise a phospholipid moiety and one or more fatty acid moieties. For example, a phospholipid can be a lipid according to formula (III):

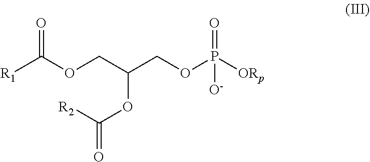

in which Rₚ represents a phospholipid moiety and R₁ and R₂ represent fatty acid moieties with or without unsaturation that may be the same or different. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids useful in the compositions and methods may be selected from the non-limiting group consisting of 1,2-distearoyl-sn-glycero-3-phosphocholine (DSPC),
1,2-dioleoyl-sn-glycero-3-phosphoethanolamine (DOPE),
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-diphytanoyl-sn-glycero-3-phosphoethanolamine (ME 16.0 PE),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), and sphingomyelin.
   In some embodiments, a nanoparticle composition includes DSPC. In certain embodiments, a nanoparticle composition includes DOPE. In some embodiments, a nanoparticle composition includes both DSPC and DOPE.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue (e.g., tumoral tissue).

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin. In some embodiments, a pharmaceutical composition for intratumoral delivery disclosed herein can comprise more than one phospholipid. When more than one phospholipid is used, such phospholipids can belong to the same phospholipid class (e.g., MSPC and DSPC) or different classes (e.g., MSPC and MSPE).

Phospholipids can be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospholipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain).

In some embodiments, the lipid composition of a pharmaceutical composition disclosed herein comprises at least one symmetric phospholipid. Symmetric phospholipids can be selected from the non-limiting group consisting of
1,2-dipropionyl-sn-glycero-3-phosphocholine (03:0 PC),
1,2-dibutyryl-sn-glycero-3-phosphocholine (04:0 PC),
1,2-dipentanoyl-sn-glycero-3-phosphocholine (05:0 PC),
1,2-dihexanoyl-sn-glycero-3-phosphocholine (06:0 PC),
1,2-diheptanoyl-sn-glycero-3-phosphocholine (07:0 PC),
1,2-dioctanoyl-sn-glycero-3-phosphocholine (08:0 PC),
1,2-dinonanoyl-sn-glycero-3-phosphocholine (09:0 PC),
1,2-didecanoyl-sn-glycero-3-phosphocholine (10:0 PC),
1,2-diundecanoyl-sn-glycero-3-phosphocholine (11:0 PC, DUPC),
1,2-dilauroyl-sn-glycero-3-phosphocholine (12:0 PC),
1,2-ditridecanoyl-sn-glycero-3-phosphocholine (13:0 PC),
1,2-dimyristoyl-sn-glycero-3-phosphocholine (14:0 PC, DMPC),
1,2-dipentadecanoyl-sn-glycero-3-phosphocholine (15:0 PC),
1,2-dipalmitoyl-sn-glycero-3-phosphocholine (16:0 PC, DPPC),
1,2-diphytanoyl-sn-glycero-3-phosphocholine (4ME 16:0 PC),
1,2-diheptadecanoyl-sn-glycero-3-phosphocholine (17:0 PC),
1,2-distearoyl-sn-glycero-3-phosphocholine (18:0 PC, DSPC),
1,2-dinonadecanoyl-sn-glycero-3-phosphocholine (19:0 PC),
1,2-diarachidoyl-sn-glycero-3-phosphocholine (20:0 PC),
1,2-dihenarachidoyl-sn-glycero-3-phosphocholine (21:0 PC),
1,2-dibehenoyl-sn-glycero-3-phosphocholine (22:0 PC),
1,2-ditricosanoyl-sn-glycero-3-phosphocholine (23:0 PC),
1,2-dilignoceroyl-sn-glycero-3-phosphocholine (24:0 PC),
1,2-dimyristoleoyl-sn-glycero-3-phosphocholine (14:1 (Δ9-Cis) PC),
1,2-dimyristelaidoyl-sn-glycero-3-phosphocholine (14:1 (Δ9-Trans) PC),
1,2-dipalmitoleoyl-sn-glycero-3-phosphocholine (16:1 (Δ9-Cis) PC),
1,2-dipalmitelaidoyl-sn-glycero-3-phosphocholine (16:1 (Δ9-Trans) PC),
1,2-dipetroselenoyl-sn-glycero-3-phosphocholine (18:1 (Δ6-Cis) PC),
1,2-dioleoyl-sn-glycero-3-phosphocholine (18:1 (Δ9-Cis) PC, DOPC),
1,2-dielaidoyl-sn-glycero-3-phosphocholine (18:1 (Δ9-Trans) PC),
1,2-dilinoleoyl-sn-glycero-3-phosphocholine (18:2 (Cis) PC, DLPC),
1,2-dilinolenoyl-sn-glycero-3-phosphocholine (18:3 (Cis) PC, DLnPC),
1,2-dieicosenoyl-sn-glycero-3-phosphocholine (20:1 (Cis) PC),
1,2-diarachidonoyl-sn-glycero-3-phosphocholine (20:4 (Cis) PC, DAPC),
1,2-dierucoyl-sn-glycero-3-phosphocholine (22:1 (Cis) PC),
1,2-didocosahexaenoyl-sn-glycero-3-phosphocholine (22:6 (Cis) PC, DHAPC),
1,2-dinervonoyl-sn-glycero-3-phosphocholine (24:1 (Cis) PC),
1,2-dihexanoyl-sn-glycero-3-phosphoethanolamine (06:0 PE),
1,2-dioctanoyl-sn-glycero-3-phosphoethanolamine (08:0 PE),
1,2-didecanoyl-sn-glycero-3-phosphoethanolamine (10:0 PE),
1,2-dilauroyl-sn-glycero-3-phosphoethanolamine (12:0 PE),
1,2-dimyristoyl-sn-glycero-3-phosphoethanolamine (14:0 PE),
1,2-dipentadecanoyl-sn-glycero-3-phosphoethanolamine (15:0 PE),
1,2-dipalmitoyl-sn-glycero-3-phosphoethanolamine (16:0 PE),
1,2-diphytanoyl-sn-glycero-3-phosphoethanolamine (4ME 16:0 PE),
1,2-diheptadecanoyl-sn-glycero-3-phosphoethanolamine (17:0 PE),
1,2-distearoyl-sn-glycero-3-phosphoethanolamine (18:0 PE, DSPE),
1,2-dipalmitoleoyl-sn-glycero-3-phosphoethanolamine (16:1 PE),
1,2-dioleoyl-sn-glycero-3-phosphoethanolamine (18:1 (Δ9-Cis) PE, DOPE),
1,2-dielaidoyl-sn-glycero-3-phosphoethanolamine (18:1 (Δ9-Trans) PE),
1,2-dilinoleoyl-sn-glycero-3-phosphoethanolamine (18:2 PE, DLPE),
1,2-dilinolenoyl-sn-glycero-3-phosphoethanolamine (18:3 PE, DLnPE),
1,2-diarachidonoyl-sn-glycero-3-phosphoethanolamine (20:4 PE, DAPE),
1,2-didocosahexaenoyl-sn-glycero-3-phosphoethanolamine (22:6 PE, DHAPE),
1,2-di-O-octadecenyl-sn-glycero-3-phosphocholine (18:0 Diether PC),
1,2-dioleoyl-sn-glycero-3-phospho-rac-(1-glycerol) sodium salt (DOPG), and

any combination thereof.

In some embodiments, the lipid composition of a pharmaceutical composition disclosed herein comprises at least one symmetric phospholipid selected from the non-limiting group consisting of DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof.

In some embodiments, the lipid composition of a pharmaceutical composition disclosed herein comprises at least one asymmetric phospholipid. Asymmetric phospholipids can be selected from the non-limiting group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-acetyl-sn-glycero-3-phosphocholine (16:0-02:0 PC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-palmitoyl-2-oleoyl-sn-glycero-3-phosphocholine (16:0-18:1 PC, POPC),
1-palmitoyl-2-linoleoyl-sn-glycero-3-phosphocholine (16:0-18:2 PC, PLPC),
1-palmitoyl-2-arachidonoyl-sn-glycero-3-phosphocholine (16:0-20:4 PC),
1-palmitoyl-2-docosahexaenoyl-sn-glycero-3-phosphocholine (14:0-22:6 PC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC),
1-stearoyl-2-oleoyl-sn-glycero-3-phosphocholine (18:0-18:1 PC, SOPC),
1-stearoyl-2-linoleoyl-sn-glycero-3-phosphocholine (18:0-18:2 PC),
1-stearoyl-2-arachidonoyl-sn-glycero-3-phosphocholine (18:0-20:4 PC),
1-stearoyl-2-docosahexaenoyl-sn-glycero-3-phosphocholine (18:0-22:6 PC),
1-oleoyl-2-myristoyl-sn-glycero-3-phosphocholine (18:1-14:0 PC, OMPC),
1-oleoyl-2-palmitoyl-sn-glycero-3-phosphocholine (18:1-16:0 PC, OPPC),
1-oleoyl-2-stearoyl-sn-glycero-3-phosphocholine (18:1-18:0 PC, OSPC),
1-palmitoyl-2-oleoyl-sn-glycero-3-phosphoethanolamine (16:0-18:1 PE, POPE),
1-palmitoyl-2-linoleoyl-sn-glycero-3-phosphoethanolamine (16:0-18:2 PE),
1-palmitoyl-2-arachidonoyl-sn-glycero-3-phosphoethanolamine (16:0-20:4 PE),
1-palmitoyl-2-docosahexaenoyl-sn-glycero-3-phosphoethanolamine (16:0-22:6 PE),
1-stearoyl-2-oleoyl-sn-glycero-3-phosphoethanolamine (18:0-18:1 PE),
1-stearoyl-2-linoleoyl-sn-glycero-3-phosphoethanolamine (18:0-18:2 PE),
1-stearoyl-2-arachidonoyl-sn-glycero-3-phosphoethanolamine (18:0-20:4 PE),
1-stearoyl-2-docosahexaenoyl-sn-glycero-3-phosphoethanolamine (18:0-22:6 PE),
1-oleoyl-2-cholesterylhemisuccinoyl-sn-glycero-3-phosphocholine (OChemsPC), and

any combination thereof.

Asymmetric lipids useful in the lipid composition can also be lysolipids. Lysolipids can be selected from the non-limiting group consisting of
1-hexanoyl-2-hydroxy-sn-glycero-3-phosphocholine (06:0 Lyso PC),
1-heptanoyl-2-hydroxy-sn-glycero-3-phosphocholine (07:0 Lyso PC),
1-octanoyl-2-hydroxy-sn-glycero-3-phosphocholine (08:0 Lyso PC),
1-nonanoyl-2-hydroxy-sn-glycero-3-phosphocholine (09:0 Lyso PC),
1-decanoyl-2-hydroxy-sn-glycero-3-phosphocholine (10:0 Lyso PC),
1-undecanoyl-2-hydroxy-sn-glycero-3-phosphocholine (11:0 Lyso PC),
1-lauroyl-2-hydroxy-sn-glycero-3-phosphocholine (12:0 Lyso PC),
1-tridecanoyl-2-hydroxy-sn-glycero-3-phosphocholine (13:0 Lyso PC),
1-myristoyl-2-hydroxy-sn-glycero-3-phosphocholine (14:0 Lyso PC),
1-pentadecanoyl-2-hydroxy-sn-glycero-3-phosphocholine (15:0 Lyso PC),
1-palmitoyl-2-hydroxy-sn-glycero-3-phosphocholine (16:0 Lyso PC),
1-heptadecanoyl-2-hydroxy-sn-glycero-3-phosphocholine (17:0 Lyso PC),
1-stearoyl-2-hydroxy-sn-glycero-3-phosphocholine (18:0 Lyso PC),
1-oleoyl-2-hydroxy-sn-glycero-3-phosphocholine (18:1 Lyso PC),
1-nonadecanoyl-2-hydroxy-sn-glycero-3-phosphocholine (19:0 Lyso PC),
1-arachidoyl-2-hydroxy-sn-glycero-3-phosphocholine (20:0 Lyso PC),
1-behenoyl-2-hydroxy-sn-glycero-3-phosphocholine (22:0 Lyso PC),
1-lignoceroyl-2-hydroxy-sn-glycero-3-phosphocholine (24:0 Lyso PC),
1-hexacosanoyl-2-hydroxy-sn-glycero-3-phosphocholine (26:0 Lyso PC),
1-myristoyl-2-hydroxy-sn-glycero-3-phosphoethanolamine (14:0 Lyso PE),
1-palmitoyl-2-hydroxy-sn-glycero-3-phosphoethanolamine (16:0 Lyso PE),
1-stearoyl-2-hydroxy-sn-glycero-3-phosphoethanolamine (18:0 Lyso PE),
1-oleoyl-2-hydroxy-sn-glycero-3-phosphoethanolamine (18:1 Lyso PE),
1-hexadecyl-sn-glycero-3-phosphocholine (C16 Lyso PC), and

any combination thereof.

In some embodiment, the lipid composition of a pharmaceutical composition disclosed herein comprises at least one asymmetric phospholipid selected from the group consisting of MPPC, MSPC, PMPC, PSPC, SMPC, SPPC, and any combination thereof. In some embodiments, the asymmetric phospholipid is 1-myristoyl-2-stearoyl-sn-glycero-3-phosphocholine (MSPC).

In some embodiments, the lipid compositions disclosed herein can contain one or more symmetric phospholipids, one or more asymmetric phospholipids, or a combination thereof. When multiple phospholipids are present, they can be present in equimolar ratios, or non-equimolar ratios.

In one embodiment, the lipid composition of a pharmaceutical composition disclosed herein comprises a total amount of phospholipid (e.g., MSPC) which ranges from about 1 mol % to about 20 mol %, from about 5 mol % to about 20 mol %, from about 10 mol % to about 20 mol %, from about 15 mol % to about 20 mol %, from about 1 mol % to about 15 mol %, from about 5 mol % to about 15 mol %, from about 10 mol % to about 15 mol %, from about 5 mol % to about 10 mol % in the lipid composition. In one embodiment, the amount of the phospholipid is from about 8 mol % to about 15 mol % in the lipid composition. In one embodiment, the amount of the phospholipid (e.g., MSPC) is about 10 mol % in the lipid composition.

In some aspects, the amount of a specific phospholipid (e.g., MSPC) is at least about 1, 2, 3, 4, 5, 6, 7, 8, 9, 10, 11, 12, 13, 14, 15, 16, 17, 18, 19 or 20 mol % in the lipid composition.

### (v) Quaternary Amine Compounds

The lipid composition of a pharmaceutical composition disclosed herein can comprise one or more quaternary amine compounds (e.g., DOTAP). The term “quaternary amine compound” is used to include those compounds having one or more quaternary amine groups (e.g., trialkylamino groups) and permanently carrying a positive charge and existing in a form of a salt. For example, the one or more quaternary amine groups can be present in a lipid or a polymer (e.g., PEG). In some embodiments, the quaternary amine compound comprises (1) a quaternary amine group and (2) at least one hydrophobic tail group comprising (i) a hydrocarbon chain, linear or branched, and saturated or unsaturated, and (ii) optionally an ether, ester, carbonyl, or ketal linkage between the quaternary amine group and the hydrocarbon chain. In some embodiments, the quaternary amine group can be a trimethylammonium group. In some embodiments, the quaternary amine compound comprises two identical hydrocarbon chains. In some embodiments, the quaternary amine compound comprises two different hydrocarbon chains.

In some embodiments, the lipid composition of a pharmaceutical composition disclosed herein comprises at least one quaternary amine compound. Quaternary amine compound can be selected from the non-limiting group consisting of
1,2-dioleoyl-3-trimethylammonium-propane (DOTAP),
N-[1-(2,3-dioleoyloxy)propyl]-N,N,N-trimethylammonium chloride (DOTMA),
1-[2-(oleoyloxy)ethyl]-2-oleyl-3-(2-hydroxyethyl)imidazolinium chloride (DOTIM),
2,3-dioleyloxy-N-[2(sperminecarboxamido)ethyl]-N,N-dimethyl-1-propanaminium trifluoroacetate (DOSPA),
N,N-distearyl-N,N-dimethylammonium bromide (DDAB),
N-(1,2-dimyristyloxyprop-3-yl)-N,N-dimethyl-N-hydroxyethyl ammonium bromide (DMRIE),
N-(1,2-dioleoyloxyprop-3-yl)-N,N-dimethyl-N-hydroxyethyl ammonium bromide (DORIE),
N,N-dioleyl-N,N-dimethylammonium chloride (DODAC),
1,2-dilauroyl-sn-glycero-3-ethylphosphocholine (DLePC),
1,2-distearoyl-3-trimethylammonium-propane (DSTAP),
1,2-dipalmitoyl-3-trimethylammonium-propane (DPTAP),
1,2-dilinoleoyl-3-trimethylammonium-propane (DLTAP),
1,2-dimyristoyl-3-trimethylammonium-propane (DMTAP)
1,2-distearoyl-sn-glycero-3-ethylphosphocholine (DSePC)
1,2-dipalmitoyl-sn-glycero-3-ethylphosphocholine (DPePC),
1,2-dimyristoyl-sn-glycero-3-ethylphosphocholine (DMePC),
1,2-dioleoyl-sn-glycero-3-ethylphosphocholine (DOePC),
1,2-di-(9Z-tetradecenoyl)-sn-glycero-3-ethylphosphocholine (14:1 EPC),
1-palmitoyl-2-oleoyl-sn-glycero-3-ethylphosphocholine (16:0-18:1 EPC),

and any combination thereof.

In one embodiment, the quaternary amine compound is 1,2-dioleoyl-3-trimethylammonium-propane (DOTAP).

Quaternary amine compounds are known in the art, such as those described in US 2013/0245107 A1, US 2014/0363493 A1, U.S. Pat. No. 8,158,601, WO 2015/123264 A1, and WO 2015/148247 A1, which are incorporated herein by reference in their entirety.

In one embodiment, the amount of the quaternary amine compound (e.g., DOTAP) in the lipid composition disclosed herein ranges from about 0.01 mol % to about 20 mol %.

In one embodiment, the amount of the quaternary amine compound (e.g., DOTAP) in the lipid composition disclosed herein ranges from about 0.5 mol % to about 20 mol %, from about 0.5 mol % to about 15 mol %, from about 0.5 mol % to about 10 mol %, from about 1 mol % to about 20 mol %, from about 1 mol % to about 15 mol %, from about 1 mol % to about 10 mol %, from about 2 mol % to about 20 mol %, from about 2 mol % to about 15 mol %, from about 2 mol % to about 10 mol %, from about 3 mol % to about 20 mol %, from about 3 mol % to about 15 mol %, from about 3 mol % to about 10 mol %, from about 4 mol % to about 20 mol %, from about 4 mol % to about 15 mol %, from about 4 mol % to about 10 mol %, from about 5 mol % to about 20 mol %, from about 5 mol % to about 15 mol %, from about 5 mol % to about 10 mol %, from about 6 mol % to about 20 mol %, from about 6 mol % to about 15 mol %, from about 6 mol % to about 10 mol %, from about 7 mol % to about 20 mol %, from about 7 mol % to about 15 mol %, from about 7 mol % to about 10 mol %, from about 8 mol % to about 20 mol %, from about 8 mol % to about 15 mol %, from about 8 mol % to about 10 mol %, from about 9 mol % to about 20 mol %, from about 9 mol % to about 15 mol %, from about 9 mol % to about 10 mol %.

In one embodiment, the amount of the quaternary amine compound (e.g., DOTAP) in the lipid composition disclosed herein ranges from about 5 mol % to about 10 mol %.

In one embodiment, the amount of the quaternary amine compound (e.g., DOTAP) in the lipid composition disclosed herein is about 5 mol %. In one embodiment, the amount of the quaternary amine compound (e.g., DOTAP) in the lipid composition disclosed herein is about 10 mol %.

In some embodiments, the amount of the quaternary amine compound (e.g., DOTAP) is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mol % in the lipid composition disclosed herein.

In one embodiment, the mole ratio of the compound of formula (I) (e.g., Compounds 18, 25, 26 or 48) to the quaternary amine compound (e.g., DOTA) is about 100:1 to about 2.5:1. In one embodiment, the mole ratio of the compound of formula (I) (e.g., Compounds 18, 25, 26 or 48) to the quaternary amine compound (e.g., DOTAP) is about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, or about 2.5:1. In one embodiment, the mole ratio of the compound of formula (I) (e.g., Compounds 18, 25, 26 or 48) to the quaternary amine compound (e.g., DOTAP) in the lipid composition disclosed herein is about 10:1.

In some aspects, the lipid composition the pharmaceutical compositions disclosed herein does not comprise a quaternary amine compound. In some aspects, the lipid composition of the pharmaceutical compositions disclosed does not comprise DOTAP.

(iii) Structural Lipids

The lipid composition of a pharmaceutical composition disclosed herein can comprise one or more structural lipids. As used herein, the term “structural lipid” refers to sterols and also to lipids containing sterol moieties. In some embodiments, the structural lipid is selected from the group consisting of cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol.

In one embodiment, the amount of the structural lipid (e.g., an sterol such as cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the amount of the structural lipid (e.g., an sterol such as cholesterol) in the lipid composition disclosed herein ranges from about 25 mol % to about 30 mol %, from about 30 mol % to about 35 mol %, or from about 35 mol % to about 40 mol %.

In one embodiment, the amount of the structural lipid (e.g., a sterol such as cholesterol) in the lipid composition disclosed herein is about 24 mol %, about 29 mol %, about 34 mol %, or about 39 mol %.

In some embodiments, the amount of the structural lipid (e.g., an sterol such as cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

In some aspects, the lipid composition component of the pharmaceutical compositions for intratumoral delivery disclosed does not comprise cholesterol.

### (iv) Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol % mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

In some aspects, the lipid composition of the pharmaceutical compositions disclosed herein does not comprise a PEG-lipid.

In some embodiments, the lipid composition disclosed herein comprises a compound of formula (I) and an asymmetric phospholipid. In some embodiments, the lipid composition comprises compound 18 and MSPC.

In some embodiments, the lipid composition disclosed herein comprises a compound of formula (I) and a quaternary amine compound. In some embodiments, the lipid composition comprises compound 18 and DOTAP.

In some embodiments, the lipid composition disclosed herein comprises a compound of formula (I), an asymmetric phospholipid, and a quaternary amine compound. In some embodiments, the lipid composition comprises compound 18, MSPC and DOTAP.

In one embodiment, the lipid composition comprises about 50 mol % of a compound of formula (I) (e.g. Compounds 18, 25, 26 or 48), about 10 mol % of DSPC or MSPC, about 34 mol % of cholesterol, about 2 mol % of PEG-DMG, and about 5 mol % of DOTAP. In one embodiment, the lipid composition comprises about 50 mol % of a compound of formula (I) (e.g. Compounds 18, 25, 26 or 48), about 10 mol % of DSPC or MSPC, about 29 mol % of cholesterol, about 2 mol % of PEG-DMG, and about 10 mol % of DOTAP.

The components of the lipid nanoparticle can be tailored for optimal delivery of the polynucleotides based on the desired outcome. As a non-limiting example, the lipid nanoparticle can comprise 40-60 mol % a compound of formula (I), 8-16 mol % phospholipid, 30-45 mol % cholesterol, 1-5 mol % PEG lipid, and optionally 1-15 mol % quaternary amine compound.

In some embodiments, the lipid nanoparticle can comprise 45-65 mol % of a compound of formula (I), 5-10 mol % phospholipid, 25-40 mol % cholesterol, 0.5-5 mol % PEG lipid, and optionally 1-15 mol % quaternary amine compound.

Non-limiting examples of nucleic acid lipid particles are disclosed in U.S. Patent Publication No. 20140121263, herein incorporated by reference in its entirety.

### (v) Other Ionizable Amino Lipids

The lipid composition of the pharmaceutical composition disclosed herein can comprise one or more ionizable amino lipids in addition to a lipid according to formula (I).

Ionizable lipids can be selected from the non-limiting group consisting of
3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA),
heptatriaconta-6,9,28,31-tetraen-19-yl 4-(dimethylamino)butanoate (DLin-MC3-DMA),
2,2-dilinoleyl-4-(2-dimethylaminoethyl)[1,3]-dioxolane (DLin-KC2-DMA),
1,2-dioleyloxy-N,N-dimethylaminopropane (DODMA), (13Z,165Z)—N,N-dimethyl-3-nonydocosa-13-16-dien-1-amine (L608),
2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3(3)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and
(2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids can also be the compounds disclosed in International Publication No. WO 2015/199952 A1, hereby incorporated by reference in its entirety. For example, the ionizable amino lipids include, but not limited to:

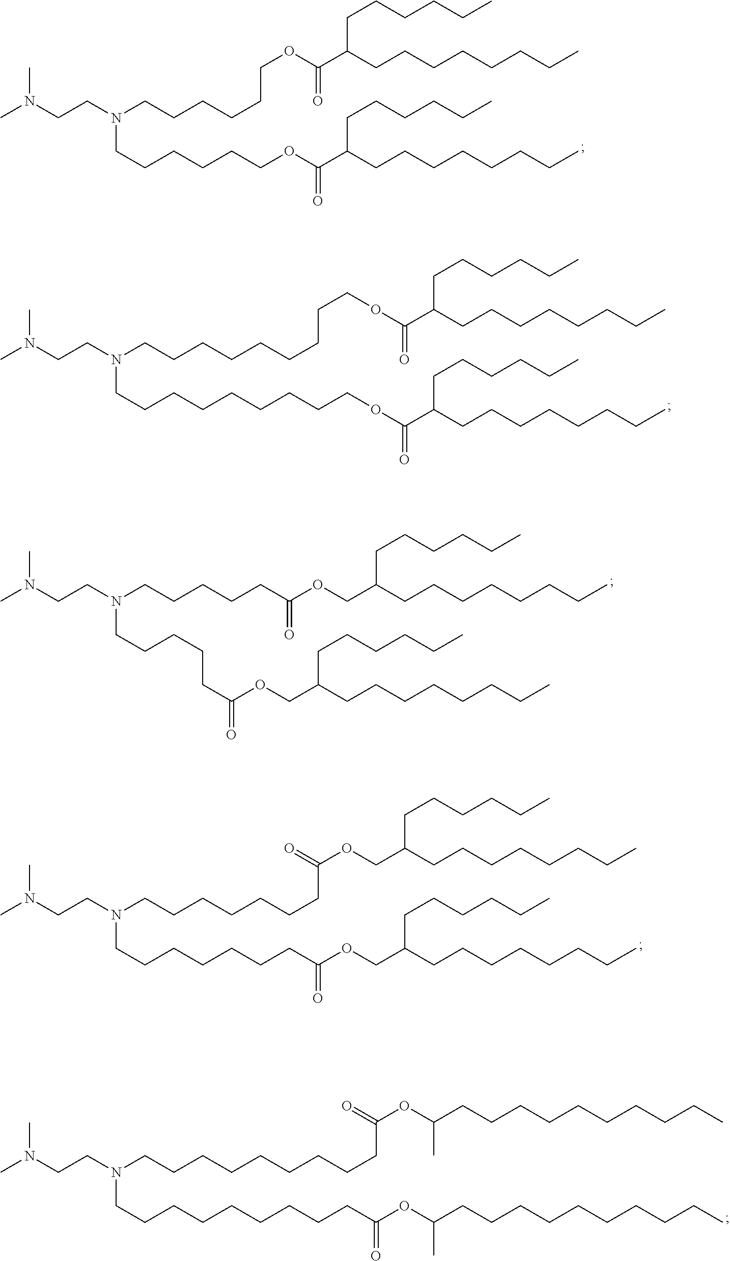

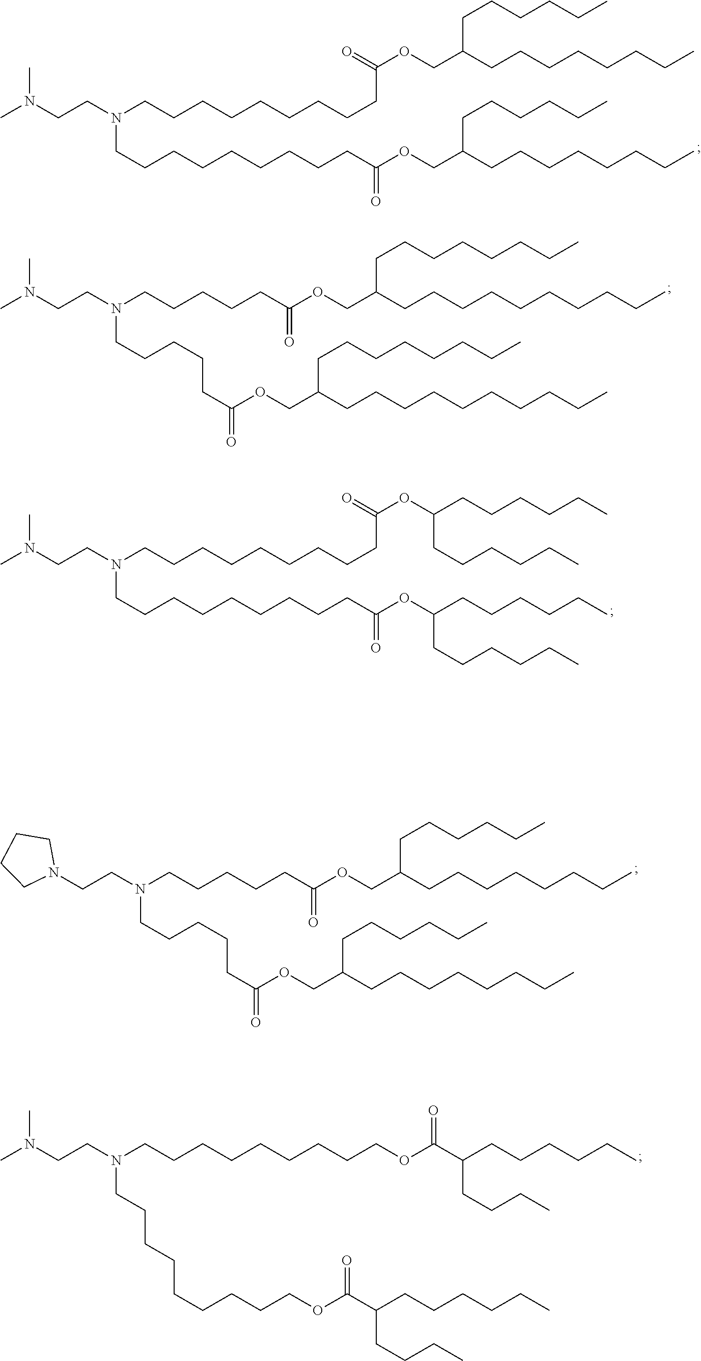

and any combination thereof.

### (vi) Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof). The lipid composition can include a buffer such as, but not limited to, citrate or phosphate at a pH of 7, salt and/or sugar. Salt and/or sugar can be included in the formulations described herein for isotonicity.

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

In one embodiment, formulations comprising the polynucleotides and lipid nanoparticles described herein can comprise 0.15 mg/ml to 2 mg/ml of the polynucleotide described herein (e.g., mRNA). In some embodiments, the formulation can further comprise 10 mM of citrate buffer and the formulation can additionally comprise up to 10% w/w of sucrose (e.g., at least 1% w/w, at least 2% w/w/, at least 3% w/w, at least 4% w/w, at least 5% w/w, at least 6% w/w, at least 7% w/w, at least 8% w/w, at least 9% w/w or 10% w/w).

(vii) Nanoparticle Compositions

In some embodiments, the pharmaceutical compositions disclosed herein are formulated as lipid nanoparticles (LNP). Accordingly, the present disclosure also provides nanoparticle compositions comprising (i) a lipid composition comprising a compound of formula (I) as described herein, and (ii) a polynucleotide encoding a therapeutic polypeptide. In such nanoparticle composition, the lipid composition disclosed herein can encapsulate the polynucleotide encoding a therapeutic polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

Nanoparticle compositions of the present disclosure comprise at least one compound according to formula (I). For example, the nanoparticle composition can include one or more of Compounds 1-20 or 25. Nanoparticle compositions can also include a variety of other components. For example, the nanoparticle composition can include one or more other lipids in addition to a lipid according to formula (I) or (II), for example (i) at least one phospholipid, (ii) at least one quaternary amine compound, (iii) at least one structural lipid, (iv) at least one PEG-lipid, or (v) any combination thereof.

In some embodiments, the nanoparticle composition comprises a compound of formula (I), (e.g., Compounds 18, 25, 26 or 48). In some embodiments, the nanoparticle composition comprises a compound of formula (I) (e.g., Compounds 18, 25, 26 or 48) and a phospholipid (e.g., DSPC or MSPC). In some embodiments, the nanoparticle composition comprises a compound of formula (I) (e.g., Compounds 18, 25, 26 or 48), a phospholipid (e.g., DSPC or MSPC), and a quaternary amine compound (e.g., DOTAP). In some embodiments, the nanoparticle composition comprises a compound of formula (I) (e.g., Compounds 18, 25, 26 or 48), and a quaternary amine compound (e.g., DOTAP).

In some embodiments, the nanoparticle composition comprises a lipid composition consisting or consisting essentially of compound of formula (I) (e.g., Compounds 18, 25, 26 or 48). In some embodiments, the nanoparticle composition comprises a lipid composition consisting or consisting essentially of a compound of formula (I) (e.g., Compounds 18, 25, 26 or 48) and a phospholipid (e.g., DSPC or MSPC). In some embodiments, the nanoparticle composition comprises a lipid composition consisting or consisting essentially of a compound of formula (I) (e.g., Compounds 18, 25, 26 or 48), a phospholipid (e.g., DSPC or MSPC), and a quaternary amine compound (e.g., DOTAP). In some embodiments, the nanoparticle composition comprises a lipid composition consisting or consisting essentially of a compound of formula (I) (e.g., Compounds 18, 25, 26 or 48), and a quaternary amine compound (e.g., DOTAP).

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a therapeutic polypeptide are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

### Other Delivery Agents

a. Liposomes, Lipoplexes, and Lipid Nanoparticles

In some embodiments, the compositions or formulations of the present disclosure comprise a delivery agent, e.g., a liposome, a lioplexes, a lipid nanoparticle, or any combination thereof. The polynucleotides described herein (e.g., a polynucleotide comprising a nucleotide sequence encoding a therapeutic polypeptide) can be formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US2013/0177638, US2013/0177637, US2013/0177636, US2013/0177635, US2013/0177634, US2013/0177633, US2013/0183375, US2013/0183373, and US2013/0183372. In some embodiments, the polynucleotides described herein can be encapsulated by the liposome and/or it can be contained in an aqueous core that can then be encapsulated by the liposome as described in, e.g., Intl. Pub. Nos. WO2012/031046, WO2012/031043, WO2012/030901, WO2012/006378, and WO2013/086526; and U.S. Pub. Nos. US2013/0189351, US2013/0195969 and US2013/0202684. Each of the references in herein incorporated by reference in its entirety.

In some embodiments, the polynucleotides described herein can be formulated in a cationic oil-in-water emulsion where the emulsion particle comprises an oil core and a cationic lipid that can interact with the polynucleotide anchoring the molecule to the emulsion particle. In some embodiments, the polynucleotides described herein can be formulated in a water-in-oil emulsion comprising a continuous hydrophobic phase in which the hydrophilic phase is dispersed. Exemplary emulsions can be made by the methods described in Intl. Pub. Nos. WO2012/006380 and WO2010/087791, each of which is herein incorporated by reference in its entirety.

In some embodiments, the polynucleotides described herein can be formulated in a lipid-polycation complex. The formation of the lipid-polycation complex can be accomplished by methods as described in, e.g., U.S. Pub. No. US2012/0178702. As a non-limiting example, the polycation can include a cationic peptide or a polypeptide such as, but not limited to, polylysine, polyornithine and/or polyarginine and the cationic peptides described in Intl. Pub. No. WO2012/013326 or U.S. Pub. No. US2013/0142818. Each of the references is herein incorporated by reference in its entirety.

In some embodiments, the polynucleotides described herein can be formulated in a lipid nanoparticle (LNP) such as those described in Intl. Pub. Nos. WO2013/123523, WO2012/170930, WO2011/127255 and WO2008/103276; and U.S. Pub. No. US2013/0171646, each of which is herein incorporated by reference in its entirety.

Lipid nanoparticle formulations typically comprise one or more lipids. In some embodiments, the lipid is a cationic or an ionizable lipid. In some embodiments, lipid nanoparticle formulations further comprise other components, including a phospholipid, a structural lipid, a quaternary amine compound, and a molecule capable of reducing particle aggregation, for example a PEG or PEG-modified lipid.

Cationic and ionizable lipids can include those as described in, e.g., Intl. Pub. Nos. WO2015/199952, WO 2015/130584, WO 2015/011633, and WO2012/040184 WO2013/126803, WO2011/153120, WO2011/149733, WO2011/090965, WO2011/043913, WO2011/022460, WO2012/061259, WO2012/054365, WO2012/044638, WO2010/080724, WO2010/021865, WO2008/103276, and WO2013/086373; U.S. Pat. Nos. 7,893,302, 7,404,969, 8,283,333, and 8,466,122; and U.S. Pub. Nos. US2011/0224447, US2012/0295832, US2015/0315112, US2010/0036115, US2012/0202871, US2013/0064894, US2013/0129785, US2013/0150625, US2013/0178541, US2013/0123338 and US2013/0225836, each of which is herein incorporated by reference in its entirety. In some embodiments, the amount of the cationic and ionizable lipids in the lipid composition ranges from about 0.01 mol % to about 99 mol %.

Exemplary ionizable lipids include, but not limited to, any one of Compounds 1-20 or 25 disclosed herein, DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl] eptadecan-8-amine, 1-[(1 S,2R)-2-hexyl cyclopropyl]-N,N-dimethylnonadecan-10-amine, N,N-dimethyl-1-[(1 S,2R)-2-octylcyclopropyl]nonadecan-10-amine, N,N-dimethyl-21-[(1 S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2 S)-2-undecyIcyclopropyl]tetradecan-5-amine, N,N-dimethyl-3-{7-[(1S,2R)-2-octylcyclopropyl]heptyl}dodecan-1-amine, 1-[(1R,2 S)-2-heptylcyclopropyl]-N,N-dimethyloctadecan-9-amine, 1-[(1 S,2R)-2-decyl cyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z, 16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1 S,2 S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)—N,N-dimethylnonacosa-11,20,2-trien-10-amine, and any combination thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC and/or MSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The quaternary amine compound as described herein include 1,2-dioleoyl-3-trimethylammonium-propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxyethyl ammonium bromide (DMRIE), N-(1,2-dioleoyloxyprop-3-yl)-N,N-dimethyl-N-hydroxyethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), and any combination thereof. In some embodiments, the amount of the quaternary amine compounds (e.g., DOTAP) in the lipid composition ranges from about 0.01 mol % to about 20 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0.1 mol % to about 5 mol %.

In some embodiments, the LNP formulations described herein can additionally comprise a permeability enhancer molecule. Non-limiting permeability enhancer molecules are described in U.S. Pub. No. US2005/0222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013/033438 or U.S. Pub. No. US2013/0196948. The LNP formulation can also contain a polymer conjugate (e.g., a water soluble conjugate) as described in, e.g., U.S. Pub. Nos. US2013/0059360, US2013/0196948, and US2013/0072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012/109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US2013/0183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013/110028, each of which is herein incorporated by reference in its entirety.

The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin (34 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013/110028, herein incorporated by reference in its entirety.

In some embodiments, the polynucleotide described herein is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments, the polynucleotides described herein are formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in Intl. Pub. No. WO2013/105101, herein incorporated by reference in its entirety.

In some embodiments, the polynucleotides described herein can be formulated for controlled release and/or targeted delivery. As used herein, “controlled release” refers to a pharmaceutical composition or compound release profile that conforms to a particular pattern of release to effect a therapeutic outcome. In one embodiment, the polynucleotides can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In some embodiments, the polynucleotides described herein can be encapsulated in a therapeutic nanoparticle, referred to herein as “therapeutic nanoparticle polynucleotides.” Therapeutic nanoparticles can be formulated by methods described in, e.g., Intl. Pub. Nos. WO2010/005740, WO2010/030763, WO2010/005721, WO2010/005723, and WO2012/054923; and U.S. Pub. Nos. US2011/0262491, US2010/0104645, US2010/0087337, US2010/0068285, US2011/0274759, US2010/0068286, US2012/0288541, US2012/0140790, US2013/0123351 and US2013/0230567; and U.S. Pat. Nos. 8,206,747, 8,293,276, 8,318,208 and 8,318,211, each of which is herein incorporated by reference in its entirety.

In some embodiments, the therapeutic nanoparticle polynucleotide can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the polynucleotides described herein can be formulated as disclosed in Intl. Pub. No. WO2010/075072 and U.S. Pub. Nos. US2010/0216804, US2011/0217377, US2012/0201859 and US2013/0150295, each of which is herein incorporated by reference in their entirety.

In some embodiments, the therapeutic nanoparticle polynucleotide can be formulated to be target specific, such as those described in Intl. Pub. Nos. WO2008/121949, WO2010/005726, WO2010/005725, WO2011/084521 and WO2011/084518; and U.S. Pub. Nos. US2010/0069426, US2012/0004293 and US2010/0104655, each of which is herein incorporated by reference in its entirety.

The LNPs can be prepared using microfluidic mixers or micromixers. Exemplary microfluidic mixers can include, but are not limited to, a slit interdigitial micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US2004/0262223 and US2012/0276209, each of which is incorporated herein by reference in their entirety.

In some embodiments, the polynucleotides described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the polynucleotides can be formulated in lipid nanoparticles using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In some embodiments, the polynucleotides described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the polynucleotides can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 μm, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO2013/082111, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US2013/0172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

As a non-limiting example modified mRNA can be formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradable, biocompatible polymers that are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C.

As a non-limiting example, the polynucleotides described herein can be formulated with the polymeric compound of PEG grafted with PLL as described in U.S. Pat. No. 6,177,274. As another non-limiting example, the polynucleotides described herein can be formulated with a block copolymer such as a PLGA-PEG block copolymer (see e.g., U.S. Pub. No. US20120004293 and U.S. Pat. Nos. 8,236,330 and 8,246,968), or a PLGA-PEG-PLGA block copolymer (see e.g., U.S. Pat. No. 6,004,573). Each of the references is herein incorporated by reference in its entirety.

In some embodiments, the polynucleotides described herein can be formulated with at least one amine-containing polymer such as, but not limited to polylysine, polyethylene imine, poly(amidoamine) dendrimers, poly(amine-co-esters) or combinations thereof. Exemplary polyamine polymers and their use as delivery agents are described in, e.g., U.S. Pat. Nos. 8,460,696, 8,236,280, each of which is herein incorporated by reference in its entirety.

In some embodiments, the polynucleotides described herein can be formulated in a biodegradable cationic lipopolymer, a biodegradable polymer, or a biodegradable copolymer, a biodegradable polyester copolymer, a biodegradable polyester polymer, a linear biodegradable copolymer, PAGA, a biodegradable cross-linked cationic multi-block copolymer or combinations thereof as described in, e.g., U.S. Pat. Nos. 6,696,038, 6,517,869, 6,267,987, 6,217,912, 6,652,886, 8,057,821, and 8,444,992; U.S. Pub. Nos. US2003/0073619, US2004/0142474, US2010/0004315, US2012/009145 and US2013/0195920; and Intl Pub. Nos. WO2006/063249 and WO2013/086322, each of which is herein incorporated by reference in its entirety.

In some embodiments, the polynucleotides disclosed herein can be formulated as a nanoparticle using a combination of polymers, lipids, and/or other biodegradable agents, such as, but not limited to, calcium phosphate. Components can be combined in a core-shell, hybrid, and/or layer-by-layer architecture, to allow for fine-tuning of the nanoparticle for delivery (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; herein incorporated by reference in their entireties). As a non-limiting example, the nanoparticle can comprise a plurality of polymers such as, but not limited to hydrophilic-hydrophobic polymers (e.g., PEG-PLGA), hydrophobic polymers (e.g., PEG) and/or hydrophilic polymers (Intl. Pub. No. WO2012/0225129, herein incorporated by reference in its entirety).

The use of core-shell nanoparticles has additionally focused on a high-throughput approach to synthesize cationic cross-linked nanogel cores and various shells (Siegwart et al., Proc Natl Acad Sci USA. 2011 108:12996-13001; herein incorporated by reference in its entirety). The complexation, delivery, and internalization of the polymeric nanoparticles can be precisely controlled by altering the chemical composition in both the core and shell components of the nanoparticle. For example, the core-shell nanoparticles can efficiently deliver siRNA to mouse hepatocytes after they covalently attach cholesterol to the nanoparticle.

In some embodiments, a hollow lipid core comprising a middle PLGA layer and an outer neutral lipid layer containing PEG can be used to delivery of the polynucleotides as described herein. In some embodiments, the lipid nanoparticles can comprise a core of the polynucleotides disclosed herein and a polymer shell, which is used to protect the polynucleotides in the core. The polymer shell can be any of the polymers described herein and are known in the art, the polymer shell can be used to protect the polynucleotides in the core.

Core-shell nanoparticles for use with the polynucleotides described herein are described in U.S. Pat. No. 8,313,777 or Intl. Pub. No. WO2013124867, each of which is herein incorporated by reference in their entirety.

w. Conjugates

In some embodiments, the compositions or formulations of the present disclosure comprise the polynucleotides described herein (e.g., a polynucleotide comprising a nucleotide sequence encoding a therapeutic polypeptide) that is covalently linked to a carrier or targeting group, or including two encoding regions that together produce a fusion protein (e.g., bearing a targeting group and therapeutic protein or peptide) as a conjugate. The conjugate can be a peptide that selectively directs the nanoparticle to neurons in a tissue or organism, or assists in crossing the blood-brain barrier.

The conjugates include a naturally occurring substance, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid, an oligonucleotide (e.g., an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

In some embodiments, the conjugate can function as a carrier for the polynucleotide disclosed herein. The conjugate can comprise a cationic polymer such as, but not limited to, polyamine, polylysine, polyalkylenimine, and polyethylenimine that can be grafted to with poly(ethylene glycol). Exemplary conjugates and their preparations are described in U.S. Pat. No. 6,586,524 and U.S. Pub. No. US2013/0211249, each of which herein is incorporated by reference in its entirety.

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent frucose, or aptamers. The ligand can be, for example, a lipopolysaccharide, or an activator of p38 MAP kinase.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein. As a non-limiting example, the targeting group can be a glutathione receptor (GR)-binding conjugate for targeted delivery across the blood-central nervous system barrier as described in, e.g., U.S. Pub. No. US2013/0216612 (herein incorporated by reference in its entirety).

In some embodiments, the conjugate can be a synergistic biomolecule-polymer conjugate, which comprises a long-acting continuous-release system to provide a greater therapeutic efficacy. The synergistic biomolecule-polymer conjugate can be those described in U.S. Pub. No. US2013/0195799. In some embodiments, the conjugate can be an aptamer conjugate as described in Intl. Pat. Pub. No. WO2012/040524. In some embodiments, the conjugate can be an amine containing polymer conjugate as described in U.S. Pat. No. 8,507,653. Each of the references is herein incorporated by reference in its entirety. In some embodiments, the polynucleotides can be conjugated to SMARTT POLYMER TECHNOLOGY® (PHASERX®, Inc. Seattle, Wash.).

In some embodiments, the polynucleotides described herein are covalently conjugated to a cell penetrating polypeptide, which can also include a signal sequence or a targeting sequence. The conjugates can be designed to have increased stability, and/or increased cell transfection; and/or altered the biodistribution (e.g., targeted to specific tissues or cell types).

In some embodiments, the polynucleotides described herein can be conjugated to an agent to enhance delivery. In some embodiments, the agent can be a monomer or polymer such as a targeting monomer or a polymer having targeting blocks as described in Intl. Pub. No. WO2011/062965. In some embodiments, the agent can be a transport agent covalently coupled to a polynucleotide as described in, e.g., U.S. Pat. Nos. 6,835,393 and 7,374,778. In some embodiments, the agent can be a membrane barrier transport enhancing agent such as those described in U.S. Pat. Nos. 7,737,108 and 8,003,129. Each of the references is herein incorporated by reference in its entirety.

### Methods of Use

The polynucleotides, pharmaceutical compositions and formulations described above are used in the preparation, manufacture and therapeutic use of to treat and/or prevent muscular dystrophy-related diseases, disorders or conditions. In some embodiments, the polynucleotides, compositions and formulations of the invention are used to treat and/or prevent Duchenne muscular dystrophy (DMD).

In some embodiments, the polynucleotides, pharmaceutical compositions and formulations of the invention are used in methods for increasing the levels of therapeutic proteins in a subject in need thereof. For instance, one aspect of the invention provides a method of alleviating the symptoms of DMD in a subject comprising the administration of a composition or formulation comprising a polynucleotide encoding a therapeutic protein to that subject (e.g, an mRNA encoding a functional component of a dystrophin polypeptide).

Replacement therapy is a potential treatment for DMD. Thus, in certain aspects of the invention, the polynucleotides, e.g., mRNA, disclosed herein comprise one or more sequences encoding a dystrophin polypeptide that is suitable for use in gene replacement therapy for DMD. In some embodiments, the present disclosure treats a lack of dystrophin or dystrophin activity, or decreased or abnormal dystrophin activity in a subject by providing a polynucleotide, e.g., mRNA, that encodes a dystrophin polypeptide to the subject. In some embodiments, the polynucleotide is sequence-optimized. In some embodiments, the polynucleotide (e.g., an mRNA) comprises a nucleic acid sequence (e.g., an ORF) encoding a dystrophin polypeptide, wherein the nucleic acid is sequence-optimized, e.g., by modifying its G/C, uridine, or thymidine content, and/or the polynucleotide comprises at least one chemically modified nucleoside. In some embodiments, the polynucleotide comprises a miRNA binding site, e.g., a miRNA binding site that binds miRNA-142.

In some embodiments, the administration of a composition or formulation comprising polynucleotide, pharmaceutical composition or formulation of the invention to a subject results in an increase in therapeutic protein in cells to a level at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% higher than the level observed prior to the administration of the composition or formulation.

In some embodiments, the administration of the polynucleotide, pharmaceutical composition or formulation of the invention results in expression of therapeutic protein in cells of the subject. In some embodiments, administering the polynucleotide, pharmaceutical composition or formulation of the invention results in an increase of therapeutic protein activity in the subject. For example, in some embodiments, the polynucleotides of the present invention are used in methods of administering a composition or formulation comprising an mRNA encoding a therapeutic polypeptide to a subject, wherein the method results in an increase of therapeutic protein activity in at least some cells of a subject.

In some embodiments, the administration of a composition or formulation comprising an mRNA encoding a therapeutic polypeptide to a subject results in an increase of therapeutic protein activity in cells subject to a level at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% or more of the activity level expected in a normal subject, e.g., a human not suffering from muscular dystrophy.

In some embodiments, the administration of the polynucleotide, pharmaceutical composition or formulation of the invention results in expression of a therapeutic protein in at least some of the cells of a subject that persists for a period of time sufficient to allow significant metabolism to occur.

In some embodiments, the expression of the encoded polypeptide is increased. In some embodiments, the polynucleotide increases therapeutic protein expression levels in cells when introduced into those cells, e.g., by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or to 100% with respect to the therapeutic protein expression level in the cells before the polypeptide is introduced in the cells.

Other aspects of the present disclosure relate to transplantation of cells containing polynucleotides to a mammalian subject. Administration of cells to mammalian subjects is known to those of ordinary skill in the art, and includes, but is not limited to, local implantation (e.g., topical or subcutaneous administration), organ delivery or systemic injection (e.g., intravenous injection or inhalation), and the formulation of cells in pharmaceutically acceptable carriers.

### Compositions and Formulations for Use

Certain aspects of the invention are directed to compositions or formulations comprising any of the polynucleotides disclosed above.

In some embodiments, the composition or formulation comprises:
(i) a polynucleotide (e.g., a RNA, e.g., an mRNA) comprising a sequence-optimized nucleotide sequence (e.g., an ORF) encoding a therapeutic polypeptide (e.g., the wild-type sequence, functional fragment, or variant thereof), wherein the polynucleotide comprises at least one chemically modified nucleobase, e.g., 5-methoxyuracil (e.g., wherein at least about 25%, at least about 30%, at least about 40%, at least about 50%, at least about 60%, at least about 70%, at least about 80%, at least about 90%, at least about 95%, at least about 99%, or 100% of the uracils are 5-methoxyuracils), and wherein the polynucleotide further comprises a miRNA binding site, e.g., a miRNA binding site that binds to miR-142 (e.g., a miR-142-3p or miR-142-5p binding site); and
(ii) a delivery agent comprising a compound having Formula (I), e.g., any of Compounds 1-20 or 25 (e.g., Compound 18 or 25).

In some embodiments, the uracil or thymine content of the ORF relative to the theoretical minimum uracil or thymine content of a nucleotide sequence encoding the therapeutic polypeptide (% U_{TM} or % T_{TM}), is between about 100% and about 150%.

In some embodiments, the polynucleotides, compositions or formulations above are used to treat and/or prevent a muscular dystrophy, e.g., Duchenne muscular dystrophy.

### Forms of Administration

The polynucleotides, pharmaceutical compositions and formulations of the invention described above can be administered by any route that results in a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration that is then covered by a dressing that occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions can be administered in a way that allows them cross the blood-brain barrier, vascular barrier, or other epithelial barrier. In some embodiments, a formulation for a route of administration can include at least one inactive ingredient.

The polynucleotides of the present invention (e.g., a polynucleotide comprising a nucleotide sequence encoding a therapeutic polypeptide or a functional fragment or variant thereof) can be delivered to a cell naked. As used herein in, “naked” refers to delivering polynucleotides free from agents that promote transfection. The naked polynucleotides can be delivered to the cell using routes of administration known in the art and described herein.

The polynucleotides of the present invention (e.g., a polynucleotide comprising a nucleotide sequence encoding a therapeutic polypeptide or a functional fragment or variant thereof) can be formulated, using the methods described herein. The formulations can contain polynucleotides that can be modified and/or unmodified. The formulations can further include, but are not limited to, cell penetration agents, a pharmaceutically acceptable carrier, a delivery agent, a bioerodible or biocompatible polymer, a solvent, and a sustained-release delivery depot. The formulated polynucleotides can be delivered to the cell using routes of administration known in the art and described herein.

The compositions can also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like.

The present disclosure encompasses the delivery of polynucleotides of the invention (e.g., a polynucleotide comprising a nucleotide sequence encoding a therapeutic polypeptide or a functional fragment or variant thereof) in forms suitable for parenteral and injectable administration. Liquid dosage forms for parenteral administration include, but are not limited to, pharmaceutically acceptable emulsions, microemulsions, solutions, suspensions, syrups, and/or elixirs. In addition to active ingredients, liquid dosage forms can comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR, alcohols, oils, modified oils, glycols, polysorbates, cyclodextrins, polymers, and/or combinations thereof.

A pharmaceutical composition for parenteral administration can comprise at least one inactive ingredient. Any or none of the inactive ingredients used can have been approved by the US Food and Drug Administration (FDA). A non-exhaustive list of inactive ingredients for use in pharmaceutical compositions for parenteral administration includes hydrochloric acid, mannitol, nitrogen, sodium acetate, sodium chloride and sodium hydroxide.

Injectable preparations, for example, sterile injectable aqueous or oleaginous suspensions can be formulated according to the known art using suitable dispersing agents, wetting agents, and/or suspending agents. Sterile injectable preparations can be sterile injectable solutions, suspensions, and/or emulsions in nontoxic parenterally acceptable diluents and/or solvents, for example, as a solution in 1,3-butanediol. Among the acceptable vehicles and solvents that can be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. The sterile formulation can also comprise adjuvants such as local anesthetics, preservatives and buffering agents.

Injectable formulations can be sterilized, for example, by filtration through a bacterial-retaining filter, and/or by incorporating sterilizing agents in the form of sterile solid compositions that can be dissolved or dispersed in sterile water or other sterile injectable medium prior to use.

Injectable formulations can be for direct injection into a region of a tissue, organ and/or subject. As a non-limiting example, a tissue, organ and/or subject can be directly injected a formulation by intramyocardial injection into the ischemic region. (See, e.g., Zangi et al. Nature Biotechnology 2013; the contents of which are herein incorporated by reference in its entirety).

In order to prolong the effect of an active ingredient, it is often desirable to slow the absorption of the active ingredient from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions that are compatible with body tissues.

### Kits and Devices

a. Kits

The invention provides a variety of kits for conveniently and/or effectively using the claimed nucleotides of the present invention. Typically, kits will comprise sufficient amounts and/or numbers of components to allow a user to perform multiple treatments of a subject(s) and/or to perform multiple experiments.

In one aspect, the present invention provides kits comprising the molecules (polynucleotides) of the invention.

Said kits can be for protein production, comprising a first polynucleotides comprising a translatable region. The kit can further comprise packaging and instructions and/or a delivery agent to form a formulation composition. The delivery agent can comprise a saline, a buffered solution, a lipidoid or any delivery agent disclosed herein.

In some embodiments, the buffer solution can include sodium chloride, calcium chloride, phosphate and/or EDTA. In another embodiment, the buffer solution can include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See, e.g., U.S. Pub. No. 2012/0258046; herein incorporated by reference in its entirety). In a further embodiment, the buffer solutions can be precipitated or it can be lyophilized. The amount of each component can be varied to enable consistent, reproducible higher concentration saline or simple buffer formulations. The components can also be varied in order to increase the stability of modified RNA in the buffer solution over a period of time and/or under a variety of conditions. In one aspect, the present invention provides kits for protein production, comprising: a polynucleotide comprising a translatable region, provided in an amount effective to produce a desired amount of a protein encoded by the translatable region when introduced into a target cell; a second polynucleotide comprising an inhibitory nucleic acid, provided in an amount effective to substantially inhibit the innate immune response of the cell; and packaging and instructions.

In one aspect, the present invention provides kits for protein production, comprising a polynucleotide comprising a translatable region, wherein the polynucleotide exhibits reduced degradation by a cellular nuclease, and packaging and instructions.

In one aspect, the present invention provides kits for protein production, comprising a polynucleotide comprising a translatable region, wherein the polynucleotide exhibits reduced degradation by a cellular nuclease, and a mammalian cell suitable for translation of the translatable region of the first nucleic acid.

b. Devices

The present invention provides for devices that can incorporate polynucleotides that encode polypeptides of interest. These devices contain in a stable formulation the reagents to synthesize a polynucleotide in a formulation available to be immediately delivered to a subject in need thereof, such as a human patient.

Devices for administration can be employed to deliver the polynucleotides of the present invention according to single, multi- or split-dosing regimens taught herein. Such devices are taught in, for example, International Application Publ. No. WO2013/151666, the contents of which are incorporated herein by reference in their entirety.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

According to the present invention, these multi-administration devices can be utilized to deliver the single, multi- or split doses contemplated herein. Such devices are taught for example in, International Application Publ. No. WO2013/151666, the contents of which are incorporated herein by reference in their entirety.

In some embodiments, the polynucleotide is administered subcutaneously or intramuscularly via at least 3 needles to three different, optionally adjacent, sites simultaneously, or within a 60 minute period (e.g., administration to 4, 5, 6, 7, 8, 9, or 10 sites simultaneously or within a 60 minute period).

c. Methods and Devices Utilizing Catheters and/or Lumens

Methods and devices using catheters and lumens can be employed to administer the polynucleotides of the present invention on a single, multi- or split dosing schedule. Such methods and devices are described in International Application Publication No. WO2013/151666, the contents of which are incorporated herein by reference in their entirety.

d. Methods and Devices Utilizing Electrical Current

Methods and devices utilizing electric current can be employed to deliver the polynucleotides of the present invention according to the single, multi- or split dosing regimens taught herein. Such methods and devices are described in International Application Publication No. WO2013/151666, the contents of which are incorporated herein by reference in their entirety.

### Definitions

In order that the present disclosure can be more readily understood, certain terms are first defined. As used in this application, except as otherwise expressly provided herein, each of the following terms shall have the meaning set forth below. Additional definitions are set forth throughout the application.

The invention includes embodiments in which exactly one member of the group is present in, employed in, or otherwise relevant to a given product or process. The invention includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleobases are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

About: The term “about” as used in connection with a numerical value throughout the specification and the claims denotes an interval of accuracy, familiar and acceptable to a person skilled in the art, such interval of accuracy is ±10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Administered in combination: As used herein, the term “administered in combination” or “combined administration” means that two or more agents are administered to a subject at the same time or within an interval such that there can be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amino acid substitution: The term “amino acid substitution” refers to replacing an amino acid residue present in a parent or reference sequence (e.g., a wild type therapeutic sequence) with another amino acid residue. An amino acid can be substituted in a parent or reference sequence (e.g., a wild type therapeutic polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position X, and Y and Z are alternative substituting amino acid residue, i.e.,

In the context of the present disclosure, substitutions (even when they referred to as amino acid substitution) are conducted at the nucleic acid level, i.e., substituting an amino acid residue with an alternative amino acid residue is conducted by substituting the codon encoding the first amino acid with a codon encoding the second amino acid.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein with respect to a disease, the term “associated with” means that the symptom, measurement, characteristic, or status in question is linked to the diagnosis, development, presence, or progression of that disease. As association can, but need not, be causatively linked to the disease. For example, symptoms, sequelae, or any effects causing a decrease in the quality of life of a patient of muscular dystrophy are considered associated with muscular dystrophy and in some embodiments of the present invention can be treated, ameliorated, or prevented by administering the polynucleotides of the present invention to a subject in need thereof.

When used with respect to two or more moieties, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It can also suggest ionic or hydrogen bonding or a hybridization based connectivity sufficiently stable such that the “associated” entities remain physically associated.

Bifunctional: As used herein, the term “bifunctional” refers to any substance, molecule or moiety that is capable of or maintains at least two functions. The functions can affect the same outcome or a different outcome. The structure that produces the function can be the same or different. For example, bifunctional modified RNAs of the present invention can encode a therapeutic peptide (a first function) while those nucleosides that comprise the encoding RNA are, in and of themselves, capable of extending the half-life of the RNA (second function). In this example, delivery of the bifunctional modified RNA to a subject suffering from a protein deficiency would produce not only a peptide or protein molecule that can ameliorate or treat a disease or conditions, but would also maintain a population modified RNA present in the subject for a prolonged period of time. In other aspects, a bifunction modified mRNA can be a quimeric molecule comprising, for example, an RNA encoding a therapeutic peptide (a first function) and a second protein either fused to first protein or co-expressed with the first protein.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present invention can be considered biologically active if even a portion of the polynucleotide is biologically active or mimics an activity considered biologically relevant.

Chimera: As used herein, “chimera” is an entity having two or more incongruous or heterogeneous parts or regions. For example, a chimeric molecule can comprise a first part comprising a therapeutic polypeptide, and a second part (e.g., genetically fused to the first part) comprising a second therapeutic protein (e.g., a protein with a distinct enzymatic activity, an antigen binding moiety, or a moiety capable of extending the plasma half life of therapeutic, for example, an Fc region of an antibody).

Sequence Optimization: The term “sequence optimization” refers to a process or series of processes by which nucleobases in a reference nucleic acid sequence are replaced with alternative nucleobases, resulting in a nucleic acid sequence with improved properties, e.g., improved protein expression or decreased immunogenicity.

In general, the goal in sequence optimization is to produce a synonymous nucleotide sequence than encodes the same polypeptide sequence encoded by the reference nucleotide sequence. Thus, there are no amino acid substitutions (as a result of codon optimization) in the polypeptide encoded by the codon optimized nucleotide sequence with respect to the polypeptide encoded by the reference nucleotide sequence.

Codon substitution: The terms “codon substitution” or “codon replacement” in the context of sequence optimization refer to replacing a codon present in a reference nucleic acid sequence with another codon. A codon can be substituted in a reference nucleic acid sequence, for example, via chemical peptide synthesis or through recombinant methods known in the art. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon.

As used herein, the terms “coding region” and “region encoding” and grammatical variants thereof, refer to an Open Reading Frame (ORF) in a polynucleotide that upon expression yields a polypeptide or protein.

Compound: As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Contacting: As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal can be performed by varied routes of administration (e.g., intravenous, intramuscular, intradermal, and subcutaneous) and can involve varied amounts of nanoparticle compositions. Moreover, more than one mammalian cell can be contacted by a nanoparticle composition.

Conservative amino acid substitution: A “conservative amino acid substitution” is one in which the amino acid residue in a protein sequence is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitution: Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can be readily identified by workers of ordinary skill. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of an polynucleotide or polypeptide or can apply to a portion, region or feature thereof.

Controlled Release: As used herein, the term “controlled release” refers to a pharmaceutical composition or compound release profile that conforms to a particular pattern of release to effect a therapeutic outcome.

Covalent Derivative: The term “covalent derivative” when referring to polypeptides include modifications of a native or starting protein with an organic proteinaceous or non-proteinaceous derivatizing agent, and/or post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Cyclic or Cyclized: As used herein, the term “cyclic” refers to the presence of a continuous loop. Cyclic molecules need not be circular, only joined to form an unbroken chain of subunits. Cyclic molecules such as the engineered RNA or mRNA of the present invention can be single units or multimers or comprise one or more components of a complex or higher order structure.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivering: As used herein, the term “delivering” means providing an entity to a destination. For example, delivering a polynucleotide to a subject can involve administering a nanoparticle composition including the polynucleotide to the subject (e.g., by an intravenous, intramuscular, intradermal, or subcutaneous route). Administration of a nanoparticle composition to a mammal or mammalian cell can involve contacting one or more cells with the nanoparticle composition.

Delivery Agent: As used herein, “delivery agent” refers to any substance that facilitates, at least in part, the in vivo, in vitro, or ex vivo delivery of a polynucleotide to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties that are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels can be located at any position in the peptides or proteins disclosed herein. They can be within the amino acids, the peptides, or proteins, or located at the N- or C-termini.

Diastereomer: As used herein, the term “diastereomer,” means stereoisomers that are not mirror images of one another and are non-superimposable on one another.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Domain: As used herein, when referring to polypeptides, the term “domain” refers to a motif of a polypeptide having one or more identifiable structural or functional characteristics or properties (e.g., binding capacity, serving as a site for protein-protein interactions).

Dosing regimen: As used herein, a “dosing regimen” or a “dosing regimen” is a schedule of administration or physician determined regimen of treatment, prophylaxis, or palliative care.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats a protein deficiency (e.g., a therapeutic deficiency), an effective amount of an agent is, for example, an amount of mRNA expressing sufficient therapeutic to ameliorate, reduce, eliminate, or prevent the signs or symptoms associated with the therapeutic deficiency, as compared to the severity of the symptom observed without administration of the agent. The term “effective amount” can be used interchangeably with “effective dose,” “therapeutically effective amount,” or “therapeutically effective dose.”

Enantiomer: As used herein, the term “enantiomer” means each individual optically active form of a compound of the invention, having an optical purity or enantiomeric excess (as determined by methods standard in the art) of at least 80% (i.e., at least 90% of one enantiomer and at most 10% of the other enantiomer), at least 90%, or at least 98%.

Encapsulate: As used herein, the term “encapsulate” means to enclose, surround or encase.

Encapsulation Efficiency: As used herein, “encapsulation efficiency” refers to the amount of a polynucleotide that becomes part of a nanoparticle composition, relative to the initial total amount of polynucleotide used in the preparation of a nanoparticle composition. For example, if 97 mg of polynucleotide are encapsulated in a nanoparticle composition out of a total 100 mg of polynucleotide initially provided to the composition, the encapsulation efficiency can be given as 97%. As used herein, “encapsulation” can refer to complete, substantial, or partial enclosure, confinement, surrounding, or encasement.

Encoded protein cleavage signal: As used herein, “encoded protein cleavage signal” refers to the nucleotide sequence that encodes a protein cleavage signal.

Engineered: As used herein, embodiments of the invention are “engineered” when they are designed to have a feature or property, whether structural or chemical, that varies from a starting point, wild type or native molecule.

Enhanced Delivery: As used herein, the term “enhanced delivery” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a polynucleotide by a nanoparticle to a target tissue of interest (e.g., mammalian liver) compared to the level of delivery of a polynucleotide by a control nanoparticle to a target tissue of interest (e.g., MC3, KC2, or DLinDMA). The level of delivery of a nanoparticle to a particular tissue can be measured by comparing the amount of protein produced in a tissue to the weight of said tissue, comparing the amount of polynucleotide in a tissue to the weight of said tissue, comparing the amount of protein produced in a tissue to the amount of total protein in said tissue, or comparing the amount of polynucleotide in a tissue to the amount of total polynucleotide in said tissue. It will be understood that the enhanced delivery of a nanoparticle to a target tissue need not be determined in a subject being treated, it can be determined in a surrogate such as an animal model (e.g., a rat model).

Exosome: As used herein, “exosome” is a vesicle secreted by mammalian cells or a complex involved in RNA degradation.

Expression: As used herein, “expression” of a nucleic acid sequence refers to one or more of the following events: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

Ex Vivo: As used herein, the term “ex vivo” refers to events that occur outside of an organism (e.g., animal, plant, or microbe or cell or tissue thereof). Ex vivo events can take place in an environment minimally altered from a natural (e.g., in vivo) environment.

Feature: As used herein, a “feature” refers to a characteristic, a property, or a distinctive element. When referring to polypeptides, “features” are defined as distinct amino acid sequence-based components of a molecule. Features of the polypeptides encoded by the polynucleotides of the present invention include surface manifestations, local conformational shape, folds, loops, half-loops, domains, half-domains, sites, termini or any combination thereof.

Formulation: As used herein, a “formulation” includes at least a polynucleotide and one or more of a carrier, an excipient, and a delivery agent.

Fragment: A “fragment,” as used herein, refers to a portion. For example, fragments of proteins can comprise polypeptides obtained by digesting full-length protein isolated from cultured cells. In some embodiments, a fragment is a subsequences of a full length protein wherein N-terminal, and/or C-terminal, and/or internal subsequences have been deleted. In some preferred aspects of the present invention, the fragments of a protein of the present invention are functional fragments.

Functional: As used herein, a “functional” biological molecule is a biological molecule in a form in which it exhibits a property and/or activity by which it is characterized. Thus, a functional fragment of a polynucleotide of the present invention is a polynucleotide capable of expressing a functional therapeutic fragment. As used herein, a functional fragment of a therapeutic refers to a fragment of wild type a therapeutic (i.e., a fragment of any of its naturally occurring isoforms), or a mutant or variant thereof, wherein the fragment retains a least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the biological activity of the corresponding full length protein.

Helper Lipid: As used herein, the term “helper lipid” refers to a compound or molecule that includes a lipidic moiety (for insertion into a lipid layer, e.g., lipid bilayer) and a polar moiety (for interaction with physiologic solution at the surface of the lipid layer). Typically, the helper lipid is a phospholipid. A function of the helper lipid is to “complement” the amino lipid and increase the fusogenicity of the bilayer and/or to help facilitate endosomal escape, e.g., of nucleic acid delivered to cells. Helper lipids are also believed to be a key structural component to the surface of the LNP.

Homology: As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present invention, the term homology encompasses both to identity and similarity.

In some embodiments, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

Identity: As used herein, the term “identity” refers to the overall monomer conservation between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources, and for alignment of both protein and nucleotide sequences. One suitable program to determine percent sequence identity is bl2seq, part of the BLAST suite of program available from the U.S. Government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity “% ID” of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

Immune response: The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. In some cases, the administration of a nanoparticle comprising a lipid component and an encapsulated therapeutic agent can trigger an immune response, which can be caused by (i) the encapsulated therapeutic agent (e.g., an mRNA), (ii) the expression product of such encapsulated therapeutic agent (e.g., a polypeptide encoded by the mRNA), (iii) the lipid component of the nanoparticle, or (iv) a combination thereof.

Inflammatory response: “Inflammatory response” refers to immune responses involving specific and non-specific defense systems. A specific defense system reaction is a specific immune system reaction to an antigen. Examples of specific defense system reactions include antibody responses. A non-specific defense system reaction is an inflammatory response mediated by leukocytes generally incapable of immunological memory, e.g., macrophages, eosinophils and neutrophils. In some aspects, an immune response includes the secretion of inflammatory cytokines, resulting in elevated inflammatory cytokine levels.

Inflammatory cytokines: The term “inflammatory cytokine” refers to cytokines that are elevated in an inflammatory response. Examples of inflammatory cytokines include interleukin-6 (IL-6), CXCL1 (chemokine (C—X—C motif) ligand 1; also known as GROα, interferon-γ (IFNγ), tumor necrosis factor α (TNFα), interferon γ-induced protein 10 (IP-10), or granulocyte-colony stimulating factor (G-CSF). The term inflammatory cytokines includes also other cytokines associated with inflammatory responses known in the art, e.g., interleukin-1 (IL-1), interleukin-8 (IL-8), interleukin-12 (IL-12), interleukin-13 (Il-13), interferon α (IFN-α), etc.

In Vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In Vivo: As used herein, the term “in vivo” refers to events that occur within an organism (e.g., animal, plant, or microbe or cell or tissue thereof).

Insertional and deletional variants: “Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid. “Deletional variants” when referring to polypeptides are those with one or more amino acids in the native or starting amino acid sequence removed. Ordinarily, deletional variants will have one or more amino acids deleted in a particular region of the molecule.

Intact: As used herein, in the context of a polypeptide, the term “intact” means retaining an amino acid corresponding to the wild type protein, e.g., not mutating or substituting the wild type amino acid. Conversely, in the context of a nucleic acid, the term “intact” means retaining a nucleobase corresponding to the wild type nucleic acid, e.g., not mutating or substituting the wild type nucleobase.

Ionizable amino lipid: The term “ionizable amino lipid” includes those lipids having one, two, three, or more fatty acid or fatty alkyl chains and a pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. Such ionizable amino lipids include, but are not limited to DLin-MC3-DMA (MC3) and (13Z,165Z)—N,N-dimethyl-3-nonydocosa-13-16-dien-1-amine (L608).

Isolated: As used herein, the term “isolated” refers to a substance or entity that has been separated from at least some of the components with which it was associated (whether in nature or in an experimental setting). Isolated substances (e.g., polynucleotides or polypeptides) can have varying levels of purity in reference to the substances from which they have been isolated. Isolated substances and/or entities can be separated from at least about 10%, about 20%, about 30%, about 40%, about 50%, about 60%, about 70%, about 80%, about 90%, or more of the other components with which they were initially associated. In some embodiments, isolated substances are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof.

A polynucleotide, vector, polypeptide, cell, or any composition disclosed herein which is “isolated” is a polynucleotide, vector, polypeptide, cell, or composition which is in a form not found in nature. Isolated polynucleotides, vectors, polypeptides, or compositions include those which have been purified to a degree that they are no longer in a form in which they are found in nature. In some aspects, a polynucleotide, vector, polypeptide, or composition which is isolated is substantially pure.

Isomer: As used herein, the term “isomer” means any tautomer, stereoisomer, enantiomer, or diastereomer of any compound of the invention. It is recognized that the compounds of the invention can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Methods of Administration: As used herein, “methods of administration” can include intravenous, intramuscular, intradermal, subcutaneous, or other methods of delivering a composition to a subject. A method of administration can be selected to target delivery (e.g., to specifically deliver) to a specific region or system of a body.

Modified: As used herein “modified” refers to a changed state or structure of a molecule of the invention. Molecules can be modified in many ways including chemically, structurally, and functionally. In some embodiments, the mRNA molecules of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Mucus: As used herein, “mucus” refers to the natural substance that is viscous and comprises mucin glycoproteins.

Nanoparticle Composition: As used herein, a “nanoparticle composition” is a composition comprising one or more lipids. Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Naturally occurring: As used herein, “naturally occurring” means existing in nature without artificial aid.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid sequence: The terms “nucleic acid sequence,” “nucleotide sequence,” or “polynucleotide sequence” are used interchangeably and refer to a contiguous nucleic acid sequence. The sequence can be either single stranded or double stranded DNA or RNA, e.g., an mRNA.

The term “nucleic acid,” in its broadest sense, includes any compound and/or substance that comprises a polymer of nucleotides. These polymers are often referred to as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

The phrase “nucleotide sequence encoding” refers to the nucleic acid (e.g., an mRNA or DNA molecule) coding sequence which encodes a polypeptide. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

Off-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Optionally substituted: Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional.

Part: As used herein, a “part” or “region” of a polynucleotide is defined as any portion of the polynucleotide that is less than the entire length of the polynucleotide.

Patient: As used herein, “patient” refers to a subject who can seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding mRNA sequences (comprising U) are considered codon-optimized nucleotide sequence of the present invention. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ΨΨC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

Polypeptide variant: As used herein, the term “polypeptide variant” refers to molecules that differ in their amino acid sequence from a native or reference sequence. The amino acid sequence variants can possess substitutions, deletions, and/or insertions at certain positions within the amino acid sequence, as compared to a native or reference sequence. Ordinarily, variants will possess at least about 50% identity, at least about 60% identity, at least about 70% identity, at least about 80% identity, at least about 90% identity, at least about 95% identity, at least about 99% identity to a native or reference sequence. In some embodiments, they will be at least about 80%, or at least about 90% identical to a native or reference sequence.

Polypeptide per unit drug (PUD): As used herein, a PUD or product per unit drug, is defined as a subdivided portion of total daily dose, usually 1 mg, pg, kg, etc., of a product (such as a polypeptide) as measured in body fluid or tissue, usually defined in concentration such as pmol/mL, mmol/mL, etc. divided by the measure in the body fluid.

Preventing: As used herein, the term “preventing” refers to partially or completely delaying onset of an infection, disease, disorder and/or condition; partially or completely delaying onset of one or more signs or symptoms, features, or clinical manifestations of a particular infection, disease, disorder, and/or condition; partially or completely delaying onset of one or more signs or symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity that is in a form predicate for that substance, molecule or entity to act as a therapeutic upon chemical or physical alteration. Prodrugs can by covalently bonded or sequestered in some way and that release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Prodrugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune prophylaxis” refers to a measure to produce active or passive immunity to prevent the spread of disease.

Protein cleavage site: As used herein, “protein cleavage site” refers to a site where controlled cleavage of the amino acid chain can be accomplished by chemical, enzymatic or photochemical means.

Protein cleavage signal: As used herein “protein cleavage signal” refers to at least one amino acid that flags or marks a polypeptide for cleavage.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Pseudouridine: As used herein, pseudouridine (ψ) refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹ψ), 1-methyl-4-thio-pseudouridine (m¹s⁴ψ) 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-β-amino-3-carboxypropyl)pseudouridine (acp³ψ), and 2′-O-methyl-pseudouridine (ψm).

Purified: As used herein, “purify,” “purified,” “purification” means to make substantially pure or clear from unwanted components, material defilement, admixture or imperfection.

Reference Nucleic Acid Sequence: The term “reference nucleic acid sequence” or “reference nucleic acid” or “reference nucleotide sequence” or “reference sequence” refers to a starting nucleic acid sequence (e.g., a RNA, e.g., an mRNA sequence) that can be sequence optimized. In some embodiments, the reference nucleic acid sequence is a wild type nucleic acid sequence, a fragment or a variant thereof. In some embodiments, the reference nucleic acid sequence is a previously sequence optimized nucleic acid sequence.

Repeated transfection: As used herein, the term “repeated transfection” refers to transfection of the same cell culture with a polynucleotide a plurality of times. The cell culture can be transfected at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times at least 18 times, at least 19 times, at least 20 times, at least 25 times, at least 30 times, at least 35 times, at least 40 times, at least 45 times, at least 50 times or more.

Salts: In some aspects, the pharmaceutical composition for intratumoral delivery disclosed herein and comprises salts of some of their lipid constituents. The term “salt” includes any anionic and cationic complex. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

Sample: As used herein, the term “sample” or “biological sample” refers to a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). A sample further can include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which can contain cellular components, such as proteins or nucleic acid molecule.

Signal Sequence: As used herein, the phrases “signal sequence,” “signal peptide,” and “transit peptide” are used interchangeably and refer to a sequence that can direct the transport or localization of a protein to a certain organelle, cell compartment, or extracellular export. The term encompasses both the signal sequence polypeptide and the nucleic acid sequence encoding the signal sequence. Thus, references to a signal sequence in the context of a nucleic acid refer in fact to the nucleic acid sequence encoding the signal sequence polypeptide.

Signal transduction pathway: A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Single unit dose: As used herein, a “single unit dose” is a dose of any therapeutic administered in one dose/at one time/single route/single point of contact, i.e., single administration event.

Split dose: As used herein, a “split dose” is the division of single unit dose or total daily dose into two or more doses.

Specific delivery: As used herein, the term “specific delivery,” “specifically deliver,” or “specifically delivering” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a polynucleotide by a nanoparticle to a target tissue of interest (e.g., mammalian liver) compared to an off-target tissue (e.g., mammalian spleen). The level of delivery of a nanoparticle to a particular tissue can be measured by comparing the amount of protein produced in a tissue to the weight of said tissue, comparing the amount of polynucleotide in a tissue to the weight of said tissue, comparing the amount of protein produced in a tissue to the amount of total protein in said tissue, or comparing the amount of polynucleotide in a tissue to the amount of total polynucleotide in said tissue. For example, for renovascular targeting, a polynucleotide is specifically provided to a mammalian kidney as compared to the liver and spleen if 1.5, 2-fold, 3-fold, 5-fold, 10-fold, 15 fold, or 20 fold more polynucleotide per 1 g of tissue is delivered to a kidney compared to that delivered to the liver or spleen following systemic administration of the polynucleotide. It will be understood that the ability of a nanoparticle to specifically deliver to a target tissue need not be determined in a subject being treated, it can be determined in a surrogate such as an animal model (e.g., a rat model).

Stable: As used herein “stable” refers to a compound that is sufficiently robust to survive isolation to a useful degree of purity from a reaction mixture, and in some cases capable of formulation into an efficacious therapeutic agent.

Stabilized: As used herein, the term “stabilize,” “stabilized,” “stabilized region” means to make or become stable.

Stereoisomer: As used herein, the term “stereoisomer” refers to all possible different isomeric as well as conformational forms that a compound can possess (e.g., a compound of any formula described herein), in particular all possible stereochemically and conformationally isomeric forms, all diastereomers, enantiomers and/or conformers of the basic molecular structure. Some compounds of the present invention can exist in different tautomeric forms, all of the latter being included within the scope of the present invention.

Subject. By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of treatment.

Substantially: As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

Substantially equal: As used herein as it relates to time differences between doses, the term means plus/minus 2%.

Substantially simultaneous: As used herein and as it relates to plurality of doses, the term means within 2 seconds.

Suffering from: An individual who is “suffering from” a disease, disorder, and/or condition has been diagnosed with or displays one or more signs or symptoms of the disease, disorder, and/or condition.

Susceptible to: An individual who is “susceptible to” a disease, disorder, and/or condition has not been diagnosed with and/or can not exhibit signs or symptoms of the disease, disorder, and/or condition but harbors a propensity to develop a disease or its signs or symptoms. In some embodiments, an individual who is susceptible to a disease, disorder, and/or condition (for example, cancer) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or other molecules of the present invention can be chemical or enzymatic.

Targeted Cells: As used herein, “targeted cells” refers to any one or more cells of interest. The cells can be found in vitro, in vivo, in situ or in the tissue or organ of an organism. The organism can be an animal, for example a mammal, a human, a subject or a patient.

Target tissue: As used herein “target tissue” refers to any one or more tissue types of interest in which the delivery of a polynucleotide would result in a desired biological and/or pharmacological effect. Examples of target tissues of interest include specific tissues, organs, and systems or groups thereof. In particular applications, a target tissue can be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues can include the liver and the spleen.

The presence of a therapeutic agent in an off-target issue can be the result of: (i) leakage of a polynucleotide from the administration site to peripheral tissue or distant off-target tissue (e.g., liver) via diffusion or through the bloodstream (e.g., a polynucleotide intended to express a polypeptide in a certain tissue would reach the liver and the polypeptide would be expressed in the liver); or (ii) leakage of an polypeptide after administration of a polynucleotide encoding such polypeptide to peripheral tissue or distant off-target tissue (e.g., liver) via diffusion or through the bloodstream (e.g., a polynucleotide would expressed a polypeptide in the target tissue, and the polypeptide would diffuse to peripheral tissue).

Targeting sequence: As used herein, the phrase “targeting sequence” refers to a sequence that can direct the transport or localization of a protein or polypeptide.

Terminus: As used herein the terms “termini” or “terminus,” when referring to polypeptides, refers to an extremity of a peptide or polypeptide. Such extremity is not limited only to the first or final site of the peptide or polypeptide but can include additional amino acids in the terminal regions. The polypeptide based molecules of the invention can be characterized as having both an N-terminus (terminated by an amino acid with a free amino group (NH₂)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides can be modified such that they begin or end, as the case can be, with a non-polypeptide based moiety such as an organic conjugate.

Therapeutic Agent: The term “therapeutic agent” refers to an agent that, when administered to a subject, has a therapeutic, diagnostic, and/or prophylactic effect and/or elicits a desired biological and/or pharmacological effect. For example, in some embodiments, an mRNA encoding a dystrophin polypeptide can be a therapeutic agent. In other embodiments, a therapeutic agent may be a therapeutic protein.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve signs or symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve signs or symptoms of, diagnose, prevent, and/or delay the onset of the infection, disease, disorder, and/or condition.

Total daily dose: As used herein, a “total daily dose” is an amount given or prescribed in 24 hr. period. The total daily dose can be administered as a single unit dose or a split dose.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors can regulate transcription of a target gene alone or in a complex with other molecules.

Transcription: As used herein, the term “transcription” refers to methods to introduce exogenous nucleic acids into a cell. Methods of transfection include, but are not limited to, chemical methods, physical treatments and cationic lipids or mixtures.

Transfection: As used herein, “transfection” refers to the introduction of a polynucleotide into a cell wherein a polypeptide encoded by the polynucleotide is expressed (e.g., mRNA) or the polypeptide modulates a cellular function (e.g., siRNA, miRNA). As used herein, “expression” of a nucleic acid sequence refers to translation of a polynucleotide (e.g., an mRNA) into a polypeptide or protein and/or post-translational modification of a polypeptide or protein.

Treating, treatment, therapy: As used herein, the term “treating” or “treatment” or “therapy” refers to partially or completely alleviating, ameliorating, improving, relieving, delaying onset of, inhibiting progression of, reducing severity of, and/or reducing incidence of one or more signs or symptoms or features of a disease, e.g., muscular dystrophy. For example, “treating” muscular dystrophy can refer to diminishing signs or symptoms associate with the disease, prolong the lifespan (increase the survival rate) of patients, reducing the severity of the disease, preventing or delaying the onset of the disease, etc. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in some way. Unmodified can, but does not always, refer to the wild type or native form of a biomolecule. Molecules can undergo a series of modifications whereby each modified molecule can serve as the “unmodified” starting molecule for a subsequent modification.

Uracil: Uracil is one of the four nucleobases in the nucleic acid of RNA, and it is represented by the letter U. Uracil can be attached to a ribose ring, or more specifically, a ribofuranose via a β-N₁-glycosidic bond to yield the nucleoside uridine. The nucleoside uridine is also commonly abbreviated according to the one letter code of its nucleobase, i.e., U. Thus, in the context of the present disclosure, when a monomer in a polynucleotide sequence is U, such U is designated interchangeably as a “uracil” or a “uridine.”

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

Variant: The term variant as used in present disclosure refers to both natural variants (e.g, polymorphisms, isoforms, etc) and artificial variants in which at least one amino acid residue in a native or starting sequence (e.g., a wild type sequence) has been removed and a different amino acid inserted in its place at the same position. These variants can de described as “substitutional variants.” The substitutions can be single, where only one amino acid in the molecule has been substituted, or they can be multiple, where two or more amino acids have been substituted in the same molecule. If amino acids are inserted or deleted, the resulting variant would be an “insertional variant” or a “deletional variant” respectively.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1: Manufacture of Polynucleotides

According to the present disclosure, the manufacture of polynucleotides and or parts or regions thereof may be accomplished utilizing the methods taught in International Application WO2014/152027 entitled “Manufacturing Methods for Production of RNA Transcripts”, the contents of which is incorporated herein by reference in its entirety.

Purification methods may include those taught in International Application WO2014/152030 and WO2014/152031, each of which is incorporated herein by reference in its entirety.

Detection and characterization methods of the polynucleotides may be performed as taught in WO2014/144039, which is incorporated herein by reference in its entirety.

Characterization of the polynucleotides of the disclosure may be accomplished using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, WO2014/144711 and WO2014/144767, the contents of each of which is incorporated herein by reference in its entirety.

### Example 2: Chimeric Polynucleotide Synthesis

### Introduction

According to the present disclosure, two regions or parts of a chimeric polynucleotide may be joined or ligated using triphosphate chemistry.

According to this method, a first region or part of 100 nucleotides or less is chemically synthesized with a 5′ monophosphate and terminal 3′desOH or blocked OH. If the region is longer than 80 nucleotides, it may be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups may be selected from any of those known in the art.

The second region or part of the chimeric polynucleotide may be synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part.

It is noted that for ligation methods, ligation with DNA T4 ligase, followed by treatment with DNAse should readily avoid concatenation.

The entire chimeric polynucleotide need not be manufactured with a phosphate-sugar backbone. If one of the regions or parts encodes a polypeptide, then it is preferable that such region or part comprise a phosphate-sugar backbone.

Ligation is then performed using any known click chemistry, orthoclick chemistry, solulink, or other bioconjugate chemistries known to those in the art.

### Synthetic Route

The chimeric polynucleotide is made using a series of starting segments. Such segments include:

(a) Capped and protected 5′ segment comprising a normal 3′OH (SEG. 1)

(b) 5′ triphosphate segment which may include the coding region of a polypeptide and comprising a normal 3′OH (SEG. 2)

(c) 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) is treated with cordycepin and then with pyrophosphatase to create the 5′monophosphate.

Segment 2 (SEG. 2) is then ligated to SEG. 3 using RNA ligase. The ligated polynucleotide is then purified and treated with pyrophosphatase to cleave the diphosphate. The treated SEG.2-SEG. 3 construct is then purified and SEG. 1 is ligated to the 5′ terminus. A further purification step of the chimeric polynucleotide may be performed.

Where the chimeric polynucleotide encodes a polypeptide, the ligated or joined segments may be represented as: 5′UTR (SEG. 1), open reading frame or ORF (SEG. 2) and 3′UTR+PolyA (SEG. 3).

The yields of each step may be as much as 90-95%.

### Example 3: PCR for cDNA Production

PCR procedures for the preparation of cDNA are performed using 2× KAPA HIFI™ HotStart ReadyMix by Kapa Biosystems (Woburn, Mass.). This system includes 2×KAPA ReadyMix12.5 μl; Forward Primer (10 μM) 0.75 μl; Reverse Primer (10 μM) 0.75 μl; Template cDNA −100 ng; and dH₂O diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reaction is cleaned up using Invitrogen's PURELINK™ PCR Micro Kit (Carlsbad, Calif.) per manufacturer's instructions (up to 5 μg). Larger reactions will require a cleanup using a product with a larger capacity. Following the cleanup, the cDNA is quantified using the NANODROP™ and analyzed by agarose gel electrophoresis to confirm the cDNA is the expected size. The cDNA is then submitted for sequencing analysis before proceeding to the in vitro transcription reaction.

### Example 4: In Vitro Transcription (IVT)

The in vitro transcription reaction generates polynucleotides containing uniformly modified polynucleotides. Such uniformly modified polynucleotides may comprise a region or part of the polynucleotides of the disclosure. The input nucleotide triphosphate (NTP) mix is made in-house using natural and un-natural NTPs.

A typical in vitro transcription reaction includes the following:

| 1 | Template cDNA | 1.0 | μg |
|---|---|---|---|
| 2 | 10x transcription buffer (400 mM Tris-HCl | 2.0 | μl |
| | pH 8.0, 190 mM MgCl₂, 50 mM DTT, | | |
| | 10 mM Spermidine) | | |
| 3 | Custom NTPs (25 mM each) | 7.2 | μl |
| 4 | RNase Inhibitor | 20 | U |
| 5 | T7 RNA polymerase | 3000 | U |
| 6 | dH₂0 | Up to 20.0 μl. and | |
| 7 | Incubation at 37° C. for 3 hr-5 hrs. | | |

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, Tex.) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

### Example 5: Enzymatic Capping

Capping of a polynucleotide is performed as follows where the mixture includes: IVT RNA 60 μg-180 μg and dH₂O up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400U); Vaccinia capping enzyme (Guanylyl transferase) (40 U); dH₂O (Up to 28 μl); and incubation at 37° C. for 30 minutes for 60 μs RNA or up to 2 hours for 180 μs of RNA.

The polynucleotide is then purified using Ambion's MEGACLEAR™ Kit (Austin, Tex.) following the manufacturer's instructions. Following the cleanup, the RNA is quantified using the NANODROP™ (ThermoFisher, Waltham, Mass.) and analyzed by agarose gel electrophoresis to confirm the RNA is the proper size and that no degradation of the RNA has occurred. The RNA product may also be sequenced by running a reverse-transcription-PCR to generate the cDNA for sequencing.

### Example 6: PolyA Tailing Reaction

Without a poly-T in the cDNA, a poly-A tailing reaction must be performed before cleaning the final product. This is done by mixing Capped IVT RNA (100 μl); RNase Inhibitor (20 U); 10× Tailing Buffer (0.5 M Tris-HCl (pH 8.0), 2.5 M NaCl, 100 mM MgCl₂)(12.0 μl); 20 mM ATP (6.0 μl); Poly-A Polymerase (20 U); dH₂O up to 123.5 μl and incubation at 37° C. for 30 min. If the poly-A tail is already in the transcript, then the tailing reaction may be skipped and proceed directly to cleanup with Ambion's MEGACLEAR™ kit (Austin, Tex.) (up to 500 μg). Poly-A Polymerase is preferably a recombinant enzyme expressed in yeast.

It should be understood that the processivity or integrity of the polyA tailing reaction may not always result in an exact size polyA tail. Hence polyA tails of approximately between 40-200 nucleotides, e.g., about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the invention.

### Example 7: Natural 5′ Caps and 5′ Cap Analogues

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5)ppp(5′) G [the ARCA cap]; G(5)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-0 methyl-transferase. Enzymes are preferably derived from a recombinant source.

When transfected into mammalian cells, the modified mRNAs have a stability of between 12-18 hours or more than 18 hours, e.g., 24, 36, 48, 60, 72 or greater than 72 hours.

### Example 8: Capping Assays

A. Protein Expression Assay

Polynucleotides encoding a polypeptide, containing any of the caps taught herein can be transfected into cells at equal concentrations. 6, 12, 24 and 36 hours post-transfection the amount of protein secreted into the culture medium can be assayed by ELISA. Synthetic polynucleotides that secrete higher levels of protein into the medium would correspond to a synthetic polynucleotide with a higher translationally-competent Cap structure.

B. Purity Analysis Synthesis

Polynucleotides encoding a polypeptide, containing any of the caps taught herein can be compared for purity using denaturing Agarose-Urea gel electrophoresis or HPLC analysis. Polynucleotides with a single, consolidated band by electrophoresis correspond to the higher purity product compared to polynucleotides with multiple bands or streaking bands. Synthetic polynucleotides with a single HPLC peak would also correspond to a higher purity product. The capping reaction with a higher efficiency would provide a more pure polynucleotide population.

C. Cytokine Analysis

Polynucleotides encoding a polypeptide, containing any of the caps taught herein can be transfected into cells at multiple concentrations. 6, 12, 24 and 36 hours post-transfection the amount of pro-inflammatory cytokines such as TNF-alpha and IFN-beta secreted into the culture medium can be assayed by ELISA. Polynucleotides resulting in the secretion of higher levels of pro-inflammatory cytokines into the medium would correspond to a polynucleotides containing an immune-activating cap structure.

D. Capping Reaction Efficiency

Polynucleotides encoding a polypeptide, containing any of the caps taught herein can be analyzed for capping reaction efficiency by LC-MS after nuclease treatment. Nuclease treatment of capped polynucleotides would yield a mixture of free nucleotides and the capped 5′-5-triphosphate cap structure detectable by LC-MS. The amount of capped product on the LC-MS spectra can be expressed as a percent of total polynucleotide from the reaction and would correspond to capping reaction efficiency. The cap structure with higher capping reaction efficiency would have a higher amount of capped product by LC-MS.

### Example 9: Agarose Gel Electrophoresis of Modified RNA or RT PCR Products

Individual polynucleotides (200-400 ng in a 20 μl volume) or reverse transcribed PCR products (200-400 ng) are loaded into a well on a non-denaturing 1.2% Agarose E-Gel (Invitrogen, Carlsbad, Calif.) and run for 12-15 minutes according to the manufacturer protocol.

### Example 10: Nanodrop Modified RNA Quantification and UV Spectral Data

Modified polynucleotides in TE buffer (1 μl) are used for Nanodrop UV absorbance readings to quantitate the yield of each polynucleotide from an chemical synthesis or in vitro transcription reaction.

### Example 11: Formulation of Modified mRNA Using Lipidoids

Polynucleotides are formulated for in vitro experiments by mixing the polynucleotides with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for siRNA-lipidoid formulations may used as a starting point. After formation of the particle, polynucleotide is added and allowed to integrate with the complex. The encapsulation efficiency is determined using a standard dye exclusion assays.

### Example 12: Lipid Nanoparticle Packaging for mRNA Delivery in Embryonic and Larval Zebrafish

### Introduction

Zebrafish provide an outstanding model with which to test potential DMD therapies (Berger and Currie, 2012; Maves, 2014). The zebrafish dmd mutant strain (also known as sapje) contains an early stop codon (Bassett et al., 2003). These zebrafish dmd null mutants show motor defects and severe muscle pathology by 4 days of development and die between 10-30 days post fertilization during the juvenile stage (Berger et al., 2010; Kawahara et al., 2011). dmd zebrafish exhibit many aspects of human DMD pathology, in particular skeletal muscle fibrosis and inflammation, including infiltration of mononuclear cells (Berger et al., 2010; Berger and Currie, 2012). dmd zebrafish have been successfully employed to screen for and test small molecule therapies and to test anti-sense morpholino therapies (Berger et al., 2011; Kawahara et al., 2011; Kawahara et al., 2014; Waugh et al., 2014).

It is demonstrated herein that zebrafish is a model for evaluating mRNA therapeutics for DMD and that mRNA therapeutics are feasible in the treatment of muscular disorders. Lipid-nanoparticle mRNA packaging technology to deliver mRNAs as therapeutics has been developed. The zebrafish animal model provides a system with which to test whether specific mRNAs can functionally modulate the DMD phenotype. The zebrafish dmd model has been used to test delivery, translation, and function of mRNA therapeutics for DMD.

### Results and Discussion

### 1-Cell Stage Yolk Injections

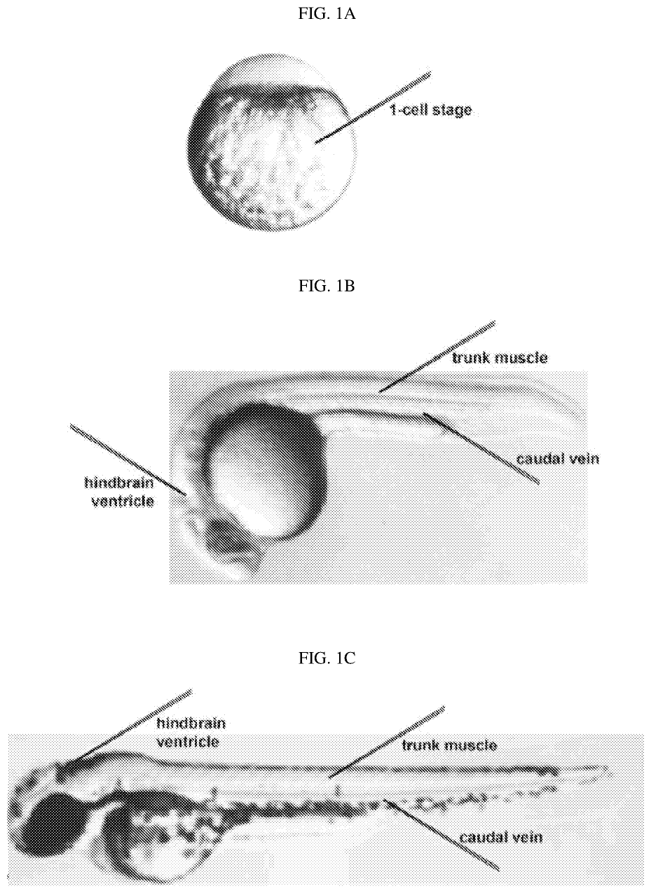

As an initial test of the ability of packaged gfp mRNA to get translated in zebrafish embryos, injections were performed at the 1-cell stage (0-1 hours post fertilization (hpf); see FIG. 1A). These injections are targeted to the yolk, and mRNAs (or other dyes or nucleic acids) are taken up through cytoplasmic streaming into the early blastomeres while the blastomeres are still connected to the yolk (Kimmel et al., 1995). 1-cell-stage injections are a standard method for expressing exogenous mRNAs in early zebrafish embryos. Wild-type fertilized 1-cell-stage embryos were injected with 1 nl of either packaged or naked gfp mRNA, through the chorion into the yolk (FIG. 1A), and then left to develop until they were examined live for GFP expression at about 24 hpf.

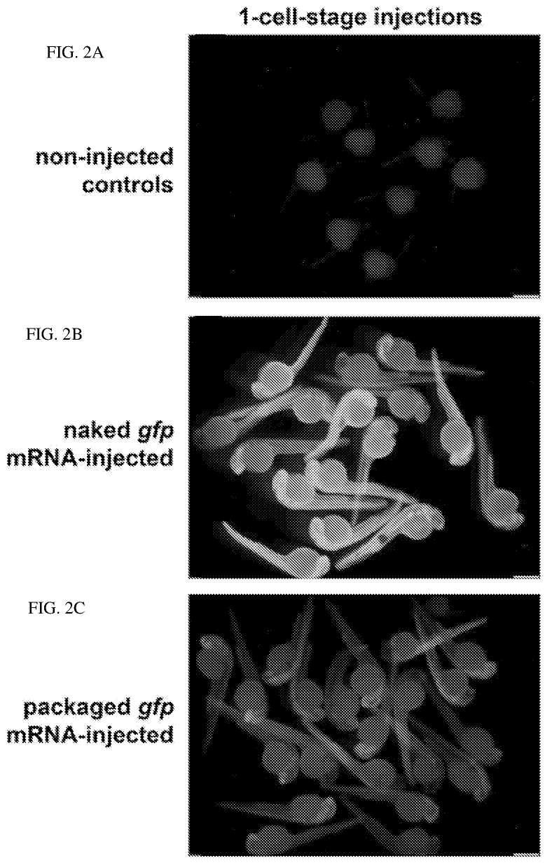

Control (non-injected) embryos showed little background auto-fluorescence at 24 hpf (FIG. 2A). All embryos injected at the 1-cell stage with naked gfp mRNA showed strong GFP expression throughout most tissues at 24 hpf (FIG. 2B; Table 1). All embryos injected at the 1-cell stage with packaged gfp mRNA also showed GFP expression, but in general exhibited less GFP expression than in embryos injected with naked gfp mRNA (FIG. 2C; Table 1). Good survival of embryos injected with either naked or packaged gfp mRNA was observed (Table 1). Similar results were seen in two independent experiments. These experiments show that packaged gfp mRNA can be translated in cells of the zebrafish embryo, although possibly not as efficiently as naked gfp mRNA. These experiments also show that the mRNA packaging formulation does not appear to be toxic to early zebrafish embryos. However, because these 1-cell-stage injections are performed prior to the formation of complete cell membranes, these experiments do not show whether the packaged mRNA formulation can be used to deliver mRNAs across cell membranes within an embryo.

### 24-Hpf-Stage Injections

Recent studies have shown that it is possible to inject materials into zebrafish larvae through multiple routes, such as muscle, blood vessel, and brain ventricle (Benard et al., 2012; Takaki et al., 2013). This experiment was designed to test whether such injections can be used to deliver gfp mRNA into zebrafish embryos and larvae. In particular, whether different microinjection routes enabled delivery of gfp mRNA to muscle or other tissues was tested.

To test whether packaged gfp mRNA can be taken up by cells within the zebrafish embryo, embryos were injected at 24 hpf in three sites: the hindbrain ventricle, the caudal vein, or the trunk skeletal muscle dorsal to the horizontal myoseptum (FIG. 1B). The hindbrain ventricle and caudal vein were chosen as injection sites based on previous literature describing methods for injecting bacteria into zebrafish embryos (Benard et al., 2012; Takaki et al., 2013). Skeletal muscle injections were chosen to test whether the packaged mRNA could be directly targeted to muscle tissue. Each embryo was injected with 1 nl of either packaged or naked gfp mRNA, for a total of 6 injection conditions plus a non-injected control group. Following injections at about 24 hpf, the embryos were left to develop for another 24 hours, anaesthetized in MESAB, and then imaged at about 48 hpf.

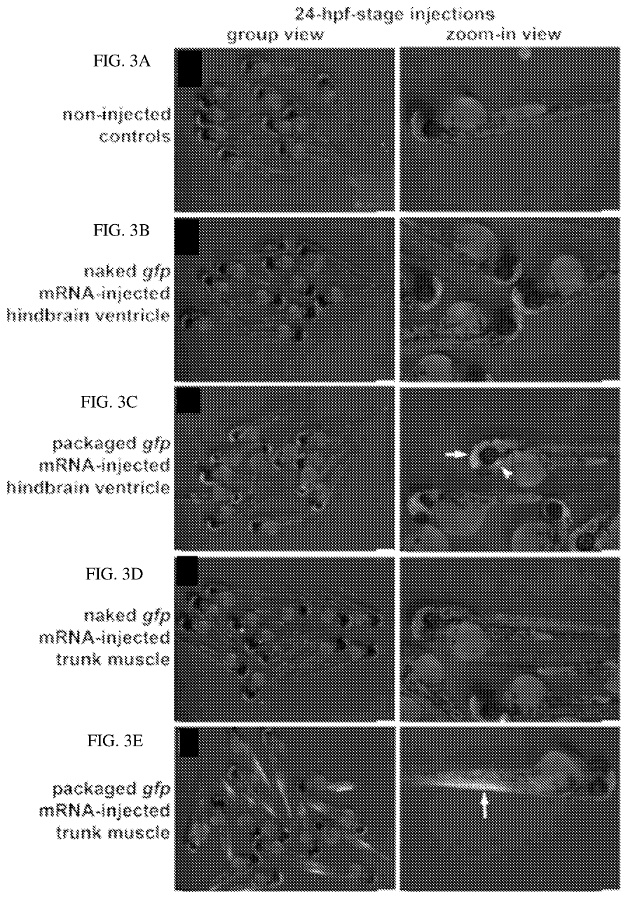

Control (non-injected) embryos show little background auto-fluorescence at 48 hpf (FIG. 3A). Injections of naked gfp mRNA into hindbrain, caudal vein, or muscle at 24 hpf led to very little if any GFP expression at 48 hpf (FIGS. 3B-3D; Table 1). In contrast to the naked gfp mRNA injections, most embryos injected with packaged RNA in the hindbrain showed localized fluorescence around the injection site in the midbrain and forebrain regions (FIG. 3E; Table 1). Embryos injected with packaged gfp mRNA in the caudal vein showed faint expression of GFP in the developing circulatory system and yolk sac (Table 1). Most embryos injected with packaged gfp mRNA into the caudal vein at 24 hpf showed diffuse expression of GFP throughout the embryo at 48 hours, but the expression was only slightly higher than background auto-fluorescence and difficult to score. All embryos injected with packaged gfp mRNA in the muscle showed GFP expression around the injection site, as well as in the spinal cord along the embryo axis centered around the injection site (FIG. 3F). Good survival of embryos injected with either naked or packaged gfp mRNA was observed, although the caudal vein injections generally led to poorer survival than the other injections (Table 1). Similar results were seen in three independent experiments for the hindbrain and trunk muscle injections. Because of the expression and survival issues with the caudal vein injections, only two independent experiments for the caudal vein injections were performed. These experiments show that packaged gfp mRNA can be taken up and translated in cells of the zebrafish embryo, unlike naked gfp mRNA, which exhibits little if any expression following 24 hpf injections.

### 48-Hpf-Stage Injections

To further test whether packaged gfp mRNA can be taken up by cells within the zebrafish embryo, embryos were injected at 48 hpf in three sites: the hindbrain ventricle, the caudal vein, or the trunk skeletal muscle dorsal to the horizontal myoseptum (FIG. 1C). As with the 24 hpf injections, each embryo was injected with 1 nl of either packaged or naked gfp mRNA, for a total of 6 injection conditions plus a non-injected control group. Following injections at about 48 hpf, the embryos were left to develop for another 24 hours, anaesthetized in MESAB, and then imaged at about 72 hpf.

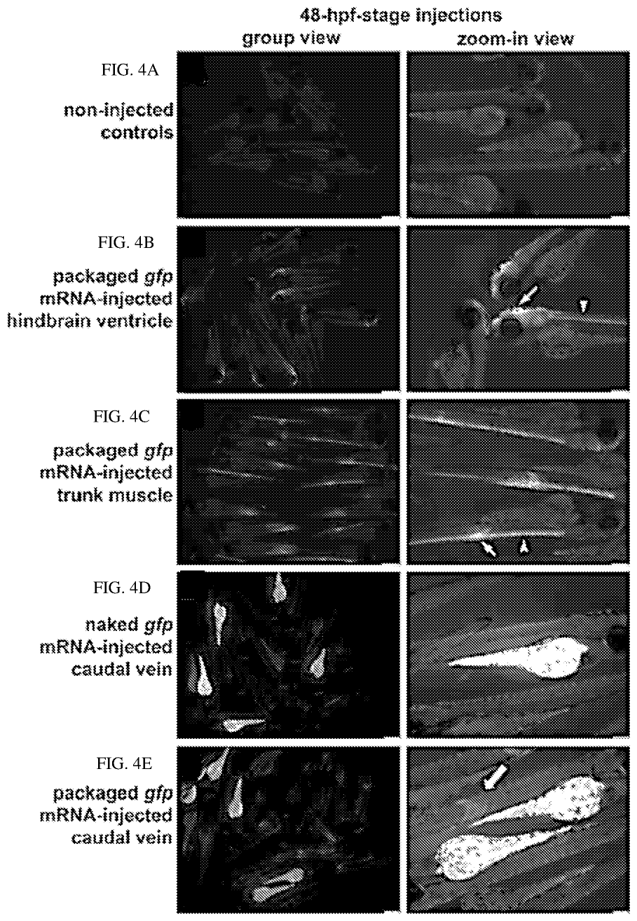

Control (non-injected) embryos showed little background live auto-fluorescence at 72 hpf (FIG. 4A). Injections of naked gfp mRNA into hindbrain or muscle at 48 hpf led to very little if any GFP expression at 72 hpf (Table 1). In contrast to the naked gfp mRNA injections, embryos injected with packaged gfp mRNA in the hindbrain showed strong GFP fluorescence in the forebrain, midbrain, hindbrain and spinal cord (FIG. 4B; Table 1). Embryos injected with packaged gfp mRNA in the trunk muscle showed GFP expression in myotomes immediately surrounding the injection site, as well as in the spinal cord along the embryo axis centered around the injection site (FIG. 4C). For the caudal vein injections, some embryos injected with naked gfp mRNA in the caudal vein showed strong expression of GFP in the yolk sac (FIG. 4D). Embryos injected with packaged gfp mRNA in the caudal vein also showed strong expression of GFP in the yolk sac (FIG. 4E). This could be caused by nicking the yolk sac during injection, a technical error. Because the yolk is a large syncytium, any nicking of the yolk, and subsequent translation of the mRNA, would lead to GFP expression throughout the yolk.

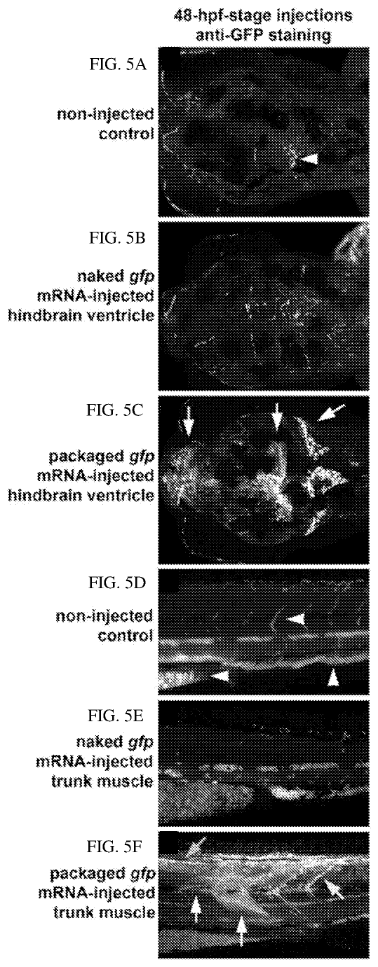

In order to more closely examine the cell types expressing GFP following these injections, embryos were fixed after the live GFP analysis, stained with anti-GFP antibody, and subjected to confocal imaging (FIGS. 5A-5F). In control and in naked gfp mRNA injected embryos, no specific GFP expression was observed; however, background auto-fluorescence in blood cells and vasculature was seen (FIGS. 5A-5B, 5D-5E). Embryos injected with packaged gfp mRNA into the hindbrain ventricle showed GFP expression in large clusters of forebrain, midbrain, and hindbrain neurons (FIG. 5C). Embryos injected with packaged gfp mRNA into trunk muscle showed GFP expression in myotomes around the injection site, and usually about 3-4 myotomes showed expression in muscle fibers (FIG. 5F). These embryos also showed strong GFP expression in the spinal cord and in neural crest cells that populate myotome boundaries (FIG. 5F). Thus, while the injections of packaged gfp mRNA into hindbrain ventricle lead to broadly labelled cells within the CNS, the injections targeting muscle lead to expression in both muscle and neural tissue.

Good survival of embryos injected with either naked or packaged gfp mRNA was observed (Table 1). Similar results were seen in three independent experiments for the hindbrain and muscle injections, and two independent experiments for the caudal vein injections. These experiments show that packaged gfp mRNA injected into the hindbrain and trunk muscle can be taken up and translated in cells of the zebrafish larva, unlike naked gfp mRNA, which exhibits little if any expression following 48 hpf injections. It was also found that injection of either packaged or naked gfp mRNA into the caudal vein at 48 hpf generated little expression of GFP in larval body cells other than the yolk sac.

These experiments yielded several results. First, these experiments, along with the 24 hpf injection experiments, show that muscle injections of packaged mRNA can lead to a high frequency of uptake and expression of protein (gfp) mRNA in trunk skeletal muscle. Second, these experiments show that different injection routes can be used to target different tissues in the zebrafish embryo or larva. Third, these experiments suggest that different tissues may be more amenable to uptake of lipid nanoparticle-packaged mRNA than others. For example, after both hindbrain and muscle injections, we see broad GFP expression in the spinal cord along the axis of the embryo, and not just nearby the injection site. Fourth, in comparing 24-hpf and 48-hpf injections, it was found that the later 48-hpf packaged gfp mRNA injections consistently showed stronger and broader GFP expression than 24-hpf injections. This is perhaps surprising, but could mean that later, more differentiated cells are more amenable to uptake of packaged mRNA, or that the more advanced circulatory system is taking up and distributing packaged mRNA better at 48 hpf.

### CONCLUSIONS

Here it was shown that packaged gfp mRNA can be injected and subsequently be translated in zebrafish embryos and larvae. The primary outcome of these pilot experiments was to demonstrate the ability to deliver exogenous mRNA expression into zebrafish larvae, in particular into skeletal muscle. Now that the ability to deliver gfp mRNA to skeletal muscle has been shown, it is expected that other mRNAs can be delivered to muscle tissue analogously. The basis for demonstrating the therapeutic functions of mRNAs in the zebrafish dmd model has now been established.

TABLE
Table 1. Results from zebrafish embryo injections
with naked and packaged gfp mRNA

| | | | | | |
|---|---|---|---|---|---|
| | | | | Number | |
| | | Number | | with GFP | |
| | | survived | | expression | Percent |
| | Number | 24 hrs post | Percent | 24 hrs post | with GFP |
| Injection type | embryos | injection | survival | injection | expression |

1-cell stage Experiment 1

| | | | | | |
|---|---|---|---|---|---|
| non-injected controls | 10 | 10 | 100 | 0 | 0 |
| naked gfp | 35 | 32 | 91 | 32 | 100 |
| packaged gfp | 70 | 65 | 93 | 65 | 100 |

1-cell stage Experiment 2

| | | | | | |
|---|---|---|---|---|---|
| non-injected controls | 10 | 10 | 100 | 0 | 0 |
| naked gfp | 17 | 16 | 94.1 | 16 | 100 |
| packaged gfp | 22 | 21 | 95.5 | 21 | 100 |

24 hpf Experiment 1

| | | | | | |
|---|---|---|---|---|---|
| non-injected controls | 10 | 10 | 100 | 0 | 0 |
| naked gfp/hindbrain | 20 | 16 | 80 | 0 | 0 |
| naked gfp/caudal vein | 20 | 16 | 80 | 0 | 0 |
| naked gfp/muscle | 20 | 17 | 85 | 4* | 23.5 |
| packaged gfp/hindbrain | 26 | 23 | 88.5 | 21 | 91 |
| packaged gfp/caudal vein | 28 | 21 | 75 | 19 | 90.5 |
| packaged gfp/muscle | 23 | 19 | 82.6 | 19 | 100 |

24 hpf Experiment 2

| | | | | | |
|---|---|---|---|---|---|
| non-injected controls | 10 | 10 | 100 | 0 | 0 |
| naked gfp/hindbrain | 20 | 14 | 70 | 0 | 0 |
| naked gfp/caudal vein | 20 | 13 | 65 | 0 | 0 |
| naked gfp/muscle | 20 | 17 | 85 | 7* | 41 |
| packaged gfp/hindbrain | 20 | 17 | 85 | 3 | 17.6 |
| packged gfp/caudal vein | 20 | 12 | 60 | 3 | 25 |
| packaged gfp/muscle | 20 | 14 | 70 | 14 | 100 |

24 hpf Experiment 3

| | | | | | |
|---|---|---|---|---|---|
| non-injected controls | 10 | 10 | 100 | 0 | 0 |
| naked gfp/hindbrain | 15 | 11 | 73 | 3 | 27.3 |
| naked gfp/muscle | 15 | 13 | 86.7 | 0 | 0 |
| packaged gfp/hindbrain | 15 | 12 | 80 | 9 | 75 |
| packaged gfp/muscle | 20 | 16 | 80 | 16 | 100 |

48 hpf Experiment 1

| | | | | | |
|---|---|---|---|---|---|
| non-injected controls | 10 | 10 | 100 | 0 | 0 |
| naked gfp/hindbrain | 15 | 15 | 100 | 1* | 6.7 |
| naked gfp/caudal vein | 15 | 15 | 100 | 1* | 6.7 |
| naked gfp/muscle | 15 | 15 | 100 | 2* | 13.3 |
| packaged gfp/hindbrain | 20 | 20 | 100 | 15 | 75 |
| packged gfp/caudal vein | 20 | 20 | 100 | 13** | 65 |
| packaged gfp/muscle | 20 | 18 | 90 | 18 | 100 |

48 hpf Experiment 2

| | | | | | |
|---|---|---|---|---|---|
| non-injected controls | 10 | 10 | 100 | 0 | 0 |
| naked gfp/hindbrain | 20 | 20 | 100 | 0 | 0 |
| naked gfp/caudal vein | 20 | 15 | 75 | 5** | 33.3 |
| naked gfp/muscle | 20 | 19 | 95 | 0 | 0 |
| packaged gfp/hindbrain | 20 | 20 | 100 | 18 | 90 |
| packaged gfp/caudal vein | 20 | 20 | 100 | 5** | 25 |
| packaged gfp/muscle | 20 | 19 | 95 | 19 | 100 |

48 hpf Experiment 3

| | | | | | |
|---|---|---|---|---|---|
| non-injected controls | 10 | 10 | 100 | 0 | 0 |
| naked gfp/hindbrain | 10 | 8 | 80 | 0 | 0 |
| naked gfp/muscle | 10 | 9 | 90 | 0 | 0 |
| packaged gfp/hindbrain | 15 | 14 | 93 | 10 | 71.4 |
| packaged gfp/muscle | 20 | 17 | 85 | 16 | 94.1 |

*only 1 or 2 cells with GFP expression in each embryo
**expression largely in yolk sac

### Materials and Methods

Naked and Packaged gfp mRNAs

Naked gfp mRNA was provided at 1.195 mg/ml and was stored at −80° C. Lipid nanoparticle-packaged gfp mRNA was provided at 1.114 mg/ml and was stored at 4° C. The packaged and naked mRNAs were diluted 1:5 from their stock concentrations in phenol red dye (0.1% phenol red and 0.2M KCl in ddH₂O) to give a final injection concentration of 2.40 ng/nl and a final injection quantity of approximately 2.4 ng per embryo.

### Zebrafish Husbandry and Microinjections

All experiments involving live zebrafish (Danio rerio) were carried out in compliance with IACUC guidelines at Seattle Children's Research Institute. Zebrafish were raised and staged as previously described (Westerfield, 2000). Time refers to hours post-fertilization (hpf) at 28.5° C. Embryos were collected from the wild-type AB strain. Embryos were raised in Embryo Medium (EM; Westerfield, 2007). Sibling control embryos were not injected with any mRNAs.

mRNA injections were performed using a Narishige IM 300 Microinjector. Borosilicate glass microcapillary injection needles with filaments (World Precision Instruments, TW100E-4, 1 mm O.D.×0.75 mm I.D.) were prepared using a micropipette puller device (Sutter Instruments Inc., Flaming/Brown p-97) with the following settings: air pressure 200; heat 475; pull 60; velocity 110; time 200. The needle tip was broken off with fine tweezers to obtain a tip opening diameter of 5-10 μm. Microinjections were set up with an injection pressure of 30-40 psi for a duration of 30 milliseconds to give a final injection volume of 1 nl. Injections into 1-cell stage embryos (0-1 hpf) were performed using agar wells to hold embryos in their chorions (Westerfield, 2000). Injections into 24 hpf and 48 hpf embryos were performed by manually dechorionating embryos, anaesthetizing embryos with tricaine methanesulfonate (MESAB; Westerfield, 2000), and individually transferring anaesthetized embryos onto a glass depression slide under a stereomicroscope. Most of the media was removed from around the embryo to prevent it from moving around during injections, but some media was left on the slide to prevent it from sticking to the glass. After the injections were performed, embryos were transferred back into EM in petri dishes.

### Imaging and Immunostaining

Following 1-cell stage, 24 hpf, or 48 hpf injections, embryos were allowed to develop another 24 hrs and then GFP expression was assessed in live embryos using an Olympus SZX16 stereomicroscope with attached Olympus DP72 camera and the cellSens Dimension imaging software. Anaesthetized embryos were imaged on a dark field background through a 488 nm GFP filter with an ISO sensitivity of 200 and an exposure time of 15s.

Following live GFP imaging, embryos injected at 24 hpf and 48 hpf were processed for whole-mount immunostaining as previously described (Bird et al., 2012). Embryos were fixed in 4% paraformaldehyde overnight at 4° C. Embryos were dehydrated in 100% methanol and stored at −20° C. Fixed embryos were rehydrated from 100% methanol into PBS with 0.25% Tween (1×5 min each: 75% MeOH in PBS-Tw, 50% MeOH, 25% MeOH, PBS-Tw). Embryos were then digested with Proteinase K at room temperature for 75 min (10 μg/ml ProtK in PBS-Tw) and washed 3×5 min in PBS-Tw to remove residual ProtK. Embryos were then blocked at room temperature for 2 hours, then put into primary antibody (1:300 anti-GFP, Roche), diluted in fish block, overnight at 4° C. The second day, embryos were rinsed in PBS-Tw 5×15 min, then transferred into secondary antibody (goat anti-mouse AlexaFluor-488, 1:300, Life Technologies/Molecular Probes), diluted in fish block, and left at 4° C. overnight. The third day, embryos were rinsed 5×15 min in PBS-Tw, and embryos were re-fixed in 4% PFA and stored at 4° C. Embryos were mounted in 70% glycerol in PBS under a coverslip and imaged using a Leica TCS SP5 confocal microscope.

### REFERENCES

Bassett D I, Bryson-Richardson R J, Daggett D F, Gautier P, Keenan D G, Currie P D. Development. 2003; 130:5851-5860.
Benard E L, van der Sar A M, Ellett F, Lieschke G J, Spaink H P, Meijer A H. J. Vis. Exp. 2012; 61:e3781.
Berger J, Berger S, Hall 1E, Lieschke G J, Currie P D. Neuromuscul Disord. 2010; 20:826-832.
Berger J, Berger S, Jacoby A S, Wilton S D, Currie P D. J Cell Mol Med. 2011; 15:2643-2651.
Berger J, Currie P D. Dis Model Mech. 2012; 5:726-732.
Bird N C, Windner S E, Devoto S H. Methods Mol Biol. 2012; 798:153-169.
Bushby K, Finkel R, Birnkrant D J, Case L E, Clemens P R, Cripe L, Kaul A, Kinnett K, McDonald C, Pandya S, Poysky J, Shapiro F, Tomersko J, Constantin C; DMD Care Considerations Working Group. Lancet Neurol. 2010; 9:77-93.
Diao J, Wang H, Chang N, Zhou X H, Zhu X, Wang J, Xiong J W. Dev Biol. 2015; 406:196-202.
Hoffman E P, Brown R H Jr, Kunkel L M. Cell. 1987; 51:919-928.
Kawahara G, Karpf J A, Myers J A, Alexander M S, Guyon J R, Kunkel L M. Proc Natl Acad Sci USA. 2011; 108:5331-5336.
Kawahara G, Gasperini M J, Myers J A, Widrick J J, Eran A, Serafini P R, Alexander M S, Pletcher M T, Morris C A, Kunkel L M. Hum Mol Genet. 2014; 23:1869-1878.
Kimmel C B, Ballard W W, Kimmel S R, Ullmann B, Schilling T. Dev Dyn. 1995; 203:253-310.
Kinnett K, Rodger S, Vroom E, Furlong P, Aartsma-Rus A, Bushby K. PLoS Curr. 2015; 7. pii: ecurrents.md.87770501 e86f36f1c71 e0a5882ed9ba1.
Kornegay J N, Spurney C F, Nghiem P P, Brinkmeyer-Langford C L, Hoffman E P, Nagaraju K. ILAR J. 2014; 55:119-149.
Maves L. Expert Opin Drug Discov. 2014; 14:1-13.
Robinson-Hamm J N, Gersbach C A. Hum Genet. 2016; 135:1029-1040.
Takaki K, Winglee K, Ramakrishnan L. Nature Protocols 2013; 8:1114-1124.
Vieira N M, Elvers I, Alexander M S, Moreira Y B, Eran A, Gomes J P, Marshall J L, Karlsson E K, Verjovski-Almeida S, Lindblad-Toh K, Kunkel L M, Zatz M. Cell. 2015; 163:1204-1213.
Waugh T A, Horstick E, Hur J, Jackson S W, Davidson A E, Li X, Dowling H. Hum Mol Genet. 2014; 23:4651-4662.
Westerfield, M. The zebrafish book. A guide for the laboratory use of zebrafish (Dario rerio). 2000; 4th ed., Univ. of Oregon Press, Eugene.

### Sequences

TABLE 5
Amino Acid Sequences

| | | SEQ |
|---|---|---|
| | | ID |
| Name | Sequence | NO: |
| Jagged1 | MRSPRTRGRSGRPLSLLLALLCALRAKVCGASGQFELEILSMQNVNGELQNGNCCGGARNPGDRKCTRDEC | 50 |
| [Homo | DTYFKVCLKEYQSRVTAGGPCSFGSGSTPVIGGNTFNLKASRGNDRNRIVLPFSFAWPRSYTLLVEAWDSS | |
| sapiens] | NDTVQPDSIIEKASHSGMINPSRQWQTLKQNTGVAHFEYQIRVTCDDYYYGFGCNKFCRPRDDFFGHYACD | |
| AAC51731 | QNGNKTCMEGWMGRECNRAICRQGCSPKHGSCKLPGDCRCQYGWQGLYCDKCIPHPGCVHGICNEPWQCLC | |
| | ETNWGGQLCDKDLNYCGTHQPCLNGGTCSNTGPDKYQCSCPEGYSGPNCEIAEHACLSDPCHNRGSCKETS | |
| | LGFECECSPGWTGPTCSTNIDDCSPNNCSHGGTCQDLVNGFKCVCPPQWTGKTCQLDANECEAKPCVNAKS | |
| | CKNLIASYYCDCLPGWMGQNCDININDCLGQCQNDASCRDLVNGYRCICPPGYAGDHCERDIDECASNPCL | |
| | DGGHCQNEINRFQCLCPTGFSGNLCQLDIDYCEPNPCQNGAQCYNRASDYFCKCPEDYEGKNCSHLKDHCR | |
| | TTPCEVIDSCTVAMASNDTPEGVRYISSNVCGPHGKCKSQSGGKFTCDCNKGFTGTYCHENINDCESNPCR | |
| | NGGTCIDGVNSYKCICSDGWEGAYCETNINDCSQNPCHNGGTCRDLVNDFYCDCKNGWKGKTCHSRDSQCD | |
| | EATCNNGGTCYDEGDAFKCMCPGGWEGTTCNIARNSSCLPNPCHNGGTCVVNGESFTCVCKEGWEGPICAQ | |
| | NTNDCSPHPCYNSGTCVDGDNWYRCECAPGFAGPDCRININECQSSPCAFGATCVDEINGYRCVCPPGHSG | |
| | AKCQEVSGRPCITMGSVIPDGAKWDDDCNTCQCLNGRIACSKVWCGPRPCLLHKGHSECPSGQSCIPILDD | |
| | QCFVHPCTGVGECRSSSLQPVKTKCTSDSYYQDNCANITFTFNKEMMSPGLTTEHICSELRNLNILKNVSA | |
| | EYSIYIACEPSPSANNEIHVAISAEDIRDDGNPIKEITDKIIDLVSKRDGNSSLIAAVAEVRVQRRPLKNR | |
| | TDFLVPLLSSVLTVAWICCLVTAFYWCLRKRRKPGSHTHSASEDNTTNNVREQLNQIKNPIEKHGANTVPI | |
| | KDYENKNSKMSKIRTHNSEVEEDDMDKHQQKARFAKQPAYTLVDREEKPPNGTPTKHPNWTNKQDNRDLES | |
| | AQSLNRMEYIV | |
| Dystrophin | MLWWEEVEDCYEREDVQKKTFTKWVNAQFSKFGKQHIENLFSDLQDGRRLLDLLEGLTGQKLPKEKGSTR | 51 |
| [Homo | VHALNNVNKALRVLQNNNVDLVNIGSTDIVDGNHKLTLGLIWNIILHWQVKNVMKNIMAGLQQTNSEKIL | |
| sapiens] | LSWVRQSTRNYPQVNVINFTTSWSDGLALNALIHSHRPDLFDWNSVVCQQSATQRLEHAFNIARYQLGIE | |
| AAA53189 | KLLDPEDVDTTYPDKKSILMYITSLFQVLPQQVSIEAIQEVEMLPRPPKVTKEEHFQLHHQMHYSQQITV | |
| | SLAQGYERTSSPKPRFKSYAYTQAAYVTTSDPTRSPFPSQHLEAPEDKSFGSSLMESEVNLDRYQTALEE | |
| | VLSWLLSAEDTLQAQGEISNDVEVVKDQFHTHEGYMMDLTAHQGRVGNILQLGSKLIGTGKLSEDEETEV | |
| | QEQMNLLNSRWECLRVASMEKQSNLHRVLMDLQNQKLKELNDWLTKTEERTRKMEEEPLGPDLEDLKRQV | |
| | QQHKVLQEDLEQEQVRVNSLTHMVVVVDESSGDHATAALEEQLKVLGDRWANICRWTEDRWVLLQDILLK | |
| | WQRLTEEQCLFSAWLSEKEDAVNKIHTTGFKDQNEMLSSLQKLAVLKADLEKKKQSMGKLYSLKQDLLST | |
| | LKNKSVTQKTEAWLDNFARCWDNLVQKLEKSTAQISQAVTTTQPSLTQTTVMETVTTVTTREQILVKHAQ | |
| | EELPPPPPQKKRQITVDSEIRKRLDVDITELHSWITRSEAVLQSPEFAIFRKEGNFSDLKEKVNAIEREK | |
| | AEKFRKLQDASRSAQALVEQMVNEGVNADSIKQASEQLNSRWIEFCQLLSERLNWLEYQNNIIAFYNQLQ | |
| | QLEQMTTTAENWLKIQPTTPSEPTAIKSQLKICKDEVNRLSGLQPQIERLKIQSIALKEKGQGPMFLDAD | |
| | FVAFTNHFKQVFSDVQAREKELQTIFDTLPPMRYQETMSAIRTWVQQSETKLSIPQLSVTDYEIMEQRLG | |
| | ELQALQSSLQEQQSGLYYLSTTVKEMSKKAPSEISRKYQSEFEEIEGRWKKLSSQLVEHCQKLEEQMNKL | |
| | RKIQNHIQTLKKWMAEVDVFLKEEWPALGDSEILKKQLKQCRLLVSDIQTIQPSLNSVNEGGQKIKNEAE | |
| | PEFASRLETELKELNTQWDHMCQQVYARKEALKGGLEKTVSLQKDLSEMHEWMTQAEEEYLERDFEYKTP | |
| | DELQKAVEEMKRAKEEAQQKEAKVKLLTESVNSVIAQAPPVAQEALKKELETLTTNYQWLCTRLNGKCKT | |
| | LEEVWACWHELLSYLEKANKWLNEVEFKLKTTENIPGGAEEISEVLDSLENLMRHSEDNPNQIRILAQTL | |
| | TDGGVMDELINEELETFNSRWRELHEEAVRRQKLLEQSIQSAQETEKSLHLIQESLTFIDKQLAAYIADK | |
| | VDAAQMPQEAQKIQSDLTSHEISLEEMKKHNQGKEAAQRVLSQIDVAQKKLQDVSMKFRLFQKPANFELR | |
| | LQESKMILDEVKMHLPALETKSVEQEVVQSQLNHCVNLYKSLSEVKSEVEMVIKTGRQIVQKKQTENPKE | |
| | LDERVTALKLHYNELGAKVTERKQQLEKCLKLSRKMRKEMNVLTEWLAATDMELTKRSAVEGMPSNLDSE | |
| | VAWGKATQKEIEKQKVHLKSITEVGEALKTVLGKKETLVEDKLSLLNSNWIAVTSRAEEWLNLLLEYQKH | |
| | METFDQNVDHITKWIIQADTLLDESEKKKPQQKEDVLKRLKAELNDIRPKVDSTRDQAANLMANRGDHCR | |
| | KLVEPQISELNHRFAAISHRIKTGKASIPLKELEQFNSDIQKLLEPLEAEIQQGVNLKEEDFNKDMNEDN | |
| | EGTVKELLQRGDNLQQRITDERKREEIKIKQQLLQTKHNALKDLRSQRRKKALEISHQWYQYKRQADDLL | |
| | KCLDDIEKKLASLPEPRDERKIKEIDRELQKKKEELNAVRRQAEGLSEDGAAMAVEPTQIQLSKRWREIE | |
| | SKFAQFRRLNFAQIHTVREETMMVMTEDMPLEISYVPSTYLTEITHVSQALLEVEQLLNAPDLCAKDFED | |
| | LFKQEESLKNIKDSLQQSSGRIDIIHSKKTAALQSATPVERVKLQEALSQLDFQWEKVNKMYKDRQGRFD | |
| | RSVEKWRRFHYDIKIFNQWLTEAEQFLRKTQIPENWEHAKYKWYLKELQDGIGQRQTVVRTLNATGEEII | |
| | QQSSKTDASILQEKLGSLNLRWQEVCKQLSDRKKRLEEQKNILSEFQRDLNEFVLWLEEADNIASIPLEP | |
| | GKEQQLKEKLEQVKLLVEELPLRQGILKQLNETGGPVLVSAPISPEEQDKLENKLKQTNLQWIKVSRALP | |
| | EKQGEIEAQIKDLGQLEKKLEDLEEQLNHLLLWLSPIRNQLEIYNQPNQEGPFDVQETEIAVQAKQPDVE | |
| | EILSKGQHLYKEKPATQPVKRKLEDLSSEWKAVNRLLQELRAKQPDLAPGLTTIGASPTQTVTLVTQPVV | |
| | TKETAISKLEMPSSLMLEVPALADFNRAWTELTDWLSLLDQVIKSQRVMVGDLEDINEMIIKQKATMQDL | |
| | EQRRPQLEELITAAQNLKNKTSNQEARTIITDRIERIQNQWDEVQEHLQNRRQQLNEMLKDSTQWLEAKE | |
| | EAEQVLGQARAKLESWKEGPYTVDAIQKKITETKQLAKDLRQWQTNVDVANDLALKLLRDYSADDTRKVH | |
| | MITENINASWRSIHKRVSEREAALEETHRLLQQFPLDLEKFLAWLTEAETTANVLQDATRKERLLEDSKG | |
| | VKELMKQWQDLQGEIEAHTDVYHNLDENSQKILRSLEGSDDAVLLQRRLDNMNFKWSELRKKSLNIRSHL | |
| | EASSDQWKRLHLSLQELLVWLQLKDDELSRQAPIGGDFPAVQKQNDVHRAFKRELKTKEPVIMSTLETVR | |
| | IFLTEQPLEGLEKLYQEPRELPPEERAQNVTRLLRKQAEEVNTEWEKLNLHSADWQRKIDETLERLQELQ | |
| | EATDELDLKLRQAEVIKGSWQPVGDLLIDSLQDHLEKVKALRGEIAPLKENVSHVNDLARQLTTLGIQLS | |
| | PYNLSTLEDLNTRWKLLQVAVEDRVRQLHEAHRDFGPASQHFLSTSVQGPWERAISPNKVPYYINHETQT | |
| | TCWDHPKMTELYQSLADLNNVRFSAYRTAMKLRRLQKALCLDLLSLSAACDALDQHNLKQNDQPMDILQI | |
| | INCLTTIYDRLEQEHNNLVNVPLCVDMCLNWLLNVYDTGRTGRIRVLSFKTGIISLCKAHLEDKYRYLFK | |
| | QVASSTGFCDQRRLGLLLHDSIQIPRQLGEVASFGGSNIEPSVRSCFQFANNKPEIEAALFLDWMRLEPQ | |
| | SMVWLPVLHRVAAAETAKHQAKCNICKECPIIGFRYRSLKHFNYDICQSCFFSGRVAKGHKMHYPMVEYC | |
| | TPTTSGEDVRDFAKVLKNKFRTKRYFAKHPRMGYLPVQTVLEGDNMETPVTLINFWPVDSAPASSPQLSH | |
| | DDTHSRIEHYASRLAEMENSNGSYLNDSISPNESIDDEHLLIQHYCQSLNQDSPLSQPRSPAQILISLES | |
| | EERGELERILADLEEENRNLQAEYDRLKQQHEHKGLSPLPSPPEMMPTSPQSPRDAELIAEAKLLRQHKG | |
| | RLEARMQILEDHNKQLESQLHRLRQLLEQPQAEAKVNGTTVSSPSTSLQRSDSSQPMLLRVVGSQTSDSM | |
| | GEEDLLSPPQDTSTGLEEVMEQLNNSFPSSRGRNTPGKPMREDTM | |
| laminin, | MPGAAGVLLLLLLSGGLGGVQAQRPQQQRQSQAHQQRGLFPAVLNLASNALITTNATCGEKGPEMYCKLV | 52 |
| alpha 2 | EHVPGQPVRNPQCRICNQNSSNPNQRHPITNAIDGKNTWWQSPSIKNGIEYHYVTITLDLQQVFQIAYVI | |
| (merosin, | VKAANSPRPGNWILERSLDDVEYKPWQYHAVTDTECLTLYNIYPRTGPPSYAKDDEVICTSFYSKIHPLE | |
| congenital | NGEIHISLINGRPSADDPSPELLEFTSARYIRLRFQRIRTLNADLMMFAHKDPREIDPIVTRRYYYSVKD | |
| muscular | ISVGGMCICYGHARACPLDPATNKSRCECEHNTCGDSCDQCCPGFHQKPWRAGTFLTKTECEACNCHGKA | |
| dystrophy | EECYYDENVARRNLSLNIRGKYIGGGVCINCTQNTAGINCETCTDGFFRPKGVSPNYPRPCQPCHCDPIG | |
| [Homo | SLNEVCVKDEKHARRGLAPGSCHCKTGFGGVSCDRCARGYTGYPDCKACNCSGLGSKNEDPCFGPCICKE | |
| sapiens] | NVEGGDCSRCKSGFFNLQEDNWKGCDECFCSGVSNRCQSSYWTYGKIQDMSGWYLTDLPGRIRVAPQQDD | |
| | LDSPQQISISNAEARQALPHSYYWSAPAPYLGNKLPAVGGQLTFTISYDLEEEEEDTERVLQLMIILEGN | |
| | DLSISTAQDEVYLHPSEEHTNVLLLKEESFTIHGTHFPVRRKEFMTVLANLKRVLLQITYSFGMDAIFRL | |
| | SSVNLESAVSYPTDGSIAAAVEVCQCPPGYTGSSCESCWPRHRRVNGTIFGGICEPCQCFGHAESCDDVT | |
| | GECLNCKDHTGGPYCDKCLPGFYGEPTKGTSEDCQPCACPLNIPSNNFSPTCHLDRSLGLICDGCPVGYT | |
| | GPRCERCAEGYFGQPSVPGGSCQPCQCNDNLDFSIPGSCDSLSGSCLICKPGTTGRYCELCADGYFGDAV | |
| | DAKNCQPCRCNAGGSFSEVCHSQTGQCECRANVQGQRCDKCKAGTFGLQSARGCVPCNCNSFGSKSFDCE | |
| | ESGQCWCQPGVTGKKCDRCAHGYFNFQEGGCTACECSHLGNNCDPKTGRCICPPNTIGEKCSKCAPNTWG | |
| | HSITTGCKACNCSTVGSLDFQCNVNTGQCNCHPKFSGAKCTECSRGHWNYPRCNLCDCFLPGTDATTCDS | |
| | ETKKCSCSDQTGQCTCKVNVEGIHCDRCRPGKFGLDAKNPLGCSSCYCFGTTTQCSEAKGLIRTWVTLKA | |
| | EQTILPLVDEALQHTTTKGIVFQHPEIVAHMDLMREDLHLEPFYWKLPEQFEGKKLMAYGGKLKYAIYFE | |
| | AREETGFSTYNPQVIIRGGTPTHARIIVRHMAAPLIGQLTRHEIEMTEKEWKYYGDDPRVHRTVTREDFL | |
| | DILYDIHYILIKATYGNFMRQSRISEISMEVAEQGRGTTMTPPADLIEKCDCPLGYSGLSCEACLPGFYR | |
| | LRSQPGGRTPGPTLGTCVPCQCNGHSSLCDPETSICQNCQHHTAGDFCERCALGYYGIVKGLPNDCQQCA | |
| | CPLISSSNNFSPSCVAEGLDDYRCTACPRGYEGQYCERCAPGYTGSPGNPGGSCQECECDPYGSLPVPCD | |
| | PVTGFCTCRPGATGRKCDGCKHWHAREGWECVFCGDECTGLLLGDLARLEQMVMSINLTGPLPAPYKMLY | |
| | GLENMTQELKHLLSPQRAPERLIQLAEGNLNTLVTEMNELLTRATKVTADGEQTGQDAERTNTRAKSLGE | |
| | FIKELARDAEAVNEKAIKLNETLGTRDEAFERNLEGLQKEIDQMIKELRRKNLETQKEIAEDELVAAEAL | |
| | LKKVKKLFGESRGENEEMEKDLREKLADYKNKVDDAWDLLREATDKIREANRLFAVNQKNMTALEKKKEA | |
| | VESGKRQIENTLKEGNDILDEANRLADEINSIIDYVEDIQTKLPPMSEELNDKIDDLSQEIKDRKLAEKV | |
| | SQAESHAAQLNDSSAVLDGILDEAKNISFNATAAFKAYSNIKDYIDEAEKVAKEAKDLAHEATKLATGPR | |
| | GLLKEDAKGCLQKSFRILNEAKKLANDVKENEDHLNGLKTRIENADARNGDLLRTLNDTLGKLSAIPNDT | |
| | AAKLQAVKDKARQANDTAKDVLAQITELHQNLDGLKKNYNKLADSVAKTNAVVKDPSKNKIIADADATVK | |
| | NLEQEADRLIDKLKPIKELEDNLKKNISEIKELINQARKQANSIKVSVSSGGDCIRTYKPEIKKGSYNNI | |
| | VVNVKTAVADNLLFYLGSAKFIDFLAIEMRKGKVSFLWDVGSGVGRVEYPDLTIDDSYWYRIVASRTGRN | |
| | GTISVRALDGPKASIVPSTHHSTSPPGYTILDVDANAMLFVGGLTGKLKKADAVRVITFTGCMGETYFDN | |
| | KPIGLWNFREKEGDCKGCTVSPQVEDSEGTIQFDGEGYALVSRPIRWYPNISTVMFKFRTFSSSALLMYL | |
| | ATRDLRDFMSVELTDGHIKVSYDLGSGMASVVSNQNHNDGKWKSFTLSRIQKQANISIVDIDTNQEENIA | |
| | TSSSGNNFGLDLKADDKIYFGGLPTLRNLSMKARPEVNLKKYSGCLKDIEISRTPYNILSSPDYVGVTKG | |
| | CSLENVYTVSFPKPGFVELSPVPIDVGTEINLSFSTKNESGIILLGSGGTPAPPRRKRRQTGQAYYAILL | |
| | NRGRLEVHLSTGARTMRKIVIRPEPNLFHDGREHSVHVERTRGIFTVQVDENRRYMQNLTVEQPIEVKKL | |
| | FVGGAPPEFQPSPLRNIPPFEGCIWNLVINSVPMDFARPVSFKNADIGRCAHQKLREDEDGAAPAEIVIQ | |
| | PEPVPTPAFPTPTPVLTHGPCAAESEPALLIGSKQFGLSRNSHIAIAFDDTKVKNRLTIELEVRTEAESG | |
| | LLFYMARINHADFATVQLRNGLPYFSYDLGSGDTHTMIPTKINDGQWHKIKIMRSKQEGILYVDGASNRT | |
| | ISPKKADILDVVGMLYVGGLPINYTTRRIGPVTYSIDGCVRNLHMAEAPADLEQPTSSFHVGTCFANAQR | |
| | GTYFDGTGFAKAVGGFKVGLDLLVEFEFRTTTTTGVLLGISSQKMDGMGIEMIDEKLMFHVDNGAGRFTA | |
| | VYDAGVPGHLCDGQWHKVTANKIKHRIELTVDGNQVEAQSPNPASTSADTNDPVFVGGFPDDLKQFGLTT | |
| | SIPFRGCIRSLKLTKGTGKPLEVNFAKALELRGVQPVSCPAN | |
| emerin | MDNYADLSDTELTTLLRRYNIPHGPVVGSTRRLYEKKIFEYETQRRRLSPPSSSAASSYSFSDLNSTRGD | 53 |
| [Homo | ADMYDLPKKEDALLYQSKGYNDDYYEESYFTTRTYGEPESAGPSRAVRQSVTSFPDADAFHHQVHDDDLL | |
| sapiens] | SSSEEECKDRERPMYGRDSAYQSITHYRPVSASRSSLDLSYYPTSSSTSFMSSSSSSSSWLTRRAIRPEN | |
| | RAPGAGLGQDRQVPLWGQLLLFLVFVIVLFFIYHFMQAEEGNPF | |
| dysferlin | MLRVFILYAENVHTPDTDISDAYCSAVFAGVKKRTKVIKNSVNPVWNEGFEWDLKGIPLDQGSELHVVVK | 54 |
| [Homo | DHETMGRNRFLGEAKVPLREVLATPSLSASFNAPLLDTKKQPTGASLVLQVSYTPLPGAVPLFPPPTPLE | |
| sapiens] | PSPTLPDLDVVADTGGEEDTEDQGLTGDEAEPFLDQSGGPGAPTTPRKLPSRPPPHYPGIKRKRSAPTSR | |
| | KLLSDKPQDFQIRVQVIEGRQLPGVNIKPVVKVTAAGQTKRTRIHKGNSPLFNETLFFNLFDSPGELFDE | |
| | PIFITVVDSRSLRTDALLGEFRMDVGTIYREPRHAYLRKWLLLSDPDDFSAGARGYLKTSLCVLGPGDEA | |
| | PLERKDPSEDKEDIESNLLRPTGVALRGAHFCLKVFRAEDLPQMDDAVMDNVKQIFGFESNKKNLVDPFV | |
| | EVSFAGKMLCSKILEKTANPQWNQNITLPAMFPSMCEKMRIRIIDWDRLTHNDIVATTYLSMSKISAPGG | |
| | EIEEEPAGAVKPSKASDLDDYLGFLPTFGPCYINLYGSPREFTGFPDPYTELNTGKGEGVAYRGRLLLSL | |
| | ETKLVEHSEQKVEDLPADDILRVEKYLRRRKYSLFAAFYSATMLQDVDDAIQFEVSIGNYGNKFDMTCLP | |
| | LASTTQYSRAVFDGCHYYYLPWGNVKPVVVLSSYWEDISHRIETQNQLLGIADRLEAGLEQVHLALKAQC | |
| | STEDVDSLVAQLTDELIAGCSQPLGDIHETPSATHLDQYLYQLRTHHLSQITEAALALKLGHSELPAALE | |
| | QAEDWLLRLRALAEEPQNSLPDIVIWMLQGDKRVAYQRVPAHQVLFSRRGANYCGKNCGKLQTIFLKYPM | |
| | EKVPGARMPVQIRVKLWFGLSVDEKEFNQFAEGKLSVFAETYENETKLALVGNWGTTGLTYPKFSDVTGK | |
| | IKLPKDSFRPSAGWTWAGDWFVCPEKTLLHDMDAGHLSFVEEVFENQTRLPGGQWIYMSDNYTDVNGEKV | |
| | LPKDDIECPLGWKWEDEEWSTDLNRAVDEQGWEYSITIPPERKPKHWVPAEKMYYTHRRRRWVRLRRRDL | |
| | SQMEALKRHRQAEAEGEGWEYASLFGWKFHLEYRKTDAFRRRRWRRRMEPLEKTGPAAVFALEGALGGVM | |
| | DDKSEDSMSVSTLSFGVNRPTISCIFDYGNRYHLRCYMYQARDLAAMDKDSFSDPYAIVSFLHQSQKTVV | |
| | VKNTLNPTWDQTLIFYEIEIFGEPATVAEQPPSIVVELYDHDTYGADEFMGRCICQPSLERMPRLAWFPL | |
| | TRGSQPSGELLASFELIQREKPAIHHIPGFEVQETSRILDESEDTDLPYPPPQREANIYMVPQNIKPALQ | |
| | RTAIEILAWGLRNMKSYQLANISSPSLVVECGGQTVQSCVIRNLRKNPNFDICTLFMEVMLPREELYCPP | |
| | ITVKVIDNRQFGRRPVVGQCTIRSLESFLCDPYSAESPSPQGGPDDVSLLSPGEDVLIDIDDKEPLIPIQ | |
| | EEEFIDWWSKFFASIGEREKCGSYLEKDFDTLKVYDTQLENVEAFEGLSDFCNTFKLYRGKTQEETEDPS | |
| | VIGEFKGLFKIYPLPEDPAIPMPPRQFHQLAAQGPQECLVRIYIVRAFGLQPKDPNGKCDPYIKISIGKK | |
| | SVSDQDNYIPCTLEPVFGKMFELTCTLPLEKDLKITLYDYDLLSKDEKIGETVVDLENRLLSKFGARCGL | |
| | PQTYCVSGPNQWRDQLRPSQLLHLFCQQHRVKAPVYRTDRVMFQDKEYSIEEIEAGRIPNPHLGPVEERL | |
| | ALHVLQQQGLVPEHVESRPLYSPLQPDIEQGKLQMWVDLFPKALGRPGPPFNITPRRARRFFLRCIIWNT | |
| | RDVILDDLSLTGEKMSDIYVKGWMIGFEEHKQKTDVHYRSLGGEGNFNWRFIFPFDYLPAEQVCTIAKKD | |
| | AFWRLDKTESKIPARVVFQIWDNDKFSFDDFLGSLQLDLNRMPKPAKTAKKCSLDQLDDAFHPEWFVSLF | |
| | EQKTVKGWWPCVAEEGEKKILAGKLEMTLEIVAESEHEERPAGQGRDEPNMNPKLEDPRRPDTSFLWFTS | |
| | PYKTMKFILWRRFRWAIILFIILFILLLFLAIFIYAFPNYAAMKLVKPFS | |
| Lamin | METPSQRRATRSGAQASSTPLSPTRITRLQEKEDLQELNDRLAVYIDRVRSLETENAGLRLRITESEEVV | 55 |
| A/C | SREVSGIKAAYEAELGDARKTLDSVAKERARLQLELSKVREEFKELKARNTKKEGDLIAAQARLKDLEAL | |
| [Homo | LNSKEAALSTALSEKRTLEGELHDLRGQVAKLEAALGEAKKQLQDEMLRRVDAENRLQTMKEELDFQKNI | |
| sapiens] | YSEELRETKRRHETRLVEIDNGKQREFESRLADALQELRAQHEDQVEQYKKELEKTYSAKLDNARQSAER | |
| | NSNLVGAAHEELQQSRIRIDSLSAQLSQLQKQLAAKEAKLRDLEDSLARERDTSRRLLAEKEREMAEMRA | |
| | RMQQQLDEYQELLDIKLALDMEIHAYRKLLEGEEERLRLSPSPTSQRSRGRASSHSSQTQGGGSVTKKRK | |
| | LESTESRSSFSQHARTSGRVAVEEVDEEGKFVRLRNKSNEDQSMGNWQIKRQNGDDPLLTYRFPPKFTLK | |
| | AGQVVTIWAAGAGATHSPPTDLVWKAQNTWGCGNSLRTALINSTGEEVAMRKLVRSVTVVEDDEDEDGDD | |
| | LLHHHHVSGSRR | |

TABLE 6
DNA Sequences

| | | SEQ |
|---|---|---|
| | | ID |
| Name | Sequence | NO: |
| AF003837.1 | CCGGGTCCTTCTCCGAGAGCCGGGCGGGCACGCGTCATTGTGTTACCTGCGGCCGGCCCGCGAGCTAGGC | 56 |
| Homo | TGGTTTTTTTTTTTTCTCCCCTCCCTCCCCCCTTTTTCCATGCAGCTGATCTAAAAGGGAATAAAAGGCT | |
| sapiens | GCGCATAATCATAATAATAAAAGAAGGGGAGCGCGAGAGAAGGAAAGAAAGCCGGGAGGTGGAAGAGGAG | |
| Jagged1 | GGGGAGCGTCTCAAAGAAGCGATCAGAATAATAAAAGGAGGCCGGGCTCTTTGCCTTCTGGAACGGGCCG | |
| (JAG1), | CTCTTGAAAGGGCTTTTGAAAAGTGGTGTTGTTTTCCAGTCGTGCATGCTCCAATCGGCGGAGTATATTA | |
| complete | GAGCCGGGACGCGGCGGCCGCAGGGGCAGCGGCGACGGCAGCACCGGCGGCAGCACCAGCGCGAACAGCA | |
| cds | GCGGCGGCGTCCCGAGTGCCCGCGGCGCGCGGCGCAGCGATGCGTTCCCCACGGACGCGCGGCCGGTCCG | |
| | GGCGCCCCCTAAGCCTCCTGCTCGCCCTGCTCTGTGCCCTGCGAGCCAAGGTGTGTGGGGCCTCGGGTCA | |
| | GTTCGAGTTGGAGATCCTGTCCATGCAGAACGTGAACGGGGAGCTGCAGAACGGGAACTGCTGCGGCGGC | |
| | GCCCGGAACCCGGGAGACCGCAAGTGCACCCGCGACGAGTGTGACACATACTTCAAAGTGTGCCTCAAGG | |
| | AGTATCAGTCCCGCGTCACGGCCGGGGGGCCCTGCAGCTTCGGCTCAGGGTCCACGCCTGTCATCGGGGG | |
| | CAACACCTTCAACCTCAAGGCCAGCCGCGGCAACGACCGCAACCGCATCGTGCTGCCTTTCAGTTTCGCC | |
| | TGGCCGAGGTCCTATACGTTGCTTGTGGAGGCGTGGGATTCCAGTAATGACACCGTTCAACCTGACAGTA | |
| | TTATTGAAAAGGCTTCTCACTCGGGCATGATCAACCCCAGCCGGCAGTGGCAGACGCTGAAGCAGAACAC | |
| | GGGCGTTGCCCACTTTGAGTATCAGATCCGCGTGACCTGTGATGACTACTACTATGGCTTTGGCTGCAAT | |
| | AAGTTCTGCCGCCCCAGAGATGACTTCTTTGGACACTATGCCTGTGACCAGAATGGCAACAAAACTTGCA | |
| | TGGAAGGCTGGATGGGCCGCGAATGTAACAGAGCTATTTGCCGACAAGGCTGCAGTCCTAAGCATGGGTC | |
| | TTGCAAACTCCCAGGTGACTGCAGGTGCCAGTACGGCTGGCAAGGCCTGTACTGTGATAAGTGCATCCCA | |
| | CACCCGGGATGCGTCCACGGCATCTGTAATGAGCCCTGGCAGTGCCTCTGTGAGACCAACTGGGGCGGCC | |
| | AGCTCTGTGACAAAGATCTCAATTACTGTGGGACTCATCAGCCGTGTCTCAACGGGGGAACTTGTAGCAA | |
| | CACAGGCCCTGACAAATATCAGTGTTCCTGCCCTGAGGGGTATTCAGGACCCAACTGTGAAATTGCTGAG | |
| | CACGCCTGCCTCTCTGATCCCTGTCACAACAGAGGCAGCTGTAAGGAGACCTCCCTGGGCTTTGAGTGTG | |
| | AGTGTTCCCCAGGCTGGACCGGCCCCACATGCTCTACAAACATTGATGACTGTTCTCCTAATAACTGTTC | |
| | CCACGGGGGCACCTGCCAGGACCTGGTTAACGGATTTAAGTGTGTGTGCCCCCCACAGTGGACTGGGAAA | |
| | ACGTGCCAGTTAGATGCAAATGAATGTGAGGCCAAACCTTGTGTAAACGCCAAATCCTGTAAGAATCTCA | |
| | TTGCCAGCTACTACTGCGACTGTCTTCCCGGCTGGATGGGTCAGAATTGTGACATAAATATTAATGACTG | |
| | CCTTGGCCAGTGTCAGAATGACGCCTCCTGTCGGGATTTGGTTAATGGTTATCGCTGTATCTGTCCACCT | |
| | GGCTATGCAGGCGATCACTGTGAGAGAGACATCGATGAATGTGCCAGCAACCCCTGTTTGGATGGGGGTC | |
| | ACTGTCAGAATGAAATCAACAGATTCCAGTGTCTGTGTCCCACTGGTTTCTCTGGAAACCTCTGTCAGCT | |
| | GGACATCGATTATTGTGAGCCTAATCCCTGCCAGAACGGTGCCCAGTGCTACAACCGTGCCAGTGACTAT | |
| | TTCTGCAAGTGCCCCGAGGACTATGAGGGCAAGAACTGCTCACACCTGAAAGACCACTGCCGCACGACCC | |
| | CCTGTGAAGTGATTGACAGCTGCACAGTGGCCATGGCTTCCAACGACACACCTGAAGGGGTGCGGTATAT | |
| | TTCCTCCAACGTCTGTGGTCCTCACGGGAAGTGCAAGAGTCAGTCGGGAGGCAAATTCACCTGTGACTGT | |
| | AACAAAGGCTTCACGGGAACATACTGCCATGAAAATATTAATGACTGTGAGAGCAACCCTTGTAGAAACG | |
| | GTGGCACTTGCATCGATGGTGTCAACTCCTACAAGTGCATCTGTAGTGACGGCTGGGAGGGGGCCTACTG | |
| | TGAAACCAATATTAATGACTGCAGCCAGAACCCCTGCCACAATGGGGGCACGTGTCGCGACCTGGTCAAT | |
| | GACTTCTACTGTGACTGTAAAAATGGGTGGAAAGGAAAGACCTGCCACTCACGTGACAGTCAGTGTGATG | |
| | AGGCCACGTGCAACAACGGTGGCACCTGCTATGATGAGGGGGATGCTTTTAAGTGCATGTGTCCTGGCGG | |
| | CTGGGAAGGAACAACCTGTAACATAGCCCGAAACAGTAGCTGCCTGCCCAACCCCTGCCATAATGGGGGC | |
| | ACATGTGTGGTCAACGGCGAGTCCTTTACGTGCGTCTGCAAGGAAGGCTGGGAGGGGCCCATCTGTGCTC | |
| | AGAATACCAATGACTGCAGCCCTCATCCCTGTTACAACAGCGGCACCTGTGTGGATGGAGACAACTGGTA | |
| | CCGGTGCGAATGTGCCCCGGGTTTTGCTGGGCCCGACTGCAGAATAAACATCAATGAATGCCAGTCTTCA | |
| | CCTTGTGCCTTTGGAGCGACCTGTGTGGATGAGATCAATGGCTACCGGTGTGTCTGCCCTCCAGGGCACA | |
| | GTGGTGCCAAGTGCCAGGAAGTTTCAGGGAGACCTTGCATCACCATGGGGAGTGTGATACCAGATGGGGC | |
| | CAAATGGGATGATGACTGTAATACCTGCCAGTGCCTGAATGGACGGATCGCCTGCTCAAAGGTCTGGTGT | |
| | GGCCCTCGACCTTGCCTGCTCCACAAAGGGCACAGCGAGTGCCCCAGCGGGCAGAGCTGCATCCCCATCC | |
| | TGGACGACCAGTGCTTCGTCCACCCCTGCACTGGTGTGGGCGAGTGTCGGTCTTCCAGTCTCCAGCCGGT | |
| | GAAGACAAAGTGCACCTCTGACTCCTATTACCAGGATAACTGTGCGAACATCACATTTACCTTTAACAAG | |
| | GAGATGATGTCACCAGGTCTTACTACGGAGCACATTTGCAGTGAATTGAGGAATTTGAATATTTTGAAGA | |
| | ATGTTTCCGCTGAATATTCAATCTACATCGCTTGCGAGCCTTCCCCTTCAGCGAACAATGAAATACATGT | |
| | GGCCATTTCTGCTGAAGATATACGGGATGATGGGAACCCGATCAAGGAAATCACTGACAAAATAATTGAT | |
| | CTTGTTAGTAAACGTGATGGAAACAGCTCGCTGATTGCTGCCGTTGCAGAAGTAAGAGTTCAGAGGCGGC | |
| | CTCTGAAGAACAGAACAGATTTCCTTGTTCCCTTGCTGAGCTCTGTCTTAACTGTGGCTTGGATCTGTTG | |
| | CTTGGTGACGGCCTTCTACTGGTGCCTGCGGAAGCGGCGGAAGCCGGGCAGCCACACACACTCAGCCTCT | |
| | GAGGACAACACCACCAACAACGTGCGGGAGCAGCTGAACCAGATCAAAAACCCCATTGAGAAACATGGGG | |
| | CCAACACGGTCCCCATCAAGGATTACGAGAACAAGAACTCCAAAATGTCTAAAATAAGGACACACAATTC | |
| | TGAAGTAGAAGAGGACGACATGGACAAACACCAGCAGAAAGCCCGGTTTGCCAAGCAGCCGGCGTATACG | |
| | CTGGTAGACAGAGAAGAGAAGCCCCCCAACGGCACGCCGACAAAACACCCAAACTGGACAAACAAACAGG | |
| | ACAACAGAGACTTGGAAAGTGCCCAGAGCTTAAACCGAATGGAGTACATCGTATAGCAGACCGCGGGCAC | |
| | TGCCGCCGCTAGGTAGAGTCTGAGGGCTTGTAGTTCTTTAAACTGTCGTGTCATACTCGAGTCTGAGGCC | |
| | GTTGCTGACTTAGAATCCCTGTGTTAATTTAAGTTTTGACAAGCTGGCTTACACTGGCAATGGTAGTTTC | |
| | TGTGGTTGGCTGGGAAATCGAGTGCCGCATCTCACAGCTATGCAAAAAGCTAGTCAACAGTACCCTGGTT | |
| | GTGTGTCCCCTTGCAGCCGACACGGTCTCGGATCAGGCTCCCAGGAGCCTGCCCAGCCCCCTGGTCTTTG | |
| | AGCTCCCACTTCTGCCAGATGTCCTAATGGTGATGCAGTCTTAGATCATAGTTTTATTTATATTTATTGA | |
| | CTCTTGAGTTGTTTTTGTATATTGGTTTTATGATGACGTACAAGTAGTTCTGTATTTGAAAGTGCCTTTG | |
| | CAGCTCAGAACCACAGCAACGATCACAAATGACTTTATTATTTATTTTTTTAATTGTATTTTTGTTGTTG | |
| | GGGGAGGGGAGACTTTGATGTCAGCAGTTGCTGGTAAAATGAAGAATTTAAAGAAAAAAATGTCAAAAGT | |
| | AGAACTTTGTATAGTTATGTAAATAATTCTTTTTTATTAATCACTGTGTATATTTGATTTATTAACTTAA | |
| | TAATCAAGAGCCTTAAAACATCATTCCTTTTTATTTATATGTATGTGTTTAGAATTGAAGGTTTTTGATA | |
| | GCATTGTAAGCGTATGGCTTTATTTTTTTGAACTCTTCTCATTACTTGTTGCCTATAAGCCAAAATTAAG | |
| | GTGTTTGAAAATAGTTTATTTTAAAACAATAGGATGGGCTTCTGTGCCCAGAATACTGATGGAATTTTTT | |
| | TTGTACGACGTCAGATGTTTAAAACACCTTCTATAGCATCACTTAAAACACGTTTTAAGGACTGACTGAG | |
| | GCAGTTTGAGGATTAGTTTAGAACAGGTTTTTTTGTTTGTTTGTTTTTTGTTTTTCTGCTTTAGACTTGA | |
| | AAAGAGACAGGCAGGTGATCTGCTGCAGAGCAGTAAGGGAACAAGTTGAGCTATGACTTAACATAGCCAA | |
| | AATGTGAGTGGTTGAATATGATTAAAAATATCAAATTAATTGTGTGAACTTGGAAGCACACCAATCTGAC | |
| | TTTGTAAATTCTGATTTCTTTTCACCATTCGTACATAATACTGAACCACTTGTAGATTTGATTTTTTTTT | |
| | TAATCTACTGCATTTAGGGAGTATTCTAATAAGCTAGTTGAATACTTGAACCATAAAATGTCCAGTAAGA | |
| | TCACTGTTTAGATTTGCCATAGAGTACACTGCCTGCCTTAAGTGAGGAAATCAAAGTGCTATTACGAAGT | |
| | TCAAGATCAAAAAGGCTTATAAAACAGAGTAATCTTGTTGGTTCACCATTGAGACCGTGAAGATACTTTG | |
| | TATTGTCCTATTAGTGTTATATGAACATACAAATGCATCTTTGATGTGTTGTTCTTGGCAATAAATTTTG | |
| | AAAAGTAATATTTATTAAATTTTTTTGTATGAAAACATGGAACAGTGTGGCTCTTCTGAGCTTACGTAGT | |
| | TCTACCGGCTTTGCCGTGTGCTTCTGCCACCCTGCTGAGTCTGTTCTGGTAATCGGGGTATAATAGGCTC | |
| | TGCCTGACAGAGGGATGGAGGAAGAACTGAAAGGCTTTTCAACCACAAAACTCATCTGGAGTTCTCAAAG | |
| | ACCTGGGGCTGCTGTGAAGCTGGAACTGCGGGAGCCCCATCTAGGGGAGCCTTGATTCCCTTGTTATTCA | |
| | ACAGCAAGTGTGAATACTGCTTGAATAAACACCACTGGATTAATGGAAAAAAAAAAAAAAAA | |
| M18533.1 | GGGATTCCCTCACTTTCCCCCTACAGGACTCAGATCTGGGAGGCAATTACCTTCGGAGAAAAACGAATAG | 57 |
| Homo | GAAAAACTGAAGTGTTACTTTTTTTAAAGCTGCTGAAGTTTGTTGGTTTCTCATTGTTTTTAAGCCTACT | |
| sapiens | GGAGCAATAAAGTTTGAAGAACTTTTACCAGGTTTTTTTTATCGCTGCCTTGATATACACTTTTCAAAAT | |
| dystrophin | GCTTTGGTGGGAAGAAGTAGAGGACTGTTATGAAAGAGAAGATGTTCAAAAGAAAACATTCACAAAATGG | |
| (DMD), | GTAAATGCACAATTTTCTAAGTTTGGGAAGCAGCATATTGAGAACCTCTTCAGTGACCTACAGGATGGGA | |
| complete | GGCGCCTCCTAGACCTCCTCGAAGGCCTGACAGGGCAAAAACTGCCAAAAGAAAAAGGATCCACAAGAGT | |
| cds | TCATGCCCTGAACAATGTCAACAAGGCACTGCGGGTTTTGCAGAACAATAATGTTGATTTAGTGAATATT | |
| | GGAAGTACTGACATCGTAGATGGAAATCATAAACTGACTCTTGGTTTGATTTGGAATATAATCCTCCACT | |
| | GGCAGGTCAAAAATGTAATGAAAAATATCATGGCTGGATTGCAACAAACCAACAGTGAAAAGATTCTCCT | |
| | GAGCTGGGTCCGACAATCAACTCGTAATTATCCACAGGTTAATGTAATCAACTTCACCACCAGCTGGTCT | |
| | GATGGCCTGGCTTTGAATGCTCTCATCCATAGTCATAGGCCAGACCTATTTGACTGGAATAGTGTGGTTT | |
| | GCCAGCAGTCAGCCACACAACGACTGGAACATGCATTCAACATCGCCAGATATCAATTAGGCATAGAGAA | |
| | ACTACTCGATCCTGAAGATGTTGATACCACCTATCCAGATAAGAAGTCCATCTTAATGTACATCACATCA | |
| | CTCTTCCAAGTTTTGCCTCAACAAGTGAGCATTGAAGCCATCCAGGAAGTGGAAATGTTGCCAAGGCCAC | |
| | CTAAAGTGACTAAAGAAGAACATTTTCAGTTACATCATCAAATGCACTATTCTCAACAGATCACGGTCAG | |
| | TCTAGCACAGGGATATGAGAGAACTTCTTCCCCTAAGCCTCGATTCAAGAGCTATGCCTACACACAGGCT | |
| | GCTTATGTCACCACCTCTGACCCTACACGGAGCCCATTTCCTTCACAGCATTTGGAAGCTCCTGAAGACA | |
| | AGTCATTTGGCAGTTCATTGATGGAGAGTGAAGTAAACCTGGACCGTTATCAAACAGCTTTAGAAGAAGT | |
| | ATTATCGTGGCTTCTTTCTGCTGAGGACACATTGCAAGCACAAGGAGAGATTTCTAATGATGTGGAAGTG | |
| | GTGAAAGACCAGTTTCATACTCATGAGGGGTACATGATGGATTTGACAGCCCATCAGGGCCGGGTTGGTA | |
| | ATATTCTACAATTGGGAAGTAAGCTGATTGGAACAGGAAAATTATCAGAAGATGAAGAAACTGAAGTACA | |
| | AGAGCAGATGAATCTCCTAAATTCAAGATGGGAATGCCTCAGGGTAGCTAGCATGGAAAAACAAAGCAAT | |
| | TTACATAGAGTTTTAATGGATCTCCAGAATCAGAAACTGAAAGAGTTGAATGACTGGCTAACAAAAACAG | |
| | AAGAAAGAACAAGGAAAATGGAGGAAGAGCCTCTTGGACCTGATCTTGAAGACCTAAAACGCCAAGTACA | |
| | ACAACATAAGGTGCTTCAAGAAGATCTAGAACAAGAACAAGTCAGGGTCAATTCTCTCACTCACATGGTG | |
| | GTGGTAGTTGATGAATCTAGTGGAGATCACGCAACTGCTGCTTTGGAAGAACAACTTAAGGTATTGGGAG | |
| | ATCGATGGGCAAACATCTGTAGATGGACAGAAGACCGCTGGGTTCTTTTACAAGACATCCTTCTCAAATG | |
| | GCAACGTCTTACTGAAGAACAGTGCCTTTTTAGTGCATGGCTTTCAGAAAAAGAAGATGCAGTGAACAAG | |
| | ATTCACACAACTGGCTTTAAAGATCAAAATGAAATGTTATCAAGTCTTCAAAAACTGGCCGTTTTAAAAG | |
| | CGGATCTAGAAAAGAAAAAGCAATCCATGGGCAAACTGTATTCACTCAAACAAGATCTTCTTTCAACACT | |
| | GAAGAATAAGTCAGTGACCCAGAAGACGGAAGCATGGCTGGATAACTTTGCCCGGTGTTGGGATAATTTA | |
| | GTCCAAAAACTTGAAAAGAGTACAGCACAGATTTCACAGGCTGTCACCACCACTCAGCCATCACTAACAC | |
| | AGACAACTGTAATGGAAACAGTAACTACGGTGACCACAAGGGAACAGATCCTGGTAAAGCATGCTCAAGA | |
| | GGAACTTCCACCACCACCTCCCCAAAAGAAGAGGCAGATTACTGTGGATTCTGAAATTAGGAAAAGGTTG | |
| | GATGTTGATATAACTGAACTTCACAGCTGGATTACTCGCTCAGAAGCTGTGTTGCAGAGTCCTGAATTTG | |
| | CAATCTTTCGGAAGGAAGGCAACTTCTCAGACTTAAAAGAAAAAGTCAATGCCATAGAGCGAGAAAAAGC | |
| | TGAGAAGTTCAGAAAACTGCAAGATGCCAGCAGATCAGCTCAGGCCCTGGTGGAACAGATGGTGAATGAG | |
| | GGTGTTAATGCAGATAGCATCAAACAAGCCTCAGAACAACTGAACAGCCGGTGGATCGAATTCTGCCAGT | |
| | TGCTAAGTGAGAGACTTAACTGGCTGGAGTATCAGAACAACATCATCGCTTTCTATAATCAGCTACAACA | |
| | ATTGGAGCAGATGACAACTACTGCTGAAAACTGGTTGAAAATCCAACCCACCACCCCATCAGAGCCAACA | |
| | GCAATTAAAAGTCAGTTAAAAATTTGTAAGGATGAAGTCAACCGGCTATCAGGTCTTCAACCTCAAATTG | |
| | AACGATTAAAAATTCAAAGCATAGCCCTGAAAGAGAAAGGACAAGGACCCATGTTCCTGGATGCAGACTT | |
| | TGTGGCCTTTACAAATCATTTTAAGCAAGTCTTTTCTGATGTGCAGGCCAGAGAGAAAGAGCTACAGACA | |
| | ATTTTTGACACTTTGCCACCAATGCGCTATCAGGAGACCATGAGTGCCATCAGGACATGGGTCCAGCAGT | |
| | CAGAAACCAAACTCTCCATACCTCAACTTAGTGTCACCGACTATGAAATCATGGAGCAGAGACTCGGGGA | |
| | ATTGCAGGCTTTACAAAGTTCTCTGCAAGAGCAACAAAGTGGCCTATACTATCTCAGCACCACTGTGAAA | |
| | GAGATGTCGAAGAAAGCGCCCTCTGAAATTAGCCGGAAATATCAATCAGAATTTGAAGAAATTGAGGGAC | |
| | GCTGGAAGAAGCTCTCCTCCCAGCTGGTTGAGCATTGTCAAAAGCTAGAGGAGCAAATGAATAAACTCCG | |
| | AAAAATTCAGAATCACATACAAACCCTGAAGAAATGGATGGCTGAAGTTGATGTTTTTCTGAAGGAGGAA | |
| | TGGCCTGCCCTTGGGGATTCAGAAATTCTAAAAAAGCAGCTGAAACAGTGCAGACTTTTAGTCAGTGATA | |
| | TTCAGACAATTCAGCCCAGTCTAAACAGTGTCAATGAAGGTGGGCAGAAGATAAAGAATGAAGCAGAGCC | |
| | AGAGTTTGCTTCGAGACTTGAGACAGAACTCAAAGAACTTAACACTCAGTGGGATCACATGTGCCAACAG | |
| | GTCTATGCCAGAAAGGAGGCCTTGAAGGGAGGTTTGGAGAAAACTGTAAGCCTCCAGAAAGATCTATCAG | |
| | AGATGCACGAATGGATGACACAAGCTGAAGAAGAGTATCTTGAGAGAGATTTTGAATATAAAACTCCAGA | |
| | TGAATTACAGAAAGCAGTTGAAGAGATGAAGAGAGCTAAAGAAGAGGCCCAACAAAAAGAAGCGAAAGTG | |
| | AAACTCCTTACTGAGTCTGTAAATAGTGTCATAGCTCAAGCTCCACCTGTAGCACAAGAGGCCTTAAAAA | |
| | AGGAACTTGAAACTCTAACCACCAACTACCAGTGGCTCTGCACTAGGCTGAATGGGAAATGCAAGACTTT | |
| | GGAAGAAGTTTGGGCATGTTGGCATGAGTTATTGTCATACTTGGAGAAAGCAAACAAGTGGCTAAATGAA | |
| | GTAGAATTTAAACTTAAAACCACTGAAAACATTCCTGGCGGAGCTGAGGAAATCTCTGAGGTGCTAGATT | |
| | CACTTGAAAATTTGATGCGACATTCAGAGGATAACCCAAATCAGATTCGCATATTGGCACAGACCCTAAC | |
| | AGATGGCGGAGTCATGGATGAGCTAATCAATGAGGAACTTGAGACATTTAATTCTCGTTGGAGGGAACTA | |
| | CATGAAGAGGCTGTAAGGAGGCAAAAGTTGCTTGAACAGAGCATCCAGTCTGCCCAGGAGACTGAAAAAT | |
| | CCTTACACTTAATCCAGGAGTCCCTCACATTCATTGACAAGCAGTTGGCAGCTTATATTGCAGACAAGGT | |
| | GGACGCAGCTCAAATGCCTCAGGAAGCCCAGAAAATCCAATCTGATTTGACAAGTCATGAGATCAGTTTA | |
| | GAAGAAATGAAGAAACATAATCAGGGGAAGGAGGCTGCCCAAAGAGTCCTGTCTCAGATTGATGTTGCAC | |
| | AGAAAAAATTACAAGATGTCTCCATGAAGTTTCGATTATTCCAGAAACCAGCCAATTTTGAGCTGCGTCT | |
| | ACAAGAAAGTAAGATGATTTTAGATGAAGTGAAGATGCACTTGCCTGCATTGGAAACAAAGAGTGTGGAA | |
| | CAGGAAGTAGTACAGTCACAGCTAAATCATTGTGTGAACTTGTATAAAAGTCTGAGTGAAGTGAAGTCTG | |
| | AAGTGGAAATGGTGATAAAGACTGGACGTCAGATTGTACAGAAAAAGCAGACGGAAAATCCCAAAGAACT | |
| | TGATGAAAGAGTAACAGCTTTGAAATTGCATTATAATGAGCTGGGAGCAAAGGTAACAGAAAGAAAGCAA | |
| | CAGTTGGAGAAATGCTTGAAATTGTCCCGTAAGATGCGAAAGGAAATGAATGTCTTGACAGAATGGCTGG | |
| | CAGCTACAGATATGGAATTGACAAAGAGATCAGCAGTTGAAGGAATGCCTAGTAATTTGGATTCTGAAGT | |
| | TGCCTGGGGAAAGGCTACTCAAAAAGAGATTGAGAAACAGAAGGTGCACCTGAAGAGTATCACAGAGGTA | |
| | GGAGAGGCCTTGAAAACAGTTTTGGGCAAGAAGGAGACGTTGGTGGAAGATAAACTCAGTCTTCTGAATA | |
| | GTAACTGGATAGCTGTCACCTCCCGAGCAGAAGAGTGGTTAAATCTTTTGTTGGAATACCAGAAACACAT | |
| | GGAAACTTTTGACCAGAATGTGGACCACATCACAAAGTGGATCATTCAGGCTGACACACTTTTGGATGAA | |
| | TCAGAGAAAAAGAAACCCCAGCAAAAAGAAGACGTGCTTAAGCGTTTAAAGGCAGAACTGAATGACATAC | |
| | GCCCAAAGGTGGACTCTACACGTGACCAAGCAGCAAACTTGATGGCAAACCGCGGTGACCACTGCAGGAA | |
| | ATTAGTAGAGCCCCAAATCTCAGAGCTCAACCATCGATTTGCAGCCATTTCACACAGAATTAAGACTGGA | |
| | AAGGCCTCCATTCCTTTGAAGGAATTGGAGCAGTTTAACTCAGATATACAAAAATTGCTTGAACCACTGG | |
| | AGGCTGAAATTCAGCAGGGGGTGAATCTGAAAGAGGAAGACTTCAATAAAGATATGAATGAAGACAATGA | |
| | GGGTACTGTAAAAGAATTGTTGCAAAGAGGAGACAACTTACAACAAAGAATCACAGATGAGAGAAAGAGA | |
| | GAGGAAATAAAGATAAAACAGCAGCTGTTACAGACAAAACATAATGCTCTCAAGGATTTGAGGTCTCAAA | |
| | GAAGAAAAAAGGCTCTAGAAATTTCTCATCAGTGGTATCAGTACAAGAGGCAGGCTGATGATCTCCTGAA | |
| | ATGCTTGGATGACATTGAAAAAAAATTAGCCAGCCTACCTGAGCCCAGAGATGAAAGGAAAATAAAGGAA | |
| | ATTGATCGGGAATTGCAGAAGAAGAAAGAGGAGCTGAATGCAGTGCGTAGGCAAGCTGAGGGCTTGTCTG | |
| | AGGATGGGGCCGCAATGGCAGTGGAGCCAACTCAGATCCAGCTCAGCAAGCGCTGGCGGGAAATTGAGAG | |
| | CAAATTTGCTCAGTTTCGAAGACTCAACTTTGCACAAATTCACACTGTCCGTGAAGAAACGATGATGGTG | |
| | ATGACTGAAGACATGCCTTTGGAAATTTCTTATGTGCCTTCTACTTATTTGACTGAAATCACTCATGTCT | |
| | CACAAGCCCTATTAGAAGTGGAACAACTTCTCAATGCTCCTGACCTCTGTGCTAAGGACTTTGAAGATCT | |
| | CTTTAAGCAAGAGGAGTCTCTGAAGAATATAAAAGATAGTCTACAACAAAGCTCAGGTCGGATTGACATT | |
| | ATTCATAGCAAGAAGACAGCAGCATTGCAAAGTGCAACGCCTGTGGAAAGGGTGAAGCTACAGGAAGCTC | |
| | TCTCCCAGCTTGATTTCCAATGGGAAAAAGTTAACAAAATGTACAAGGACCGACAAGGGCGATTTGACAG | |
| | ATCTGTTGAGAAATGGCGGCGTTTTCATTATGATATAAAGATATTTAATCAGTGGCTAACAGAAGCTGAA | |
| | CAGTTTCTCAGAAAGACACAAATTCCTGAGAATTGGGAACATGCTAAATACAAATGGTATCTTAAGGAAC | |
| | TCCAGGATGGCATTGGGCAGCGGCAAACTGTTGTCAGAACATTGAATGCAACTGGGGAAGAAATAATTCA | |
| | GCAATCCTCAAAAACAGATGCCAGTATTCTACAGGAAAAATTGGGAAGCCTGAATCTGCGGTGGCAGGAG | |
| | GTCTGCAAACAGCTGTCAGACAGAAAAAAGAGGCTAGAAGAACAAAAGAATATCTTGTCAGAATTTCAAA | |
| | GAGATTTAAATGAATTTGTTTTATGGTTGGAGGAAGCAGATAACATTGCTAGTATCCCACTTGAACCTGG | |
| | AAAAGAGCAGCAACTAAAAGAAAAGCTTGAGCAAGTCAAGTTACTGGTGGAAGAGTTGCCCCTGCGCCAG | |
| | GGAATTCTCAAACAATTAAATGAAACTGGAGGACCCGTGCTTGTAAGTGCTCCCATAAGCCCAGAAGAGC | |
| | AAGATAAACTTGAAAATAAGCTCAAGCAGACAAATCTCCAGTGGATAAAGGTTTCCAGAGCTTTACCTGA | |
| | GAAACAAGGAGAAATTGAAGCTCAAATAAAAGACCTTGGGCAGCTTGAAAAAAAGCTTGAAGACCTTGAA | |
| | GAGCAGTTAAATCATCTGCTGCTGTGGTTATCTCCTATTAGGAATCAGTTGGAAATTTATAACCAACCAA | |
| | ACCAAGAAGGACCATTTGACGTTCAGGAAACTGAAATAGCAGTTCAAGCTAAACAACCGGATGTGGAAGA | |
| | GATTTTGTCTAAAGGGCAGCATTTGTACAAGGAAAAACCAGCCACTCAGCCAGTGAAGAGGAAGTTAGAA | |
| | GATCTGAGCTCTGAGTGGAAGGCGGTAAACCGTTTACTTCAAGAGCTGAGGGCAAAGCAGCCTGACCTAG | |
| | CTCCTGGACTGACCACTATTGGAGCCTCTCCTACTCAGACTGTTACTCTGGTGACACAACCTGTGGTTAC | |
| | TAAGGAAACTGCCATCTCCAAACTAGAAATGCCATCTTCCTTGATGTTGGAGGTACCTGCTCTGGCAGAT | |
| | TTCAACCGGGCTTGGACAGAACTTACCGACTGGCTTTCTCTGCTTGATCAAGTTATAAAATCACAGAGGG | |
| | TGATGGTGGGTGACCTTGAGGATATCAACGAGATGATCATCAAGCAGAAGGCAACAATGCAGGATTTGGA | |
| | ACAGAGGCGTCCCCAGTTGGAAGAACTCATTACCGCTGCCCAAAATTTGAAAAACAAGACCAGCAATCAA | |
| | GAGGCTAGAACAATCATTACGGATCGAATTGAAAGAATTCAGAATCAGTGGGATGAAGTACAAGAACACC | |
| | TTCAGAACCGGAGGCAACAGTTGAATGAAATGTTAAAGGATTCAACACAATGGCTGGAAGCTAAGGAAGA | |
| | AGCTGAGCAGGTCTTAGGACAGGCCAGAGCCAAGCTTGAGTCATGGAAGGAGGGTCCCTATACAGTAGAT | |
| | GCAATCCAAAAGAAAATCACAGAAACCAAGCAGTTGGCCAAAGACCTCCGCCAGTGGCAGACAAATGTAG | |
| | ATGTGGCAAATGACTTGGCCCTGAAACTTCTCCGGGATTATTCTGCAGATGATACCAGAAAAGTCCACAT | |
| | GATAACAGAGAATATCAATGCCTCTTGGAGAAGCATTCATAAAAGGGTGAGTGAGCGAGAGGCTGCTTTG | |
| | GAAGAAACTCATAGATTACTGCAACAGTTCCCCCTGGACCTGGAAAAGTTTCTTGCCTGGCTTACAGAAG | |
| | CTGAAACAACTGCCAATGTCCTACAGGATGCTACCCGTAAGGAAAGGCTCCTAGAAGACTCCAAGGGAGT | |
| | AAAAGAGCTGATGAAACAATGGCAAGACCTCCAAGGTGAAATTGAAGCTCACACAGATGTTTATCACAAC | |
| | CTGGATGAAAACAGCCAAAAAATCCTGAGATCCCTGGAAGGTTCCGATGATGCAGTCCTGTTACAAAGAC | |
| | GTTTGGATAACATGAACTTCAAGTGGAGTGAACTTCGGAAAAAGTCTCTCAACATTAGGTCCCATTTGGA | |
| | AGCCAGTTCTGACCAGTGGAAGCGTCTGCACCTTTCTCTGCAGGAACTTCTGGTGTGGCTACAGCTGAAA | |
| | GATGATGAATTAAGCCGGCAGGCACCTATTGGAGGCGACTTTCCAGCAGTTCAGAAGCAGAACGATGTAC | |
| | ATAGGGCCTTCAAGAGGGAATTGAAAACTAAAGAACCTGTAATCATGAGTACTCTTGAGACTGTACGAAT | |
| | ATTTCTGACAGAGCAGCCTTTGGAAGGACTAGAGAAACTCTACCAGGAGCCCAGAGAGCTGCCTCCTGAG | |
| | GAGAGAGCCCAGAATGTCACTCGGCTTCTACGAAAGCAGGCTGAGGAGGTCAATACTGAGTGGGAAAAAT | |
| | TGAACCTGCACTCCGCTGACTGGCAGAGAAAAATAGATGAGACCCTTGAAAGACTCCAGGAACTTCAAGA | |
| | GGCCACGGATGAGCTGGACCTCAAGCTGCGCCAAGCTGAGGTGATCAAGGGATCCTGGCAGCCCGTGGGC | |
| | GATCTCCTCATTGACTCTCTCCAAGATCACCTCGAGAAAGTCAAGGCACTTCGAGGAGAAATTGCGCCTC | |
| | TGAAAGAGAACGTGAGCCACGTCAATGACCTTGCTCGCCAGCTTACCACTTTGGGCATTCAGCTCTCACC | |
| | GTATAACCTCAGCACTCTGGAAGACCTGAACACCAGATGGAAGCTTCTGCAGGTGGCCGTCGAGGACCGA | |
| | GTCAGGCAGCTGCATGAAGCCCACAGGGACTTTGGTCCAGCATCTCAGCACTTTCTTTCCACGTCTGTCC | |
| | AGGGTCCCTGGGAGAGAGCCATCTCGCCAAACAAAGTGCCCTACTATATCAACCACGAGACTCAAACAAC | |
| | TTGCTGGGACCATCCCAAAATGACAGAGCTCTACCAGTCTTTAGCTGACCTGAATAATGTCAGATTCTCA | |
| | GCTTATAGGACTGCCATGAAACTCCGAAGACTGCAGAAGGCCCTTTGCTTGGATCTCTTGAGCCTGTCAG | |
| | CTGCATGTGATGCCTTGGACCAGCACAACCTCAAGCAAAATGACCAGCCCATGGATATCCTGCAGATTAT | |
| | TAATTGTTTGACCACTATTTATGACCGCCTGGAGCAAGAGCACAACAATTTGGTCAACGTCCCTCTCTGC | |
| | GTGGATATGTGTCTGAACTGGCTGCTGAATGTTTATGATACGGGACGAACAGGGAGGATCCGTGTCCTGT | |
| | CTTTTAAAACTGGCATCATTTCCCTGTGTAAAGCACATTTGGAAGACAAGTACAGATACCTTTTCAAGCA | |
| | AGTGGCAAGTTCAACAGGATTTTGTGACCAGCGCAGGCTGGGCCTCCTTCTGCATGATTCTATCCAAATT | |
| | CCAAGACAGTTGGGTGAAGTTGCATCCTTTGGGGGCAGTAACATTGAGCCAAGTGTCCGGAGCTGCTTCC | |
| | AATTTGCTAATAATAAGCCAGAGATCGAAGCGGCCCTCTTCCTAGACTGGATGAGACTGGAACCCCAGTC | |
| | CATGGTGTGGCTGCCCGTCCTGCACAGAGTGGCTGCTGCAGAAACTGCCAAGCATCAGGCCAAATGTAAC | |
| | ATCTGCAAAGAGTGTCCAATCATTGGATTCAGGTACAGGAGTCTAAAGCACTTTAATTATGACATCTGCC | |
| | AAAGCTGCTTTTTTTCTGGTCGAGTTGCAAAAGGCCATAAAATGCACTATCCCATGGTGGAATATTGCAC | |
| | TCCGACTACATCAGGAGAAGATGTTCGAGACTTTGCCAAGGTACTAAAAAACAAArTTCGAACCAAAAGG | |
| | TATTTTGCGAAGCATCCCCGAATGGGCTACCTGCCAGTGCAGACTGTCTTAGAGGGGGACAACATGGAAA | |
| | CTCCCGTTACTCTGATCAACTTCTGGCCAGTAGATTCTGCGCCTGCCTCGTCCCCTCAGCTTTCACACGA | |
| | TGATACTCATTCACGCATTGAACATTATGCTAGCAGGCTAGCAGAAATGGAAAACAGCAATGGATCTTAT | |
| | CTAAATGATAGCATCTCTCCTAATGAGAGCATAGATGATGAACATTTGTTAATCCAGCATTACTGCCAAA | |
| | GTTTGAACCAGGACTCCCCCCTGAGCCAGCCTCGTAGTCCTGCCCAGATCTTGATTTCCTTAGAGAGTGA | |
| | GGAAAGAGGGGAGCTAGAGAGAATCCTAGCAGATCTTGAGGAAGAAAACAGGAATCTGCAAGCAGAATAT | |
| | GACCGTCTAAAGCAGCAGCACGAACATAAAGGCCTGTCCCCACTGCCGTCCCCTCCTGAAATGATGCCCA | |
| | CCTCTCCCCAGAGTCCCCGGGATGCTGAGCTCATTGCTGAGGCCAAGCTACTGCGTCAACACAAAGGCCG | |
| | CCTGGAAGCCAGGATGCAAATCCTGGAAGACCACAATAAACAGCTGGAGTCACAGTTACACAGGCTAAGG | |
| | CAGCTGCTGGAGCAACCCCAGGCAGAGGCCAAAGTGAATGGCACAACGGTGTCCTCTCCTTCTACCTCTC | |
| | TACAGAGGTCCGACAGCAGTCAGCCTATGCTGCTCCGAGTGGTTGGCAGTCAAACTTCGGACTCCATGGG | |
| | TGAGGAAGATCTTCTCAGTCCTCCCCAGGACACAAGCACAGGGTTAGAGGAGGTGATGGAGCAACTCAAC | |
| | AACTCCTTCCCTAGTTCAAGAGGAAGAAATACCCCTGGAAAGCCAATGAGAGAGGACACAATGTAGGAAG | |
| | TCTTTTCCACATGGCAGATGATTTGGGCAGAGCGATGGAGTCCTTAGTATCAGTCATGACAGATGAAGAA | |
| | GGAGCAGAATAAATGTTTTACAACTCCTGATTCCCGCATGGTTTTTATAATATTCATACAACAAAGAGGA | |
| | TTAGACAGTAAGAGTTTACAAGAAATAAATCTATATTTTTGTGAAGGGTAGTGGTATTATACTGTAGATT | |
| | TCAGTAGTTTCTAAGTCTGTTATTGTTTTGTTAACAATGGCAGGTTTTACACGTCTATGCAATTGTACAA | |
| | AAAAGTTATAAGAAAACTACATGTAAAATCTTGATAGCTAAATAACTTGCCATTTCTTTATATGGAACGC | |
| | ATTTTGGGTTGTTTAAAAATTTATAACAGTTATAAAGAAAGATTGTAAACTAAAGTGTGCTTTATAAAAA | |
| | AAAGTTGTTTATAAAAACCCCTAAAAACAAAACAAACACACACACACACACATACACACACACACACAAA | |
| | ACTTTGAGGCAGCGCATTGTTTTGCATCCTTTTGGCGTGATATCCATATGAAATTCATGGCTTTTTCTTT | |
| | TTTTGCATATTAAAGATAAGACTTCCTCTACCACCACACCAAATGACTACTACACACTGCTCATTTGAGA | |
| | ACTGTCAGCTGAGTGGGGCAGGCTTGAGTTTTCATTTCATATATCTATATGTCTATAAGTATATAAATAC | |
| | TATAGTTATATAGATAAAGAGATACGAATTTCTATAGACTGACTTTTTCCATTTTTTAAATGTTCATGTC | |
| | ACATCCTAATAGAAAGAAATTACTTCTAGTCAGTCATCCAGGCTTACCTGCTTGGTCTAGAATGGATTTT | |
| | TCCCGGAGCCGGAAGCCAGGAGGAAACTACACCACACTAAAACATTGTCTACAGCrCCAGATGTTTCTCA | |
| | TTTTAAACAACTTTCCACTGACAACGAAAGTAAAGTAAAGTATTGGATTTTTTTAAAGGGAACATGTGAA | |
| | TGAATACACAGGACTTATTATATCAGAGTGAGTAATCGGTTGGTTGGTTGATTGATTGATTGATTGATAC | |
| | ATTCAGCTTCCTGCTGCTAGCAATGCCACGATTTAGATTTAATGATGCTTCAGTGGAAATCAATCAGAAG | |
| | GTATTCTGACCTTGTGAACATCAGAAGGTATTTTTTAACTCCCAAGCAGTAGCAGGACGATGATAGGGCT | |
| | GGAGGGCTATGGATTCCCAGCCCATCCCTGTGAAGGAGTAGGCCACTCTTTAAGTGAAGGATTGGATGAT | |
| | TGTTCATAATACATAAAGTTCTCTGTAATTACAACTAAATTATTATGCCCTCTTCTCACAGTCAAAAGGA | |
| | ACTGGGTGGTTTGGTTTTTGTTGCTTTTTTAGATTTATTGTCCCATGTGGGATGAGTTTTTAAATGCCAC | |
| | AAGACATAATTTAAAATAAATAAACTTTGGGAAAAGGTGTAAGACAGTAGCCCCATCACATTTGTGATAC | |
| | TGACAGGTATCAACCCAGAAGCCCATGAACTGTGTTTCCATCCTTTGCATTTCTCTGCGAGTAGTTCCAC | |
| | ACAGGTTTGTAAGTAAGTAAGAAAGAAGGCAAATTGATTCAAATGTTACAAAAAAACCCTTCTTGGTGGA | |
| | TTAGACAGGTTAAATATATAAACAAACAAACAAAAATTGCTCAAAAAAGAGGAGAAAAGCTCAAGAGGAA | |
| | AAGCTAAGGACTGGTAGGAAAAAGCTTTACTCTTTCATGCCATTTTATTTCTTTTTGATTTTTAAATCAT | |
| | TCATTCAATAGATACCACCGTGTGACCTATAATTTTGCAAATCTGTTACCTCTGACATCAAGTGTAATTA | |
| | GCTTTTGGAGAGTGGGCTGACATCAAGTGTAATTAGCTTTTGGAGAGTGGGTTTTGTCCATTATTAATAA | |
| | TTAATTAATTAACATCAAACACGGCTTCTCATGCTATTTCTACCTCACTTTGGTTTTGGGGTGTTCCTGA | |
| | TAATTGTGCACACCTGAGTTCACAGCTTCACCACTTGTCCATTGCGTTATTTTCTTTTTCCTTTATAATT | |
| | CTTTCTTTTTCCTTCATAATTTTCAAAAGAAAACCCAAAGCTCTAAGGTAACAAATTACCAAATTACATG | |
| | AAGATTTGGTTTTTGTCTTGCATTTTTTTCCTTTATGTGACGCTGGACCTTTTCTTTACCCAAGGATTTT | |
| | TAAAACTCAGATTTAAAACAAGGGGTTACTTTACATCCTACTAAGAAGTTTAAGTAAGTAAGTTTCATTC | |
| | TAAAATCAGAGGTAAATAGAGTGCATAAATAATTTTGTTTTAATCTTTTTGTTTTTCTTTTAGACACATT | |
| | AGCTCTGGAGTGAGTCTGTCATAATATTTGAACAAAAATTGAGAGCTTTATTGCTGCATTTTAAGCATAA | |
| | TTAATTTGGACATTATTTCGTGTTGTGTTCTTTATAACCACCGAGTATTAAACTGTAAATCATAATGTAA | |
| | CTGAAGCATAAACATCACATGGCATGTTTTGTCATTGTTTTCAGGTACTGAGTTCTTACTTGAGTATCAT | |
| | AATATATTGTGTTTTAACACCAACACTGTAACATTTACGAATTATTTTTTTAAACTTCAGTTTTACTGCA | |
| | TTTTCACAACATATCAGACTTCACCAAATATATGCCTTACTATTGTATTATAGTACTGCTTTACTGTGTA | |
| | TCTCAATAAAGCACGCAGTTATGTTAC | |
| Homo | TTCCCCAGCAGCTGCTGCTCGCTCAGCTCACAAGCCAAGGCCAGGGGACAGGGCGGCAGCGACTCCTCTG | 58 |
| sapiens | GCTCCCGAGAAGTGGATCCGGTCGCGGCCACTACGATGCCGGGAGCCGCCGGGGTCCTCCTCCTTCTGCT | |
| laminin | GCTCTCCGGAGGCCTCGGGGGCGTACAGGCGCAGCGGCCGCAGCAGCAGCGGCAGTCACAGGCACATCAG | |
| subunit | CAAAGAGGTTTATTCCCTGCTGTCCTGAATCTTGCTTCTAATGCTCTTATCACGACCAATGCAACATGTG | |
| alpha 2 | GAGAAAAAGGACCTGAAATGTACTGCAAATTGGTAGAACATGTCCCTGGGCAGCCTGTGAGGAACCCGCA | |
| (LAMA2), | GTGTCGAATCTGCAATCAAAACAGCAGCAATCCAAACCAGAGACACCCGATTACAAATGCTATTGATGGA | |
| transcript | AAGAACACTTGGTGGCAGAGTCCCAGTATTAAGAATGGAATCGAATACCATTATGTGACAATTACCCTGG | |
| variant 2 | ATTTACAGCAGGTGTTCCAGATCGCGTATGTGATTGTGAAGGCAGCTAACTCCCCCCGGCCTGGAAACTG | |
| | GATTTTGGAACGCTCTCTTGATGATGTTGAATACAAGCCCTGGCAGTATCATGCTGTGACAGACACGGAG | |
| | TGCCTAACGCTTTACAATATTTATCCCCGCACTGGGCCACCGTCATATGCCAAAGATGATGAGGTCATCT | |
| | GCACTTCATTTTACTCCAAGATACACCCCTTAGAAAATGGAGAGATTCACATCTCTTTAATCAATGGGAG | |
| | ACCAAGTGCCGATGATCCTTCTCCAGAACTGCTAGAATTTACCTCCGCTCGCTATATTCGCCTGAGATTT | |
| | CAGAGGATCCGCACACTGAATGCTGACTTGATGATGTTTGCTCACAAAGACCCAAGAGAAATTGACCCCA | |
| | TTGTCACCAGAAGATATTACTACTCGGTCAAGGATATTTCAGTTGGAGGGATGTGCATCTGCTATGGTCA | |
| | TGCCAGGGCTTGTCCACTTGATCCAGCGACAAATAAATCTCGCTGTGAGTGTGAGCATAACACATGTGGC | |
| | GATAGCTGTGATCAGTGCTGTCCAGGATTCCATCAGAAACCCTGGAGAGCTGGAACTTTTCTAACTAAAA | |
| | CTGAATGTGAAGCATGCAATTGTCATGGAAAAGCTGAAGAATGCTATTATGATGAAAATGTTGCCAGAAG | |
| | AAATCTGAGTTTGAATATACGTGGAAAGTACATTGGAGGGGGTGTCTGCATTAATTGTACCCAAAACACT | |
| | GCTGGTATAAACTGCGAGACATGTACTGATGGCTTCTTCAGACCCAAAGGGGTATCTCCAAATTATCCAA | |
| | GGCCATGCCAGCCATGTCATTGCGATCCAATTGGTTCCTTAAATGAAGTCTGTGTCAAGGATGAGAAACA | |
| | TGCTCGACGAGGTTTGGCACCTGGATCCTGTCATTGCAAAACTGGTTTTGGAGGTGTGAGCTGTGATCGG | |
| | TGTGCCAGGGGCTACACTGGCTACCCGGACTGCAAAGCCTGTAACTGCAGTGGGTTAGGGAGCAAAAATG | |
| | AGGATCCTTGTTTTGGCCCCTGTATCTGCAAGGAAAATGTTGAAGGAGGAGACTGTAGTCGTTGCAAATC | |
| | CGGCTTCTTCAATTTGCAAGAGGATAATTGGAAAGGCTGCGATGAGTGTTTCTGTTCAGGGGTTTCAAAC | |
| | AGATGTCAGAGTTCCTACTGGACCTATGGCAAAATACAAGATATGAGTGGCTGGTATCTGACTGACCTTC | |
| | CTGGCCGCATTCGAGTGGCTCCCCAGCAGGACGACTTGGACTCACCTCAGCAGATCAGCATCAGTAACGC | |
| | GGAGGCCCGGCAAGCCCTGCCGCACAGCTACTACTGGAGCGCGCCGGCTCCCTATCTGGGAAACAAACTC | |
| | CCAGCAGTAGGAGGACAGTTGACATTTACCATATCATATGACCTTGAAGAAGAGGAAGAAGATACAGAAC | |
| | GTGTTCTCCAGCTTATGATTATCTTAGAGGGTAATGACTTGAGCATCAGCACAGCCCAAGATGAGGTGTA | |
| | CCTGCACCCATCTGAAGAACATACTAATGTATTGTTACTTAAAGAAGAATCATTTACCATACATGGCACA | |
| | CATTTTCCAGTCCGTAGAAAGGAATTTATGACAGTGCTTGCGAATTTGAAGAGAGTCCTCCTACAAATCA | |
| | CATACAGCTTTGGGATGGATGCCATCTTCAGGTTGAGCTCTGTTAACCTTGAATCCGCTGTCTCCTATCC | |
| | TACTGATGGAAGCATTGCAGCAGCTGTAGAAGTGTGTCAGTGCCCACCAGGGTATACTGGCTCCTCTTGT | |
| | GAATCTTGTTGGCCTAGGCACAGGCGAGTTAACGGCACTATTTTTGGTGGCATCTGTGAGCCATGTCAGT | |
| | GCTTTGGTCATGCGGAGTCCTGTGATGACGTCACTGGAGAATGCCTGAACTGTAAGGATCACACAGGTGG | |
| | CCCATATTGTGATAAATGTCTTCCTGGTTTCTATGGCGAGCCTACTAAAGGAACCTCTGAAGACTGTCAA | |
| | CCCTGTGCCTGTCCACTCAATATCCCATCCAATAACTTTAGCCCAACGTGCCATTTAGACCGGAGTCTTG | |
| | GATTGATCTGTGATGGATGCCCTGTCGGGTACACAGGACCACGCTGTGAGAGGTGTGCAGAAGGCTATTT | |
| | TGGACAACCCTCTGTACCTGGAGGATCATGTCAGCCATGCCAATGCAATGACAACCTTGACTTCTCCATC | |
| | CCTGGCAGCTGTGACAGCTTGTCTGGCTCCTGTCTGATATGTAAACCAGGTACAACAGGCCGGTACTGTG | |
| | AGCTCTGTGCTGATGGATATTTTGGAGATGCAGTTGATGCGAAGAACTGTCAGCCCTGTCGCTGTAATGC | |
| | CGGTGGCTCTTTCTCTGAGGTTTGCCACAGTCAAACTGGACAGTGTGAGTGCAGAGCCAACGTTCAGGGT | |
| | CAGAGATGTGACAAATGCAAGGCTGGGACCTTTGGCCTACAATCAGCAAGGGGCTGTGTTCCCTGCAACT | |
| | GCAATTCTTTTGGGTCTAAGTCATTCGACTGTGAAGAGAGTGGACAATGTTGGTGCCAACCTGGAGTCAC | |
| | AGGGAAGAAATGTGACCGCTGTGCCCACGGCTATTTCAACTTCCAAGAAGGAGGCTGCACAGCTTGTGAA | |
| | TGTTCTCATCTGGGTAATAATTGTGACCCAAAGACTGGGCGATGCATTTGCCCTCCCAATACCATTGGAG | |
| | AGAAATGTTCTAAATGTGCACCCAATACCTGGGGCCACAGCATTACCACTGGTTGTAAGGCTTGTAACTG | |
| | CAGCACAGTGGGATCCTTGGATTTCCAATGCAATGTAAATACAGGCCAATGCAACTGTCATCCAAAATTC | |
| | TCTGGTGCAAAATGTACAGAGTGCAGTCGAGGTCACTGGAACTACCCTCGCTGCAATCTCTGTGACTGCT | |
| | TCCTCCCTGGGACAGATGCCACAACCTGTGATTCAGAGACTAAAAAATGCTCCTGTAGTGATCAAACTGG | |
| | GCAGTGCACTTGTAAGGTGAATGTGGAAGGCATCCACTGTGACAGATGCCGGCCTGGCAAATTCGGACTC | |
| | GATGCCAAGAATCCACTTGGCTGCAGCAGCTGCTATTGCTTCGGCACTACTACCCAGTGCTCTGAAGCAA | |
| | AAGGACTGATCCGGACGTGGGTGACTCTGAAGGCTGAGCAGACCATTCTACCCCTGGTAGATGAGGCTCT | |
| | GCAGCACACGACCACCAAGGGCATTGTTTTTCAACATCCAGAGATTGTTGCCCACATGGACCTGATGAGA | |
| | GAAGATCTCCATTTGGAACCTTTTTATTGGAAACTTCCAGAACAATTTGAAGGAAAGAAGTTGATGGCCT | |
| | ATGGGGGCAAACTCAAGTATGCAATCTATTTCGAGGCTCGGGAAGAAACAGGTTTCTCTACATATAATCC | |
| | TCAAGTGATCATTCGAGGTGGGACACCTACTCATGCTAGAATTATCGTCAGGCATATGGCTGCTCCTCTG | |
| | ATTGGCCAATTGACAAGGCATGAAATTGAAATGACAGAGAAAGAATGGAAATATTATGGGGATGATCCTC | |
| | GAGTCCATAGAACTGTGACCCGAGAAGACTTCTTGGATATACTATATGATATTCATTACATTCTTATCAA | |
| | AGCTACTTATGGAAATTTCATGCGACAAAGCAGGATTTCTGAAATCTCAATGGAGGTAGCTGAACAAGGA | |
| | CGTGGAACAACAATGACTCCTCCAGCTGACTTGATTGAAAAATGTGATTGTCCCCTGGGCTATTCTGGCC | |
| | TGTCCTGTGAGGCATGCTTGCCGGGATTTTATCGACTGCGTTCTCAACCAGGTGGCCGCACCCCTGGACC | |
| | AACCCTGGGCACCTGTGTTCCATGTCAATGTAATGGACACAGCAGCCTGTGTGACCCTGAAACATCGATA | |
| | TGCCAGAATTGTCAACATCACACTGCTGGTGACTTCTGTGAACGATGTGCTCTTGGATACTATGGAATTG | |
| | TCAAGGGATTGCCAAATGACTGTCAGCAATGTGCCTGCCCTCTGATTTCTTCCAGTAACAATTTCAGCCC | |
| | CTCTTGTGTCGCAGAAGGACTTGACGACTACCGCTGCACGGCTTGTCCACGGGGATATGAAGGCCAGTAC | |
| | TGTGAAAGGTGTGCCCCTGGCTATACTGGCAGTCCAGGCAACCCTGGAGGCTCCTGCCAAGAATGTGAGT | |
| | GTGATCCCTATGGCTCACTGCCTGTGCCCTGTGACCCTGTCACAGGATTCTGCACGTGCCGACCTGGAGC | |
| | CACGGGAAGGAAGTGTGACGGCTGCAAGCACTGGCATGCACGCGAGGGCTGGGAGTGTGTTTTTTGTGGA | |
| | GATGAGTGCACTGGCCTTCTTCTCGGTGACTTGGCTCGCCTGGAGCAGATGGTCATGAGCATCAACCTCA | |
| | CTGGTCCGCTGCCTGCGCCATATAAAATGCTGTATGGTCTTGAAAATATGACTCAGGAGCTAAAGCACTT | |
| | GCTGTCACCTCAGCGGGCCCCAGAGAGGCTTATTCAGCTGGCAGAGGGCAATCTGAATACACTCGTGACC | |
| | GAAATGAACGAGCTGCTGACCAGGGCTACCAAAGTGACAGCAGATGGCGAGCAGACCGGACAGGATGCTG | |
| | AGAGGACCAACACAAGAGCAAAGTCCCTGGGAGAATTCATTAAGGAGCTTGCCCGGGATGCAGAAGCTGT | |
| | AAATGAAAAAGCTATAAAACTAAATGAAACTCTAGGAACTCGAGACGAGGCCTTTGAGAGAAATTTGGAA | |
| | GGGCTTCAGAAAGAGATTGACCAGATGATTAAAGAACTGAGGAGGAAAAATCTAGAGACACAAAAGGAAA | |
| | TTGCTGAAGATGAGTTGGTAGCTGCAGAAGCCCTTCTGAAAAAAGTGAAGAAGCTGTTTGGAGAGTCCCG | |
| | GGGGGAAAATGAAGAAATGGAGAAGGATCTCCGGGAAAAACTGGCTGACTACAAAAACAAAGTTGATGAT | |
| | GCTTGGGACCTTTTGAGAGAAGCCACAGATAAAATCAGAGAAGCTAATCGCCTATTTGCAGTAAATCAGA | |
| | AAAACATGACTGCATTGGAGAAAAAGAAGGAGGCTGTTGAAAGCGGCAAACGACAAATTGAGAACACTTT | |
| | AAAAGAGGGCAATGACATACTCGATGAAGCCAACCGTCTTGCAGATGAAATCAACTCCATCATAGACTAT | |
| | GTTGAAGACATCCAAACTAAATTGCCACCTATGTCTGAGGAGCTTAATGATAAAATAGATGACCTCTCCC | |
| | AAGAAATAAAGGACAGGAAGCTTGCTGAGAAGGTGTCCCAGGCTGAGAGCCACGCAGCTCAGTTGAATGA | |
| | CTCATCTGCTGTCCTTGATGGAATCCTTGATGAGGCTAAAAACATCTCCTTCAATGCCACTGCAGCCTTC | |
| | AAAGCTTACAGCAATATTAAGGACTATATTGATGAAGCTGAGAAAGTTGCCAAAGAAGCCAAAGATCTTG | |
| | CACATGAAGCTACAAAACTGGCAACAGGTCCTCGGGGTTTATTAAAGGAAGATGCCAAAGGCTGTCTTCA | |
| | GAAAAGCTTCAGGATTCTTAACGAAGCCAAGAAGTTAGCAAATGATGTAAAAGAAAATGAAGACCATCTA | |
| | AATGGCTTAAAAACCAGGATAGAAAATGCTGATGCTAGAAATGGGGATCTCTTGAGAACTTTGAATGACA | |
| | CTTTGGGAAAGTTATCAGCTATTCCAAATGATACAGCTGCTAAACTGCAAGCTGTTAAGGACAAAGCCAG | |
| | ACAAGCCAACGACACAGCTAAAGATGTACTGGCACAGATTACAGAGCTCCACCAGAACCTCGATGGCCTG | |
| | AAGAAGAATTACAATAAACTAGCAGACAGCGTCGCCAAAACGAATGCTGTGGTTAAAGATCCTTCCAAGA | |
| | ACAAAATCATTGCCGATGCAGATGCCACTGTCAAAAATTTAGAACAGGAAGCTGACCGGCTAATAGATAA | |
| | ACTCAAACCCATCAAGGAACTTGAGGATAACCTAAAGAAAAACATCTCTGAGATAAAGGAATTGATAAAC | |
| | CAAGCTCGGAAACAAGCCAATTCTATCAAAGTATCTGTGTCTTCAGGAGGTGACTGCATTCGAACATACA | |
| | AACCAGAAATCAAGAAAGGAAGTTACAATAATATTGTTGTCAACGTAAAGACAGCTGTTGCTGATAACCT | |
| | CCTCTTTTATCTTGGAAGTGCCAAATTTATTGACTTTCTGGCTATAGAAATGCGTAAAGGCAAAGTCAGC | |
| | TTCCTCTGGGATGTTGGATCTGGAGTTGGACGTGTAGAGTACCCAGATTTGACTATTGATGACTCATATT | |
| | GGTACCGTATCGTAGCATCAAGAACTGGGAGAAATGGAACTATTTCTGTGAGAGCCCTGGATGGACCCAA | |
| | AGCCAGCATTGTGCCCAGCACACACCATTCGACGTCTCCTCCAGGGTACACGATTCTAGATGTGGATGCA | |
| | AATGCAATGCTGTTTGTTGGTGGCCTGACTGGGAAATTAAAGAAGGCTGATGCTGTACGTGTGATTACAT | |
| | TCACTGGCTGCATGGGAGAAACATACTTTGACAACAAACCTATAGGTTTGTGGAATTTCCGAGAAAAAGA | |
| | AGGTGACTGCAAAGGATGCACTGTCAGTCCTCAGGTGGAAGATAGTGAGGGGACTATTCAATTTGATGGA | |
| | GAAGGTTATGCATTGGTCAGCCGTCCCATTCGCTGGTACCCCAACATCTCCACTGTCATGTTCAAGTTCA | |
| | GAACATTTTCTTCGAGTGCTCTTCTGATGTATCTTGCCACACGAGACCTGAGAGATTTCATGAGTGTGGA | |
| | GCTCACTGATGGGCACATAAAAGTCAGTTACGATCTGGGCTCAGGAATGGCTTCCGTTGTCAGCAATCAA | |
| | AACCATAATGATGGGAAATGGAAATCATTCACTCTGTCAAGAATTCAAAAACAAGCCAATATATCAATTG | |
| | TAGATATAGATACTAATCAGGAGGAGAATATAGCAACTTCGTCTTCTGGAAACAACTTTGGTCTTGACTT | |
| | GAAAGCAGATGACAAAATATATTTTGGTGGCCTGCCAACGCTGAGAAACTTGAGGCCAGAAGTAAATCTG | |
| | AAGAAATATTCCGGCTGCCTCAAAGATATTGAAATTTCAAGAACTCCGTACAATATACTCAGTAGTCCCG | |
| | ATTATGTTGGTGTTACCAAAGGATGTTCCCTGGAGAATGTTTACACAGTTAGCTTTCCTAAGCCTGGTTT | |
| | TGTGGAGCTCTCCCCTGTGCCAATTGATGTAGGAACAGAAATCAACCTGTCATTCAGCACCAAGAATGAG | |
| | TCCGGCATCATTCTTTTGGGAAGTGGAGGGACACCAGCACCACCTAGGAGAAAACGAAGGCAGACTGGAC | |
| | AGGCCTATTATGTAATACTCCTCAACAGGGGCCGTCTGGAAGTGCATCTCTCCACAGGGGCACGAACAAT | |
| | GAGGAAAATTGTGATCAGACCAGAGCCGAATCTGTTTCATGATGGAAGAGAACATTCCGTTCATGTAGAG | |
| | CGAACTAGAGGCATCTTTACAGTTCAAGTGGATGAAAACAGAAGATACATGCAAAACCTGACAGTTGAAC | |
| | AGCCTATCGAAGTTAAAAAGCTTTTCGTTGGGGGTGCTCCACCTGAATTTCAACCTTCCCCACTCAGAAA | |
| | TATTCCTCCTTTTGAAGGCTGCATATGGAATCTTGTTATTAACTCTGTCCCCATGGACTTTGCAAGGCCT | |
| | GTGTCCTTCAAAAATGCTGACATTGGTCGCTGTGCCCATCAGAAACTCCGTGAAGATGAAGATGGAGCAG | |
| | CTCCAGCTGAAATAGTTATCCAGCCTGAGCCAGTTCCCACCCCAGCCTTTCCTACGCCCACCCCAGTTCT | |
| | GACACATGGTCCTTGTGCTGCAGAATCAGAACCAGCTCTTTTGATAGGGAGCAAGCAGTTCGGGCTTTCA | |
| | AGAAACAGTCACATTGCAATTGCATTTGATGACACCAAAGTTAAAAACCGTCTCACAATTGAGTTGGAAG | |
| | TAAGAACCGAAGCTGAATCCGGCTTGCTTTTTTACATGGCTCGCATCAATCATGCTGATTTTGCAACAGT | |
| | TCAGCTGAGAAATGGATTGCCCTACTTCAGCTATGACTTGGGGAGTGGGGACACCCACACCATGATCCCC | |
| | ACCAAAATCAATGATGGCCAGTGGCACAAGATTAAGATAATGAGAAGTAAGCAAGAAGGAATTCTTTATG | |
| | TAGATGGGGCTTCCAACAGAACCATCAGTCCCAAAAAAGCCGACATCCTGGATGTCGTGGGAATGCTGTA | |
| | TGTTGGTGGGTTACCCATCAACTACACTACCCGAAGAATTGGTCCAGTGACCTATAGCATTGATGGCTGC | |
| | GTCAGGAATCTCCACATGGCAGAGGCCCCTGCCGATCTGGAACAACCCACCTCCAGCTTCCATGTTGGGA | |
| | CATGTTTTGCAAATGCTCAGAGGGGAACATATTTTGACGGAACCGGTTTTGCCAAAGCAGTTGGTGGATT | |
| | CAAAGTGGGATTGGACCTTCTTGTAGAATTTGAATTCCGCACAACTACAACGACTGGAGTTCTTCTGGGG | |
| | ATCAGTAGTCAAAAAATGGATGGAATGGGTATTGAAATGATTGATGAAAAGTTGATGTTTCATGTGGACA | |
| | ATGGTGCGGGCAGATTCACTGCTGTCTATGATGCTGGGGTTCCAGGGCATTTGTGTGATGGACAATGGCA | |
| | TAAAGTCACTGCCAACAAGATCAAACACCGCATTGAGCTCACAGTCGATGGGAACCAGGTGGAAGCCCAA | |
| | AGCCCAAACCCAGCATCTACATCAGCTGACACAAATGACCCTGTGTTTGTTGGAGGCTTCCCAGATGACC | |
| | TCAAGCAGTTTGGCCTAACAACCAGTATTCCGTTCCGAGGTTGCATCAGATCCCTGAAGCTCACCAAAGG | |
| | CACAGGCAAGCCACTGGAGGTTAATTTTGCCAAGGCCCTGGAACTGAGGGGCGTTCAACCTGTATCATGC | |
| | CCAGCCAACTAATAAAAATAAGTGTAACCCCAGGAAGAGTCTGTCAAAACAAGTATATCAAGTAAAACAA | |
| | ACAAATATATTTTACCTATATATGTTAATTAAACTAATTTGTGCATGTACATAGAATTCTTTCTGTATTC | |
| | AGATGGTGCTAATTCAGACTCCAGACTGAATTTTAATTCAAGTTCTTTCTCAAGTCTATAAATAATATTA | |
| | AACTGATTATTTCATTCTAAAAAAAAAAAAAAAAAA | |
| Homo | CACGGCCGGTCTGTGCCGGCTGCTCCCGCGGTTAGGTCCCGCCCCGCGCAGCGCGCGCAGCCTGCGGAGC | 59 |
| sapiens | CAGCGGCCGTGACGCGACAACGATTCGGCTGTGACGCGACAACGATTCGGCTGTGACGCGAGCGCGGCCG | |
| emerin | CTCCCGATGCGCTCGTGCCGCCCCCGCCGTGCTCCTCGGCAGCCGTTGCTCGGCCGGTTTTGGTAGGCCC | |
| (EMD) | GGGCCGCCGCCAGGCCTCCGCCTGAGCCCGCACCCGCCATGGACAACTACGCAGATCTTTCGGATACCGA | |
| | GCTGACCACCTTGCTGCGCCGGTACAACATCCCGCACGGGCCTGTAGTAGGATCAACTCGTAGGCTTTAC | |
| | GAGAAGAAGATCTTCGAGTACGAGACCCAGAGGCGGCGGCTCTCGCCCCCCAGCTCGTCCGCCGCCTCCT | |
| | CTTATAGCTTCTCTGACTTGAATTCGACTAGAGGGGATGCAGATATGTATGATCTTCCCAAGAAAGAGGA | |
| | CGCTTTACTCTACCAGAGCAAGGGCTACAATGACGACTACTATGAAGAGAGCTACTTCACCACCAGGACT | |
| | TATGGGGAGCCCGAGTCTGCCGGCCCGTCCAGGGCTGTCCGCCAGTCAGTGACTTCATTCCCAGATGCTG | |
| | ACGCTTTCCATCACCAGGTGCATGATGACGATCTTTTGTCTTCTTCTGAAGAGGAGTGCAAGGATAGGGA | |
| | ACGCCCCATGTACGGCCGGGACAGTGCCTACCAGAGCATCACGCACTACCGCCCTGTTTCAGCCTCCAGG | |
| | AGCTCCCTGGACCTGTCCTATTATCCTACTTCCTCCTCCACCTCTTTTATGTCCTCCTCATCATCTTCCT | |
| | CTTCATGGCTCACCCGCCGTGCCATCCGGCCTGAAAACCGTGCTCCTGGGGCTGGGCTGGGCCAGGATCG | |
| | CCAGGTCCCGCTCTGGGGCCAGCTGCTGCTTTTCCTGGTCTTTGTGATCGTCCTCTTCTTCATTTACCAC | |
| | TTCATGCAGGCTGAAGAAGGCAACCCCTTCTAGAGGGAGCCATGAGGGTCTGGGCTTCAGAGCTAGGTCT | |
| | TTGGGGAAGTCCTGGCTGACTGCCTTAGCAGTGGGGGTGGGGGTGGGGGCAGGGGCAGGGGCTTTATGTG | |
| | TTTTTGCTTGGGGGGCGCTGGGCCTAGCCCAGAGTAGTGCTTGCTCCCCCTGCCTTGTCCCACCAGGGAG | |
| | GCAGCAGACTCAGGCCCTCCATGGTCCTCTTTGTCATTTTGTTGACATGCATTCCTCCTTTTGTCATCTT | |
| | GTTGGGGGGAGGGGATTAACCAAAGGCCACCCTGACTTTGTTTTTGTGGACACACAATAAAAGCCCCGTT | |
| | TATTTGTAAAAAAAAAAAAAAAAAAAAAAAAAAAAAAAAA | |
| Homo | TCGACCGCCCAGCCAGGTGCAAAATGCCGTGTCATTGGGAGACTCCGCAGCCGGAGCATTAGATTACAGC | 60 |
| sapiens | TCGACGGAGCTCGGGAAGGGCGGCGGGGGTGGAAGATGAGCAGAAGCCCCTGTTCTCGGAACGCCGGCTG | |
| dysferlin, | ACAAGCGGGGTGAGCGCAGGCGGGGCGGGGACCCAGCCTAGCCCACTGGAGCAGCCGGGGGTGGCCCGTT | |
| complete | CCCCTTTAAGAGCAACTGCTCTAAGCCAGGAGCCAGAGATTCGAGCCGGCCTCGCCCAGCCAGCCCTCTC | |
| cds | CAGCGAGGGGACCCACAAGCGGCGCCTCGGCCCTCCCGACCTTTCCGAGCCCTCTTTGCGCCCTGGGCGC | |
| | ACGGGGCCCTACACGCGCCAAGCATGCTGAGGGTCTTCATCCTCTATGCCGAGAACGTCCACACACCCGA | |
| | CACCGACATCAGCGATGCCTACTGCTCCGCGGTGTTTGCAGGGGTGAAGAAGAGAACCAAAGTCATCAAG | |
| | AACAGCGTGAACCCTGTATGGAATGAGGGATTTGAATGGGACCTCAAGGGCATCCCCCTGGACCAGGGCT | |
| | CTGAGCTTCATGTGGTGGTCAAAGACCATGAGACGATGGGGAGGAACAGGTTCCTGGGGGAAGCCAAGGT | |
| | CCCACTCCGAGAGGTCCTCGCCACCCCTAGTCTGTCCGCCAGCTTCAATGCCCCCCTGCTGGACACCAAG | |
| | AAGCAGCCCACAGGGGCCTCGCTGGTCCTGCAGGTGTCCTACACACCGCTGCCTGGAGCTGTGCCCCTGT | |
| | TCCCGCCCCCTACTCCTCTGGAGCCCTCCCCGACTCTGCCTGACCTGGATGTAGTGGCAGACACAGGAGG | |
| | AGAGGAAGACACAGAGGACCAGGGACTCACTGGAGATGAGGCGGAGCCATTCCTGGATCAAAGCGGAGGC | |
| | CCGGGGGCTCCCACCACCCCAAGGAAACTACCTTCACGTCCTCCGCCCCACTACCCCGGGATCAAAAGAA | |
| | AGCGAAGTGCGCCTACATCTAGAAAGCTGCTGTCAGACAAACCGCAGGATTTCCAGATCAGGGTCCAGGT | |
| | GATCGAGGGGCGCCAGCTGCCGGGGGTGAACATCAAGCCTGTGGTCAAGGTTACCGCTGCAGGGCAGACC | |
| | AAGCGGACGCGGATCCACAAGGGAAACAGCCCACTCTTCAATGAGACTCTTTTCTTCAACTTGTTTGACT | |
| | CTCCTGGGGAGCTGTTTGATGAGCCCATCTTTATCACGGTGGTAGACTCTCGTTCTCTCAGGACAGATGC | |
| | TCTCCTCGGGGAGTTCCGGATGGACGTGGGCACCATTTACAGAGAGCCCCGGCACGCCTATCTCAGGAAG | |
| | TGGCTGCTGCTCTCAGACCCTGATGACTTCTCTGCTGGGGCCAGAGGCTACCTGAAAACAAGCCTTTGTG | |
| | TGCTGGGGCCTGGGGACGAAGCGCCTCTGGAGAGAAAAGACCCCTCTGAAGACAAGGAGGACATTGAAAG | |
| | CAACCTGCTCCGGCCCACAGGCGTAGCCCTGCGAGGAGCCCACTTCTGCCTGAAGGTCTTCCGGGCCGAG | |
| | GACTTGCCGCAGATGGACGATGCCGTGATGGACAACGTGAAACAGATCTTTGGCTTCGAGAGTAACAAGA | |
| | AGAACTTGGTGGACCCCTTTGTGGAGGTCAGCTTTGCGGGGAAAATGCTGTGCAGCAAGATCTTGGAGAA | |
| | GACGGCCAACCCTCAGTGGAACCAGAACATCACACTGCCTGCCATGTTTCCCTCCATGTGCGAAAAAATG | |
| | AGGATTCGTATCATAGACTGGGACCGCCTGACTCACAATGACATCGTGGCTACCACCTACCTGAGTATGT | |
| | CGAAAATCTCTGCCCCTGGAGGAGAAATAGAAGAGGAGCCTGCAGGTGCTGTCAAGCCTTCGAAAGCCTC | |
| | AGACTTGGATGACTACCTGGGCTTCCTCCCCACTTTTGGGCCCTGCTACATCAACCTCTATGGCAGTCCC | |
| | AGAGAGTTCACAGGCTTCCCAGACCCCTACACAGAGCTCAACACAGGCAAGGGGGAAGGTGTGGCTTATC | |
| | GTGGCCGGCTTCTGCTCTCCCTGGAGACCAAGCTGGTGGAGCACAGTGAACAGAAGGTGGAGGACCTTCC | |
| | TGCGGATGACATCCTCCGGGTGGAGAAGTACCTTAGGAGGCGCAAGTACTCCCTGTTTGCGGCCTTCTAC | |
| | TCAGCCACCATGCTGCAGGATGTGGATGATGCCATCCAGTTTGAGGTCAGCATCGGGAACTACGGGAACA | |
| | AGTTCGACATGACCTGCCTGCCGCTGGCCTCCACCACTCAGTACAGCCGTGCAGTCTTTGACGGGTGCCA | |
| | CTACTACTACCTACCCTGGGGTAACGTGAAACCTGTGGTGGTGCTGTCATCCTACTGGGAGGACATCAGC | |
| | CATAGAATCGAGACTCAGAACCAGCTGCTTGGGATTGCTGACCGGCTGGAAGCTGGCCTGGAGCAGGTCC | |
| | ACCTGGCCCTGAAGGCGCAGTGCTCCACGGAGGACGTGGACTCGCTGGTGGCTCAGCTGACGGATGAGCT | |
| | CATCGCAGGCTGCAGCCAGCCTCTGGGTGACATCCATGAGACACCCTCTGCCACCCACCTGGACCAGTAC | |
| | CTGTACCAGCTGCGCACCCATCACCTGAGCCAAATCACTGAGGCTGCCCTGGCCCTGAAGCTCGGCCACA | |
| | GTGAGCTCCCTGCAGCTCTGGAGCAGGCGGAGGACTGGCTCCTGCGTCTGCGTGCCCTGGCAGAGGAGCC | |
| | CCAGAACAGCCTGCCGGACATCGTCATCTGGATGCTGCAGGGAGACAAGCGTGTGGCATACCAGCGGGTG | |
| | CCCGCCCACCAAGTCCTCTTCTCCCGGCGGGGTGCCAACTACTGTGGCAAGAATTGTGGGAAGCTACAGA | |
| | CAATCTTTCTGAAATATCCGATGGAGAAGGTGCCTGGCGCCCGGATGCCAGTGCAGATACGGGTCAAGCT | |
| | GTGGTTTGGGCTCTCTGTGGATGAGAAGGAGTTCAACCAGTTTGCTGAGGGGAAGCTGTCTGTCTTTGCT | |
| | GAAACCTATGAGAACGAGACTAAGTTGGCCCTTGTTGGGAACTGGGGCACAACGGGCCTCACCTACCCCA | |
| | AGTTTTCTGACGTCACGGGCAAGATCAAGCTACCCAAGGACAGCTTCCGCCCCTCGGCCGGCTGGACCTG | |
| | GGCTGGAGATTGGTTCGTGTGTCCGGAGAAGACTCTGCTCCATGACATGGACGCCGGTCACCTGAGCTTC | |
| | GTGGAAGAGGTGTTTGAGAACCAGACCCGGCTTCCCGGAGGCCAGTGGATCTACATGAGTGACAACTACA | |
| | CCGATGTGAACGGGGAGAAGGTGCTTCCCAAGGATGACATTGAGTGCCCACTGGGCTGGAAGTGGGAAGA | |
| | TGAGGAATGGTCCACAGACCTCAACCGGGCTGTCGATGAGCAAGGCTGGGAGTATAGCATCACCATCCCC | |
| | CCGGAGCGGAAGCCGAAGCACTGGGTCCCTGCTGAGAAGATGTACTACACACACCGACGGCGGCGCTGGG | |
| | TGCGCCTGCGCAGGAGGGATCTCAGCCAAATGGAAGCACTGAAAAGGCACAGGCAGGCGGAGGCGGAGGG | |
| | CGAGGGCTGGGAGTACGCCTCTCTTTTTGGCTGGAAGTTCCACCTCGAGTACCGCAAGACAGATGCCTTC | |
| | CGCCGCCGCCGCTGGCGCCGTCGCATGGAGCCACTGGAGAAGACGGGGCCTGCAGCTGTGTTTGCCCTTG | |
| | AGGGGGCCCTGGGCGGCGTGATGGATGACAAGAGTGAAGATTCCATGTCCGTCTCCACCTTGAGCTTCGG | |
| | TGTGAACAGACCCACGATTTCCTGCATATTCGACTATGGGAACCGCTACCATCTACGCTGCTACATGTAC | |
| | CAGGCCCGGGACCTGGCTGCGATGGACAAGGACTCTTTTTCTGATCCCTATGCCATCGTCTCCTTCCTGC | |
| | ACCAGAGCCAGAAGACGGTGGTGGTGAAGAACACCCTTAACCCCACCTGGGACCAGACGCTCATCTTCTA | |
| | CGAGATCGAGATCTTTGGCGAGCCGGCCACAGTTGCTGAGCAACCGCCCAGCATTGTGGTGGAGCTGTAC | |
| | GACCATGACACTTATGGTGCAGACGAGTTTATGGGTCGCTGCATCTGTCAACCGAGTCTGGAACGGATGC | |
| | CACGGCTGGCCTGGTTCCCACTGACGAGGGGCAGCCAGCCGTCGGGGGAGCTGCTGGCCTCTTTTGAGCT | |
| | CATCCAGAGAGAGAAGCCGGCCATCCACCATATTCCTGGTTTTGAGGTGCAGGAGACATCAAGGATCCTG | |
| | GATGAGTCTGAGGACACAGACCTGCCCTACCCACCACCCCAGAGGGAGGCCAACATCTACATGGTTCCTC | |
| | AGAACATCAAGCCAGCGCTCCAGCGTACCGCCATCGAGATCCTGGCATGGGGCCTGCGGAACATGAAGAG | |
| | TTACCAGCTGGCCAACATCTCCTCCCCCAGCCTCGTGGTAGAGTGTGGGGGCCAGACGGTGCAGTCCTGT | |
| | GTCATCAGGAACCTCCGGAAGAACCCCAACTTTGACATCTGCACCCTCTTCATGGAAGTGATGCTGCCCA | |
| | GGGAGGAGCTCTACTGCCCCCCCATCACCGTCAAGGTCATCGATAACCGCCAGTTTGGCCGCCGGCCTGT | |
| | GGTGGGCCAGTGTACCATCCGCTCCCTGGAGAGCTTCCTGTGTGACCCCTACTCGGCGGAGAGTCCATCC | |
| | CCACAGGGTGGCCCAGACGATGTGAGCCTACTCAGTCCTGGGGAAGACGTGCTCATCGACATTGATGACA | |
| | AGGAGCCCCTCATCCCCATCCAGGAGGAAGAGTTCATCGATTGGTGGAGCAAATTCTTTGCCTCCATAGG | |
| | GGAGAGGGAAAAGTGCGGCTCCTACCTGGAGAAGGATTTTGACACCCTGAAGGTCTATGACACACAGCTG | |
| | GAGAATGTGGAGGCCTTTGAGGGCCTGTCTGACTTTTGTAACACCTTCAAGCTGTACCGGGGCAAGACGC | |
| | AGGAGGAGACAGAAGATCCATCTGTGATTGGTGAATTTAAGGGCCTCTTCAAAATTTATCCCCTCCCAGA | |
| | AGACCCAGCCATCCCCATGCCCCCAAGACAGTTCCACCAGCTGGCCGCCCAGGGACCCCAGGAGTGCTTG | |
| | GTCCGTATCTACATTGTCCGAGCATTTGGCCTGCAGCCCAAGGACCCCAATGGAAAGTGTGATCCTTACA | |
| | TCAAGATCTCCATAGGGAAGAAATCAGTGAGTGACCAGGATAACTACATCCCCTGCACGCTGGAGCCCGT | |
| | ATTTGGAAAGATGTTCGAGCTGACCTGCACTCTGCCTCTGGAGAAGGACCTAAAGATCACTCTCTATGAC | |
| | TATGACCTCCTCTCCAAGGACGAAAAGATCGGTGAGACGGTCGTCGACCTGGAGAACAGGCTGCTGTCCA | |
| | AGTTTGGGGCTCGCTGTGGACTCCCACAGACCTACTGTGTCTCTGGACCGAACCAGTGGCGGGACCAGCT | |
| | CCGCCCCTCCCAGCTCCTCCACCTCTTCTGCCAGCAGCATAGAGTCAAGGCACCTGTGTACCGGACAGAC | |
| | CGTGTAATGTTTCAGGATAAAGAATATTCCATTGAAGAGATAGAGGCTGGCAGGATCCCAAACCCACACC | |
| | TGGGCCCAGTGGAGGAGCGTCTGGCTCTGCATGTGCTTCAGCAGCAGGGCCTGGTCCCGGAGCACGTGGA | |
| | GTCACGGCCCCTCTACAGCCCCCTGCAGCCAGACATCGAGCAGGGGAAGCTGCAGATGTGGGTCGACCTA | |
| | TTTCCGAAGGCCCTGGGGCGGCCTGGACCTCCCTTCAACATCACCCCACGGAGAGCCAGAAGGTTTTTCC | |
| | TGCGTTGTATTATCTGGAATACCAGAGATGTGATCCTGGATGACCTGAGCCTCACGGGGGAGAAGATGAG | |
| | CGACATTTATGTGAAAGGTTGGATGATTGGCTTTGAAGAACACAAGCAAAAGACAGACGTGCATTATCGT | |
| | TCCCTGGGAGGTGAAGGCAACTTCAACTGGAGGTTCATTTTCCCCTTCGACTACCTGCCAGCTGAGCAAG | |
| | TCTGTACCATTGCCAAGAAGGATGCCTTCTGGAGGCTGGACAAGACTGAGAGCAAAATCCCAGCACGAGT | |
| | GGTGTTCCAGATCTGGGACAATGACAAGTTCTCCTTTGATGATTTTCTGGGCTCCCTGCAGCTCGATCTC | |
| | AACCGCATGCCCAAGCCAGCCAAGACAGCCAAGAAGTGCTCCTTGGACCAGCTGGATGATGCTTTCCACC | |
| | CAGAATGGTTTGTGTCCCTTTTTGAGCAGAAAACAGTGAAGGGCTGGTGGCCCTGTGTAGCAGAAGAGGG | |
| | TGAGAAGAAAATACTGGCGGGCAAGCTGGAAATGACCTTGGAGATTGTAGCAGAGAGTGAGCATGAGGAG | |
| | CGGCCTGCTGGCCAGGGCCGGGATGAGCCCAACATGAACCCTAAGCTTGAGGACCCAAGGCGCCCCGACA | |
| | CCTCCTTCCTGTGGTTTACCTCCCCATACAAGACCATGAAGTTCATCCTGTGGCGGCGTTTCCGGTGGGC | |
| | CATCATCCTCTTCATCATCCTCTTCATCCTGCTGCTGTTCCTGGCCATCTTCATCTACGCCTTCCCGAAC | |
| | TATGCTGCCATGAAGCTGGTGAAGCCCTTCAGCTGAGGACTCTCCTGCCCTGTAGAAGGGGCCGTGGGGT | |
| | CCCCTCCAGCATGGGACTGGCCTGCCTCCTCCGCCCAGCTCGGCGAGCTCCTCCAGACCTCCTAGGCCTG | |
| | ATTGTCCTGCCAGGGTGGGCAGACAGACAGATGGACCGGCCCACACTCCCAGAGTTGCTAACATGGAGCT | |
| | CTGAGATCACCCCACTTCCATCATTTCCTTCTCCCCCAACCCAACGCTTTTTTGGATCAGCTCAGACATA | |
| | TTTCAGTATAAAACAGTTGGAACCACAAAAAAAAAAAAAAAAAAAAAAAAA | |
| Homo | AGGAGCAAGCCGAGAGCCAGCCGGCCGGCGCACTCCGACTCCGAGCAGTCTCTGTCCTTCGACCCGAGCC | 61 |
| sapiens | CCGCGCCCTTTCCGGGACCCCTGCCCCGCGGGCAGCGCTGCCAACCTGCCGGCCATGGAGACCCCGTCCC | |
| lamin A/C | AGCGGCGCGCCACCCGCAGCGGGGCGCAGGCCAGCTCCACTCCGCTGTCGCCCACCCGCATCACCCGGCT | |
| (LMNA) | GCAGGAGAAGGAGGACCTGCAGGAGCTCAATGATCGCTTGGCGGTCTACATCGACCGTGTGCGCTCGCTG | |
| gene, | GAAACGGAGAACGCAGGGCTGCGCCTTCGCATCACCGAGTCTGAAGAGGTGGTCAGCCGCGAGGTGTCCG | |
| complete | GCATCAAGGCCGCCTACGAGGCCGAGCTCGGGGATGCCCGCAAGACCCTTGACTCAGTAGCCAAGGAGCG | |
| cds | CGCCCGCCTGCAGCTGGAGCTGAGCAAAGTGCGTGAGGAGTTTAAGGAGCTGAAAGCGCGGTGAGTTCGC | |
| | CCAGGTGGCTGCGTGCCTGGCGGGGAGTGGAGAGGGCGGCGGGCCGGCGCCCCTGGCCGGCCGCAGGAAG | |
| | GGAGTGAGAGGGCCTGGAGGCCGATAACTTTGCNNNNNNNNNNNNNNNNNNNNNNNNNNNNNNNNNNNNN | |
| | NNNNNNNNNNNNNNNNNNNNNNNNNNNNNNNNNNNNNNNNNNNNNNNNNNNNNNNNNNNNNNNCTGGTAA | |
| | TTGCAGGCATAGCAGCGCCAGCCCCCATGGCTGACCTCCTGGGAGCCTGGCACTGTCTAGGCACACAGAC | |
| | TCCTTCTCTTAAATCTACTCTCCCCTCTCTTCTTTAGCAATACCAAGAAGGAGGGTGACCTGATAGCTGC | |
| | TCAGGCTCGGCTGAAGGACCTGGAGGCTCTGCTGAACTCCAAGGAGGCCGCACTGAGCACTGCTCTCAGT | |
| | GAGAAGCGCACGCTGGAGGGCGAGCTGCATGATCTGCGGGGCCAGGTGGCCAAGGTGAGGCCACCCTGCA | |
| | GGGCCCACCCATGGCCCCACCTAACACATGTACACTCACTCTTCTACCTAGGCCCTCCCCCATGTGGTGC | |
| | CTGGTCTGACCTGTCACCTGATTTCAGAGCCATTCACCTGTCCTAGAGTCATTTTACCCACTGAGGTCAC | |
| | ATCTTATCCNNNNNNNNNNNNNNNNNNNNNNNNNNNNNNNNNNNNNNNNNNNNNNNNNNNNNNNNNNNNN | |
| | NNNNNNNNNNNNNNNNNNNNNNNNNNNNNNNNNNNNNNNGTCACCCAGGCTGGAGTGCAGTAGTGCGATC | |
| | TCGGCTCACTGCAACCTCCACCTCCTGGATTCAAGCGATTCTTGTGCCTCAGCCTCCTGAGTAGCTGGGA | |
| | CTACAGGCGTGTGCCACCATCATGCCTGGCTACTTTTTTGTATTAGATATATATTTTCTCTCTTAGCACA | |
| | GTACCTACCAAGAGTGAGTGAGTAGATGTCCTGACCCCTGCAGGCATCCAAGGCCCTCCTTCCCTGGACC | |
| | TGTTTCCACATGTGTGAAGGGGTGCACAGGCAGCAGCCCACCTCTCAGCTTCCTTCCAGTTCTTGTGTTC | |
| | TGTGACCCCTTTTCCTCATCTCTGCCTGCTTCCTCACAGCTTGAGGCAGCCCTAGGTGAGGCCAAGAAGC | |
| | AACTTCAGGATGAGATGCTGCGGCGGGTGGATGCTGAGAACAGGCTGCAGACCATGAAGGAGGAACTGGA | |
| | CTTCCAGAAGAACATCTACAGTGAGGTGGGGACTGTGCTTTGCAAGCCAGAGGGCTGGGGCTGGGTGATG | |
| | ACAGACTTGGGCTGGGCTAGGGGGGACCAGCTGTGTGCAGAGCTCGCCTTCCTGAGTCCCTTGCCCTAGT | |
| | GGACAGGGAGTTGGGGGTGGCCAGCACTCAGCTCCCAGGTTAAAGTGGGGCTGGTAGTGGCTCATGGAGT | |
| | AGGGCTGGGCAGGGAGCCCCGCCCCTGGGTCTTGGCCTCCCAGGAACTAATTCTGATTTTGGTTTCTGTG | |
| | TCCTTCCTCCAACCCTTCCAGGAGCTGCGTGAGACCAAGCGCCGTCATGAGACCCGACTGGTGGAGATTG | |
| | ACAATGGGAAGCAGCGTGAGTTTGAGAGCCGGCTGGCGGATGCGCTGCAGGAACTGCGGGCCCAGCATGA | |
| | GGACCAGGTGGAGCAGTATAAGAAGGAGCTGGAGAAGACTTATTCTGCCAAGGTGCTTGCTCTCGATTGG | |
| | TTCCCTCACTGCCTCTGCCCTTGGCAGCCCTACCCTTACCCACGCTGGGCTATGCCTTCTGGGGATCAGG | |
| | CAGATGGTGGCAGGGAGCTCAGGGTGGCCCAGGACCTGGGGCTGTAGCAGTGATGCCCAACTCAGGCCTG | |
| | TGCCTCCACCCCTCCCAGTCACCACAGTCCTAACCCTTTGTCCTCCCCTCCAGCTGGACAATGCCAGGCA | |
| | GTCTGCTGAGAGGAACAGCAACCTGGTGGGGGCTGCCCACGAGGAGCTGCAGCAGTCGCGCATCCGCATC | |
| | GACAGCCTCTCTGCCCAGCTCAGCCAGCTCCAGAAGCAGGTGATACCCCACCTCACCCCTCTCTCCAGGG | |
| | GCCTAGAGTCTGGGCCGGATGCAGGCTGGAAGCCCAGGGTTGGGGGTGGGGGTGGGGGTGGGAGGTTCCT | |
| | GAGGAGGAGAGGGATGAAAAGTGTCCCCACAACCACAGAGAAGGGTCGCAGGATGTGGAGTCAGATGGCC | |
| | TGTGTGCTGTTTCTGTACACTCTTACCTCACCTTCACTTCTCAGGGCTTTGGTTTTCCCATTCGAAAATG | |
| | GAGGCTGTTCTTAATCTCCCTAACTCAGAGTTGCCACAGGACTCTGCAATGTGAGGTGTTAAAAGCATCA | |
| | GTATTTTTCTAGTTGGCTGTGCTATTTGTGACAGGAGAAAAAGTCTAGCCTCAGAACGAGAGGTTTCAGT | |
| | TAGACAAGGGGAAGGACTTCCCAGTTGCCAGCCAAGACTATGTTTAGAGCTTGTGATGTTCAGAGCTGGC | |
| | TCTGATGAGGGCTCTGGGGAAGCTCTGATTGCAGATCCTGGAGAGAGTAGCCAGGTGTCTCCTACACCGA | |
| | CCCACGTCCCTCCTTCCCCATACTTAGGGCCCTTGGGAGCTCACCAAACCCTCCCACCCCCCTTCAGCTG | |
| | GCAGCCAAGGAGGCGAAGCTTCGAGACCTGGAGGACTCACTGGCCCGTGAGCGGGACACCAGCCGGCGGC | |
| | TGCTGGCGGAAAAGGAGCGGGAGATGGCCGAGATGCGGGCAAGGATGCAGCAGCAGCTGGACGAGTACCA | |
| | GGAGCTTCTGGACATCAAGCTGGCCCTGGACATGGAGATCCACGCCTACCGCAAGCTCTTGGAGGGCGAG | |
| | GAGGAGAGGTGGGCTGGGGAGACGTCGGGGAGGTGCTGGCAGTGTCCTCTGGCCGGCAACTGGCCTTGAC | |
| | TAGACCCCCACTTGGTCTCCCTCTCCCCAGGCTACGCCTGTCCCCCAGCCCTACCTCGCAGCGCAGCCGT | |
| | GGCCGTGCTTCCTCTCACTCATCCCAGACACAGGGTGGGGGCAGCGTCACCAAAAAGCGCAAACTGGAGT | |
| | CCACTGAGAGCCGCAGCAGCTTCTCACAGCACGCACGCACTAGCGGGCGCGTGGCCGTGGAGGAGGTGGA | |
| | TGAGGAGGGCAAGTTTGTCCGGCTGCGCAACAAGTCCAATGAGGTAGGCTCCTGCTCAGGGTCTAAGGGG | |
| | ATACAGCTGCATCAGGGAGAGAGTGGCAAGACAGAAGGATGGCATGTGGAGAGAGGAACATCCTTGCCCT | |
| | CAGAGGGTGGACCAGGGTGAGCCTGTATATCTCCTCCACNNNNNNNNNNNNNNNNNNNNNNNNNNNNNNN | |
| | NNNNNNNNNNNNNNNNNNNNNNNNNNNNNNNNNNNNNNNNNNNNNNNNNNNNNNNNNNNNNNNNNNNNNG | |
| | CTGCGTATGTGTCCACAGATCATGGCTATTATCCCCGGGGGAAGGGCAGTGACAGGGGTGTGTGTAGATG | |
| | GAAGGAGAGGCCTCAATTGCAGGCAGGCAGAGGGCTGGGCCTTTGAGCAAGATACACCCAAGAGCCTGGG | |
| | TGAGCCTCCCCGACCTTCCTCTTCCCTATCTTCCCGGCAGGACCAGTCCATGGGCAATTGGCAGATCAAG | |
| | CGCCAGAATGGAGATGATCCCTTGCTGACTTACCGGTTCCCACCAAAGTTCACCCTGAAGGCTGGGCAGG | |
| | TGGTGACGGTGAGTGGCAGGGCGCTTGGGACTCTGGGGAGGCCTTGGGTGGCGATGGGAGCGCTGGGGTA | |
| | AGTGTCCTTTTCTCCTCTCCAGATCTGGGCTGCAGGAGCTGGGGCCACCCACAGCCCCCCTACCGACCTG | |
| | GTGTGGAAGGCACAGAACACCTGGGGCTGCGGGAACAGCCTGCGTACGGCTCTCATCAACTCCACTGGGG | |
| | AAGTAAGTAGGCCTGGGCCTGGCTGCTTGCTGGACGAGGCTCCCCCTGATGGCCAACATCGGAGCCAGCT | |
| | GCCCCCAACCCAAGTTTGCCAATTCAGGGCCCCTTTCTAGAGCTCTCTGTTGCAGGCTCCAGACTTCTCN | |
| | NNNNNNNNNNNNNNNNNNNNNNNNNNNNNNNNNNNNNNNNNNNNNNNNNNNNNNNNNNNNNNNNNNNNNN | |
| | NNNNNNNNNNNNNNNNNNNNNNNNNNNNNNNNNNNNNNNNNNNNNNNNNNNNNNNNNTGAGTTCCTTAGC | |
| | TCCATCACCACAGAGGACAGAGTAAGCAGCAGGCCGGACAAAGGGCAGGCCACAAGAAAAGTTGCAGGTG | |
| | GTCACTGGGGTAGACATGCTGTACAACCCTTCCCTGGCCCTGACCCTTGGACCTGGTTCCATGTCCCCAC | |
| | CAGGAAGTGGCCATACGCAAGCTGGTGCGCTCAGTGACTGTGGTTGAGGACGACGAGGATGAGGATGGAG | |
| | ATGACCTGCTCCATCACCACCATGTGAGTGGTAGCCGCCGCTGAGGCCGAGCCTGCACTGGGGCCACCCA | |
| | GCCAGGCCTGGGGGCAGCCTCTCCCCAGCCTCCCCGTGCCAAAAATCTTTTCATTAAAGAATGTTTTGGA | |
| | ACTTTACTCGCTGGCCTGGCCTTTCTTCTCTCTCCTCCCTATACCTTGAACAGGGAACCCAGGTGTCTGG | |
| | GTGCCCTACTCTGGTAAGGAAGGGAG | |

TABLE 7
RNA Sequences

| | | SEQ |
|---|---|---|
| | | ID |
| Name | Sequence | NO: |
| Homo | CCGGGUCCUUCUCCGAGAGCCGGGCGGGCACGCGUCAUUGUGUUACCUGCGGCCGGCCCGCGAGCUAGGC | 62 |
| sapiens | UGGUUUUUUUUUUUUCUCCCCUCCCUCCCCCCUUUUUCCAUGCAGCUGAUCUAAAAGGGAAUAAAAGGCU | |
| Jagged1 | GCGCAUAAUCAUAAUAAUAAAAGAAGGGGAGCGCGAGAGAAGGAAAGAAAGCCGGGAGGUGGAAGAGGAG | |
| (JAG1), | GGGGAGCGUCUCAAAGAAGCGAUCAGAAUAAUAAAAGGAGGCCGGGCUCUUUGCCUUCUGGAACGGGCCG | |
| complete | CUCUUGAAAGGGCUUUUGAAAAGUGGUGUUGUUUUCCAGUCGUGCAUGCUCCAAUCGGCGGAGUAUAUUA | |
| cds | GAGCCGGGACGCGGCGGCCGCAGGGGCAGCGGCGACGGCAGCACCGGCGGCAGCACCAGCGCGAACAGCA | |
| | GCGGCGGCGUCCCGAGUGCCCGCGGCGCGCGGCGCAGCGAUGCGUUCCCCACGGACGCGCGGCCGGUCCG | |
| | GGCGCCCCCUAAGCCUCCUGCUCGCCCUGCUCUGUGCCCUGCGAGCCAAGGUGUGUGGGGCCUCGGGUCA | |
| | GUUCGAGUUGGAGAUCCUGUCCAUGCAGAACGUGAACGGGGAGCUGCAGAACGGGAACUGCUGCGGCGGC | |
| | GCCCGGAACCCGGGAGACCGCAAGUGCACCCGCGACGAGUGUGACACAUACUUCAAAGUGUGCCUCAAGG | |
| | AGUAUCAGUCCCGCGUCACGGCCGGGGGGCCCUGCAGCUUCGGCUCAGGGUCCACGCCUGUCAUCGGGGG | |
| | CAACACCUUCAACCUCAAGGCCAGCCGCGGCAACGACCGCAACCGCAUCGUGCUGCCUUUCAGUUUCGCC | |
| | UGGCCGAGGUCCUAUACGUUGCUUGUGGAGGCGUGGGAUUCCAGUAAUGACACCGUUCAACCUGACAGUA | |
| | UUAUUGAAAAGGCUUCUCACUCGGGCAUGAUCAACCCCAGCCGGCAGUGGCAGACGCUGAAGCAGAACAC | |
| | GGGCGUUGCCCACUUUGAGUAUCAGAUCCGCGUGACCUGUGAUGACUACUACUAUGGCUUUGGCUGCAAU | |
| | AAGUUCUGCCGCCCCAGAGAUGACUUCUUUGGACACUAUGCCUGUGACCAGAAUGGCAACAAPACUUGCA | |
| | UGGAAGGCUGGAUGGGCCGCGAAUGUAACAGAGCUAUUUGCCGACAAGGCUGCAGUCCUAAGCAUGGGUC | |
| | UUGCAAACUCCCAGGUGACUGCAGGUGCCAGUACGGCUGGCAAGGCCUGUACUGUGAUAAGUGCAUCCCA | |
| | CACCCGGGAUGCGUCCACGGCAUCUGUAAUGAGCCCUGGCAGUGCCUCUGUGAGACCAACUGGGGCGGCC | |
| | AGCUCUGUGACAAAGAUCUCAAUUACUGUGGGACUCAUCAGCCGUGUCUCAACGGGGGAACUUGUAGCAA | |
| | CACAGGCCCUGACAAAUAUCAGUGUUCCUGCCCUGAGGGGUAUUCAGGACCCAACUGUGAAAUUGCUGAG | |
| | CACGCCUGCCUCUCUGAUCCCUGUCACAACAGAGGCAGCUGUAAGGAGACCUCCCUGGGCUUUGAGUGUG | |
| | AGUGUUCCCCAGGCUGGACCGGCCCCACAUGCUCUACAAACAUUGAUGACUGUUCUCCUAAUAACUGUUC | |
| | CCACGGGGGCACCUGCCAGGACCUGGUUAACGGAUUUAAGUGUGUGUGCCCCCCACAGUGGACUGGGAAA | |
| | ACGUGCCAGUUAGAUGCAAAUGAAUGUGAGGCCAAACCUUGUGUAAACGCCAAAUCCUGUAAGAAUCUCA | |
| | UUGCCAGCUACUACUGCGACUGUCUUCCCGGCUGGAUGGGUCAGAAUUGUGACAUAAAUAUUAAUGACUG | |
| | CCUUGGCCAGUGUCAGAAUGACGCCUCCUGUCGGGAUUUGGUUAAUGGUUAUCGCUGUAUCUGUCCACCU | |
| | GGCUAUGCAGGCGAUCACUGUGAGAGAGACAUCGAUGAAUGUGCCAGCAACCCCUGUUUGGAUGGGGGUC | |
| | ACUGUCAGAAUGAAAUCAACAGAUUCCAGUGUCUGUGUCCCACUGGUUUCUCUGGAAACCUCUGUCAGCU | |
| | GGACAUCGAUUAUUGUGAGCCUAAUCCCUGCCAGAACGGUGCCCAGUGCUACAACCGUGCCAGUGACUAU | |
| | UUCUGCAAGUGCCCCGAGGACUAUGAGGGCAAGAACUGCUCACACCUGAAAGACCACUGCCGCACGACCC | |
| | CCUGUGAAGUGAUUGACAGCUGCACAGUGGCCAUGGCUUCCAACGACACACCUGAAGGGGUGCGGUAUAU | |
| | UUCCUCCAACGUCUGUGGUCCUCACGGGAAGUGCAAGAGUCAGUCGGGAGGCAAAUUCACCUGUGACUGU | |
| | AACAAAGGCUUCACGGGAACAUACUGCCAUGAAAAUAUUAAUGACUGUGAGAGCAACCCUUGUAGAAACG | |
| | GUGGCACUUGCAUCGAUGGUGUCAACUCCUACAAGUGCAUCUGUAGUGACGGCUGGGAGGGGGCCUACUG | |
| | UGAAACCAAUAUUAAUGACUGCAGCCAGAACCCCUGCCACAAUGGGGGCACGUGUCGCGACCUGGUCAAU | |
| | GACUUCUACUGUGACUGUAAAAAUGGGUGGAAAGGAAAGACCUGCCACUCACGUGACAGUCAGUGUGAUG | |
| | AGGCCACGUGCAACAACGGUGGCACCUGCUAUGAUGAGGGGGAUGCUUUUAAGUGCAUGUGUCCUGGCGG | |
| | CUGGGAAGGAACAACCUGUAACAUAGCCCGAAACAGUAGCUGCCUGCCCAACCCCUGCCAUAAUGGGGGC | |
| | ACAUGUGUGGUCAACGGCGAGUCCUUUACGUGCGUCUGCAAGGAAGGCUGGGAGGGGCCCAUCUGUGCUC | |
| | AGAAUACCAAUGACUGCAGCCCUCAUCCCUGUUACAACAGCGGCACCUGUGUGGAUGGAGACAACUGGUA | |
| | CCGGUGCGAAUGUGCCCCGGGUUUUGCUGGGCCCGACUGCAGAAUAAACAUCAAUGAAUGCCAGUCUUCA | |
| | CCUUGUGCCUUUGGAGCGACCUGUGUGGAUGAGAUCAAUGGCUACCGGUGUGUCUGCCCUCCAGGGCACA | |
| | GUGGUGCCAAGUGCCAGGAAGUUUCAGGGAGACCUUGCAUCACCAUGGGGAGUGUGAUACCAGAUGGGGC | |
| | CAAAUGGGAUGAUGACUGUAAUACCUGCCAGUGCCUGAAUGGACGGAUCGCCUGCUCAAAGGUCUGGUGU | |
| | GGCCCUCGACCUUGCCUGCUCCACAAAGGGCACAGCGAGUGCCCCAGCGGGCAGAGCUGCAUCCCCAUCC | |
| | UGGACGACCAGUGCUUCGUCCACCCCUGCACUGGUGUGGGCGAGUGUCGGUCUUCCAGUCUCCAGCCGGU | |
| | GAAGACAAAGUGCACCUCUGACUCCUAUUACCAGGAUAACUGUGCGAACAUCACAUUUACCUUUAACAAG | |
| | GAGAUGAUGUCACCAGGUCUUACUACGGAGCACAUUUGCAGUGAAUUGAGGAAUUUGAAUAUUUUGAAGA | |
| | AUGUUUCCGCUGAAUAUUCAAUCUACAUCGCUUGCGAGCCUUCCCCUUCAGCGAACAAUGAAAUACAUGU | |
| | GGCCAUUUCUGCUGAAGAUAUACGGGAUGAUGGGAACCCGAUCAAGGAAAUCACUGACAAAAUAAUUGAU | |
| | CUUGUUAGUAAACGUGAUGGAAACAGCUCGCUGAUUGCUGCCGUUGCAGAAGUAAGAGUUCAGAGGCGGC | |
| | CUCUGAAGAACAGAACAGAUUUCCUUGUUCCCUUGCUGAGCUCUGUCUUAACUGUGGCUUGGAUCUGUUG | |
| | CUUGGUGACGGCCUUCUACUGGUGCCUGCGGAAGCGGCGGAAGCCGGGCAGCCACACACACUCAGCCUCU | |
| | GAGGACAACACCACCAACAACGUGCGGGAGCAGCUGAACCAGAUCAAAAACCCCAUUGAGAAACAUGGGG | |
| | CCAACACGGUCCCCAUCAAGGAUUACGAGAACAAGAACUCCAAAAUGUCUAAAAUAAGGACACACAAUUC | |
| | UGAAGUAGAAGAGGACGACAUGGACAAACACCAGCAGAAAGCCCGGUUUGCCAAGCAGCCGGCGUAUACG | |
| | CUGGUAGACAGAGAAGAGAAGCCCCCCAACGGCACGCCGACAAAACACCCAAACUGGACAAACAAACAGG | |
| | ACAACAGAGACUUGGAAAGUGCCCAGAGCUUAAACCGAAUGGAGUACAUCGUAUAGCAGACCGCGGGCAC | |
| | UGCCGCCGCUAGGUAGAGUCUGAGGGCUUGUAGUUCUUUAAACUGUCGUGUCAUACUCGAGUCUGAGGCC | |
| | GUUGCUGACUUAGAAUCCCUGUGUUAAUUUAAGUUUUGACAAGCUGGCUUACACUGGCAAUGGUAGUUUC | |
| | UGUGGUUGGCUGGGAAAUCGAGUGCCGCAUCUCACAGCUAUGCAAAAAGCUAGUCAACAGUACCCUGGUU | |
| | GUGUGUCCCCUUGCAGCCGACACGGUCUCGGAUCAGGCUCCCAGGAGCCUGCCCAGCCCCCUGGUCUUUG | |
| | AGCUCCCACUUCUGCCAGAUGUCCUAAUGGUGAUGCAGUCUUAGAUCAUAGUUUUAUUUAUAUUUAUUGA | |
| | CUCUUGAGUUGUUUUUGUAUAUUGGUUUUAUGAUGACGUACAAGUAGUUCUGUAUUUGAAAGUGCCUUUG | |
| | CAGCUCAGAACCACAGCAACGAUCACAAAUGACUUUAUUAUUUAUUUUUUUAAUUGUAUUUUUGUUGUUG | |
| | GGGGAGGGGAGACUUUGAUGUCAGCAGUUGCUGGUAAAAUGAAGAAUUUAAAGAAAAAAAUGUCAAAAGU | |
| | AGAACUUUGUAUAGUUAUGUAAAUAAUUCUUUUUUAUUAAUCACUGUGUAUAUUUGAUUUAUUAACUUAA | |
| | UAAUCAAGAGCCUUAAAACAUCAUUCCUUUUUAUUUAUAUGUAUGUGUUUAGAAUUGAAGGUUUUUGAUA | |
| | GCAUUGUAAGCGUAUGGCUUUAUUUUUUUGAACUCUUCUCAUUACUUGUUGCCUAUAAGCCAAAAUUAAG | |
| | GUGUUUGAAAAUAGUUUAUUUUAAAACAAUAGGAUGGGCUUCUGUGCCCAGAAUACUGAUGGAAUUUUUU | |
| | UUGUACGACGUCAGAUGUUUAAAACACCUUCUAUAGCAUCACUUAAAACACGUUUUAAGGACUGACUGAG | |
| | GCAGUUUGAGGAUUAGUUUAGAACAGGUUUUUUUGUUUGUUUGUUUUUUGUUUUUCUGCUUUAGACUUGA | |
| | AAAGAGACAGGCAGGUGAUCUGCUGCAGAGCAGUAAGGGAACAAGUUGAGCUAUGACUUAACAUAGCCAA | |
| | AAUGUGAGUGGUUGAAUAUGAUUAAAAAUAUCAAAUUAAUUGUGUGAACUUGGAAGCACACCAAUCUGAC | |
| | UUUGUAAAUUCUGAUUUCUUUUCACCAUUCGUACAUAAUACUGAACCACUUGUAGAUUUGAUUUUUUUUU | |
| | UAAUCUACUGCAUUUAGGGAGUAUUCUAAUAAGCUAGUUGAAUACUUGAACCAUAAAAUGUCCAGUAAGA | |
| | UCACUGUUUAGAUUUGCCAUAGAGUACACUGCCUGCCUUAAGUGAGGAAAUCAAAGUGCUAUUACGAAGU | |
| | UCAAGAUCAAAAAGGCUUAUAAAACAGAGUAAUCUUGUUGGUUCACCAUUGAGACCGUGAAGAUACUUUG | |
| | UAUUGUCCUAUUAGUGUUAUAUGAACAUACAAAUGCAUCUUUGAUGUGUUGUUCUUGGCAAUAAAUUUUG | |
| | AAAAGUAAUAUUUAUUAAAUUUUUUUGUAUGAAAACAUGGAACAGUGUGGCUCUUCUGAGCUUACGUAGU | |
| | UCUACCGGCUUUGCCGUGUGCUUCUGCCACCCUGCUGAGUCUGUUCUGGUAAUCGGGGUAUAAUAGGCUC | |
| | UGCCUGACAGAGGGAUGGAGGAAGAACUGAAAGGCUUUUCAACCACAAAACUCAUCUGGAGUUCUCAAAG | |
| | ACCUGGGGCUGCUGUGAAGCUGGAACUGCGGGAGCCCCAUCUAGGGGAGCCUUGAUUCCCUUGUUAUUCA | |
| | ACAGCAAGUGUGAAUACUGCUUGAAUAAACACCACUGGAUUAAUGGAAAAkAkAPAPAPAPA | |
| Homo | GGGAUUCCCUCACUUUCCCCCUACAGGACUCAGAUCUGGGAGGCAAUUACCUUCGGAGAAAAACGAAUAG | 63 |
| sapiens | GAAAAACUGAAGUGUUACUUUUUUUAAAGCUGCUGAAGUUUGUUGGUUUCUCAUUGUUUUUAAGCCUACU | |
| dystrophin | GGAGCAAUAAAGUUUGAAGAACUUUUACCAGGUUUUUUUUAUCGCUGCCUUGAUAUACACUUUUCAAAAU | |
| (DMD), | GCUUUGGUGGGAAGAAGUAGAGGACUGUUAUGAAAGAGAAGAUGUUCAAAAGAAAACAUUCACAAAAUGG | |
| complete | GUAAAUGCACAAUUUUCUAAGUUUGGGAAGCAGCAUAUUGAGAACCUCUUCAGUGACCUACAGGAUGGGA | |
| cds | GGCGCCUCCUAGACCUCCUCGAAGGCCUGACAGGGCAAAAACUGCCAAAAGAAAAAGGAUCCACAAGAGU | |
| | UCAUGCCCUGAACAAUGUCAACAAGGCACUGCGGGUUUUGCAGAACAAUAAUGUUGAUUUAGUGAAUAUU | |
| | GGAAGUACUGACAUCGUAGAUGGAAAUCAUAAACUGACUCUUGGUUUGAUUUGGAAUAUAAUCCUCCACU | |
| | GGCAGGUCAAAAAUGUAAUGAAAAAUAUCAUGGCUGGAUUGCAACAAACCAACAGUGAAAAGAUUCUCCU | |
| | GAGCUGGGUCCGACAAUCAACUCGUAAUUAUCCACAGGUUAAUGUAAUCAACUUCACCACCAGCUGGUCU | |
| | GAUGGCCUGGCUUUGAAUGCUCUCAUCCAUAGUCAUAGGCCAGACCUAUUUGACUGGAAUAGUGUGGUUU | |
| | GCCAGCAGUCAGCCACACAACGACUGGAACAUGCAUUCAACAUCGCCAGAUAUCAAUUAGGCAUAGAGAA | |
| | ACUACUCGAUCCUGAAGAUGUUGAUACCACCUAUCCAGAUAAGAAGUCCAUCUUAAUGUACAUCACAUCA | |
| | CUCUUCCAAGUUUUGCCUCAACAAGUGAGCAUUGAAGCCAUCCAGGAAGUGGAAAUGUUGCCAAGGCCAC | |
| | CUAAAGUGACUAAAGAAGAACAUUUUCAGUUACAUCAUCAAAUGCACUAUUCUCAACAGAUCACGGUCAG | |
| | UCUAGCACAGGGAUAUGAGAGAACUUCUUCCCCUAAGCCUCGAUUCAAGAGCUAUGCCUACACACAGGCU | |
| | GCUUAUGUCACCACCUCUGACCCUACACGGAGCCCAUUUCCUUCACAGCAUUUGGAAGCUCCUGAAGACA | |
| | AGUCAUUUGGCAGUUCAUUGAUGGAGAGUGAAGUAAACCUGGACCGUUAUCAAACAGCUUUAGAAGAAGU | |
| | AUUAUCGUGGCUUCUUUCUGCUGAGGACACAUUGCAAGCACAAGGAGAGAUUUCUAAUGAUGUGGAAGUG | |
| | GUGAAAGACCAGUUUCAUACUCAUGAGGGGUACAUGAUGGAUUUGACAGCCCAUCAGGGCCGGGUUGGUA | |
| | AUAUUCUACAAUUGGGAAGUAAGCUGAUUGGAACAGGAAAAUUAUCAGAAGAUGAAGAAACUGAAGUACA | |
| | AGAGCAGAUGAAUCUCCUAAAUUCAAGAUGGGAAUGCCUCAGGGUAGCUAGCAUGGAAAAACAAAGCAAU | |
| | UUACAUAGAGUUUUAAUGGAUCUCCAGAAUCAGAAACUGAAAGAGUUGAAUGACUGGCUAACAAAAACAG | |
| | AAGAAAGAACAAGGAAAAUGGAGGAAGAGCCUCUUGGACCUGAUCUUGAAGACCUAAAACGCCAAGUACA | |
| | ACAACAUAAGGUGCUUCAAGAAGAUCUAGAACAAGAACAAGUCAGGGUCAAUUCUCUCACUCACAUGGUG | |
| | GUGGUAGUUGAUGAAUCUAGUGGAGAUCACGCAACUGCUGCUUUGGAAGAACAACUUAAGGUAUUGGGAG | |
| | AUCGAUGGGCAAACAUCUGUAGAUGGACAGAAGACCGCUGGGUUCUUUUACAAGACAUCCUUCUCAAAUG | |
| | GCAACGUCUUACUGAAGAACAGUGCCUUUUUAGUGCAUGGCUUUCAGAAAAAGAAGAUGCAGUGAACAAG | |
| | AUUCACACAACUGGCUUUAAAGAUCAAAAUGAAAUGUUAUCAAGUCUUCAAAAACUGGCCGUUUUAAAAG | |
| | CGGAUCUAGAAAAGAAAAAGCAAUCCAUGGGCAAACUGUAUUCACUCAAACAAGAUCUUCUUUCAACACU | |
| | GAAGAAUAAGUCAGUGACCCAGAAGACGGAAGCAUGGCUGGAUAACUUUGCCCGGUGUUGGGAUAAUUUA | |
| | GUCCAAAAACUUGAAAAGAGUACAGCACAGAUUUCACAGGCUGUCACCACCACUCAGCCAUCACUAACAC | |
| | AGACAACUGUAAUGGAAACAGUAACUACGGUGACCACAAGGGAACAGAUCCUGGUAAAGCAUGCUCAAGA | |
| | GGAACUUCCACCACCACCUCCCCAAAAGAAGAGGCAGAUUACUGUGGAUUCUGAAAUUAGGAAAAGGUUG | |
| | GAUGUUGAUAUAACUGAACUUCACAGCUGGAUUACUCGCUCAGAAGCUGUGUUGCAGAGUCCUGAAUUUG | |
| | CAAUCUUUCGGAAGGAAGGCAACUUCUCAGACUUAAAAGAAAAAGUCAAUGCCAUAGAGCGAGAAAAAGC | |
| | UGAGAAGUUCAGAAAACUGCAAGAUGCCAGCAGAUCAGCUCAGGCCCUGGUGGAACAGAUGGUGAAUGAG | |
| | GGUGUUAAUGCAGAUAGCAUCAAACAAGCCUCAGAACAACUGAACAGCCGGUGGAUCGAAUUCUGCCAGU | |
| | UGCUAAGUGAGAGACUUAACUGGCUGGAGUAUCAGAACAACAUCAUCGCUUUCUAUAAUCAGCUACAACA | |
| | AUUGGAGCAGAUGACAACUACUGCUGAAAACUGGUUGAAAAUCCAACCCACCACCCCAUCAGAGCCAACA | |
| | GCAAUUAAAAGUCAGUUAAAAAUUUGUAAGGAUGAAGUCAACCGGCUAUCAGGUCUUCAACCUCAAAUUG | |
| | AACGAUUAAAAAUUCAAAGCAUAGCCCUGAAAGAGAAAGGACAAGGACCCAUGUUCCUGGAUGCAGACUU | |
| | UGUGGCCUUUACAAAUCAUUUUAAGCAAGUCUUUUCUGAUGUGCAGGCCAGAGAGAAAGAGCUACAGACA | |
| | AUUUUUGACACUUUGCCACCAAUGCGCUAUCAGGAGACCAUGAGUGCCAUCAGGACAUGGGUCCAGCAGU | |
| | CAGAAACCAAACUCUCCAUACCUCAACUUAGUGUCACCGACUAUGAAAUCAUGGAGCAGAGACUCGGGGA | |
| | AUUGCAGGCUUUACAAAGUUCUCUGCAAGAGCAACAAAGUGGCCUAUACUAUCUCAGCACCACUGUGAAA | |
| | GAGAUGUCGAAGAAAGCGCCCUCUGAAAUUAGCCGGAAAUAUCAAUCAGAAUUUGAAGAAAUUGAGGGAC | |
| | GCUGGAAGAAGCUCUCCUCCCAGCUGGUUGAGCAUUGUCAAAAGCUAGAGGAGCAAAUGAAUAAACUCCG | |
| | AAAAAUUCAGAAUCACAUACAAACCCUGAAGAAAUGGAUGGCUGAAGUUGAUGUUUUUCUGAAGGAGGAA | |
| | UGGCCUGCCCUUGGGGAUUCAGAAAUUCUAAAAAAGCAGCUGAAACAGUGCAGACUUUUAGUCAGUGAUA | |
| | UUCAGACAAUUCAGCCCAGUCUAAACAGUGUCAAUGAAGGUGGGCAGAAGAUAAAGAAUGAAGCAGAGCC | |
| | AGAGUUUGCUUCGAGACUUGAGACAGAACUCAAAGAACUUAACACUCAGUGGGAUCACAUGUGCCAACAG | |
| | GUCUAUGCCAGAAAGGAGGCCUUGAAGGGAGGUUUGGAGAAAACUGUAAGCCUCCAGAAAGAUCUAUCAG | |
| | AGAUGCACGAAUGGAUGACACAAGCUGAAGAAGAGUAUCUUGAGAGAGAUUUUGAAUAUAAAACUCCAGA | |
| | UGAAUUACAGAAAGCAGUUGAAGAGAUGAAGAGAGCUAAAGAAGAGGCCCAACAAAAAGAAGCGAAAGUG | |
| | AAACUCCUUACUGAGUCUGUAAAUAGUGUCAUAGCUCAAGCUCCACCUGUAGCACAAGAGGCCUUAAAAA | |
| | AGGAACUUGAAACUCUAACCACCAACUACCAGUGGCUCUGCACUAGGCUGAAUGGGAAAUGCAAGACUUU | |
| | GGAAGAAGUUUGGGCAUGUUGGCAUGAGUUAUUGUCAUACUUGGAGAAAGCAAACAAGUGGCUAAAUGAA | |
| | GUAGAAUUUAAACUUAAAACCACUGAAAACAUUCCUGGCGGAGCUGAGGAAAUCUCUGAGGUGCUAGAUU | |
| | CACUUGAAAAUUUGAUGCGACAUUCAGAGGAUAACCCAAAUCAGAUUCGCAUAUUGGCACAGACCCUAAC | |
| | AGAUGGCGGAGUCAUGGAUGAGCUAAUCAAUGAGGAACUUGAGACAUUUAAUUCUCGUUGGAGGGAACUA | |
| | CAUGAAGAGGCUGUAAGGAGGCAAAAGUUGCUUGAACAGAGCAUCCAGUCUGCCCAGGAGACUGAAAAAU | |
| | CCUUACACUUAAUCCAGGAGUCCCUCACAUUCAUUGACAAGCAGUUGGCAGCUUAUAUUGCAGACAAGGU | |
| | GGACGCAGCUCAAAUGCCUCAGGAAGCCCAGAAAAUCCAAUCUGAUUUGACAAGUCAUGAGAUCAGUUUA | |
| | GAAGAAAUGAAGAAACAUAAUCAGGGGAAGGAGGCUGCCCAAAGAGUCCUGUCUCAGAUUGAUGUUGCAC | |
| | AGAAAAAAUUACAAGAUGUCUCCAUGAAGUUUCGAUUAUUCCAGAAACCAGCCAAUUUUGAGCUGCGUCU | |
| | ACAAGAAAGUAAGAUGAUUUUAGAUGAAGUGAAGAUGCACUUGCCUGCAUUGGAAACAAAGAGUGUGGAA | |
| | CAGGAAGUAGUACAGUCACAGCUAAAUCAUUGUGUGAACUUGUAUAAAAGUCUGAGUGAAGUGAAGUCUG | |
| | AAGUGGAAAUGGUGAUAAAGACUGGACGUCAGAUUGUACAGAAAAAGCAGACGGAAAAUCCCAAAGAACU | |
| | UGAUGAAAGAGUAACAGCUUUGAAAUUGCAUUAUAAUGAGCUGGGAGCAAAGGUAACAGAAAGAAAGCAA | |
| | CAGUUGGAGAAAUGCUUGAAAUUGUCCCGUAAGAUGCGAAAGGAAAUGAAUGUCUUGACAGAAUGGCUGG | |
| | CAGCUACAGAUAUGGAAUUGACAAAGAGAUCAGCAGUUGAAGGAAUGCCUAGUAAUUUGGAUUCUGAAGU | |
| | UGCCUGGGGAAAGGCUACUCAAAAAGAGAUUGAGAAACAGAAGGUGCACCUGAAGAGUAUCACAGAGGUA | |
| | GGAGAGGCCUUGAAAACAGUUUUGGGCAAGAAGGAGACGUUGGUGGAAGAUAAACUCAGUCUUCUGAAUA | |
| | GUAACUGGAUAGCUGUCACCUCCCGAGCAGAAGAGUGGUUAAAUCUUUUGUUGGAAUACCAGAAACACAU | |
| | GGAAACUUUUGACCAGAAUGUGGACCACAUCACAAAGUGGAUCAUUCAGGCUGACACACUUUUGGAUGAA | |
| | UCAGAGAAAAAGAAACCCCAGCAAAAAGAAGACGUGCUUAAGCGUUUAAAGGCAGAACUGAAUGACAUAC | |
| | GCCCAAAGGUGGACUCUACACGUGACCAAGCAGCAAACUUGAUGGCAAACCGCGGUGACCACUGCAGGAA | |
| | AUUAGUAGAGCCCCAAAUCUCAGAGCUCAACCAUCGAUUUGCAGCCAUUUCACACAGAAUUAAGACUGGA | |
| | AAGGCCUCCAUUCCUUUGAAGGAAUUGGAGCAGUUUAACUCAGAUAUACAAAAAUUGCUUGAACCACUGG | |
| | AGGCUGAAAUUCAGCAGGGGGUGAAUCUGAAAGAGGAAGACUUCAAUAAAGAUAUGAAUGAAGACAAUGA | |
| | GGGUACUGUAAAAGAAUUGUUGCAAAGAGGAGACAACUUACAACAAAGAAUCACAGAUGAGAGAAAGAGA | |
| | GAGGAAAUAAAGAUAAAACAGCAGCUGUUACAGACAAAACAUAAUGCUCUCAAGGAUUUGAGGUCUCAAA | |
| | GAAGAAAAAAGGCUCUAGAAAUUUCUCAUCAGUGGUAUCAGUACAAGAGGCAGGCUGAUGAUCUCCUGAA | |
| | AUGCUUGGAUGACAUUGAAAAAAAAUUAGCCAGCCUACCUGAGCCCAGAGAUGAAAGGAAAAUAAAGGAA | |
| | AUUGAUCGGGAAUUGCAGAAGAAGAAAGAGGAGCUGAAUGCAGUGCGUAGGCAAGCUGAGGGCUUGUCUG | |
| | AGGAUGGGGCCGCAAUGGCAGUGGAGCCAACUCAGAUCCAGCUCAGCAAGCGCUGGCGGGAAAUUGAGAG | |
| | CAAAUUUGCUCAGUUUCGAAGACUCAACUUUGCACAAAUUCACACUGUCCGUGAAGAAACGAUGAUGGUG | |
| | AUGACUGAAGACAUGCCUUUGGAAAUUUCUUAUGUGCCUUCUACUUAUUUGACUGAAAUCACUCAUGUCU | |
| | CACAAGCCCUAUUAGAAGUGGAACAACUUCUCAAUGCUCCUGACCUCUGUGCUAAGGACUUUGAAGAUCU | |
| | CUUUAAGCAAGAGGAGUCUCUGAAGAAUAUAAAAGAUAGUCUACAACAAAGCUCAGGUCGGAUUGACAUU | |
| | AUUCAUAGCAAGAAGACAGCAGCAUUGCAAAGUGCAACGCCUGUGGAAAGGGUGAAGCUACAGGAAGCUC | |
| | UCUCCCAGCUUGAUUUCCAAUGGGAAAAAGUUAACAAAAUGUACAAGGACCGACAAGGGCGAUUUGACAG | |
| | AUCUGUUGAGAAAUGGCGGCGUUUUCAUUAUGAUAUAAAGAUAUUUAAUCAGUGGCUAACAGAAGCUGAA | |
| | CAGUUUCUCAGAAAGACACAAAUUCCUGAGAAUUGGGAACAUGCUAAAUACAAAUGGUAUCUUAAGGAAC | |
| | UCCAGGAUGGCAUUGGGCAGCGGCAAACUGUUGUCAGAACAUUGAAUGCAACUGGGGAAGAAAUAAUUCA | |
| | GCAAUCCUCAAAAACAGAUGCCAGUAUUCUACAGGAAAAAUUGGGAAGCCUGAAUCUGCGGUGGCAGGAG | |
| | GUCUGCAAACAGCUGUCAGACAGAAAAAAGAGGCUAGAAGAACAAAAGAAUAUCUUGUCAGAAUUUCAAA | |
| | GAGAUUUAAAUGAAUUUGUUUUAUGGUUGGAGGAAGCAGAUAACAUUGCUAGUAUCCCACUUGAACCUGG | |
| | AAAAGAGCAGCAACUAAAAGAAAAGCUUGAGCAAGUCAAGUUACUGGUGGAAGAGUUGCCCCUGCGCCAG | |
| | GGAAUUCUCAAACAAUUAAAUGAAACUGGAGGACCCGUGCUUGUAAGUGCUCCCAUAAGCCCAGAAGAGC | |
| | AAGAUAAACUUGAAAAUAAGCUCAAGCAGACAAAUCUCCAGUGGAUAAAGGUUUCCAGAGCUUUACCUGA | |
| | GAAACAAGGAGAAAUUGAAGCUCAAAUAAAAGACCUUGGGCAGCUUGAAAAAAAGCUUGAAGACCUUGAA | |
| | GAGCAGUUAAAUCAUCUGCUGCUGUGGUUAUCUCCUAUUAGGAAUCAGUUGGAAAUUUAUAACCAACCAA | |
| | ACCAAGAAGGACCAUUUGACGUUCAGGAAACUGAAAUAGCAGUUCAAGCUAAACAACCGGAUGUGGAAGA | |
| | GAUUUUGUCUAAAGGGCAGCAUUUGUACAAGGAAAAACCAGCCACUCAGCCAGUGAAGAGGAAGUUAGAA | |
| | GAUCUGAGCUCUGAGUGGAAGGCGGUAAACCGUUUACUUCAAGAGCUGAGGGCAAAGCAGCCUGACCUAG | |
| | CUCCUGGACUGACCACUAUUGGAGCCUCUCCUACUCAGACUGUUACUCUGGUGACACAACCUGUGGUUAC | |
| | UAAGGAAACUGCCAUCUCCAAACUAGAAAUGCCAUCUUCCUUGAUGUUGGAGGUACCUGCUCUGGCAGAU | |
| | UUCAACCGGGCUUGGACAGAACUUACCGACUGGCUUUCUCUGCUUGAUCAAGUUAUAAAAUCACAGAGGG | |
| | UGAUGGUGGGUGACCUUGAGGAUAUCAACGAGAUGAUCAUCAAGCAGAAGGCAACAAUGCAGGAUUUGGA | |
| | ACAGAGGCGUCCCCAGUUGGAAGAACUCAUUACCGCUGCCCAAAAUUUGAAAAACAAGACCAGCAAUCAA | |
| | GAGGCUAGAACAAUCAUUACGGAUCGAAUUGAAAGAAUUCAGAAUCAGUGGGAUGAAGUACAAGAACACC | |
| | UUCAGAACCGGAGGCAACAGUUGAAUGAAAUGUUAAAGGAUUCAACACAAUGGCUGGAAGCUAAGGAAGA | |
| | AGCUGAGCAGGUCUUAGGACAGGCCAGAGCCAAGCUUGAGUCAUGGAAGGAGGGUCCCUAUACAGUAGAU | |
| | GCAAUCCAAAAGAAAAUCACAGAAACCAAGCAGUUGGCCAAAGACCUCCGCCAGUGGCAGACAAAUGUAG | |
| | AUGUGGCAAAUGACUUGGCCCUGAAACUUCUCCGGGAUUAUUCUGCAGAUGAUACCAGAAAAGUCCACAU | |
| | GAUAACAGAGAAUAUCAAUGCCUCUUGGAGAAGCAUUCAUAAAAGGGUGAGUGAGCGAGAGGCUGCUUUG | |
| | GAAGAAACUCAUAGAUUACUGCAACAGUUCCCCCUGGACCUGGAAAAGUUUCUUGCCUGGCUUACAGAAG | |
| | CUGAAACAACUGCCAAUGUCCUACAGGAUGCUACCCGUAAGGAAAGGCUCCUAGAAGACUCCAAGGGAGU | |
| | AAAAGAGCUGAUGAAACAAUGGCAAGACCUCCAAGGUGAAAUUGAAGCUCACACAGAUGUUUAUCACAAC | |
| | CUGGAUGAAAACAGCCAAAAAAUCCUGAGAUCCCUGGAAGGUUCCGAUGAUGCAGUCCUGUUACAAAGAC | |
| | GUUUGGAUAACAUGAACUUCAAGUGGAGUGAACUUCGGAAAAAGUCUCUCAACAUUAGGUCCCAUUUGGA | |
| | AGCCAGUUCUGACCAGUGGAAGCGUCUGCACCUUUCUCUGCAGGAACUUCUGGUGUGGCUACAGCUGAAA | |
| | GAUGAUGAAUUAAGCCGGCAGGCACCUAUUGGAGGCGACUUUCCAGCAGUUCAGAAGCAGAACGAUGUAC | |
| | AUAGGGCCUUCAAGAGGGAAUUGAAAACUAAAGAACCUGUAAUCAUGAGUACUCUUGAGACUGUACGAAU | |
| | AUUUCUGACAGAGCAGCCUUUGGAAGGACUAGAGAAACUCUACCAGGAGCCCAGAGAGCUGCCUCCUGAG | |
| | GAGAGAGCCCAGAAUGUCACUCGGCUUCUACGAAAGCAGGCUGAGGAGGUCAAUACUGAGUGGGAAAAAU | |
| | UGAACCUGCACUCCGCUGACUGGCAGAGAAAAAUAGAUGAGACCCUUGAAAGACUCCAGGAACUUCAAGA | |
| | GGCCACGGAUGAGCUGGACCUCAAGCUGCGCCAAGCUGAGGUGAUCAAGGGAUCCUGGCAGCCCGUGGGC | |
| | GAUCUCCUCAUUGACUCUCUCCAAGAUCACCUCGAGAAAGUCAAGGCACUUCGAGGAGAAAUUGCGCCUC | |
| | UGAAAGAGAACGUGAGCCACGUCAAUGACCUUGCUCGCCAGCUUACCACUUUGGGCAUUCAGCUCUCACC | |
| | GUAUAACCUCAGCACUCUGGAAGACCUGAACACCAGAUGGAAGCUUCUGCAGGUGGCCGUCGAGGACCGA | |
| | GUCAGGCAGCUGCAUGAAGCCCACAGGGACUUUGGUCCAGCAUCUCAGCACUUUCUUUCCACGUCUGUCC | |
| | AGGGUCCCUGGGAGAGAGCCAUCUCGCCAAACAAAGUGCCCUACUAUAUCAACCACGAGACUCAAACAAC | |
| | UUGCUGGGACCAUCCCAAAAUGACAGAGCUCUACCAGUCUUUAGCUGACCUGAAUAAUGUCAGAUUCUCA | |
| | GCUUAUAGGACUGCCAUGAAACUCCGAAGACUGCAGAAGGCCCUUUGCUUGGAUCUCUUGAGCCUGUCAG | |
| | CUGCAUGUGAUGCCUUGGACCAGCACAACCUCAAGCAAAAUGACCAGCCCAUGGAUAUCCUGCAGAUUAU | |
| | UAAUUGUUUGACCACUAUUUAUGACCGCCUGGAGCAAGAGCACAACAAUUUGGUCAACGUCCCUCUCUGC | |
| | GUGGAUAUGUGUCUGAACUGGCUGCUGAAUGUUUAUGAUACGGGACGAACAGGGAGGAUCCGUGUCCUGU | |
| | CUUUUAAAACUGGCAUCAUUUCCCUGUGUAAAGCACAUUUGGAAGACAAGUACAGAUACCUUUUCAAGCA | |
| | AGUGGCAAGUUCAACAGGAUUUUGUGACCAGCGCAGGCUGGGCCUCCUUCUGCAUGAUUCUAUCCAAAUU | |
| | CCAAGACAGUUGGGUGAAGUUGCAUCCUUUGGGGGCAGUAACAUUGAGCCAAGUGUCCGGAGCUGCUUCC | |
| | AAUUUGCUAAUAAUAAGCCAGAGAUCGAAGCGGCCCUCUUCCUAGACUGGAUGAGACUGGAACCCCAGUC | |
| | CAUGGUGUGGCUGCCCGUCCUGCACAGAGUGGCUGCUGCAGAAACUGCCAAGCAUCAGGCCAAAUGUAAC | |
| | AUCUGCAAAGAGUGUCCAAUCAUUGGAUUCAGGUACAGGAGUCUAAAGCACUUUAAUUAUGACAUCUGCC | |
| | AAAGCUGCUUUUUUUCUGGUCGAGUUGCAAAAGGCCAUAAAAUGCACUAUCCCAUGGUGGAAUAUUGCAC | |
| | UCCGACUACAUCAGGAGAAGAUGUUCGAGACUUUGCCAAGGUACUAAAAAACAAAUUUCGAACCAAAAGG | |
| | UAUUUUGCGAAGCAUCCCCGAAUGGGCUACCUGCCAGUGCAGACUGUCUUAGAGGGGGACAACAUGGAAA | |
| | CUCCCGUUACUCUGAUCAACUUCUGGCCAGUAGAUUCUGCGCCUGCCUCGUCCCCUCAGCUUUCACACGA | |
| | UGAUACUCAUUCACGCAUUGAACAUUAUGCUAGCAGGCUAGCAGAAAUGGAAAACAGCAAUGGAUCUUAU | |
| | CUAAAUGAUAGCAUCUCUCCUAAUGAGAGCAUAGAUGAUGAACAUUUGUUAAUCCAGCAUUACUGCCAAA | |
| | GUUUGAACCAGGACUCCCCCCUGAGCCAGCCUCGUAGUCCUGCCCAGAUCUUGAUUUCCUUAGAGAGUGA | |
| | GGAAAGAGGGGAGCUAGAGAGAAUCCUAGCAGAUCUUGAGGAAGAAAACAGGAAUCUGCAAGCAGAAUAU | |
| | GACCGUCUAAAGCAGCAGCACGAACAUAAAGGCCUGUCCCCACUGCCGUCCCCUCCUGAAAUGAUGCCCA | |
| | CCUCUCCCCAGAGUCCCCGGGAUGCUGAGCUCAUUGCUGAGGCCAAGCUACUGCGUCAACACAAAGGCCG | |
| | CCUGGAAGCCAGGAUGCAAAUCCUGGAAGACCACAAUAAACAGCUGGAGUCACAGUUACACAGGCUAAGG | |
| | CAGCUGCUGGAGCAACCCCAGGCAGAGGCCAAAGUGAAUGGCACAACGGUGUCCUCUCCUUCUACCUCUC | |
| | UACAGAGGUCCGACAGCAGUCAGCCUAUGCUGCUCCGAGUGGUUGGCAGUCAAACUUCGGACUCCAUGGG | |
| | UGAGGAAGAUCUUCUCAGUCCUCCCCAGGACACAAGCACAGGGUUAGAGGAGGUGAUGGAGCAACUCAAC | |
| | AACUCCUUCCCUAGUUCAAGAGGAAGAAAUACCCCUGGAAAGCCAAUGAGAGAGGACACAAUGUAGGAAG | |
| | UCUUUUCCACAUGGCAGAUGAUUUGGGCAGAGCGAUGGAGUCCUUAGUAUCAGUCAUGACAGAUGAAGAA | |
| | GGAGCAGAAUAAAUGUUUUACAACUCCUGAUUCCCGCAUGGUUUUUAUAAUAUUCAUACAACAAAGAGGA | |
| | UUAGACAGUAAGAGUUUACAAGAAAUAAAUCUAUAUUUUUGUGAAGGGUAGUGGUAUUAUACUGUAGAUU | |
| | UCAGUAGUUUCUAAGUCUGUUAUUGUUUUGUUAACAAUGGCAGGUUUUACACGUCUAUGCAAUUGUACAA | |
| | AAAAGUUAUAAGAAAACUACAUGUAAAAUCUUGAUAGCUAAAUAACUUGCCAUUUCUUUAUAUGGAACGC | |
| | AUUUUGGGUUGUUUAAAAAUUUAUAACAGUUAUAAAGAAAGAUUGUAAACUAAAGUGUGCUUUAUAAAAA | |
| | AAAGUUGUUUAUAAAAACCCCUAAAAACAAAACAAACACACACACACACACAUACACACACACACACAAA | |
| | ACUUUGAGGCAGCGCAUUGUUUUGCAUCCUUUUGGCGUGAUAUCCAUAUGAAAUUCAUGGCUUUUUCUUU | |
| | UUUUGCAUAUUAAAGAUAAGACUUCCUCUACCACCACACCAAAUGACUACUACACACUGCUCAUUUGAGA | |
| | ACUGUCAGCUGAGUGGGGCAGGCUUGAGUUUUCAUUUCAUAUAUCUAUAUGUCUAUAAGUAUAUAAAUAC | |
| | UAUAGUUAUAUAGAUAAAGAGAUACGAAUUUCUAUAGACUGACUUUUUCCAUUUUUUAAAUGUUCAUGUC | |
| | ACAUCCUAAUAGAAAGAAAUUACUUCUAGUCAGUCAUCCAGGCUUACCUGCUUGGUCUAGAAUGGAUUUU | |
| | UCCCGGAGCCGGAAGCCAGGAGGAAACUACACCACACUAAAACAUUGUCUACAGCUCCAGAUGUUUCUCA | |
| | UUUUAAACAACUUUCCACUGACAACGAAAGUAAAGUAAAGUAUUGGAUUUUUUUAAAGGGAACAUGUGAA | |
| | UGAAUACACAGGACUUAUUAUAUCAGAGUGAGUAAUCGGUUGGUUGGUUGAUUGAUUGAUUGAUUGAUAC | |
| | AUUCAGCUUCCUGCUGCUAGCAAUGCCACGAUUUAGAUUUAAUGAUGCUUCAGUGGAAAUCAAUCAGAAG | |
| | GUAUUCUGACCUUGUGAACAUCAGAAGGUAUUUUUUAACUCCCAAGCAGUAGCAGGACGAUGAUAGGGCU | |
| | GGAGGGCUAUGGAUUCCCAGCCCAUCCCUGUGAAGGAGUAGGCCACUCUUUAAGUGAAGGAUUGGAUGAU | |
| | UGUUCAUAAUACAUAAAGUUCUCUGUAAUUACAACUAAAUUAUUAUGCCCUCUUCUCACAGUCAAAAGGA | |
| | ACUGGGUGGUUUGGUUUUUGUUGCUUUUUUAGAUUUAUUGUCCCAUGUGGGAUGAGUUUUUAAAUGCCAC | |
| | AAGACAUAAUUUAAAAUAAAUAAACUUUGGGAAAAGGUGUAAGACAGUAGCCCCAUCACAUUUGUGAUAC | |
| | UGACAGGUAUCAACCCAGAAGCCCAUGAACUGUGUUUCCAUCCUUUGCAUUUCUCUGCGAGUAGUUCCAC | |
| | ACAGGUUUGUAAGUAAGUAAGAAAGAAGGCAAAUUGAUUCAAAUGUUACAAAAAAACCCUUCUUGGUGGA | |
| | UUAGACAGGUUAAAUAUAUAAACAAACAAACAAAAAUUGCUCAAAAAAGAGGAGAAAAGCUCAAGAGGAA | |
| | AAGCUAAGGACUGGUAGGAAAAAGCUUUACUCUUUCAUGCCAUUUUAUUUCUUUUUGAUUUUUAAAUCAU | |
| | UCAUUCAAUAGAUACCACCGUGUGACCUAUAAUUUUGCAAAUCUGUUACCUCUGACAUCAAGUGUAAUUA | |
| | GCUUUUGGAGAGUGGGCUGACAUCAAGUGUAAUUAGCUUUUGGAGAGUGGGUUUUGUCCAUUAUUAAUAA | |
| | UUAAUUAAUUAACAUCAAACACGGCUUCUCAUGCUAUUUCUACCUCACUUUGGUUUUGGGGUGUUCCUGA | |
| | UAAUUGUGCACACCUGAGUUCACAGCUUCACCACUUGUCCAUUGCGUUAUUUUCUUUUUCCUUUAUAAUU | |
| | CUUUCUUUUUCCUUCAUAAUUUUCAAAAGAAAACCCAAAGCUCUAAGGUAACAAAUUACCAAAUUACAUG | |
| | AAGAUUUGGUUUUUGUCUUGCAUUUUUUUCCUUUAUGUGACGCUGGACCUUUUCUUUACCCAAGGAUUUU | |
| | UAAAACUCAGAUUUAAAACAAGGGGUUACUUUACAUCCUACUAAGAAGUUUAAGUAAGUAAGUUUCAUUC | |
| | UAAAAUCAGAGGUAAAUAGAGUGCAUAAAUAAUUUUGUUUUAAUCUUUUUGUUUUUCUUUUAGACACAUU | |
| | AGCUCUGGAGUGAGUCUGUCAUAAUAUUUGAACAAAAAUUGAGAGCUUUAUUGCUGCAUUUUAAGCAUAA | |
| | UUAAUUUGGACAUUAUUUCGUGUUGUGUUCUUUAUAACCACCGAGUAUUAAACUGUAAAUCAUAAUGUAA | |
| | CUGAAGCAUAAACAUCACAUGGCAUGUUUUGUCAUUGUUUUCAGGUACUGAGUUCUUACUUGAGUAUCAU | |
| | AAUAUAUUGUGUUUUAACACCAACACUGUAACAUUUACGAAUUAUUUUUUUAAACUUCAGUUUUACUGCA | |
| | UUUUCACAACAUAUCAGACUUCACCAAAUAUAUGCCUUACUAUUGUAUUAUAGUACUGCUUUACUGUGUA | |
| | UCUCAAUAAAGCACGCAGUUAUGUUAC | |
| Homo | UUCCCCAGCAGCUGCUGCUCGCUCAGCUCACAAGCCAAGGCCAGGGGACAGGGCGGCAGCGACUCCUCUG | 64 |
| sapiens | GCUCCCGAGAAGUGGAUCCGGUCGCGGCCACUACGAUGCCGGGAGCCGCCGGGGUCCUCCUCCUUCUGCU | |
| laminin | GCUCUCCGGAGGCCUCGGGGGCGUACAGGCGCAGCGGCCGCAGCAGCAGCGGCAGUCACAGGCACAUCAG | |
| subunit | CAAAGAGGUUUAUUCCCUGCUGUCCUGAAUCUUGCUUCUAAUGCUCUUAUCACGACCAAUGCAACAUGUG | |
| alpha 2 | GAGAAAAAGGACCUGAAAUGUACUGCAAAUUGGUAGAACAUGUCCCUGGGCAGCCUGUGAGGAACCCGCA | |
| (LAMA2), | GUGUCGAAUCUGCAAUCAAAACAGCAGCAAUCCAAACCAGAGACACCCGAUUACAAAUGCUAUUGAUGGA | |
| transcript | AAGAACACUUGGUGGCAGAGUCCCAGUAUUAAGAAUGGAAUCGAAUACCAUUAUGUGACAAUUACCCUGG | |
| variant 2 | AUUUACAGCAGGUGUUCCAGAUCGCGUAUGUGAUUGUGAAGGCAGCUAACUCCCCCCGGCCUGGAAACUG | |
| | GAUUUUGGAACGCUCUCUUGAUGAUGUUGAAUACAAGCCCUGGCAGUAUCAUGCUGUGACAGACACGGAG | |
| | UGCCUAACGCUUUACAAUAUUUAUCCCCGCACUGGGCCACCGUCAUAUGCCAAAGAUGAUGAGGUCAUCU | |
| | GCACUUCAUUUUACUCCAAGAUACACCCCUUAGAAAAUGGAGAGAUUCACAUCUCUUUAAUCAAUGGGAG | |
| | ACCAAGUGCCGAUGAUCCUUCUCCAGAACUGCUAGAAUUUACCUCCGCUCGCUAUAUUCGCCUGAGAUUU | |
| | CAGAGGAUCCGCACACUGAAUGCUGACUUGAUGAUGUUUGCUCACAAAGACCCAAGAGAAAUUGACCCCA | |
| | UUGUCACCAGAAGAUAUUACUACUCGGUCAAGGAUAUUUCAGUUGGAGGGAUGUGCAUCUGCUAUGGUCA | |
| | UGCCAGGGCUUGUCCACUUGAUCCAGCGACAAAUAAAUCUCGCUGUGAGUGUGAGCAUAACACAUGUGGC | |
| | GAUAGCUGUGAUCAGUGCUGUCCAGGAUUCCAUCAGAAACCCUGGAGAGCUGGAACUUUUCUAACUAAAA | |
| | CUGAAUGUGAAGCAUGCAAUUGUCAUGGAAAAGCUGAAGAAUGCUAUUAUGAUGAAAAUGUUGCCAGAAG | |
| | AAAUCUGAGUUUGAAUAUACGUGGAAAGUACAUUGGAGGGGGUGUCUGCAUUAAUUGUACCCAAAACACU | |
| | GCUGGUAUAAACUGCGAGACAUGUACUGAUGGCUUCUUCAGACCCAAAGGGGUAUCUCCAAAUUAUCCAA | |
| | GGCCAUGCCAGCCAUGUCAUUGCGAUCCAAUUGGUUCCUUAAAUGAAGUCUGUGUCAAGGAUGAGAAACA | |
| | UGCUCGACGAGGUUUGGCACCUGGAUCCUGUCAUUGCAAAACUGGUUUUGGAGGUGUGAGCUGUGAUCGG | |
| | UGUGCCAGGGGCUACACUGGCUACCCGGACUGCAAAGCCUGUAACUGCAGUGGGUUAGGGAGCAAAAAUG | |
| | AGGAUCCUUGUUUUGGCCCCUGUAUCUGCAAGGAAAAUGUUGAAGGAGGAGACUGUAGUCGUUGCAAAUC | |
| | CGGCUUCUUCAAUUUGCAAGAGGAUAAUUGGAAAGGCUGCGAUGAGUGUUUCUGUUCAGGGGUUUCAAAC | |
| | AGAUGUCAGAGUUCCUACUGGACCUAUGGCAAAAUACAAGAUAUGAGUGGCUGGUAUCUGACUGACCUUC | |
| | CUGGCCGCAUUCGAGUGGCUCCCCAGCAGGACGACUUGGACUCACCUCAGCAGAUCAGCAUCAGUAACGC | |
| | GGAGGCCCGGCAAGCCCUGCCGCACAGCUACUACUGGAGCGCGCCGGCUCCCUAUCUGGGAAACAAACUC | |
| | CCAGCAGUAGGAGGACAGUUGACAUUUACCAUAUCAUAUGACCUUGAAGAAGAGGAAGAAGAUACAGAAC | |
| | GUGUUCUCCAGCUUAUGAUUAUCUUAGAGGGUAAUGACUUGAGCAUCAGCACAGCCCAAGAUGAGGUGUA | |
| | CCUGCACCCAUCUGAAGAACAUACUAAUGUAUUGUUACUUAAAGAAGAAUCAUUUACCAUACAUGGCACA | |
| | CAUUUUCCAGUCCGUAGAAAGGAAUUUAUGACAGUGCUUGCGAAUUUGAAGAGAGUCCUCCUACAAAUCA | |
| | CAUACAGCUUUGGGAUGGAUGCCAUCUUCAGGUUGAGCUCUGUUAACCUUGAAUCCGCUGUCUCCUAUCC | |
| | UACUGAUGGAAGCAUUGCAGCAGCUGUAGAAGUGUGUCAGUGCCCACCAGGGUAUACUGGCUCCUCUUGU | |
| | GAAUCUUGUUGGCCUAGGCACAGGCGAGUUAACGGCACUAUUUUUGGUGGCAUCUGUGAGCCAUGUCAGU | |
| | GCUUUGGUCAUGCGGAGUCCUGUGAUGACGUCACUGGAGAAUGCCUGAACUGUAAGGAUCACACAGGUGG | |
| | CCCAUAUUGUGAUAAAUGUCUUCCUGGUUUCUAUGGCGAGCCUACUAAAGGAACCUCUGAAGACUGUCAA | |
| | CCCUGUGCCUGUCCACUCAAUAUCCCAUCCAAUAACUUUAGCCCAACGUGCCAUUUAGACCGGAGUCUUG | |
| | GAUUGAUCUGUGAUGGAUGCCCUGUCGGGUACACAGGACCACGCUGUGAGAGGUGUGCAGAAGGCUAUUU | |
| | UGGACAACCCUCUGUACCUGGAGGAUCAUGUCAGCCAUGCCAAUGCAAUGACAACCUUGACUUCUCCAUC | |
| | CCUGGCAGCUGUGACAGCUUGUCUGGCUCCUGUCUGAUAUGUAAACCAGGUACAACAGGCCGGUACUGUG | |
| | AGCUCUGUGCUGAUGGAUAUUUUGGAGAUGCAGUUGAUGCGAAGAACUGUCAGCCCUGUCGCUGUAAUGC | |
| | CGGUGGCUCUUUCUCUGAGGUUUGCCACAGUCAAACUGGACAGUGUGAGUGCAGAGCCAACGUUCAGGGU | |
| | CAGAGAUGUGACAAAUGCAAGGCUGGGACCUUUGGCCUACAAUCAGCAAGGGGCUGUGUUCCCUGCAACU | |
| | GCAAUUCUUUUGGGUCUAAGUCAUUCGACUGUGAAGAGAGUGGACAAUGUUGGUGCCAACCUGGAGUCAC | |
| | AGGGAAGAAAUGUGACCGCUGUGCCCACGGCUAUUUCAACUUCCAAGAAGGAGGCUGCACAGCUUGUGAA | |
| | UGUUCUCAUCUGGGUAAUAAUUGUGACCCAAAGACUGGGCGAUGCAUUUGCCCUCCCAAUACCAUUGGAG | |
| | AGAAAUGUUCUAAAUGUGCACCCAAUACCUGGGGCCACAGCAUUACCACUGGUUGUAAGGCUUGUAACUG | |
| | CAGCACAGUGGGAUCCUUGGAUUUCCAAUGCAAUGUAAAUACAGGCCAAUGCAACUGUCAUCCAAAAUUC | |
| | UCUGGUGCAAAAUGUACAGAGUGCAGUCGAGGUCACUGGAACUACCCUCGCUGCAAUCUCUGUGACUGCU | |
| | UCCUCCCUGGGACAGAUGCCACAACCUGUGAUUCAGAGACUAAAAAAUGCUCCUGUAGUGAUCAAACUGG | |
| | GCAGUGCACUUGUAAGGUGAAUGUGGAAGGCAUCCACUGUGACAGAUGCCGGCCUGGCAAAUUCGGACUC | |
| | GAUGCCAAGAAUCCACUUGGCUGCAGCAGCUGCUAUUGCUUCGGCACUACUACCCAGUGCUCUGAAGCAA | |
| | AAGGACUGAUCCGGACGUGGGUGACUCUGAAGGCUGAGCAGACCAUUCUACCCCUGGUAGAUGAGGCUCU | |
| | GCAGCACACGACCACCAAGGGCAUUGUUUUUCAACAUCCAGAGAUUGUUGCCCACAUGGACCUGAUGAGA | |
| | GAAGAUCUCCAUUUGGAACCUUUUUAUUGGAAACUUCCAGAACAAUUUGAAGGAAAGAAGUUGAUGGCCU | |
| | AUGGGGGCAAACUCAAGUAUGCAAUCUAUUUCGAGGCUCGGGAAGAAACAGGUUUCUCUACAUAUAAUCC | |
| | UCAAGUGAUCAUUCGAGGUGGGACACCUACUCAUGCUAGAAUUAUCGUCAGGCAUAUGGCUGCUCCUCUG | |
| | AUUGGCCAAUUGACAAGGCAUGAAAUUGAAAUGACAGAGAAAGAAUGGAAAUAUUAUGGGGAUGAUCCUC | |
| | GAGUCCAUAGAACUGUGACCCGAGAAGACUUCUUGGAUAUACUAUAUGAUAUUCAUUACAUUCUUAUCAA | |
| | AGCUACUUAUGGAAAUUUCAUGCGACAAAGCAGGAUUUCUGAAAUCUCAAUGGAGGUAGCUGAACAAGGA | |
| | CGUGGAACAACAAUGACUCCUCCAGCUGACUUGAUUGAAAAAUGUGAUUGUCCCCUGGGCUAUUCUGGCC | |
| | UGUCCUGUGAGGCAUGCUUGCCGGGAUUUUAUCGACUGCGUUCUCAACCAGGUGGCCGCACCCCUGGACC | |
| | AACCCUGGGCACCUGUGUUCCAUGUCAAUGUAAUGGACACAGCAGCCUGUGUGACCCUGAAACAUCGAUA | |
| | UGCCAGAAUUGUCAACAUCACACUGCUGGUGACUUCUGUGAACGAUGUGCUCUUGGAUACUAUGGAAUUG | |
| | UCAAGGGAUUGCCAAAUGACUGUCAGCAAUGUGCCUGCCCUCUGAUUUCUUCCAGUAACAAUUUCAGCCC | |
| | CUCUUGUGUCGCAGAAGGACUUGACGACUACCGCUGCACGGCUUGUCCACGGGGAUAUGAAGGCCAGUAC | |
| | UGUGAAAGGUGUGCCCCUGGCUAUACUGGCAGUCCAGGCAACCCUGGAGGCUCCUGCCAAGAAUGUGAGU | |
| | GUGAUCCCUAUGGCUCACUGCCUGUGCCCUGUGACCCUGUCACAGGAUUCUGCACGUGCCGACCUGGAGC | |
| | CACGGGAAGGAAGUGUGACGGCUGCAAGCACUGGCAUGCACGCGAGGGCUGGGAGUGUGUUUUUUGUGGA | |
| | GAUGAGUGCACUGGCCUUCUUCUCGGUGACUUGGCUCGCCUGGAGCAGAUGGUCAUGAGCAUCAACCUCA | |
| | CUGGUCCGCUGCCUGCGCCAUAUAAAAUGCUGUAUGGUCUUGAAAAUAUGACUCAGGAGCUAAAGCACUU | |
| | GCUGUCACCUCAGCGGGCCCCAGAGAGGCUUAUUCAGCUGGCAGAGGGCAAUCUGAAUACACUCGUGACC | |
| | GAAAUGAACGAGCUGCUGACCAGGGCUACCAAAGUGACAGCAGAUGGCGAGCAGACCGGACAGGAUGCUG | |
| | AGAGGACCAACACAAGAGCAAAGUCCCUGGGAGAAUUCAUUAAGGAGCUUGCCCGGGAUGCAGAAGCUGU | |
| | AAAUGAAAAAGCUAUAAAACUAAAUGAAACUCUAGGAACUCGAGACGAGGCCUUUGAGAGAAAUUUGGAA | |
| | GGGCUUCAGAAAGAGAUUGACCAGAUGAUUAAAGAACUGAGGAGGAAAAAUCUAGAGACACAAAAGGAAA | |
| | UUGCUGAAGAUGAGUUGGUAGCUGCAGAAGCCCUUCUGAAAAAAGUGAAGAAGCUGUUUGGAGAGUCCCG | |
| | GGGGGAAAAUGAAGAAAUGGAGAAGGAUCUCCGGGAAAAACUGGCUGACUACAAAAACAAAGUUGAUGAU | |
| | GCUUGGGACCUUUUGAGAGAAGCCACAGAUAAAAUCAGAGAAGCUAAUCGCCUAUUUGCAGUAAAUCAGA | |
| | AAAACAUGACUGCAUUGGAGAAAAAGAAGGAGGCUGUUGAAAGCGGCAAACGACAAAUUGAGAACACUUU | |
| | AAAAGAGGGCAAUGACAUACUCGAUGAAGCCAACCGUCUUGCAGAUGAAAUCAACUCCAUCAUAGACUAU | |
| | GUUGAAGACAUCCAAACUAAAUUGCCACCUAUGUCUGAGGAGCUUAAUGAUAAAAUAGAUGACCUCUCCC | |
| | AAGAAAUAAAGGACAGGAAGCUUGCUGAGAAGGUGUCCCAGGCUGAGAGCCACGCAGCUCAGUUGAAUGA | |
| | CUCAUCUGCUGUCCUUGAUGGAAUCCUUGAUGAGGCUAAAAACAUCUCCUUCAAUGCCACUGCAGCCUUC | |
| | AAAGCUUACAGCAAUAUUAAGGACUAUAUUGAUGAAGCUGAGAAAGUUGCCAAAGAAGCCAAAGAUCUUG | |
| | CACAUGAAGCUACAAAACUGGCAACAGGUCCUCGGGGUUUAUUAAAGGAAGAUGCCAAAGGCUGUCUUCA | |
| | GAAAAGCUUCAGGAUUCUUAACGAAGCCAAGAAGUUAGCAAAUGAUGUAAAAGAAAAUGAAGACCAUCUA | |
| | AAUGGCUUAAAAACCAGGAUAGAAAAUGCUGAUGCUAGAAAUGGGGAUCUCUUGAGAACUUUGAAUGACA | |
| | CUUUGGGAAAGUUAUCAGCUAUUCCAAAUGAUACAGCUGCUAAACUGCAAGCUGUUAAGGACAAAGCCAG | |
| | ACAAGCCAACGACACAGCUAAAGAUGUACUGGCACAGAUUACAGAGCUCCACCAGAACCUCGAUGGCCUG | |
| | AAGAAGAAUUACAAUAAACUAGCAGACAGCGUCGCCAAAACGAAUGCUGUGGUUAAAGAUCCUUCCAAGA | |
| | ACAAAAUCAUUGCCGAUGCAGAUGCCACUGUCAAAAAUUUAGAACAGGAAGCUGACCGGCUAAUAGAUAA | |
| | ACUCAAACCCAUCAAGGAACUUGAGGAUAACCUAAAGAAAAACAUCUCUGAGAUAAAGGAAUUGAUAAAC | |
| | CAAGCUCGGAAACAAGCCAAUUCUAUCAAAGUAUCUGUGUCUUCAGGAGGUGACUGCAUUCGAACAUACA | |
| | AACCAGAAAUCAAGAAAGGAAGUUACAAUAAUAUUGUUGUCAACGUAAAGACAGCUGUUGCUGAUAACCU | |
| | CCUCUUUUAUCUUGGAAGUGCCAAAUUUAUUGACUUUCUGGCUAUAGAAAUGCGUAAAGGCAAAGUCAGC | |
| | UUCCUCUGGGAUGUUGGAUCUGGAGUUGGACGUGUAGAGUACCCAGAUUUGACUAUUGAUGACUCAUAUU | |
| | GGUACCGUAUCGUAGCAUCAAGAACUGGGAGAAAUGGAACUAUUUCUGUGAGAGCCCUGGAUGGACCCAA | |
| | AGCCAGCAUUGUGCCCAGCACACACCAUUCGACGUCUCCUCCAGGGUACACGAUUCUAGAUGUGGAUGCA | |
| | AAUGCAAUGCUGUUUGUUGGUGGCCUGACUGGGAAAUUAAAGAAGGCUGAUGCUGUACGUGUGAUUACAU | |
| | UCACUGGCUGCAUGGGAGAAACAUACUUUGACAACAAACCUAUAGGUUUGUGGAAUUUCCGAGAAAAAGA | |
| | AGGUGACUGCAAAGGAUGCACUGUCAGUCCUCAGGUGGAAGAUAGUGAGGGGACUAUUCAAUUUGAUGGA | |
| | GAAGGUUAUGCAUUGGUCAGCCGUCCCAUUCGCUGGUACCCCAACAUCUCCACUGUCAUGUUCAAGUUCA | |
| | GAACAUUUUCUUCGAGUGCUCUUCUGAUGUAUCUUGCCACACGAGACCUGAGAGAUUUCAUGAGUGUGGA | |
| | GCUCACUGAUGGGCACAUAAAAGUCAGUUACGAUCUGGGCUCAGGAAUGGCUUCCGUUGUCAGCAAUCAA | |
| | AACCAUAAUGAUGGGAAAUGGAAAUCAUUCACUCUGUCAAGAAUUCAAAAACAAGCCAAUAUAUCAAUUG | |
| | UAGAUAUAGAUACUAAUCAGGAGGAGAAUAUAGCAACUUCGUCUUCUGGAAACAACUUUGGUCUUGACUU | |
| | GAAAGCAGAUGACAAAAUAUAUUUUGGUGGCCUGCCAACGCUGAGAAACUUGAGGCCAGAAGUAAAUCUG | |
| | AAGAAAUAUUCCGGCUGCCUCAAAGAUAUUGAAAUUUCAAGAACUCCGUACAAUAUACUCAGUAGUCCCG | |
| | AUUAUGUUGGUGUUACCAAAGGAUGUUCCCUGGAGAAUGUUUACACAGUUAGCUUUCCUAAGCCUGGUUU | |
| | UGUGGAGCUCUCCCCUGUGCCAAUUGAUGUAGGAACAGAAAUCAACCUGUCAUUCAGCACCAAGAAUGAG | |
| | UCCGGCAUCAUUCUUUUGGGAAGUGGAGGGACACCAGCACCACCUAGGAGAAAACGAAGGCAGACUGGAC | |
| | AGGCCUAUUAUGUAAUACUCCUCAACAGGGGCCGUCUGGAAGUGCAUCUCUCCACAGGGGCACGAACAAU | |
| | GAGGAAAAUUGUGAUCAGACCAGAGCCGAAUCUGUUUCAUGAUGGAAGAGAACAUUCCGUUCAUGUAGAG | |
| | CGAACUAGAGGCAUCUUUACAGUUCAAGUGGAUGAAAACAGAAGAUACAUGCAAAACCUGACAGUUGAAC | |
| | AGCCUAUCGAAGUUAAAAAGCUUUUCGUUGGGGGUGCUCCACCUGAAUUUCAACCUUCCCCACUCAGAAA | |
| | UAUUCCUCCUUUUGAAGGCUGCAUAUGGAAUCUUGUUAUUAACUCUGUCCCCAUGGACUUUGCAAGGCCU | |
| | GUGUCCUUCAAAAAUGCUGACAUUGGUCGCUGUGCCCAUCAGAAACUCCGUGAAGAUGAAGAUGGAGCAG | |
| | CUCCAGCUGAAAUAGUUAUCCAGCCUGAGCCAGUUCCCACCCCAGCCUUUCCUACGCCCACCCCAGUUCU | |
| | GACACAUGGUCCUUGUGCUGCAGAAUCAGAACCAGCUCUUUUGAUAGGGAGCAAGCAGUUCGGGCUUUCA | |
| | AGAAACAGUCACAUUGCAAUUGCAUUUGAUGACACCAAAGUUAAAAACCGUCUCACAAUUGAGUUGGAAG | |
| | UAAGAACCGAAGCUGAAUCCGGCUUGCUUUUUUACAUGGCUCGCAUCAAUCAUGCUGAUUUUGCAACAGU | |
| | UCAGCUGAGAAAUGGAUUGCCCUACUUCAGCUAUGACUUGGGGAGUGGGGACACCCACACCAUGAUCCCC | |
| | ACCAAAAUCAAUGAUGGCCAGUGGCACAAGAUUAAGAUAAUGAGAAGUAAGCAAGAAGGAAUUCUUUAUG | |
| | UAGAUGGGGCUUCCAACAGAACCAUCAGUCCCAAAAAAGCCGACAUCCUGGAUGUCGUGGGAAUGCUGUA | |
| | UGUUGGUGGGUUACCCAUCAACUACACUACCCGAAGAAUUGGUCCAGUGACCUAUAGCAUUGAUGGCUGC | |
| | GUCAGGAAUCUCCACAUGGCAGAGGCCCCUGCCGAUCUGGAACAACCCACCUCCAGCUUCCAUGUUGGGA | |
| | CAUGUUUUGCAAAUGCUCAGAGGGGAACAUAUUUUGACGGAACCGGUUUUGCCAAAGCAGUUGGUGGAUU | |
| | CAAAGUGGGAUUGGACCUUCUUGUAGAAUUUGAAUUCCGCACAACUACAACGACUGGAGUUCUUCUGGGG | |
| | AUCAGUAGUCAAAAAAUGGAUGGAAUGGGUAUUGAAAUGAUUGAUGAAAAGUUGAUGUUUCAUGUGGACA | |
| | AUGGUGCGGGCAGAUUCACUGCUGUCUAUGAUGCUGGGGUUCCAGGGCAUUUGUGUGAUGGACAAUGGCA | |
| | UAAAGUCACUGCCAACAAGAUCAAACACCGCAUUGAGCUCACAGUCGAUGGGAACCAGGUGGAAGCCCAA | |
| | AGCCCAAACCCAGCAUCUACAUCAGCUGACACAAAUGACCCUGUGUUUGUUGGAGGCUUCCCAGAUGACC | |
| | UCAAGCAGUUUGGCCUAACAACCAGUAUUCCGUUCCGAGGUUGCAUCAGAUCCCUGAAGCUCACCAAAGG | |
| | CACAGGCAAGCCACUGGAGGUUAAUUUUGCCAAGGCCCUGGAACUGAGGGGCGUUCAACCUGUAUCAUGC | |
| | CCAGCCAACUAAUAAAAAUAAGUGUAACCCCAGGAAGAGUCUGUCAAAACAAGUAUAUCAAGUAAAACAA | |
| | ACAAAUAUAUUUUACCUAUAUAUGUUAAUUAAACUAAUUUGUGCAUGUACAUAGAAUUCUUUCUGUAUUC | |
| | AGAUGGUGCUAAUUCAGACUCCAGACUGAAUUUUAAUUCAAGUUCUUUCUCAAGUCUAUAAAUAAUAUUA | |
| | AACUGAUUAUUUCAUUCUAAAAAAAAAAAAAAAAAA | |
| Homo | CACGGCCGGUCUGUGCCGGCUGCUCCCGCGGUUAGGUCCCGCCCCGCGCAGCGCGCGCAGCCUGCGGAGC | 65 |
| sapiens | CAGCGGCCGUGACGCGACAACGAUUCGGCUGUGACGCGACAACGAUUCGGCUGUGACGCGAGCGCGGCCG | |
| emerin | CUCCCGAUGCGCUCGUGCCGCCCCCGCCGUGCUCCUCGGCAGCCGUUGCUCGGCCGGUUUUGGUAGGCCC | |
| (EMD) | GGGCCGCCGCCAGGCCUCCGCCUGAGCCCGCACCCGCCAUGGACAACUACGCAGAUCUUUCGGAUACCGA | |
| | GCUGACCACCUUGCUGCGCCGGUACAACAUCCCGCACGGGCCUGUAGUAGGAUCAACUCGUAGGCUUUAC | |
| | GAGAAGAAGAUCUUCGAGUACGAGACCCAGAGGCGGCGGCUCUCGCCCCCCAGCUCGUCCGCCGCCUCCU | |
| | CUUAUAGCUUCUCUGACUUGAAUUCGACUAGAGGGGAUGCAGAUAUGUAUGAUCUUCCCAAGAAAGAGGA | |
| | CGCUUUACUCUACCAGAGCAAGGGCUACAAUGACGACUACUAUGAAGAGAGCUACUUCACCACCAGGACU | |
| | UAUGGGGAGCCCGAGUCUGCCGGCCCGUCCAGGGCUGUCCGCCAGUCAGUGACUUCAUUCCCAGAUGCUG | |
| | ACGCUUUCCAUCACCAGGUGCAUGAUGACGAUCUUUUGUCUUCUUCUGAAGAGGAGUGCAAGGAUAGGGA | |
| | ACGCCCCAUGUACGGCCGGGACAGUGCCUACCAGAGCAUCACGCACUACCGCCCUGUUUCAGCCUCCAGG | |
| | AGCUCCCUGGACCUGUCCUAUUAUCCUACUUCCUCCUCCACCUCUUUUAUGUCCUCCUCAUCAUCUUCCU | |
| | CUUCAUGGCUCACCCGCCGUGCCAUCCGGCCUGAAAACCGUGCUCCUGGGGCUGGGCUGGGCCAGGAUCG | |
| | CCAGGUCCCGCUCUGGGGCCAGCUGCUGCUUUUCCUGGUCUUUGUGAUCGUCCUCUUCUUCAUUUACCAC | |
| | UUCAUGCAGGCUGAAGAAGGCAACCCCUUCUAGAGGGAGCCAUGAGGGUCUGGGCUUCAGAGCUAGGUCU | |
| | UUGGGGAAGUCCUGGCUGACUGCCUUAGCAGUGGGGGUGGGGGUGGGGGCAGGGGCAGGGGCUUUAUGUG | |
| | UUUUUGCUUGGGGGGCGCUGGGCCUAGCCCAGAGUAGUGCUUGCUCCCCCUGCCUUGUCCCACCAGGGAG | |
| | GCAGCAGACUCAGGCCCUCCAUGGUCCUCUUUGUCAUUUUGUUGACAUGCAUUCCUCCUUUUGUCAUCUU | |
| | GUUGGGGGGAGGGGAUUAACCAAAGGCCACCCUGACUUUGUUUUUGUGGACACACAAUAAAAGCCCCGUU | |
| | UAUUUGUAAAAAAAAAAAAAAAAAAAAAAAAAAAAAAAAA | |
| Homo | UCGACCGCCCAGCCAGGUGCAAAAUGCCGUGUCAUUGGGAGACUCCGCAGCCGGAGCAUUAGAUUACAGC | 66 |
| sapiens | UCGACGGAGCUCGGGAAGGGCGGCGGGGGUGGAAGAUGAGCAGAAGCCCCUGUUCUCGGAACGCCGGCUG | |
| dysferlin, | ACAAGCGGGGUGAGCGCAGGCGGGGCGGGGACCCAGCCUAGCCCACUGGAGCAGCCGGGGGUGGCCCGUU | |
| complete | CCCCUUUAAGAGCAACUGCUCUAAGCCAGGAGCCAGAGAUUCGAGCCGGCCUCGCCCAGCCAGCCCUCUC | |
| cds | CAGCGAGGGGACCCACAAGCGGCGCCUCGGCCCUCCCGACCUUUCCGAGCCCUCUUUGCGCCCUGGGCGC | |
| | ACGGGGCCCUACACGCGCCAAGCAUGCUGAGGGUCUUCAUCCUCUAUGCCGAGAACGUCCACACACCCGA | |
| | CACCGACAUCAGCGAUGCCUACUGCUCCGCGGUGUUUGCAGGGGUGAAGAAGAGAACCAAAGUCAUCAAG | |
| | AACAGCGUGAACCCUGUAUGGAAUGAGGGAUUUGAAUGGGACCUCAAGGGCAUCCCCCUGGACCAGGGCU | |
| | CUGAGCUUCAUGUGGUGGUCAAAGACCAUGAGACGAUGGGGAGGAACAGGUUCCUGGGGGAAGCCAAGGU | |
| | CCCACUCCGAGAGGUCCUCGCCACCCCUAGUCUGUCCGCCAGCUUCAAUGCCCCCCUGCUGGACACCAAG | |
| | AAGCAGCCCACAGGGGCCUCGCUGGUCCUGCAGGUGUCCUACACACCGCUGCCUGGAGCUGUGCCCCUGU | |
| | UCCCGCCCCCUACUCCUCUGGAGCCCUCCCCGACUCUGCCUGACCUGGAUGUAGUGGCAGACACAGGAGG | |
| | AGAGGAAGACACAGAGGACCAGGGACUCACUGGAGAUGAGGCGGAGCCAUUCCUGGAUCAAAGCGGAGGC | |
| | CCGGGGGCUCCCACCACCCCAAGGAAACUACCUUCACGUCCUCCGCCCCACUACCCCGGGAUCAAAAGAA | |
| | AGCGAAGUGCGCCUACAUCUAGAAAGCUGCUGUCAGACAAACCGCAGGAUUUCCAGAUCAGGGUCCAGGU | |
| | GAUCGAGGGGCGCCAGCUGCCGGGGGUGAACAUCAAGCCUGUGGUCAAGGUUACCGCUGCAGGGCAGACC | |
| | AAGCGGACGCGGAUCCACAAGGGAAACAGCCCACUCUUCAAUGAGACUCUUUUCUUCAACUUGUUUGACU | |
| | CUCCUGGGGAGCUGUUUGAUGAGCCCAUCUUUAUCACGGUGGUAGACUCUCGUUCUCUCAGGACAGAUGC | |
| | UCUCCUCGGGGAGUUCCGGAUGGACGUGGGCACCAUUUACAGAGAGCCCCGGCACGCCUAUCUCAGGAAG | |
| | UGGCUGCUGCUCUCAGACCCUGAUGACUUCUCUGCUGGGGCCAGAGGCUACCUGAAAACAAGCCUUUGUG | |
| | UGCUGGGGCCUGGGGACGAAGCGCCUCUGGAGAGAAAAGACCCCUCUGAAGACAAGGAGGACAUUGAAAG | |
| | CAACCUGCUCCGGCCCACAGGCGUAGCCCUGCGAGGAGCCCACUUCUGCCUGAAGGUCUUCCGGGCCGAG | |
| | GACUUGCCGCAGAUGGACGAUGCCGUGAUGGACAACGUGAAACAGAUCUUUGGCUUCGAGAGUAACAAGA | |
| | AGAACUUGGUGGACCCCUUUGUGGAGGUCAGCUUUGCGGGGAAAAUGCUGUGCAGCAAGAUCUUGGAGAA | |
| | GACGGCCAACCCUCAGUGGAACCAGAACAUCACACUGCCUGCCAUGUUUCCCUCCAUGUGCGAAAAAAUG | |
| | AGGAUUCGUAUCAUAGACUGGGACCGCCUGACUCACAAUGACAUCGUGGCUACCACCUACCUGAGUAUGU | |
| | CGAAAAUCUCUGCCCCUGGAGGAGAAAUAGAAGAGGAGCCUGCAGGUGCUGUCAAGCCUUCGAAAGCCUC | |
| | AGACUUGGAUGACUACCUGGGCUUCCUCCCCACUUUUGGGCCCUGCUACAUCAACCUCUAUGGCAGUCCC | |
| | AGAGAGUUCACAGGCUUCCCAGACCCCUACACAGAGCUCAACACAGGCAAGGGGGAAGGUGUGGCUUAUC | |
| | GUGGCCGGCUUCUGCUCUCCCUGGAGACCAAGCUGGUGGAGCACAGUGAACAGAAGGUGGAGGACCUUCC | |
| | UGCGGAUGACAUCCUCCGGGUGGAGAAGUACCUUAGGAGGCGCAAGUACUCCCUGUUUGCGGCCUUCUAC | |
| | UCAGCCACCAUGCUGCAGGAUGUGGAUGAUGCCAUCCAGUUUGAGGUCAGCAUCGGGAACUACGGGAACA | |
| | AGUUCGACAUGACCUGCCUGCCGCUGGCCUCCACCACUCAGUACAGCCGUGCAGUCUUUGACGGGUGCCA | |
| | CUACUACUACCUACCCUGGGGUAACGUGAAACCUGUGGUGGUGCUGUCAUCCUACUGGGAGGACAUCAGC | |
| | CAUAGAAUCGAGACUCAGAACCAGCUGCUUGGGAUUGCUGACCGGCUGGAAGCUGGCCUGGAGCAGGUCC | |
| | ACCUGGCCCUGAAGGCGCAGUGCUCCACGGAGGACGUGGACUCGCUGGUGGCUCAGCUGACGGAUGAGCU | |
| | CAUCGCAGGCUGCAGCCAGCCUCUGGGUGACAUCCAUGAGACACCCUCUGCCACCCACCUGGACCAGUAC | |
| | CUGUACCAGCUGCGCACCCAUCACCUGAGCCAAAUCACUGAGGCUGCCCUGGCCCUGAAGCUCGGCCACA | |
| | GUGAGCUCCCUGCAGCUCUGGAGCAGGCGGAGGACUGGCUCCUGCGUCUGCGUGCCCUGGCAGAGGAGCC | |
| | CCAGAACAGCCUGCCGGACAUCGUCAUCUGGAUGCUGCAGGGAGACAAGCGUGUGGCAUACCAGCGGGUG | |
| | CCCGCCCACCAAGUCCUCUUCUCCCGGCGGGGUGCCAACUACUGUGGCAAGAAUUGUGGGAAGCUACAGA | |
| | CAAUCUUUCUGAAAUAUCCGAUGGAGAAGGUGCCUGGCGCCCGGAUGCCAGUGCAGAUACGGGUCAAGCU | |
| | GUGGUUUGGGCUCUCUGUGGAUGAGAAGGAGUUCAACCAGUUUGCUGAGGGGAAGCUGUCUGUCUUUGCU | |
| | GAAACCUAUGAGAACGAGACUAAGUUGGCCCUUGUUGGGAACUGGGGCACAACGGGCCUCACCUACCCCA | |
| | AGUUUUCUGACGUCACGGGCAAGAUCAAGCUACCCAAGGACAGCUUCCGCCCCUCGGCCGGCUGGACCUG | |
| | GGCUGGAGAUUGGUUCGUGUGUCCGGAGAAGACUCUGCUCCAUGACAUGGACGCCGGUCACCUGAGCUUC | |
| | GUGGAAGAGGUGUUUGAGAACCAGACCCGGCUUCCCGGAGGCCAGUGGAUCUACAUGAGUGACAACUACA | |
| | CCGAUGUGAACGGGGAGAAGGUGCUUCCCAAGGAUGACAUUGAGUGCCCACUGGGCUGGAAGUGGGAAGA | |
| | UGAGGAAUGGUCCACAGACCUCAACCGGGCUGUCGAUGAGCAAGGCUGGGAGUAUAGCAUCACCAUCCCC | |
| | CCGGAGCGGAAGCCGAAGCACUGGGUCCCUGCUGAGAAGAUGUACUACACACACCGACGGCGGCGCUGGG | |
| | UGCGCCUGCGCAGGAGGGAUCUCAGCCAAAUGGAAGCACUGAAAAGGCACAGGCAGGCGGAGGCGGAGGG | |
| | CGAGGGCUGGGAGUACGCCUCUCUUUUUGGCUGGAAGUUCCACCUCGAGUACCGCAAGACAGAUGCCUUC | |
| | CGCCGCCGCCGCUGGCGCCGUCGCAUGGAGCCACUGGAGAAGACGGGGCCUGCAGCUGUGUUUGCCCUUG | |
| | AGGGGGCCCUGGGCGGCGUGAUGGAUGACAAGAGUGAAGAUUCCAUGUCCGUCUCCACCUUGAGCUUCGG | |
| | UGUGAACAGACCCACGAUUUCCUGCAUAUUCGACUAUGGGAACCGCUACCAUCUACGCUGCUACAUGUAC | |
| | CAGGCCCGGGACCUGGCUGCGAUGGACAAGGACUCUUUUUCUGAUCCCUAUGCCAUCGUCUCCUUCCUGC | |
| | ACCAGAGCCAGAAGACGGUGGUGGUGAAGAACACCCUUAACCCCACCUGGGACCAGACGCUCAUCUUCUA | |
| | CGAGAUCGAGAUCUUUGGCGAGCCGGCCACAGUUGCUGAGCAACCGCCCAGCAUUGUGGUGGAGCUGUAC | |
| | GACCAUGACACUUAUGGUGCAGACGAGUUUAUGGGUCGCUGCAUCUGUCAACCGAGUCUGGAACGGAUGC | |
| | CACGGCUGGCCUGGUUCCCACUGACGAGGGGCAGCCAGCCGUCGGGGGAGCUGCUGGCCUCUUUUGAGCU | |
| | CAUCCAGAGAGAGAAGCCGGCCAUCCACCAUAUUCCUGGUUUUGAGGUGCAGGAGACAUCAAGGAUCCUG | |
| | GAUGAGUCUGAGGACACAGACCUGCCCUACCCACCACCCCAGAGGGAGGCCAACAUCUACAUGGUUCCUC | |
| | AGAACAUCAAGCCAGCGCUCCAGCGUACCGCCAUCGAGAUCCUGGCAUGGGGCCUGCGGAACAUGAAGAG | |
| | UUACCAGCUGGCCAACAUCUCCUCCCCCAGCCUCGUGGUAGAGUGUGGGGGCCAGACGGUGCAGUCCUGU | |
| | GUCAUCAGGAACCUCCGGAAGAACCCCAACUUUGACAUCUGCACCCUCUUCAUGGAAGUGAUGCUGCCCA | |
| | GGGAGGAGCUCUACUGCCCCCCCAUCACCGUCAAGGUCAUCGAUAACCGCCAGUUUGGCCGCCGGCCUGU | |
| | GGUGGGCCAGUGUACCAUCCGCUCCCUGGAGAGCUUCCUGUGUGACCCCUACUCGGCGGAGAGUCCAUCC | |
| | CCACAGGGUGGCCCAGACGAUGUGAGCCUACUCAGUCCUGGGGAAGACGUGCUCAUCGACAUUGAUGACA | |
| | AGGAGCCCCUCAUCCCCAUCCAGGAGGAAGAGUUCAUCGAUUGGUGGAGCAAAUUCUUUGCCUCCAUAGG | |
| | GGAGAGGGAAAAGUGCGGCUCCUACCUGGAGAAGGAUUUUGACACCCUGAAGGUCUAUGACACACAGCUG | |
| | GAGAAUGUGGAGGCCUUUGAGGGCCUGUCUGACUUUUGUAACACCUUCAAGCUGUACCGGGGCAAGACGC | |
| | AGGAGGAGACAGAAGAUCCAUCUGUGAUUGGUGAAUUUAAGGGCCUCUUCAAAAUUUAUCCCCUCCCAGA | |
| | AGACCCAGCCAUCCCCAUGCCCCCAAGACAGUUCCACCAGCUGGCCGCCCAGGGACCCCAGGAGUGCUUG | |
| | GUCCGUAUCUACAUUGUCCGAGCAUUUGGCCUGCAGCCCAAGGACCCCAAUGGAAAGUGUGAUCCUUACA | |
| | UCAAGAUCUCCAUAGGGAAGAAAUCAGUGAGUGACCAGGAUAACUACAUCCCCUGCACGCUGGAGCCCGU | |
| | AUUUGGAAAGAUGUUCGAGCUGACCUGCACUCUGCCUCUGGAGAAGGACCUAAAGAUCACUCUCUAUGAC | |
| | UAUGACCUCCUCUCCAAGGACGAAAAGAUCGGUGAGACGGUCGUCGACCUGGAGAACAGGCUGCUGUCCA | |
| | AGUUUGGGGCUCGCUGUGGACUCCCACAGACCUACUGUGUCUCUGGACCGAACCAGUGGCGGGACCAGCU | |
| | CCGCCCCUCCCAGCUCCUCCACCUCUUCUGCCAGCAGCAUAGAGUCAAGGCACCUGUGUACCGGACAGAC | |
| | CGUGUAAUGUUUCAGGAUAAAGAAUAUUCCAUUGAAGAGAUAGAGGCUGGCAGGAUCCCAAACCCACACC | |
| | UGGGCCCAGUGGAGGAGCGUCUGGCUCUGCAUGUGCUUCAGCAGCAGGGCCUGGUCCCGGAGCACGUGGA | |
| | GUCACGGCCCCUCUACAGCCCCCUGCAGCCAGACAUCGAGCAGGGGAAGCUGCAGAUGUGGGUCGACCUA | |
| | UUUCCGAAGGCCCUGGGGCGGCCUGGACCUCCCUUCAACAUCACCCCACGGAGAGCCAGAAGGUUUUUCC | |
| | UGCGUUGUAUUAUCUGGAAUACCAGAGAUGUGAUCCUGGAUGACCUGAGCCUCACGGGGGAGAAGAUGAG | |
| | CGACAUUUAUGUGAAAGGUUGGAUGAUUGGCUUUGAAGAACACAAGCAAAAGACAGACGUGCAUUAUCGU | |
| | UCCCUGGGAGGUGAAGGCAACUUCAACUGGAGGUUCAUUUUCCCCUUCGACUACCUGCCAGCUGAGCAAG | |
| | UCUGUACCAUUGCCAAGAAGGAUGCCUUCUGGAGGCUGGACAAGACUGAGAGCAAAAUCCCAGCACGAGU | |
| | GGUGUUCCAGAUCUGGGACAAUGACAAGUUCUCCUUUGAUGAUUUUCUGGGCUCCCUGCAGCUCGAUCUC | |
| | AACCGCAUGCCCAAGCCAGCCAAGACAGCCAAGAAGUGCUCCUUGGACCAGCUGGAUGAUGCUUUCCACC | |
| | CAGAAUGGUUUGUGUCCCUUUUUGAGCAGAAAACAGUGAAGGGCUGGUGGCCCUGUGUAGCAGAAGAGGG | |
| | UGAGAAGAAAAUACUGGCGGGCAAGCUGGAAAUGACCUUGGAGAUUGUAGCAGAGAGUGAGCAUGAGGAG | |
| | CGGCCUGCUGGCCAGGGCCGGGAUGAGCCCAACAUGAACCCUAAGCUUGAGGACCCAAGGCGCCCCGACA | |
| | CCUCCUUCCUGUGGUUUACCUCCCCAUACAAGACCAUGAAGUUCAUCCUGUGGCGGCGUUUCCGGUGGGC | |
| | CAUCAUCCUCUUCAUCAUCCUCUUCAUCCUGCUGCUGUUCCUGGCCAUCUUCAUCUACGCCUUCCCGAAC | |
| | UAUGCUGCCAUGAAGCUGGUGAAGCCCUUCAGCUGAGGACUCUCCUGCCCUGUAGAAGGGGCCGUGGGGU | |
| | CCCCUCCAGCAUGGGACUGGCCUGCCUCCUCCGCCCAGCUCGGCGAGCUCCUCCAGACCUCCUAGGCCUG | |
| | AUUGUCCUGCCAGGGUGGGCAGACAGACAGAUGGACCGGCCCACACUCCCAGAGUUGCUAACAUGGAGCU | |
| | CUGAGAUCACCCCACUUCCAUCAUUUCCUUCUCCCCCAACCCAACGCUUUUUUGGAUCAGCUCAGACAUA | |
| | UUUCAGUAUAAAACAGUUGGAACCACAAAAAAAAAAAAAAAAAAAAAAAAA | |
| Homo | AGGAGCAAGCCGAGAGCCAGCCGGCCGGCGCACUCCGACUCCGAGCAGUCUCUGUCCUUCGACCCGAGCC | 67 |
| sapiens | CCGCGCCCUUUCCGGGACCCCUGCCCCGCGGGCAGCGCUGCCAACCUGCCGGCCAUGGAGACCCCGUCCC | |
| lamin A/C | AGCGGCGCGCCACCCGCAGCGGGGCGCAGGCCAGCUCCACUCCGCUGUCGCCCACCCGCAUCACCCGGCU | |
| (LMNA) | GCAGGAGAAGGAGGACCUGCAGGAGCUCAAUGAUCGCUUGGCGGUCUACAUCGACCGUGUGCGCUCGCUG | |
| gene, | GAAACGGAGAACGCAGGGCUGCGCCUUCGCAUCACCGAGUCUGAAGAGGUGGUCAGCCGCGAGGUGUCCG | |
| complete | GCAUCAAGGCCGCCUACGAGGCCGAGCUCGGGGAUGCCCGCAAGACCCUUGACUCAGUAGCCAAGGAGCG | |
| cds | CGCCCGCCUGCAGCUGGAGCUGAGCAAAGUGCGUGAGGAGUUUAAGGAGCUGAAAGCGCGGUGAGUUCGC | |
| | CCAGGUGGCUGCGUGCCUGGCGGGGAGUGGAGAGGGCGGCGGGCCGGCGCCCCUGGCCGGCCGCAGGAAG | |
| | GGAGUGAGAGGGCCUGGAGGCCGAUAACUUUGCNNNNNNNNNNNNNNNNNNNNNNNNNNNNNNNNNNNNN | |
| | NNNNNNNNNNNNNNNNNNNNNNNNNNNNNNNNNNNNNNNNNNNNNNNNNNNNNNNNNNNNNNNCUGGUAA | |
| | UUGCAGGCAUAGCAGCGCCAGCCCCCAUGGCUGACCUCCUGGGAGCCUGGCACUGUCUAGGCACACAGAC | |
| | UCCUUCUCUUAAAUCUACUCUCCCCUCUCUUCUUUAGCAAUACCAAGAAGGAGGGUGACCUGAUAGCUGC | |
| | UCAGGCUCGGCUGAAGGACCUGGAGGCUCUGCUGAACUCCAAGGAGGCCGCACUGAGCACUGCUCUCAGU | |
| | GAGAAGCGCACGCUGGAGGGCGAGCUGCAUGAUCUGCGGGGCCAGGUGGCCAAGGUGAGGCCACCCUGCA | |
| | GGGCCCACCCAUGGCCCCACCUAACACAUGUACACUCACUCUUCUACCUAGGCCCUCCCCCAUGUGGUGC | |
| | CUGGUCUGACCUGUCACCUGAUUUCAGAGCCAUUCACCUGUCCUAGAGUCAUUUUACCCACUGAGGUCAC | |
| | AUCUUAUCCNNNNNNNNNNNNNNNNNNNNNNNNNNNNNNNNNNNNNNNNNNNNNNNNNNNNNNNNNNNNN | |
| | NNNNNNNNNNNNNNNNNNNNNNNNNNNNNNNNNNNNNNNGUCACCCAGGCUGGAGUGCAGUAGUGCGAUC | |
| | UCGGCUCACUGCAACCUCCACCUCCUGGAUUCAAGCGAUUCUUGUGCCUCAGCCUCCUGAGUAGCUGGGA | |
| | CUACAGGCGUGUGCCACCAUCAUGCCUGGCUACUUUUUUGUAUUAGAUAUAUAUUUUCUCUCUUAGCACA | |
| | GUACCUACCAAGAGUGAGUGAGUAGAUGUCCUGACCCCUGCAGGCAUCCAAGGCCCUCCUUCCCUGGACC | |
| | UGUUUCCACAUGUGUGAAGGGGUGCACAGGCAGCAGCCCACCUCUCAGCUUCCUUCCAGUUCUUGUGUUC | |
| | UGUGACCCCUUUUCCUCAUCUCUGCCUGCUUCCUCACAGCUUGAGGCAGCCCUAGGUGAGGCCAAGAAGC | |
| | AACUUCAGGAUGAGAUGCUGCGGCGGGUGGAUGCUGAGAACAGGCUGCAGACCAUGAAGGAGGAACUGGA | |
| | CUUCCAGAAGAACAUCUACAGUGAGGUGGGGACUGUGCUUUGCAAGCCAGAGGGCUGGGGCUGGGUGAUG | |
| | ACAGACUUGGGCUGGGCUAGGGGGGACCAGCUGUGUGCAGAGCUCGCCUUCCUGAGUCCCUUGCCCUAGU | |
| | GGACAGGGAGUUGGGGGUGGCCAGCACUCAGCUCCCAGGUUAAAGUGGGGCUGGUAGUGGCUCAUGGAGU | |
| | AGGGCUGGGCAGGGAGCCCCGCCCCUGGGUCUUGGCCUCCCAGGAACUAAUUCUGAUUUUGGUUUCUGUG | |
| | UCCUUCCUCCAACCCUUCCAGGAGCUGCGUGAGACCAAGCGCCGUCAUGAGACCCGACUGGUGGAGAUUG | |
| | ACAAUGGGAAGCAGCGUGAGUUUGAGAGCCGGCUGGCGGAUGCGCUGCAGGAACUGCGGGCCCAGCAUGA | |
| | GGACCAGGUGGAGCAGUAUAAGAAGGAGCUGGAGAAGACUUAUUCUGCCAAGGUGCUUGCUCUCGAUUGG | |
| | UUCCCUCACUGCCUCUGCCCUUGGCAGCCCUACCCUUACCCACGCUGGGCUAUGCCUUCUGGGGAUCAGG | |
| | CAGAUGGUGGCAGGGAGCUCAGGGUGGCCCAGGACCUGGGGCUGUAGCAGUGAUGCCCAACUCAGGCCUG | |
| | UGCCUCCACCCCUCCCAGUCACCACAGUCCUAACCCUUUGUCCUCCCCUCCAGCUGGACAAUGCCAGGCA | |
| | GUCUGCUGAGAGGAACAGCAACCUGGUGGGGGCUGCCCACGAGGAGCUGCAGCAGUCGCGCAUCCGCAUC | |
| | GACAGCCUCUCUGCCCAGCUCAGCCAGCUCCAGAAGCAGGUGAUACCCCACCUCACCCCUCUCUCCAGGG | |
| | GCCUAGAGUCUGGGCCGGAUGCAGGCUGGAAGCCCAGGGUUGGGGGUGGGGGUGGGGGUGGGAGGUUCCU | |
| | GAGGAGGAGAGGGAUGAAAAGUGUCCCCACAACCACAGAGAAGGGUCGCAGGAUGUGGAGUCAGAUGGCC | |
| | UGUGUGCUGUUUCUGUACACUCUUACCUCACCUUCACUUCUCAGGGCUUUGGUUUUCCCAUUCGAAAAUG | |
| | GAGGCUGUUCUUAAUCUCCCUAACUCAGAGUUGCCACAGGACUCUGCAAUGUGAGGUGUUAAAAGCAUCA | |
| | GUAUUUUUCUAGUUGGCUGUGCUAUUUGUGACAGGAGAAAAAGUCUAGCCUCAGAACGAGAGGUUUCAGU | |
| | UAGACAAGGGGAAGGACUUCCCAGUUGCCAGCCAAGACUAUGUUUAGAGCUUGUGAUGUUCAGAGCUGGC | |
| | UCUGAUGAGGGCUCUGGGGAAGCUCUGAUUGCAGAUCCUGGAGAGAGUAGCCAGGUGUCUCCUACACCGA | |
| | CCCACGUCCCUCCUUCCCCAUACUUAGGGCCCUUGGGAGCUCACCAAACCCUCCCACCCCCCUUCAGCUG | |
| | GCAGCCAAGGAGGCGAAGCUUCGAGACCUGGAGGACUCACUGGCCCGUGAGCGGGACACCAGCCGGCGGC | |
| | UGCUGGCGGAAAAGGAGCGGGAGAUGGCCGAGAUGCGGGCAAGGAUGCAGCAGCAGCUGGACGAGUACCA | |
| | GGAGCUUCUGGACAUCAAGCUGGCCCUGGACAUGGAGAUCCACGCCUACCGCAAGCUCUUGGAGGGCGAG | |
| | GAGGAGAGGUGGGCUGGGGAGACGUCGGGGAGGUGCUGGCAGUGUCCUCUGGCCGGCAACUGGCCUUGAC | |
| | UAGACCCCCACUUGGUCUCCCUCUCCCCAGGCUACGCCUGUCCCCCAGCCCUACCUCGCAGCGCAGCCGU | |
| | GGCCGUGCUUCCUCUCACUCAUCCCAGACACAGGGUGGGGGCAGCGUCACCAAAAAGCGCAAACUGGAGU | |
| | CCACUGAGAGCCGCAGCAGCUUCUCACAGCACGCACGCACUAGCGGGCGCGUGGCCGUGGAGGAGGUGGA | |
| | UGAGGAGGGCAAGUUUGUCCGGCUGCGCAACAAGUCCAAUGAGGUAGGCUCCUGCUCAGGGUCUAAGGGG | |
| | AUACAGCUGCAUCAGGGAGAGAGUGGCAAGACAGAAGGAUGGCAUGUGGAGAGAGGAACAUCCUUGCCCU | |
| | CAGAGGGUGGACCAGGGUGAGCCUGUAUAUCUCCUCCACNNNNNNNNNNNNNNNNNNNNNNNNNNNNNNN | |
| | NNNNNNNNNNNNNNNNNNNNNNNNNNNNNNNNNNNNNNNNNNNNNNNNNNNNNNNNNNNNNNNNNNNNNG | |
| | CUGCGUAUGUGUCCACAGAUCAUGGCUAUUAUCCCCGGGGGAAGGGCAGUGACAGGGGUGUGUGUAGAUG | |
| | GAAGGAGAGGCCUCAAUUGCAGGCAGGCAGAGGGCUGGGCCUUUGAGCAAGAUACACCCAAGAGCCUGGG | |
| | UGAGCCUCCCCGACCUUCCUCUUCCCUAUCUUCCCGGCAGGACCAGUCCAUGGGCAAUUGGCAGAUCAAG | |
| | CGCCAGAAUGGAGAUGAUCCCUUGCUGACUUACCGGUUCCCACCAAAGUUCACCCUGAAGGCUGGGCAGG | |
| | UGGUGACGGUGAGUGGCAGGGCGCUUGGGACUCUGGGGAGGCCUUGGGUGGCGAUGGGAGCGCUGGGGUA | |
| | AGUGUCCUUUUCUCCUCUCCAGAUCUGGGCUGCAGGAGCUGGGGCCACCCACAGCCCCCCUACCGACCUG | |
| | GUGUGGAAGGCACAGAACACCUGGGGCUGCGGGAACAGCCUGCGUACGGCUCUCAUCAACUCCACUGGGG | |
| | AAGUAAGUAGGCCUGGGCCUGGCUGCUUGCUGGACGAGGCUCCCCCUGAUGGCCAACAUCGGAGCCAGCU | |
| | GCCCCCAACCCAAGUUUGCCAAUUCAGGGCCCCUUUCUAGAGCUCUCUGUUGCAGGCUCCAGACUUCUCN | |
| | NNNNNNNNNNNNNNNNNNNNNNNNNNNNNNNNNNNNNNNNNNNNNNNNNNNNNNNNNNNNNNNNNNNNNN | |
| | NNNNNNNNNNNNNNNNNNNNNNNNNNNNNNNNNNNNNNNNNNNNNNNNNNNNNNNNNUGAGUUCCUUAGC | |
| | UCCAUCACCACAGAGGACAGAGUAAGCAGCAGGCCGGACAAAGGGCAGGCCACAAGAAAAGUUGCAGGUG | |
| | GUCACUGGGGUAGACAUGCUGUACAACCCUUCCCUGGCCCUGACCCUUGGACCUGGUUCCAUGUCCCCAC | |
| | CAGGAAGUGGCCAUACGCAAGCUGGUGCGCUCAGUGACUGUGGUUGAGGACGACGAGGAUGAGGAUGGAG | |
| | AUGACCUGCUCCAUCACCACCAUGUGAGUGGUAGCCGCCGCUGAGGCCGAGCCUGCACUGGGGCCACCCA | |
| | GCCAGGCCUGGGGGCAGCCUCUCCCCAGCCUCCCCGUGCCAAAAAUCUUUUCAUUAAAGAAUGUUUUGGA | |
| | ACUUUACUCGCUGGCCUGGCCUUUCUUCUCUCUCCUCCCUAUACCUUGAACAGGGAACCCAGGUGUCUGG | |
| | GUGCCCUACUCUGGUAAGGAAGGGAG | |

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The breadth and scope of the present invention should not be limited by any of the above-described exemplary embodiments, but should be defined only in accordance with the following claims and their equivalents.

All publications, patent applications, patents, and other references mentioned herein are incorporated by reference in their entirety. In case of conflict, the present specification, including definitions, will control.

1.-89. (canceled)

90. A pharmaceutical composition comprising a lipid nanoparticle, wherein
the lipid nanoparticle comprises a compound having Formula (I): 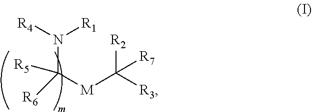
or a salt or isomer thereof, wherein:
R₁ is selected from the group consisting of C5-30 alkyl, C5-20 alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
wherein the lipid nanoparticle comprises an mRNA that comprises an open reading frame (ORF) encoding a JAG1 polypeptide, wherein the composition is suitable for administration to a human subject in need of treatment for Duchenne muscular dystrophy.

91. The pharmaceutical composition of claim 90, wherein the compound is of Formula (IA): 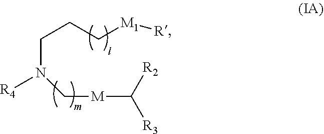
or a salt or isomer thereof, wherein
l is selected from 1, 2, 3, 4, and 5;
m is selected from 5, 6, 7, 8, and 9;
M₁ is a bond or M′;
R₄ is unsubstituted C1-3 alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl;
M and M′ are independently selected from —C(O)O—, —OC(O)—, —C(O)N(R′)—, —P(O)(OR′)O—, —S—S—, an aryl group, and a heteroaryl group; and
R₂ and R₃ are independently selected from the group consisting of H, C₁₋₁₄ alkyl, and C₂₋₁₄ alkenyl.

92. The pharmaceutical composition of claim 90, wherein m is 5, 7, or 9.

93. The pharmaceutical composition of claim 90, wherein the compound is of Formula (II) 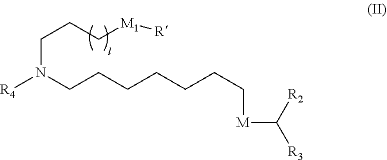
or a salt or isomer thereof, wherein
l is selected from 1, 2, 3, 4, and 5;
M₁ is a bond or M′;
R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl;
M and M′ are independently selected from —C(O)O—, —OC(O)—, —C(O)N(R′)—, —P(O)(OR′)O—, —S—S—, an aryl group, and a heteroaryl group; and
R₂ and R₃ are independently selected from the group consisting of H, C₁₋₁₄ alkyl, and C₂₋₁₄ alkenyl.

94. The pharmaceutical composition of claim 91, wherein M₁ is M′.

95. The pharmaceutical composition of claim 94, wherein M and M′ are independently —C(O)O— or —OC(O)—.

96. The pharmaceutical composition of claim 91, wherein 1 is 1, 3, or 5.

97. The pharmaceutical composition of claim 90, wherein the compound is selected from the group consisting of Compounds 1-20 or 25, salts and stereoisomers thereof, and any combination thereof.

98. The pharmaceutical composition of claim 97, wherein the compound is Compound 18, a salt or a stereoisomer thereof, or any combination thereof.

99. A method of expressing a JAG1 polypeptide in a human subject in need thereof comprising administering to the subject an effective amount of the pharmaceutical composition of claim 90, wherein the pharmaceutical composition is suitable for administrating as a single dose or as a plurality of single unit doses to the subject.

100. A method of treating, preventing or delaying the onset and/or progression of Duchenne muscular dystrophy signs or symptoms in a human subject in need thereof comprising administering to the subject an effective amount of the pharmaceutical composition of claim 90, wherein the administration treats, prevents or delays the onset and/or progression of one or more of the signs or symptoms of Duchenne muscular dystrophy in the subject.

101. A method for the treatment of Duchenne muscular dystrophy, comprising administering to a human subject suffering from Duchenne muscular dystrophy an intravenous dose of the pharmaceutical composition of claim 90.

102. A method of increasing dystrophin levels in a human subject comprising administering to the subject an effective amount of the pharmaceutical composition of claim 90, wherein the administration increases dystrophin levels in the subject.

103. The method of claim 102, wherein dystrophin levels are increased by at least a 25% relative to baseline levels.


SEQUENCE LISTING (67 sequences)

SEQ ID NO: 1 - DNA, 47 bp, Artificial Sequence
  Synthetic Polynucleotide
gggaaataagagagaaaagaagagtaagaagaaatataagagccacc

SEQ ID NO: 2 - DNA, 47 bp, Artificial Sequence
  Synthetic Polynucleotide
gggagatcagagagaaaagaagagtaagaagaaatataagagccacc

SEQ ID NO: 3 - DNA, 145 bp, Artificial Sequence
  Synthetic Polynucleotide
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcaac

SEQ ID NO: 4 - RNA, 42 nt, Artificial Sequence
  Synthetic Polynucleotide
gggagacaagcuuggcauuccgguacuguugguaaagccacc

SEQ ID NO: 5 - DNA, 47 bp, Artificial Sequence; identical to SEQ ID NO: 2
  Synthetic Polynucleotide

SEQ ID NO: 6 - DNA, 145 bp, Artificial Sequence; identical to SEQ ID NO: 3
  Synthetic Polynucleotide

SEQ ID NO: 7 - RNA, 42 nt, Artificial Sequence; identical to SEQ ID NO: 4
  Synthetic Polynucleotide

SEQ ID NO: 8 - DNA, 47 bp, Artificial Sequence
  Synthetic Polynucleotide
gggaattaacagagaaaagaagagtaagaagaaatataagagccacc

SEQ ID NO: 9 - DNA, 47 bp, Artificial Sequence
  Synthetic Polynucleotide
gggaaattagacagaaaagaagagtaagaagaaatataagagccacc

SEQ ID NO: 10 - DNA, 47 bp, Artificial Sequence
  Synthetic Polynucleotide
gggaaataagagagtaaagaacagtaagaagaaatataagagccacc

SEQ ID NO: 11 - DNA, 47 bp, Artificial Sequence
  Synthetic Polynucleotide
gggaaaaaagagagaaaagaagactaagaagaaatataagagccacc

SEQ ID NO: 12 - DNA, 47 bp, Artificial Sequence
  Synthetic Polynucleotide
gggaaataagagagaaaagaagagtaagaagatatataagagccacc

SEQ ID NO: 13 - DNA, 47 bp, Artificial Sequence
  Synthetic Polynucleotide
gggaaataagagacaaaacaagagtaagaagaaatataagagccacc

SEQ ID NO: 14 - DNA, 47 bp, Artificial Sequence
  Synthetic Polynucleotide
gggaaattagagagtaaagaacagtaagtagaattaaaagagccacc

SEQ ID NO: 15 - DNA, 47 bp, Artificial Sequence
  Synthetic Polynucleotide
gggaaataagagagaatagaagagtaagaagaaatataagagccacc

SEQ ID NO: 16 - DNA, 47 bp, Artificial Sequence
  Synthetic Polynucleotide
gggaaataagagagaaaagaagagtaagaagaaaattaagagccacc

SEQ ID NO: 17 - DNA, 47 bp, Artificial Sequence
  Synthetic Polynucleotide
gggaaataagagagaaaagaagagtaagaagaaatttaagagccacc

SEQ ID NO: 18 - DNA, 92 bp, Artificial Sequence
  Synthetic Polynucleotide
tcaagcttttggaccctcgtacagaagctaatacgactcactatagggaaataagagagaaaagaagagtaagaagaaatataagagccacc

SEQ ID NO: 19 - DNA, 142 bp, Artificial Sequence
  Synthetic Polynucleotide
tgataatagtccataaagtaggaaacactacagctggagcctcggtggccatgcttcttgccccttgggcctccccccagcccctcctccccttcctgcacccgtacccccgtggtctttgaataaagtctgagtgggcggc

SEQ ID NO: 20 - DNA, 142 bp, Artificial Sequence
  Synthetic Polynucleotide
tgataataggctggagcctcggtggctccataaagtaggaaacactacacatgcttcttgccccttgggcctccccccagcccctcctccccttcctgcacccgtacccccgtggtctttgaataaagtctgagtgggcggc

SEQ ID NO: 21 - DNA, 142 bp, Artificial Sequence
  Synthetic Polynucleotide
tgataataggctggagcctcggtggccatgcttcttgccccttccataaagtaggaaacactacatgggcctccccccagcccctcctccccttcctgcacccgtacccccgtggtctttgaataaagtctgagtgggcggc

SEQ ID NO: 22 - DNA, 142 bp, Artificial Sequence
  Synthetic Polynucleotide
tgataataggctggagcctcggtggccatgcttcttgccccttgggcctccccccagtccataaagtaggaaacactacacccctcctccccttcctgcacccgtacccccgtggtctttgaataaagtctgagtgggcggc

SEQ ID NO: 23 - DNA, 142 bp, Artificial Sequence
  Synthetic Polynucleotide
tgataataggctggagcctcggtggccatgcttcttgccccttgggcctccccccagcccctcctccccttctccataaagtaggaaacactacactgcacccgtacccccgtggtctttgaataaagtctgagtgggcggc

SEQ ID NO: 24 - DNA, 142 bp, Artificial Sequence
  Synthetic Polynucleotide
tgataataggctggagcctcggtggccatgcttcttgccccttgggcctccccccagcccctcctccccttcctgcacccgtaccccctccataaagtaggaaacactacagtggtctttgaataaagtctgagtgggcggc

SEQ ID NO: 25 - DNA, 142 bp, Artificial Sequence
  Synthetic Polynucleotide
tgataataggctggagcctcggtggccatgcttcttgccccttgggcctccccccagcccctcctccccttcctgcacccgtacccccgtggtctttgaataaagttccataaagtaggaaacactacactgagtgggcggc

SEQ ID NO: 26 - DNA, 371 bp, Artificial Sequence
  Synthetic Polynucleotide
gcgcctgcccacctgccaccgactgctggaacccagccagtgggagggcctggcccaccagagtcctgctccctcactcctcgccccgccccctgtcccagagtcccacctgggggctctctccacccttctcagagttccagtttcaaccagagttccaaccaatgggctccatcctctggattctggccaatgaaatatctccctggcagggtcctcttcttttcccagagctccaccccaaccaggagctctagttaatggagagctcccagcacactcggagcttgtgctttgtctccacgcaaagcgataaataaaagcattggtggcctttggtctttgaataaagcctgagtaggaagtctaga

SEQ ID NO: 27 - DNA, 568 bp, Artificial Sequence
  Synthetic Polynucleotide
gcccctgccgctcccacccccacccatctgggccccgggttcaagagagagcggggtctgatctcgtgtagccatatagagtttgcttctgagtgtctgctttgtttagtagaggtgggcaggaggagctgaggggctggggctggggtgttgaagttggctttgcatgcccagcgatgcgcctccctgtgggatgtcatcaccctgggaaccgggagtggcccttggctcactgtgttctgcatggtttggatctgaattaattgtcctttcttctaaatcccaaccgaacttcttccaacctccaaactggctgtaaccccaaatccaagccattaactacacctgacagtagcaattgtctgattaatcactggccccttgaagacagcagaatgtccctttgcaatgaggaggagatctgggctgggcgggccagctggggaagcatttgactatctggaacttgtgtgtgcctcctcaggtatggcagtgactcacctggttttaataaaacaacctgcaacatctcatggtctttgaataaagcctgagtaggaagtctaga

SEQ ID NO: 28 - DNA, 289 bp, Artificial Sequence
  Synthetic Polynucleotide
acacactccacctccagcacgcgacttctcaggacgacgaatcttctcaatgggggggcggctgagctccagccaccccgcagtcactttctttgtaacaacttccgttgctgccatcgtaaactgacacagtgtttataacgtgtacatacattaacttattacctcattttgttatttttcgaaacaaagccctgtggaagaaaatggaaaacttgaagaagcattaaagtcattctgttaagctgcgtaaatggtctttgaataaagcctgagtaggaagtctaga

SEQ ID NO: 29 - DNA, 379 bp, Artificial Sequence
  Synthetic Polynucleotide
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaatggtctttgaataaagcctgagtaggaagtctaga

SEQ ID NO: 30 - DNA, 118 bp, Artificial Sequence
  Synthetic Polynucleotide
gctgccttctgcggggcttgccttctggccatgcccttcttctctcccttgcacctgtacctcttggtctttgaataaagcctgagtaggaaggcggccgctcgagcatgcatctaga

SEQ ID NO: 31 - DNA, 908 bp, Artificial Sequence
  Synthetic Polynucleotide
gccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctccatggtctttgaataaagcctgagtaggaaggcggccgctcgagcatgcatctaga

SEQ ID NO: 32 - DNA, 835 bp, Artificial Sequence
  Synthetic Polynucleotide
actcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 33 - DNA, 297 bp, Artificial Sequence
  Synthetic Polynucleotide
cgccgccgcccgggccccgcagtcgagggtcgtgagcccaccccgtccatggtgctaagcgggcccgggtcccacacggccagcaccgctgctcactcggacgacgccctgggcctgcacctctccagctcctcccacggggtccccgtagccccggcccccgcccagccccaggtctccccaggccctccgcaggctgcccggcctccctccccctgcagccatcccaaggctcctgacctacctggcccctgagctctggagcaagccctgacccaataaaggctttgaacccat

SEQ ID NO: 34 - DNA, 602 bp, Artificial Sequence
  Synthetic Polynucleotide
ggggctagagccctctccgcacagcgtggagacggggcaaggaggggggttattaggattggtggttttgttttgctttgtttaaagccgtgggaaaatggcacaactttacctctgtgggagatgcaacactgagagccaaggggtgggagttgggataatttttatataaaagaagtttttccactttgaattgctaaaagtggcatttttcctatgtgcagtcactcctctcatttctaaaatagggacgtggccaggcacggtggctcatgcctgtaatcccagcactttgggaggccgaggcaggcggctcacgaggtcaggagatcgagactatcctggctaacacggtaaaaccctgtctctactaaaagtacaaaaaattagctgggcgtggtggtgggcacctgtagtcccagctactcgggaggctgaggcaggagaaaggcatgaatccaagaggcagagcttgcagtgagctgagatcacgccattgcactccagcctgggcaacagtgttaagactctgtctcaaatataaataaataaataaataaataaataaataaataaaaataaagcgagatgttgccctcaaa

SEQ ID NO: 35 - DNA, 785 bp, Artificial Sequence
  Synthetic Polynucleotide
ggccctgccccgtcggactgcccccagaaagcctcctgccccctgccagtgaagtccttcagtgagcccctccccagccagcccttccctggccccgccggatgtataaatgtaaaaatgaaggaattacattttatatgtgagcgagcaagccggcaagcgagcacagtattatttctccatcccctccctgcctgctccttggcacccccatgctgccttcagggagacaggcagggagggcttggggctgcacctcctaccctcccaccagaacgcaccccactgggagagctggtggtgcagccttcccctccctgtataagacactttgccaaggctctcccctctcgccccatccctgcttgcccgctcccacagcttcctgagggctaattctgggaagggagagttctttgctgcccctgtctggaagacgtggctctgggtgaggtaggcgggaaaggatggagtgttttagttcttgggggaggccaccccaaaccccagccccaactccaggggcacctatgagatggccatgctcaacccccctcccagacaggccctccctgtctccagggcccccaccgaggttcccagggctggagacttcctctggtaaacattcctccagcctcccctcccctggggacgccaaggaggtgggccacacccaggaagggaaagcgggcagccccgttttggggacgtgaacgttttaataatttttgctgaattcctttacaactaaataacacagatattgttataaataaaattgt

SEQ ID NO: 36 - DNA, 3001 bp, Artificial Sequence
  Synthetic Polynucleotide
atattaaggatcaagctgttagctaataatgccacctctgcagttttgggaacaggcaaataaagtatcagtatacatggtgatgtacatctgtagcaaagctcttggagaaaatgaagactgaagaaagcaaagcaaaaactgtatagagagatttttcaaaagcagtaatccctcaattttaaaaaaggattgaaaattctaaatgtctttctgtgcatattttttgtgttaggaatcaaaagtattttataaaaggagaaagaacagcctcattttagatgtagtcctgttggattttttatgcctcctcagtaaccagaaatgttttaaaaaactaagtgtttaggatttcaagacaacattatacatggctctgaaatatctgacacaatgtaaacattgcaggcacctgcattttatgttttttttttcaacaaatgtgactaatttgaaacttttatgaacttctgagctgtccccttgcaattcaaccgcagtttgaattaatcatatcaaatcagttttaattttttaaattgtacttcagagtctatatttcaagggcacattttctcactactattttaatacattaaaggactaaataatctttcagagatgctggaaacaaatcatttgctttatatgtttcattagaataccaatgaaacatacaacttgaaaattagtaatagtatttttgaagatcccatttctaattggagatctctttaatttcgatcaacttataatgtgtagtactatattaagtgcacttgagtggaattcaacatttgactaataaaatgagttcatcatgttggcaagtgatgtggcaattatctctggtgacaaaagagtaaaatcaaatatttctgcctgttacaaatatcaaggaagacctgctactatgaaatagatgacattaatctgtcttcactgtttataatacggatggattttttttcaaatcagtgtgtgttttgaggtcttatgtaattgatgacatttgagagaaatggtggctttttttagctacctctttgttcatttaagcaccagtaaagatcatgtctttttatagaagtgtagattttctttgtgactttgctatcgtgcctaaagctctaaatataggtgaatgtgtgatgaatactcagattatttgtctctctatataattagtttggtactaagtttctcaaaaaattattaacacatgaaagacaatctctaaaccagaaaaagaagtagtacaaattttgttactgtaatgctcgcgtttagtgagtttaaaacacacagtatcttttggttttataatcagtttctattttgctgtgcctgagattaagatctgtgtatgtgtgtgtgtgtgtgtgtgcgtttgtgtgttaaagcagaaaagacttttttaaaagttttaagtgataaatgcaatttgttaattgatcttagatcactagtaaactcagggctgaattataccatgtatattctattagaagaaagtaaacaccatctttattcctgccctttttcttctctcaaagtagttgtagttatatctagaaagaagcaattttgatttcttgaaaaggtagttcctgcactcagtttaaactaaaaataatcatacttggattttatttatttttgtcatagtaaaaattttaatttatatatatttttatttagtattatcttattctttgctatttgccaatcctttgtcatcaattgtgttaaatgaattgaaaattcatgccctgttcattttattttactttattggttaggatatttaaaggatttttgtatatataatttcttaaattaatattccaaaaggttagtggacttagattataaattatggcaaaaatctaaaaacaacaaaaatgatttttatacattctatttcattattcctctttttccaataagtcatacaattggtagatatgacttattttatttttgtattattcactatatctttatgatatttaagtataaataattaaaaaaatttattgtaccttatagtctgtcaccaaaaaaaaaaaattatctgtaggtagtgaaatgctaatgttgatttgtctttaagggcttgttaactatcctttattttctcatttgtcttaaattaggagtttgtgtttaaattactcatctaagcaaaaaatgtatataaatcccattactgggtatatacccaaaggattataaatcatgctgctataaagacacatgcacacgtatgtttattgcagcactattcacaatagcaaagacttggaaccaacccaaatgtccatcaatgatagacttgattaagaaaatgtgcacatatacaccatggaatactatgcagccataaaaaaggatgagttcatgtcctttgtagggacatggataaagctggaaaccatcattctgagcaaactattgcaaggacagaaaaccaaacactgcatgttctcactcataggtgggaattgaacaatgagaacacttggacacaaggtggggaacaccacacaccagggcctgtcatggggtggggggagtggggagggatagcattaggagatatacctaatgtaaatgatgagttaatgggtgcagcacaccaacatggcacatgtatacatatgtagcaaacctgcacgttgtgcacatgtaccctagaacttaaagtataattaaaaaaaaaaagaaaacagaagctatttataaagaagttatttgctgaaataaatgtgatctttcccattaaaaaaataaagaaattttggggtaaaaaaacacaatatattgtattcttgaaaaattctaagagagtggatgtgaagtgttctcaccacaaaagtgataactaattgaggtaatgcacatattaattagaaagattttgtcattccacaatgtatatatacttaaaaatatgttatacacaataaatacatacattaaaaaataagtaaatgta

SEQ ID NO: 37 - DNA, 1037 bp, Artificial Sequence
  Synthetic Polynucleotide
cccaccctgcacgccggcaccaaaccctgtcctcccacccctccccactcatcactaaacagagtaaaatgtgatgcgaattttcccgaccaacctgattcgctagattttttttaaggaaaagcttggaaagccaggacacaacgctgctgcctgctttgtgcagggtcctccggggctcagccctgagttggcatcacctgcgcagggccctctggggctcagccctgagctagtgtcacctgcacagggccctctgaggctcagccctgagctggcgtcacctgtgcagggccctctggggctcagccctgagctggcctcacctgggttccccaccccgggctctcctgccctgccctcctgcccgccctccctcctgcctgcgcagctccttccctaggcacctctgtgctgcatcccaccagcctgagcaagacgccctctcggggcctgtgccgcactagcctccctctcctctgtccccatagctggtttttcccaccaatcctcacctaacagttactttacaattaaactcaaagcaagctcttctcctcagcttggggcagccattggcctctgtctcgttttgggaaaccaaggtcaggaggccgttgcagacataaatctcggcgactcggccccgtctcctgagggtcctgctggtgaccggcctggaccttggccctacagccctggaggccgctgctgaccagcactgaccccgacctcagagagtactcgcaggggcgctggctgcactcaagaccctcgagattaacggtgctaaccccgtctgctcctccctcccgcagagactggggcctggactggacatgagagccccttggtgccacagagggctgtgtcttactagaaacaacgcaaacctctccttcctcagaatagtgatgtgttcgacgttttatcaaaggccccctttctatgttcatgttagttttgctccttctgtgtttttttctgaaccatatccatgttgctgacttttccaaataaaggttttcactcctctc

SEQ ID NO: 38 - DNA, 577 bp, Artificial Sequence
  Synthetic Polynucleotide
agaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctcc

SEQ ID NO: 39 - DNA, 2212 bp, Artificial Sequence
  Synthetic Polynucleotide
ctccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataatccgcagtggcctcctaatttccttctttggagttgggggaggggtagacatggggaaggggctttggggtgatgggcttgccttccattcctgccctttccctccccactattctcttctagatccctccataaccccactcccctttctctcacccttcttataccgcaaacctttctacttcctctttcattttctattcttgcaatttccttgcaccttttccaaatcctcttctcccctgcaataccatacaggcaatccacgtgcacaacacacacacacactcttcacatctggggttgtccaaacctcatacccactccccttcaagcccatccactctccaccccctggatgccctgcacttggtggcggtgggatgctcatggatactgggagggtgaggggagtggaacccgtgaggaggacctgggggcctctccttgaactgacatgaagggtcatctggcctctgctcccttctcacccacgctgacctcctgccgaaggagcaacgcaacaggagaggggtctgctgagcctggcgagggtctgggagggaccaggaggaaggcgtgctccctgctcgctgtcctggccctgggggagtgagggagacagacacctgggagagctgtggggaaggcactcgcaccgtgctcttgggaaggaaggagacctggccctgctcaccacggactgggtgcctcgacctcctgaatccccagaacacaacccccctgggctggggtggtctggggaaccatcgtgcccccgcctcccgcctactcctttttaagctt

SEQ ID NO: 40 - DNA, 729 bp, Artificial Sequence
  Synthetic Polynucleotide
ttggccaggcctgaccctcttggacctttcttctttgccgacaaccactgcccagcagcctctgggacctcggggtcccagggaacccagtccagcctcctggctgttgacttcccattgctcttggagccaccaatcaaagagattcaaagagattcctgcaggccagaggcggaacacacctttatggctggggctctccgtggtgttctggacccagcccctggagacaccattcacttttactgctttgtagtgactcgtgctctccaacctgtcttcctgaaaaaccaaggcccccttcccccacctcttccatggggtgagacttgagcagaacaggggcttccccaagttgcccagaaagactgtctgggtgagaagccatggccagagcttctcccaggcacaggtgttgcaccagggacttctgcttcaagttttggggtaaagacacctggatcagactccaagggctgccctgagtctgggacttctgcctccatggctggtcatgagagcaaaccgtagtcccctggagacagcgactccagagaacctcttgggagacagaagaggcatctgtgcacagctcgatcttctacttgcctgtggggaggggagtgacaggtccacacaccacactgggtcaccctgtcctggatgcctctgaagagagggacagaccgtcagaaactggagagtttctattaaaggtcatttaaacca

SEQ ID NO: 41 - DNA, 847 bp, Artificial Sequence
  Synthetic Polynucleotide
tcctccgggaccccagccctcaggattcctgatgctccaaggcgactgatgggcgctggatgaagtggcacagtcagcttccctgggggctggtgtcatgttgggctcctggggcgggggcacggcctggcatttcacgcattgctgccaccccaggtccacctgtctccactttcacagcctccaagtctgtggctcttcccttctgtcctccgaggggcttgccttctctcgtgtccagtgaggtgctcagtgatcggcttaacttagagaagcccgccccctccccttctccgtctgtcccaagagggtctgctctgagcctgcgttcctaggtggctcggcctcagctgcctgggttgtggccgccctagcatcctgtatgcccacagctactggaatccccgctgctgctccgggccaagcttctggttgattaatgagggcatggggtggtccctcaagaccttcccctaccttttgtggaaccagtgatgcctcaaagacagtgtcccctccacagctgggtgccaggggcaggggatcctcagtatagccggtgaaccctgataccaggagcctgggcctccctgaacccctggcttccagccatctcatcgccagcctcctcctggacctcttggcccccagccccttccccacacagccccagaagggtcccagagctgaccccactccaggacctaggcccagcccctcagcctcatctggagcccctgaagaccagtcccacccacctttctggcctcatctgacactgctccgcatcctgctgtgtgtcctgttccatgttccggttccatccaaatacactttctggaacaaa

SEQ ID NO: 42 - DNA, 110 bp, Artificial Sequence
  Synthetic Polynucleotide
gctggagcctcggtggccatgcttcttgccccttgggcctccccccagcccctcctccccttcctgcacccgtacccccgtggtctttgaataaagtctgagtgggcggc

SEQ ID NO: 43 - DNA, 119 bp, Artificial Sequence
  Synthetic Polynucleotide
tgataataggctggagcctcggtggccatgcttcttgccccttgggcctccccccagcccctcctccccttcctgcacccgtacccccgtggtctttgaataaagtctgagtgggcggc

SEQ ID NO: 44 - RNA, 87 nt, Artificial Sequence
  Synthetic Polynucleotide
gacagugcagucacccauaaaguagaaagcacuacuaacagcacuggaggguguaguguuuccuacuuuauggaugaguguacugug

SEQ ID NO: 45 - RNA, 23 nt, Artificial Sequence
  Synthetic Polynucleotide
uguaguguuuccuacuuuaugga

SEQ ID NO: 46 - RNA, 23 nt, Artificial Sequence
  Synthetic Polynucleotide
uccauaaaguaggaaacacuaca

SEQ ID NO: 47 - RNA, 21 nt, Artificial Sequence
  Synthetic Polynucleotide
cauaaaguagaaagcacuacu

SEQ ID NO: 48 - RNA, 21 nt, Artificial Sequence
  Synthetic Polynucleotide
aguagugcuuucuacuuuaug

SEQ ID NO: 49 - DNA, 18 bp, Artificial Sequence
  Synthetic Polynucleotide
attgggcacccgtaaggg

SEQ ID NO: 50 - protein, 1218 aa, Homo sapiens [sequence omitted]

SEQ ID NO: 51 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 52 - protein, 3122 aa, Homo sapiens
MPGAAGVLLLLLLSGGLGGVQAQRPQQQRQSQAHQQRGLFPAVLNLASNALITTNATCGEKGPEMYCKLVEHVPGQPVRNPQCRICNQNSSNPNQRHPITNAIDGKNTWWQSPSIKNGIEYHYVTITLDLQQVFQIAYVIVKAANSPRPGNWILERSLDDVEYKPWQYHAVTDTECLTLYNIYPRTGPPSYAKDDEVICTSFYSKIHPLENGEIHISLINGRPSADDPSPELLEFTSARYIRLRFQRIRTLNADLMMFAHKDPREIDPIVTRRYYYSVKDISVGGMCICYGHARACPLDPATNKSRCECEHNTCGDSCDQCCPGFHQKPWRAGTFLTKTECEACNCHGKAEECYYDENVARRNLSLNIRGKYIGGGVCINCTQNTAGINCETCTDGFFRPKGVSPNYPRPCQPCHCDPIGSLNEVCVKDEKHARRGLAPGSCHCKTGFGGVSCDRCARGYTGYPDCKACNCSGLGSKNEDPCFGPCICKENVEGGDCSRCKSGFFNLQEDNWKGCDECFCSGVSNRCQSSYWTYGKIQDMSGWYLTDLPGRIRVAPQQDDLDSPQQISISNAEARQALPHSYYWSAPAPYLGNKLPAVGGQLTFTISYDLEEEEEDTERVLQLMIILEGNDLSISTAQDEVYLHPSEEHTNVLLLKEESFTIHGTHFPVRRKEFMTVLANLKRVLLQITYSFGMDAIFRLSSVNLESAVSYPTDGSIAAAVEVCQCPPGYTGSSCESCWPRHRRVNGTIFGGICEPCQCFGHAESCDDVTGECLNCKDHTGGPYCDKCLPGFYGEPTKGTSEDCQPCACPLNIPSNNFSPTCHLDRSLGLICDGCPVGYTGPRCERCAEGYFGQPSVPGGSCQPCQCNDNLDFSIPGSCDSLSGSCLICKPGTTGRYCELCADGYFGDAVDAKNCQPCRCNAGGSFSEVCHSQTGQCECRANVQGQRCDKCKAGTFGLQSARGCVPCNCNSFGSKSFDCEESGQCWCQPGVTGKKCDRCAHGYFNFQEGGCTACECSHLGNNCDPKTGRCICPPNTIGEKCSKCAPNTWGHSITTGCKACNCSTVGSLDFQCNVNTGQCNCHPKFSGAKCTECSRGHWNYPRCNLCDCFLPGTDATTCDSETKKCSCSDQTGQCTCKVNVEGIHCDRCRPGKFGLDAKNPLGCSSCYCFGTTTQCSEAKGLIRTWVTLKAEQTILPLVDEALQHTTTKGIVFQHPEIVAHMDLMREDLHLEPFYWKLPEQFEGKKLMAYGGKLKYAIYFEAREETGFSTYNPQVIIRGGTPTHARIIVRHMAAPLIGQLTRHEIEMTEKEWKYYGDDPRVHRTVTREDFLDILYDIHYILIKATYGNFMRQSRISEISMEVAEQGRGTTMTPPADLIEKCDCPLGYSGLSCEACLPGFYRLRSQPGGRTPGPTLGTCVPCQCNGHSSLCDPETSICQNCQHHTAGDFCERCALGYYGIVKGLPNDCQQCACPLISSSNNFSPSCVAEGLDDYRCTACPRGYEGQYCERCAPGYTGSPGNPGGSCQECECDPYGSLPVPCDPVTGFCTCRPGATGRKCDGCKHWHAREGWECVFCGDECTGLLLGDLARLEQMVMSINLTGPLPAPYKMLYGLENMTQELKHLLSPQRAPERLIQLAEGNLNTLVTEMNELLTRATKVTADGEQTGQDAERTNTRAKSLGEFIKELARDAEAVNEKAIKLNETLGTRDEAFERNLEGLQKEIDQMIKELRRKNLETQKEIAEDELVAAEALLKKVKKLFGESRGENEEMEKDLREKLADYKNKVDDAWDLLREATDKIREANRLFAVNQKNMTALEKKKEAVESGKRQIENTLKEGNDILDEANRLADEINSIIDYVEDIQTKLPPMSEELNDKIDDLSQEIKDRKLAEKVSQAESHAAQLNDSSAVLDGILDEAKNISFNATAAFKAYSNIKDYIDEAEKVAKEAKDLAHEATKLATGPRGLLKEDAKGCLQKSFRILNEAKKLANDVKENEDHLNGLKTRIENADARNGDLLRTLNDTLGKLSAIPNDTAAKLQAVKDKARQANDTAKDVLAQITELHQNLDGLKKNYNKLADSVAKTNAVVKDPSKNKIIADADATVKNLEQEADRLIDKLKPIKELEDNLKKNISEIKELINQARKQANSIKVSVSSGGDCIRTYKPEIKKGSYNNIVVNVKTAVADNLLFYLGSAKFIDFLAIEMRKGKVSFLWDVGSGVGRVEYPDLTIDDSYWYRIVASRTGRNGTISVRALDGPKASIVPSTHHSTSPPGYTILDVDANAMLFVGGLTGKLKKADAVRVITFTGCMGETYFDNKPIGLWNFREKEGDCKGCTVSPQVEDSEGTIQFDGEGYALVSRPIRWYPNISTVMFKFRTFSSSALLMYLATRDLRDFMSVELTDGHIKVSYDLGSGMASVVSNQNHNDGKWKSFTLSRIQKQANISIVDIDTNQEENIATSSSGNNFGLDLKADDKIYFGGLPTLRNLSMKARPEVNLKKYSGCLKDIEISRTPYNILSSPDYVGVTKGCSLENVYTVSFPKPGFVELSPVPIDVGTEINLSFSTKNESGIILLGSGGTPAPPRRKRRQTGQAYYAILLNRGRLEVHLSTGARTMRKIVIRPEPNLFHDGREHSVHVERTRGIFTVQVDENRRYMQNLTVEQPIEVKKLFVGGAPPEFQPSPLRNIPPFEGCIWNLVINSVPMDFARPVSFKNADIGRCAHQKLREDEDGAAPAEIVIQPEPVPTPAFPTPTPVLTHGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTGKPLEVNFAKALELRGVQPVSCPAN

SEQ ID NO: 53 - protein, 254 aa, Homo sapiens
MDNYADLSDTELTTLLRRYNIPHGPVVGSTRRLYEKKIFEYETQRRRLSPPSSSAASSYSFSDLNSTRGDADMYDLPKKEDALLYQSKGYNDDYYEESYFTTRTYGEPESAGPSRAVRQSVTSFPDADAFHHQVHDDDLLSSSEEECKDRERPMYGRDSAYQSITHYRPVSASRSSLDLSYYPTSSSTSFMSSSSSSSSWLTRRAIRPENRAPGAGLGQDRQVPLWGQLLLFLVFVIVLFFIYHFMQAEEGNPF

SEQ ID NO: 54 - protein, 2080 aa, Homo sapiens
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADTGGEEDTEDQGLTGDEAEPFLDQSGGPGAPTTPRKLPSRPPPHYPGIKRKRSAPTSRKLLSDKPQDFQIRVQVIEGRQLPGVNIKPVVKVTAAGQTKRTRIHKGNSPLFNETLFFNLFDSPGELFDEPIFITVVDSRSLRTDALLGEFRMDVGTIYREPRHAYLRKWLLLSDPDDFSAGARGYLKTSLCVLGPGDEAPLERKDPSEDKEDIESNLLRPTGVALRGAHFCLKVFRAEDLPQMDDAVMDNVKQIFGFESNKKNLVDPFVEVSFAGKMLCSKILEKTANPQWNQNITLPAMFPSMCEKMRIRIIDWDRLTHNDIVATTYLSMSKISAPGGEIEEEPAGAVKPSKASDLDDYLGFLPTFGPCYINLYGSPREFTGFPDPYTELNTGKGEGVAYRGRLLLSLETKLVEHSEQKVEDLPADDILRVEKYLRRRKYSLFAAFYSATMLQDVDDAIQFEVSIGNYGNKFDMTCLPLASTTQYSRAVFDGCHYYYLPWGNVKPVVVLSSYWEDISHRIETQNQLLGIADRLEAGLEQVHLALKAQCSTEDVDSLVAQLTDELIAGCSQPLGDIHETPSATHLDQYLYQLRTHHLSQITEAALALKLGHSELPAALEQAEDWLLRLRALAEEPQNSLPDIVIWMLQGDKRVAYQRVPAHQVLFSRRGANYCGKNCGKLQTIFLKYPMEKVPGARMPVQIRVKLWFGLSVDEKEFNQFAEGKLSVFAETYENETKLALVGNWGTTGLTYPKFSDVTGKIKLPKDSFRPSAGWTWAGDWFVCPEKTLLHDMDAGHLSFVEEVFENQTRLPGGQWIYMSDNYTDVNGEKVLPKDDIECPLGWKWEDEEWSTDLNRAVDEQGWEYSITIPPERKPKHWVPAEKMYYTHRRRRWVRLRRRDLSQMEALKRHRQAEAEGEGWEYASLFGWKFHLEYRKTDAFRRRRWRRRMEPLEKTGPAAVFALEGALGGVMDDKSEDSMSVSTLSFGVNRPTISCIFDYGNRYHLRCYMYQARDLAAMDKDSFSDPYAIVSFLHQSQKTVVVKNTLNPTWDQTLIFYEIEIFGEPATVAEQPPSIVVELYDHDTYGADEFMGRCICQPSLERMPRLAWFPLTRGSQPSGELLASFELIQREKPAIHHIPGFEVQETSRILDESEDTDLPYPPPQREANIYMVPQNIKPALQRTAIEILAWGLRNMKSYQLANISSPSLVVECGGQTVQSCVIRNLRKNPNFDICTLFMEVMLPREELYCPPITVKVIDNRQFGRRPVVGQCTIRSLESFLCDPYSAESPSPQGGPDDVSLLSPGEDVLIDIDDKEPLIPIQEEEFIDWWSKFFASIGEREKCGSYLEKDFDTLKVYDTQLENVEAFEGLSDFCNTFKLYRGKTQEETEDPSVIGEFKGLFKIYPLPEDPAIPMPPRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

SEQ ID NO: 55 - protein, 572 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHVSGSRR

SEQ ID NO: 56 - DNA, 5942 bp, Homo sapiens
ccgggtccttctccgagagccgggcgggcacgcgtcattgtgttacctgcggccggcccgcgagctaggctggttttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccgcgaatgtaacagagctatttgccgacaaggctgcagtcctaagcatgggtcttgcaaactcccaggtgactgcaggtgccagtacggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttggatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataattgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattacgagaacaagaactccaaaatgtctaaaataaggacacacaattctgaagtagaagaggacgacatggacaaacaccagcagaaagcccggtttgccaagcagccggcgtatacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttatttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaattttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatctgactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaacatggaacagtgtggctcttctgagcttacgtagttctaccggctttgccgtgtgcttctgccaccctgctgagtctgttctggtaatcggggtataataggctctgcctgacagagggatggaggaagaactgaaaggcttttcaaccacaaaactcatctggagttctcaaagacctggggctgctgtgaagctggaactgcgggagccccatctaggggagccttgattcccttgttattcaacagcaagtgtgaatactgcttgaataaacaccactggattaatggaaaaaaaaaaaaaaaa

SEQ ID NO: 57 - DNA, 13957 bp, Homo sapiens
gggattccctcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagctgcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagagagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaagacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccgagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 58 - DNA, 9696 bp, Homo sapiens
ttccccagcagctgctgctcgctcagctcacaagccaaggccaggggacagggcggcagcgactcctctggctcccgagaagtggatccggtcgcggccactacgatgccgggagccgccggggtcctcctccttctgctgctctccggaggcctcgggggcgtacaggcgcagcggccgcagcagcagcggcagtcacaggcacatcagcaaagaggtttattccctgctgtcctgaatcttgcttctaatgctcttatcacgaccaatgcaacatgtggagaaaaaggacctgaaatgtactgcaaattggtagaacatgtccctgggcagcctgtgaggaacccgcagtgtcgaatctgcaatcaaaacagcagcaatccaaaccagagacacccgattacaaatgctattgatggaaagaacacttggtggcagagtcccagtattaagaatggaatcgaataccattatgtgacaattaccctggatttacagcaggtgttccagatcgcgtatgtgattgtgaaggcagctaactccccccggcctggaaactggattttggaacgctctcttgatgatgttgaatacaagccctggcagtatcatgctgtgacagacacggagtgcctaacgctttacaatatttatccccgcactgggccaccgtcatatgccaaagatgatgaggtcatctgcacttcattttactccaagatacaccccttagaaaatggagagattcacatctctttaatcaatgggagaccaagtgccgatgatccttctccagaactgctagaatttacctccgctcgctatattcgcctgagatttcagaggatccgcacactgaatgctgacttgatgatgtttgctcacaaagacccaagagaaattgaccccattgtcaccagaagatattactactcggtcaaggatatttcagttggagggatgtgcatctgctatggtcatgccagggcttgtccacttgatccagcgacaaataaatctcgctgtgagtgtgagcataacacatgtggcgatagctgtgatcagtgctgtccaggattccatcagaaaccctggagagctggaacttttctaactaaaactgaatgtgaagcatgcaattgtcatggaaaagctgaagaatgctattatgatgaaaatgttgccagaagaaatctgagtttgaatatacgtggaaagtacattggagggggtgtctgcattaattgtacccaaaacactgctggtataaactgcgagacatgtactgatggcttcttcagacccaaaggggtatctccaaattatccaaggccatgccagccatgtcattgcgatccaattggttccttaaatgaagtctgtgtcaaggatgagaaacatgctcgacgaggtttggcacctggatcctgtcattgcaaaactggttttggaggtgtgagctgtgatcggtgtgccaggggctacactggctacccggactgcaaagcctgtaactgcagtgggttagggagcaaaaatgaggatccttgttttggcccctgtatctgcaaggaaaatgttgaaggaggagactgtagtcgttgcaaatccggcttcttcaatttgcaagaggataattggaaaggctgcgatgagtgtttctgttcaggggtttcaaacagatgtcagagttcctactggacctatggcaaaatacaagatatgagtggctggtatctgactgaccttcctggccgcattcgagtggctccccagcaggacgacttggactcacctcagcagatcagcatcagtaacgcggaggcccggcaagccctgccgcacagctactactggagcgcgccggctccctatctgggaaacaaactcccagcagtaggaggacagttgacatttaccatatcatatgaccttgaagaagaggaagaagatacagaacgtgttctccagcttatgattatcttagagggtaatgacttgagcatcagcacagcccaagatgaggtgtacctgcacccatctgaagaacatactaatgtattgttacttaaagaagaatcatttaccatacatggcacacattttccagtccgtagaaaggaatttatgacagtgcttgcgaatttgaagagagtcctcctacaaatcacatacagctttgggatggatgccatcttcaggttgagctctgttaaccttgaatccgctgtctcctatcctactgatggaagcattgcagcagctgtagaagtgtgtcagtgcccaccagggtatactggctcctcttgtgaatcttgttggcctaggcacaggcgagttaacggcactatttttggtggcatctgtgagccatgtcagtgctttggtcatgcggagtcctgtgatgacgtcactggagaatgcctgaactgtaaggatcacacaggtggcccatattgtgataaatgtcttcctggtttctatggcgagcctactaaaggaacctctgaagactgtcaaccctgtgcctgtccactcaatatcccatccaataactttagcccaacgtgccatttagaccggagtcttggattgatctgtgatggatgccctgtcgggtacacaggaccacgctgtgagaggtgtgcagaaggctattttggacaaccctctgtacctggaggatcatgtcagccatgccaatgcaatgacaaccttgacttctccatccctggcagctgtgacagcttgtctggctcctgtctgatatgtaaaccaggtacaacaggccggtactgtgagctctgtgctgatggatattttggagatgcagttgatgcgaagaactgtcagccctgtcgctgtaatgccggtggctctttctctgaggtttgccacagtcaaactggacagtgtgagtgcagagccaacgttcagggtcagagatgtgacaaatgcaaggctgggacctttggcctacaatcagcaaggggctgtgttccctgcaactgcaattcttttgggtctaagtcattcgactgtgaagagagtggacaatgttggtgccaacctggagtcacagggaagaaatgtgaccgctgtgcccacggctatttcaacttccaagaaggaggctgcacagcttgtgaatgttctcatctgggtaataattgtgacccaaagactgggcgatgcatttgccctcccaataccattggagagaaatgttctaaatgtgcacccaatacctggggccacagcattaccactggttgtaaggcttgtaactgcagcacagtgggatccttggatttccaatgcaatgtaaatacaggccaatgcaactgtcatccaaaattctctggtgcaaaatgtacagagtgcagtcgaggtcactggaactaccctcgctgcaatctctgtgactgcttcctccctgggacagatgccacaacctgtgattcagagactaaaaaatgctcctgtagtgatcaaactgggcagtgcacttgtaaggtgaatgtggaaggcatccactgtgacagatgccggcctggcaaattcggactcgatgccaagaatccacttggctgcagcagctgctattgcttcggcactactacccagtgctctgaagcaaaaggactgatccggacgtgggtgactctgaaggctgagcagaccattctacccctggtagatgaggctctgcagcacacgaccaccaagggcattgtttttcaacatccagagattgttgcccacatggacctgatgagagaagatctccatttggaacctttttattggaaacttccagaacaatttgaaggaaagaagttgatggcctatgggggcaaactcaagtatgcaatctatttcgaggctcgggaagaaacaggtttctctacatataatcctcaagtgatcattcgaggtgggacacctactcatgctagaattatcgtcaggcatatggctgctcctctgattggccaattgacaaggcatgaaattgaaatgacagagaaagaatggaaatattatggggatgatcctcgagtccatagaactgtgacccgagaagacttcttggatatactatatgatattcattacattcttatcaaagctacttatggaaatttcatgcgacaaagcaggatttctgaaatctcaatggaggtagctgaacaaggacgtggaacaacaatgactcctccagctgacttgattgaaaaatgtgattgtcccctgggctattctggcctgtcctgtgaggcatgcttgccgggattttatcgactgcgttctcaaccaggtggccgcacccctggaccaaccctgggcacctgtgttccatgtcaatgtaatggacacagcagcctgtgtgaccctgaaacatcgatatgccagaattgtcaacatcacactgctggtgacttctgtgaacgatgtgctcttggatactatggaattgtcaagggattgccaaatgactgtcagcaatgtgcctgccctctgatttcttccagtaacaatttcagcccctcttgtgtcgcagaaggacttgacgactaccgctgcacggcttgtccacggggatatgaaggccagtactgtgaaaggtgtgcccctggctatactggcagtccaggcaaccctggaggctcctgccaagaatgtgagtgtgatccctatggctcactgcctgtgccctgtgaccctgtcacaggattctgcacgtgccgacctggagccacgggaaggaagtgtgacggctgcaagcactggcatgcacgcgagggctgggagtgtgttttttgtggagatgagtgcactggccttcttctcggtgacttggctcgcctggagcagatggtcatgagcatcaacctcactggtccgctgcctgcgccatataaaatgctgtatggtcttgaaaatatgactcaggagctaaagcacttgctgtcacctcagcgggccccagagaggcttattcagctggcagagggcaatctgaatacactcgtgaccgaaatgaacgagctgctgaccagggctaccaaagtgacagcagatggcgagcagaccggacaggatgctgagaggaccaacacaagagcaaagtccctgggagaattcattaaggagcttgcccgggatgcagaagctgtaaatgaaaaagctataaaactaaatgaaactctaggaactcgagacgaggcctttgagagaaatttggaagggcttcagaaagagattgaccagatgattaaagaactgaggaggaaaaatctagagacacaaaaggaaattgctgaagatgagttggtagctgcagaagcccttctgaaaaaagtgaagaagctgtttggagagtcccggggggaaaatgaagaaatggagaaggatctccgggaaaaactggctgactacaaaaacaaagttgatgatgcttgggaccttttgagagaagccacagataaaatcagagaagctaatcgcctatttgcagtaaatcagaaaaacatgactgcattggagaaaaagaaggaggctgttgaaagcggcaaacgacaaattgagaacactttaaaagagggcaatgacatactcgatgaagccaaccgtcttgcagatgaaatcaactccatcatagactatgttgaagacatccaaactaaattgccacctatgtctgaggagcttaatgataaaatagatgacctctcccaagaaataaaggacaggaagcttgctgagaaggtgtcccaggctgagagccacgcagctcagttgaatgactcatctgctgtccttgatggaatccttgatgaggctaaaaacatctccttcaatgccactgcagccttcaaagcttacagcaatattaaggactatattgatgaagctgagaaagttgccaaagaagccaaagatcttgcacatgaagctacaaaactggcaacaggtcctcggggtttattaaaggaagatgccaaaggctgtcttcagaaaagcttcaggattcttaacgaagccaagaagttagcaaatgatgtaaaagaaaatgaagaccatctaaatggcttaaaaaccaggatagaaaatgctgatgctagaaatggggatctcttgagaactttgaatgacactttgggaaagttatcagctattccaaatgatacagctgctaaactgcaagctgttaaggacaaagccagacaagccaacgacacagctaaagatgtactggcacagattacagagctccaccagaacctcgatggcctgaagaagaattacaataaactagcagacagcgtcgccaaaacgaatgctgtggttaaagatccttccaagaacaaaatcattgccgatgcagatgccactgtcaaaaatttagaacaggaagctgaccggctaatagataaactcaaacccatcaaggaacttgaggataacctaaagaaaaacatctctgagataaaggaattgataaaccaagctcggaaacaagccaattctatcaaagtatctgtgtcttcaggaggtgactgcattcgaacatacaaaccagaaatcaagaaaggaagttacaataatattgttgtcaacgtaaagacagctgttgctgataacctcctcttttatcttggaagtgccaaatttattgactttctggctatagaaatgcgtaaaggcaaagtcagcttcctctgggatgttggatctggagttggacgtgtagagtacccagatttgactattgatgactcatattggtaccgtatcgtagcatcaagaactgggagaaatggaactatttctgtgagagccctggatggacccaaagccagcattgtgcccagcacacaccattcgacgtctcctccagggtacacgattctagatgtggatgcaaatgcaatgctgtttgttggtggcctgactgggaaattaaagaaggctgatgctgtacgtgtgattacattcactggctgcatgggagaaacatactttgacaacaaacctataggtttgtggaatttccgagaaaaagaaggtgactgcaaaggatgcactgtcagtcctcaggtggaagatagtgaggggactattcaatttgatggagaaggttatgcattggtcagccgtcccattcgctggtaccccaacatctccactgtcatgttcaagttcagaacattttcttcgagtgctcttctgatgtatcttgccacacgagacctgagagatttcatgagtgtggagctcactgatgggcacataaaagtcagttacgatctgggctcaggaatggcttccgttgtcagcaatcaaaaccataatgatgggaaatggaaatcattcactctgtcaagaattcaaaaacaagccaatatatcaattgtagatatagatactaatcaggaggagaatatagcaacttcgtcttctggaaacaactttggtcttgacttgaaagcagatgacaaaatatattttggtggcctgccaacgctgagaaacttgaggccagaagtaaatctgaagaaatattccggctgcctcaaagatattgaaatttcaagaactccgtacaatatactcagtagtcccgattatgttggtgttaccaaaggatgttccctggagaatgtttacacagttagctttcctaagcctggttttgtggagctctcccctgtgccaattgatgtaggaacagaaatcaacctgtcattcagcaccaagaatgagtccggcatcattcttttgggaagtggagggacaccagcaccacctaggagaaaacgaaggcagactggacaggcctattatgtaatactcctcaacaggggccgtctggaagtgcatctctccacaggggcacgaacaatgaggaaaattgtgatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctcgcatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattccgcacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacaggcaagccactggaggttaattttgccaaggccctggaactgaggggcgttcaacctgtatcatgcccagccaactaataaaaataagtgtaaccccaggaagagtctgtcaaaacaagtatatcaagtaaaacaaacaaatatattttacctatatatgttaattaaactaatttgtgcatgtacatagaattctttctgtattcagatggtgctaattcagactccagactgaattttaattcaagttctttctcaagtctataaataatattaaactgattatttcattctaaaaaaaaaaaaaaaaaa

SEQ ID NO: 59 - DNA, 1370 bp, Homo sapiens
cacggccggtctgtgccggctgctcccgcggttaggtcccgccccgcgcagcgcgcgcagcctgcggagccagcggccgtgacgcgacaacgattcggctgtgacgcgacaacgattcggctgtgacgcgagcgcggccgctcccgatgcgctcgtgccgcccccgccgtgctcctcggcagccgttgctcggccggttttggtaggcccgggccgccgccaggcctccgcctgagcccgcacccgccatggacaactacgcagatctttcggataccgagctgaccaccttgctgcgccggtacaacatcccgcacgggcctgtagtaggatcaactcgtaggctttacgagaagaagatcttcgagtacgagacccagaggcggcggctctcgccccccagctcgtccgccgcctcctcttatagcttctctgacttgaattcgactagaggggatgcagatatgtatgatcttcccaagaaagaggacgctttactctaccagagcaagggctacaatgacgactactatgaagagagctacttcaccaccaggacttatggggagcccgagtctgccggcccgtccagggctgtccgccagtcagtgacttcattcccagatgctgacgctttccatcaccaggtgcatgatgacgatcttttgtcttcttctgaagaggagtgcaaggatagggaacgccccatgtacggccgggacagtgcctaccagagcatcacgcactaccgccctgtttcagcctccaggagctccctggacctgtcctattatcctacttcctcctccacctcttttatgtcctcctcatcatcttcctcttcatggctcacccgccgtgccatccggcctgaaaaccgtgctcctggggctgggctgggccaggatcgccaggtcccgctctggggccagctgctgcttttcctggtctttgtgatcgtcctcttcttcatttaccacttcatgcaggctgaagaaggcaaccccttctagagggagccatgagggtctgggcttcagagctaggtctttggggaagtcctggctgactgccttagcagtgggggtgggggtgggggcaggggcaggggctttatgtgtttttgcttggggggcgctgggcctagcccagagtagtgcttgctccccctgccttgtcccaccagggaggcagcagactcaggccctccatggtcctctttgtcattttgttgacatgcattcctccttttgtcatcttgttggggggaggggattaaccaaaggccaccctgactttgtttttgtggacacacaataaaagccccgtttatttgtaaaaaaaaaaaaaaaaaaaaaaaaaaaaaaaaa

SEQ ID NO: 60 - DNA, 6911 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 61 - DNA, 5066 bp, Homo sapiens
  misc_feature (594)..(693): n is a, c, g, or t
  misc_feature (1130)..(1229): n is a, c, g, or t
  misc_feature (3750)..(3849): n is a, c, g, or t
  misc_feature (4480)..(4607): n is a, c, g, or t
aggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcggtgagttcgcccaggtggctgcgtgcctggcggggagtggagagggcggcgggccggcgcccctggccggccgcaggaagggagtgagagggcctggaggccgataactttgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctggtaattgcaggcatagcagcgccagcccccatggctgacctcctgggagcctggcactgtctaggcacacagactccttctcttaaatctactctcccctctcttctttagcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaaggtgaggccaccctgcagggcccacccatggccccacctaacacatgtacactcactcttctacctaggccctcccccatgtggtgcctggtctgacctgtcacctgatttcagagccattcacctgtcctagagtcattttacccactgaggtcacatcttatccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtcacccaggctggagtgcagtagtgcgatctcggctcactgcaacctccacctcctggattcaagcgattcttgtgcctcagcctcctgagtagctgggactacaggcgtgtgccaccatcatgcctggctacttttttgtattagatatatattttctctcttagcacagtacctaccaagagtgagtgagtagatgtcctgacccctgcaggcatccaaggccctccttccctggacctgtttccacatgtgtgaaggggtgcacaggcagcagcccacctctcagcttccttccagttcttgtgttctgtgaccccttttcctcatctctgcctgcttcctcacagcttgaggcagccctaggtgaggccaagaagcaacttcaggatgagatgctgcggcgggtggatgctgagaacaggctgcagaccatgaaggaggaactggacttccagaagaacatctacagtgaggtggggactgtgctttgcaagccagagggctggggctgggtgatgacagacttgggctgggctaggggggaccagctgtgtgcagagctcgccttcctgagtcccttgccctagtggacagggagttgggggtggccagcactcagctcccaggttaaagtggggctggtagtggctcatggagtagggctgggcagggagccccgcccctgggtcttggcctcccaggaactaattctgattttggtttctgtgtccttcctccaacccttccaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaaggtgcttgctctcgattggttccctcactgcctctgcccttggcagccctacccttacccacgctgggctatgccttctggggatcaggcagatggtggcagggagctcagggtggcccaggacctggggctgtagcagtgatgcccaactcaggcctgtgcctccacccctcccagtcaccacagtcctaaccctttgtcctcccctccagctggacaatgccaggcagtctgctgagaggaacagcaacctggtgggggctgcccacgaggagctgcagcagtcgcgcatccgcatcgacagcctctctgcccagctcagccagctccagaagcaggtgataccccacctcacccctctctccaggggcctagagtctgggccggatgcaggctggaagcccagggttgggggtgggggtgggggtgggaggttcctgaggaggagagggatgaaaagtgtccccacaaccacagagaagggtcgcaggatgtggagtcagatggcctgtgtgctgtttctgtacactcttacctcaccttcacttctcagggctttggttttcccattcgaaaatggaggctgttcttaatctccctaactcagagttgccacaggactctgcaatgtgaggtgttaaaagcatcagtatttttctagttggctgtgctatttgtgacaggagaaaaagtctagcctcagaacgagaggtttcagttagacaaggggaaggacttcccagttgccagccaagactatgtttagagcttgtgatgttcagagctggctctgatgagggctctggggaagctctgattgcagatcctggagagagtagccaggtgtctcctacaccgacccacgtccctccttccccatacttagggcccttgggagctcaccaaaccctcccaccccccttcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggtgggctggggagacgtcggggaggtgctggcagtgtcctctggccggcaactggccttgactagacccccacttggtctccctctccccaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggtaggctcctgctcagggtctaaggggatacagctgcatcagggagagagtggcaagacagaaggatggcatgtggagagaggaacatccttgccctcagagggtggaccagggtgagcctgtatatctcctccacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctgcgtatgtgtccacagatcatggctattatccccgggggaagggcagtgacaggggtgtgtgtagatggaaggagaggcctcaattgcaggcaggcagagggctgggcctttgagcaagatacacccaagagcctgggtgagcctccccgaccttcctcttccctatcttcccggcaggaccagtccatgggcaattggcagatcaagcgccagaatggagatgatcccttgctgacttaccggttcccaccaaagttcaccctgaaggctgggcaggtggtgacggtgagtggcagggcgcttgggactctggggaggccttgggtggcgatgggagcgctggggtaagtgtccttttctcctctccagatctgggctgcaggagctggggccacccacagcccccctaccgacctggtgtggaaggcacagaacacctggggctgcgggaacagcctgcgtacggctctcatcaactccactggggaagtaagtaggcctgggcctggctgcttgctggacgaggctccccctgatggccaacatcggagccagctgcccccaacccaagtttgccaattcagggcccctttctagagctctctgttgcaggctccagacttctcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgagttccttagctccatcaccacagaggacagagtaagcagcaggccggacaaagggcaggccacaagaaaagttgcaggtggtcactggggtagacatgctgtacaacccttccctggccctgacccttggacctggttccatgtccccaccaggaagtggccatacgcaagctggtgcgctcagtgactgtggttgaggacgacgaggatgaggatggagatgacctgctccatcaccaccatgtgagtggtagccgccgctgaggccgagcctgcactggggccacccagccaggcctgggggcagcctctccccagcctccccgtgccaaaaatcttttcattaaagaatgttttggaactttactcgctggcctggcctttcttctctctcctccctataccttgaacagggaacccaggtgtctgggtgccctactctggtaaggaagggag

SEQ ID NO: 62 - RNA, 5942 nt, Homo sapiens
ccggguccuucuccgagagccgggcgggcacgcgucauuguguuaccugcggccggcccgcgagcuaggcugguuuuuuuuuuuucuccccucccuccccccuuuuuccaugcagcugaucuaaaagggaauaaaaggcugcgcauaaucauaauaauaaaagaaggggagcgcgagagaaggaaagaaagccgggagguggaagaggagggggagcgucucaaagaagcgaucagaauaauaaaaggaggccgggcucuuugccuucuggaacgggccgcucuugaaagggcuuuugaaaagugguguuguuuuccagucgugcaugcuccaaucggcggaguauauuagagccgggacgcggcggccgcaggggcagcggcgacggcagcaccggcggcagcaccagcgcgaacagcagcggcggcgucccgagugcccgcggcgcgcggcgcagcgaugcguuccccacggacgcgcggccgguccgggcgcccccuaagccuccugcucgcccugcucugugcccugcgagccaagguguguggggccucgggucaguucgaguuggagauccuguccaugcagaacgugaacggggagcugcagaacgggaacugcugcggcggcgcccggaacccgggagaccgcaagugcacccgcgacgagugugacacauacuucaaagugugccucaaggaguaucagucccgcgucacggccggggggcccugcagcuucggcucaggguccacgccugucaucgggggcaacaccuucaaccucaaggccagccgcggcaacgaccgcaaccgcaucgugcugccuuucaguuucgccuggccgagguccuauacguugcuuguggaggcgugggauuccaguaaugacaccguucaaccugacaguauuauugaaaaggcuucucacucgggcaugaucaaccccagccggcaguggcagacgcugaagcagaacacgggcguugcccacuuugaguaucagauccgcgugaccugugaugacuacuacuauggcuuuggcugcaauaaguucugccgccccagagaugacuucuuuggacacuaugccugugaccagaauggcaacaaaacuugcauggaaggcuggaugggccgcgaauguaacagagcuauuugccgacaaggcugcaguccuaagcaugggucuugcaaacucccaggugacugcaggugccaguacggcuggcaaggccuguacugugauaagugcaucccacacccgggaugcguccacggcaucuguaaugagcccuggcagugccucugugagaccaacuggggcggccagcucugugacaaagaucucaauuacugugggacucaucagccgugucucaacgggggaacuuguagcaacacaggcccugacaaauaucaguguuccugcccugagggguauucaggacccaacugugaaauugcugagcacgccugccucucugaucccugucacaacagaggcagcuguaaggagaccucccugggcuuugagugugaguguuccccaggcuggaccggccccacaugcucuacaaacauugaugacuguucuccuaauaacuguucccacgggggcaccugccaggaccugguuaacggauuuaagugugugugccccccacaguggacugggaaaacgugccaguuagaugcaaaugaaugugaggccaaaccuuguguaaacgccaaauccuguaagaaucucauugccagcuacuacugcgacugucuucccggcuggaugggucagaauugugacauaaauauuaaugacugccuuggccagugucagaaugacgccuccugucgggauuugguuaaugguuaucgcuguaucuguccaccuggcuaugcaggcgaucacugugagagagacaucgaugaaugugccagcaaccccuguuuggaugggggucacugucagaaugaaaucaacagauuccagugucugugucccacugguuucucuggaaaccucugucagcuggacaucgauuauugugagccuaaucccugccagaacggugcccagugcuacaaccgugccagugacuauuucugcaagugccccgaggacuaugagggcaagaacugcucacaccugaaagaccacugccgcacgacccccugugaagugauugacagcugcacaguggccauggcuuccaacgacacaccugaaggggugcgguauauuuccuccaacgucugugguccucacgggaagugcaagagucagucgggaggcaaauucaccugugacuguaacaaaggcuucacgggaacauacugccaugaaaauauuaaugacugugagagcaacccuuguagaaacgguggcacuugcaucgauggugucaacuccuacaagugcaucuguagugacggcugggagggggccuacugugaaaccaauauuaaugacugcagccagaaccccugccacaaugggggcacgugucgcgaccuggucaaugacuucuacugugacuguaaaaauggguggaaaggaaagaccugccacucacgugacagucagugugaugaggccacgugcaacaacgguggcaccugcuaugaugagggggaugcuuuuaagugcauguguccuggcggcugggaaggaacaaccuguaacauagcccgaaacaguagcugccugcccaaccccugccauaaugggggcacauguguggucaacggcgaguccuuuacgugcgucugcaaggaaggcugggaggggcccaucugugcucagaauaccaaugacugcagcccucaucccuguuacaacagcggcaccuguguggauggagacaacugguaccggugcgaaugugccccggguuuugcugggcccgacugcagaauaaacaucaaugaaugccagucuucaccuugugccuuuggagcgaccuguguggaugagaucaauggcuaccggugugucugcccuccagggcacaguggugccaagugccaggaaguuucagggagaccuugcaucaccauggggagugugauaccagauggggccaaaugggaugaugacuguaauaccugccagugccugaauggacggaucgccugcucaaaggucugguguggcccucgaccuugccugcuccacaaagggcacagcgagugccccagcgggcagagcugcauccccauccuggacgaccagugcuucguccaccccugcacuggugugggcgagugucggucuuccagucuccagccggugaagacaaagugcaccucugacuccuauuaccaggauaacugugcgaacaucacauuuaccuuuaacaaggagaugaugucaccaggucuuacuacggagcacauuugcagugaauugaggaauuugaauauuuugaagaauguuuccgcugaauauucaaucuacaucgcuugcgagccuuccccuucagcgaacaaugaaauacauguggccauuucugcugaagauauacgggaugaugggaacccgaucaaggaaaucacugacaaaauaauugaucuuguuaguaaacgugauggaaacagcucgcugauugcugccguugcagaaguaagaguucagaggcggccucugaagaacagaacagauuuccuuguucccuugcugagcucugucuuaacuguggcuuggaucuguugcuuggugacggccuucuacuggugccugcggaagcggcggaagccgggcagccacacacacucagccucugaggacaacaccaccaacaacgugcgggagcagcugaaccagaucaaaaaccccauugagaaacauggggccaacacgguccccaucaaggauuacgagaacaagaacuccaaaaugucuaaaauaaggacacacaauucugaaguagaagaggacgacauggacaaacaccagcagaaagcccgguuugccaagcagccggcguauacgcugguagacagagaagagaagccccccaacggcacgccgacaaaacacccaaacuggacaaacaaacaggacaacagagacuuggaaagugcccagagcuuaaaccgaauggaguacaucguauagcagaccgcgggcacugccgccgcuagguagagucugagggcuuguaguucuuuaaacugucgugucauacucgagucugaggccguugcugacuuagaaucccuguguuaauuuaaguuuugacaagcuggcuuacacuggcaaugguaguuucugugguuggcugggaaaucgagugccgcaucucacagcuaugcaaaaagcuagucaacaguacccugguuguguguccccuugcagccgacacggucucggaucaggcucccaggagccugcccagcccccuggucuuugagcucccacuucugccagauguccuaauggugaugcagucuuagaucauaguuuuauuuauauuuauugacucuugaguuguuuuuguauauugguuuuaugaugacguacaaguaguucuguauuugaaagugccuuugcagcucagaaccacagcaacgaucacaaaugacuuuauuauuuauuuuuuuaauuguauuuuuguuguugggggaggggagacuuugaugucagcaguugcugguaaaaugaagaauuuaaagaaaaaaaugucaaaaguagaacuuuguauaguuauguaaauaauucuuuuuuauuaaucacuguguauauuugauuuauuaacuuaauaaucaagagccuuaaaacaucauuccuuuuuauuuauauguauguguuuagaauugaagguuuuugauagcauuguaagcguauggcuuuauuuuuuugaacucuucucauuacuuguugccuauaagccaaaauuaagguguuugaaaauaguuuauuuuaaaacaauaggaugggcuucugugcccagaauacugauggaauuuuuuuuguacgacgucagauguuuaaaacaccuucuauagcaucacuuaaaacacguuuuaaggacugacugaggcaguuugaggauuaguuuagaacagguuuuuuuguuuguuuguuuuuuguuuuucugcuuuagacuugaaaagagacaggcaggugaucugcugcagagcaguaagggaacaaguugagcuaugacuuaacauagccaaaaugugagugguugaauaugauuaaaaauaucaaauuaauugugugaacuuggaagcacaccaaucugacuuuguaaauucugauuucuuuucaccauucguacauaauacugaaccacuuguagauuugauuuuuuuuuuaaucuacugcauuuagggaguauucuaauaagcuaguugaauacuugaaccauaaaauguccaguaagaucacuguuuagauuugccauagaguacacugccugccuuaagugaggaaaucaaagugcuauuacgaaguucaagaucaaaaaggcuuauaaaacagaguaaucuuguugguucaccauugagaccgugaagauacuuuguauuguccuauuaguguuauaugaacauacaaaugcaucuuugauguguuguucuuggcaauaaauuuugaaaaguaauauuuauuaaauuuuuuuguaugaaaacauggaacaguguggcucuucugagcuuacguaguucuaccggcuuugccgugugcuucugccacccugcugagucuguucugguaaucgggguauaauaggcucugccugacagagggauggaggaagaacugaaaggcuuuucaaccacaaaacucaucuggaguucucaaagaccuggggcugcugugaagcuggaacugcgggagccccaucuaggggagccuugauucccuuguuauucaacagcaagugugaauacugcuugaauaaacaccacuggauuaauggaaaaaaaaaaaaaaaa

SEQ ID NO: 63 - RNA, 13957 nt, Homo sapiens
gggauucccucacuuucccccuacaggacucagaucugggaggcaauuaccuucggagaaaaacgaauaggaaaaacugaaguguuacuuuuuuuaaagcugcugaaguuuguugguuucucauuguuuuuaagccuacuggagcaauaaaguuugaagaacuuuuaccagguuuuuuuuaucgcugccuugauauacacuuuucaaaaugcuuuggugggaagaaguagaggacuguuaugaaagagaagauguucaaaagaaaacauucacaaaauggguaaaugcacaauuuucuaaguuugggaagcagcauauugagaaccucuucagugaccuacaggaugggaggcgccuccuagaccuccucgaaggccugacagggcaaaaacugccaaaagaaaaaggauccacaagaguucaugcccugaacaaugucaacaaggcacugcggguuuugcagaacaauaauguugauuuagugaauauuggaaguacugacaucguagauggaaaucauaaacugacucuugguuugauuuggaauauaauccuccacuggcaggucaaaaauguaaugaaaaauaucauggcuggauugcaacaaaccaacagugaaaagauucuccugagcuggguccgacaaucaacucguaauuauccacagguuaauguaaucaacuucaccaccagcuggucugauggccuggcuuugaaugcucucauccauagucauaggccagaccuauuugacuggaauagugugguuugccagcagucagccacacaacgacuggaacaugcauucaacaucgccagauaucaauuaggcauagagaaacuacucgauccugaagauguugauaccaccuauccagauaagaaguccaucuuaauguacaucacaucacucuuccaaguuuugccucaacaagugagcauugaagccauccaggaaguggaaauguugccaaggccaccuaaagugacuaaagaagaacauuuucaguuacaucaucaaaugcacuauucucaacagaucacggucagucuagcacagggauaugagagaacuucuuccccuaagccucgauucaagagcuaugccuacacacaggcugcuuaugucaccaccucugacccuacacggagcccauuuccuucacagcauuuggaagcuccugaagacaagucauuuggcaguucauugauggagagugaaguaaaccuggaccguuaucaaacagcuuuagaagaaguauuaucguggcuucuuucugcugaggacacauugcaagcacaaggagagauuucuaaugauguggaaguggugaaagaccaguuucauacucaugagggguacaugauggauuugacagcccaucagggccggguugguaauauucuacaauugggaaguaagcugauuggaacaggaaaauuaucagaagaugaagaaacugaaguacaagagcagaugaaucuccuaaauucaagaugggaaugccucaggguagcuagcauggaaaaacaaagcaauuuacauagaguuuuaauggaucuccagaaucagaaacugaaagaguugaaugacuggcuaacaaaaacagaagaaagaacaaggaaaauggaggaagagccucuuggaccugaucuugaagaccuaaaacgccaaguacaacaacauaaggugcuucaagaagaucuagaacaagaacaagucagggucaauucucucacucacaugguggugguaguugaugaaucuaguggagaucacgcaacugcugcuuuggaagaacaacuuaagguauugggagaucgaugggcaaacaucuguagauggacagaagaccgcuggguucuuuuacaagacauccuucucaaauggcaacgucuuacugaagaacagugccuuuuuagugcauggcuuucagaaaaagaagaugcagugaacaagauucacacaacuggcuuuaaagaucaaaaugaaauguuaucaagucuucaaaaacuggccguuuuaaaagcggaucuagaaaagaaaaagcaauccaugggcaaacuguauucacucaaacaagaucuucuuucaacacugaagaauaagucagugacccagaagacggaagcauggcuggauaacuuugcccgguguugggauaauuuaguccaaaaacuugaaaagaguacagcacagauuucacaggcugucaccaccacucagccaucacuaacacagacaacuguaauggaaacaguaacuacggugaccacaagggaacagauccugguaaagcaugcucaagaggaacuuccaccaccaccuccccaaaagaagaggcagauuacuguggauucugaaauuaggaaaagguuggauguugauauaacugaacuucacagcuggauuacucgcucagaagcuguguugcagaguccugaauuugcaaucuuucggaaggaaggcaacuucucagacuuaaaagaaaaagucaaugccauagagcgagaaaaagcugagaaguucagaaaacugcaagaugccagcagaucagcucaggcccugguggaacagauggugaaugaggguguuaaugcagauagcaucaaacaagccucagaacaacugaacagccgguggaucgaauucugccaguugcuaagugagagacuuaacuggcuggaguaucagaacaacaucaucgcuuucuauaaucagcuacaacaauuggagcagaugacaacuacugcugaaaacugguugaaaauccaacccaccaccccaucagagccaacagcaauuaaaagucaguuaaaaauuuguaaggaugaagucaaccggcuaucaggucuucaaccucaaauugaacgauuaaaaauucaaagcauagcccugaaagagaaaggacaaggacccauguuccuggaugcagacuuuguggccuuuacaaaucauuuuaagcaagucuuuucugaugugcaggccagagagaaagagcuacagacaauuuuugacacuuugccaccaaugcgcuaucaggagaccaugagugccaucaggacauggguccagcagucagaaaccaaacucuccauaccucaacuuagugucaccgacuaugaaaucauggagcagagacucggggaauugcaggcuuuacaaaguucucugcaagagcaacaaaguggccuauacuaucucagcaccacugugaaagagaugucgaagaaagcgcccucugaaauuagccggaaauaucaaucagaauuugaagaaauugagggacgcuggaagaagcucuccucccagcugguugagcauugucaaaagcuagaggagcaaaugaauaaacuccgaaaaauucagaaucacauacaaacccugaagaaauggauggcugaaguugauguuuuucugaaggaggaauggccugcccuuggggauucagaaauucuaaaaaagcagcugaaacagugcagacuuuuagucagugauauucagacaauucagcccagucuaaacagugucaaugaaggugggcagaagauaaagaaugaagcagagccagaguuugcuucgagacuugagacagaacucaaagaacuuaacacucagugggaucacaugugccaacaggucuaugccagaaaggaggccuugaagggagguuuggagaaaacuguaagccuccagaaagaucuaucagagaugcacgaauggaugacacaagcugaagaagaguaucuugagagagauuuugaauauaaaacuccagaugaauuacagaaagcaguugaagagaugaagagagcuaaagaagaggcccaacaaaaagaagcgaaagugaaacuccuuacugagucuguaaauagugucauagcucaagcuccaccuguagcacaagaggccuuaaaaaaggaacuugaaacucuaaccaccaacuaccaguggcucugcacuaggcugaaugggaaaugcaagacuuuggaagaaguuugggcauguuggcaugaguuauugucauacuuggagaaagcaaacaaguggcuaaaugaaguagaauuuaaacuuaaaaccacugaaaacauuccuggcggagcugaggaaaucucugaggugcuagauucacuugaaaauuugaugcgacauucagaggauaacccaaaucagauucgcauauuggcacagacccuaacagauggcggagucauggaugagcuaaucaaugaggaacuugagacauuuaauucucguuggagggaacuacaugaagaggcuguaaggaggcaaaaguugcuugaacagagcauccagucugcccaggagacugaaaaauccuuacacuuaauccaggagucccucacauucauugacaagcaguuggcagcuuauauugcagacaagguggacgcagcucaaaugccucaggaagcccagaaaauccaaucugauuugacaagucaugagaucaguuuagaagaaaugaagaaacauaaucaggggaaggaggcugcccaaagaguccugucucagauugauguugcacagaaaaaauuacaagaugucuccaugaaguuucgauuauuccagaaaccagccaauuuugagcugcgucuacaagaaaguaagaugauuuuagaugaagugaagaugcacuugccugcauuggaaacaaagaguguggaacaggaaguaguacagucacagcuaaaucauugugugaacuuguauaaaagucugagugaagugaagucugaaguggaaauggugauaaagacuggacgucagauuguacagaaaaagcagacggaaaaucccaaagaacuugaugaaagaguaacagcuuugaaauugcauuauaaugagcugggagcaaagguaacagaaagaaagcaacaguuggagaaaugcuugaaauugucccguaagaugcgaaaggaaaugaaugucuugacagaauggcuggcagcuacagauauggaauugacaaagagaucagcaguugaaggaaugccuaguaauuuggauucugaaguugccuggggaaaggcuacucaaaaagagauugagaaacagaaggugcaccugaagaguaucacagagguaggagaggccuugaaaacaguuuugggcaagaaggagacguugguggaagauaaacucagucuucugaauaguaacuggauagcugucaccucccgagcagaagagugguuaaaucuuuuguuggaauaccagaaacacauggaaacuuuugaccagaauguggaccacaucacaaaguggaucauucaggcugacacacuuuuggaugaaucagagaaaaagaaaccccagcaaaaagaagacgugcuuaagcguuuaaaggcagaacugaaugacauacgcccaaagguggacucuacacgugaccaagcagcaaacuugauggcaaaccgcggugaccacugcaggaaauuaguagagccccaaaucucagagcucaaccaucgauuugcagccauuucacacagaauuaagacuggaaaggccuccauuccuuugaaggaauuggagcaguuuaacucagauauacaaaaauugcuugaaccacuggaggcugaaauucagcagggggugaaucugaaagaggaagacuucaauaaagauaugaaugaagacaaugaggguacuguaaaagaauuguugcaaagaggagacaacuuacaacaaagaaucacagaugagagaaagagagaggaaauaaagauaaaacagcagcuguuacagacaaaacauaaugcucucaaggauuugaggucucaaagaagaaaaaaggcucuagaaauuucucaucagugguaucaguacaagaggcaggcugaugaucuccugaaaugcuuggaugacauugaaaaaaaauuagccagccuaccugagcccagagaugaaaggaaaauaaaggaaauugaucgggaauugcagaagaagaaagaggagcugaaugcagugcguaggcaagcugagggcuugucugaggauggggccgcaauggcaguggagccaacucagauccagcucagcaagcgcuggcgggaaauugagagcaaauuugcucaguuucgaagacucaacuuugcacaaauucacacuguccgugaagaaacgaugauggugaugacugaagacaugccuuuggaaauuucuuaugugccuucuacuuauuugacugaaaucacucaugucucacaagcccuauuagaaguggaacaacuucucaaugcuccugaccucugugcuaaggacuuugaagaucucuuuaagcaagaggagucucugaagaauauaaaagauagucuacaacaaagcucaggucggauugacauuauucauagcaagaagacagcagcauugcaaagugcaacgccuguggaaagggugaagcuacaggaagcucucucccagcuugauuuccaaugggaaaaaguuaacaaaauguacaaggaccgacaagggcgauuugacagaucuguugagaaauggcggcguuuucauuaugauauaaagauauuuaaucaguggcuaacagaagcugaacaguuucucagaaagacacaaauuccugagaauugggaacaugcuaaauacaaaugguaucuuaaggaacuccaggauggcauugggcagcggcaaacuguugucagaacauugaaugcaacuggggaagaaauaauucagcaauccucaaaaacagaugccaguauucuacaggaaaaauugggaagccugaaucugcgguggcaggaggucugcaaacagcugucagacagaaaaaagaggcuagaagaacaaaagaauaucuugucagaauuucaaagagauuuaaaugaauuuguuuuaugguuggaggaagcagauaacauugcuaguaucccacuugaaccuggaaaagagcagcaacuaaaagaaaagcuugagcaagucaaguuacugguggaagaguugccccugcgccagggaauucucaaacaauuaaaugaaacuggaggacccgugcuuguaagugcucccauaagcccagaagagcaagauaaacuugaaaauaagcucaagcagacaaaucuccaguggauaaagguuuccagagcuuuaccugagaaacaaggagaaauugaagcucaaauaaaagaccuugggcagcuugaaaaaaagcuugaagaccuugaagagcaguuaaaucaucugcugcugugguuaucuccuauuaggaaucaguuggaaauuuauaaccaaccaaaccaagaaggaccauuugacguucaggaaacugaaauagcaguucaagcuaaacaaccggauguggaagagauuuugucuaaagggcagcauuuguacaaggaaaaaccagccacucagccagugaagaggaaguuagaagaucugagcucugaguggaaggcgguaaaccguuuacuucaagagcugagggcaaagcagccugaccuagcuccuggacugaccacuauuggagccucuccuacucagacuguuacucuggugacacaaccugugguuacuaaggaaacugccaucuccaaacuagaaaugccaucuuccuugauguuggagguaccugcucuggcagauuucaaccgggcuuggacagaacuuaccgacuggcuuucucugcuugaucaaguuauaaaaucacagagggugauggugggugaccuugaggauaucaacgagaugaucaucaagcagaaggcaacaaugcaggauuuggaacagaggcguccccaguuggaagaacucauuaccgcugcccaaaauuugaaaaacaagaccagcaaucaagaggcuagaacaaucauuacggaucgaauugaaagaauucagaaucagugggaugaaguacaagaacaccuucagaaccggaggcaacaguugaaugaaauguuaaaggauucaacacaauggcuggaagcuaaggaagaagcugagcaggucuuaggacaggccagagccaagcuugagucauggaaggagggucccuauacaguagaugcaauccaaaagaaaaucacagaaaccaagcaguuggccaaagaccuccgccaguggcagacaaauguagauguggcaaaugacuuggcccugaaacuucuccgggauuauucugcagaugauaccagaaaaguccacaugauaacagagaauaucaaugccucuuggagaagcauucauaaaagggugagugagcgagaggcugcuuuggaagaaacucauagauuacugcaacaguucccccuggaccuggaaaaguuucuugccuggcuuacagaagcugaaacaacugccaauguccuacaggaugcuacccguaaggaaaggcuccuagaagacuccaagggaguaaaagagcugaugaaacaauggcaagaccuccaaggugaaauugaagcucacacagauguuuaucacaaccuggaugaaaacagccaaaaaauccugagaucccuggaagguuccgaugaugcaguccuguuacaaagacguuuggauaacaugaacuucaaguggagugaacuucggaaaaagucucucaacauuaggucccauuuggaagccaguucugaccaguggaagcgucugcaccuuucucugcaggaacuucugguguggcuacagcugaaagaugaugaauuaagccggcaggcaccuauuggaggcgacuuuccagcaguucagaagcagaacgauguacauagggccuucaagagggaauugaaaacuaaagaaccuguaaucaugaguacucuugagacuguacgaauauuucugacagagcagccuuuggaaggacuagagaaacucuaccaggagcccagagagcugccuccugaggagagagcccagaaugucacucggcuucuacgaaagcaggcugaggaggucaauacugagugggaaaaauugaaccugcacuccgcugacuggcagagaaaaauagaugagacccuugaaagacuccaggaacuucaagaggccacggaugagcuggaccucaagcugcgccaagcugaggugaucaagggauccuggcagcccgugggcgaucuccucauugacucucuccaagaucaccucgagaaagucaaggcacuucgaggagaaauugcgccucugaaagagaacgugagccacgucaaugaccuugcucgccagcuuaccacuuugggcauucagcucucaccguauaaccucagcacucuggaagaccugaacaccagauggaagcuucugcagguggccgucgaggaccgagucaggcagcugcaugaagcccacagggacuuugguccagcaucucagcacuuucuuuccacgucuguccagggucccugggagagagccaucucgccaaacaaagugcccuacuauaucaaccacgagacucaaacaacuugcugggaccaucccaaaaugacagagcucuaccagucuuuagcugaccugaauaaugucagauucucagcuuauaggacugccaugaaacuccgaagacugcagaaggcccuuugcuuggaucucuugagccugucagcugcaugugaugccuuggaccagcacaaccucaagcaaaaugaccagcccauggauauccugcagauuauuaauuguuugaccacuauuuaugaccgccuggagcaagagcacaacaauuuggucaacgucccucucugcguggauaugugucugaacuggcugcugaauguuuaugauacgggacgaacagggaggauccguguccugucuuuuaaaacuggcaucauuucccuguguaaagcacauuuggaagacaaguacagauaccuuuucaagcaaguggcaaguucaacaggauuuugugaccagcgcaggcugggccuccuucugcaugauucuauccaaauuccaagacaguugggugaaguugcauccuuugggggcaguaacauugagccaaguguccggagcugcuuccaauuugcuaauaauaagccagagaucgaagcggcccucuuccuagacuggaugagacuggaaccccaguccaugguguggcugcccguccugcacagaguggcugcugcagaaacugccaagcaucaggccaaauguaacaucugcaaagaguguccaaucauuggauucagguacaggagucuaaagcacuuuaauuaugacaucugccaaagcugcuuuuuuucuggucgaguugcaaaaggccauaaaaugcacuaucccaugguggaauauugcacuccgacuacaucaggagaagauguucgagacuuugccaagguacuaaaaaacaaauuucgaaccaaaagguauuuugcgaagcauccccgaaugggcuaccugccagugcagacugucuuagagggggacaacauggaaacucccguuacucugaucaacuucuggccaguagauucugcgccugccucguccccucagcuuucacacgaugauacucauucacgcauugaacauuaugcuagcaggcuagcagaaauggaaaacagcaauggaucuuaucuaaaugauagcaucucuccuaaugagagcauagaugaugaacauuuguuaauccagcauuacugccaaaguuugaaccaggacuccccccugagccagccucguaguccugcccagaucuugauuuccuuagagagugaggaaagaggggagcuagagagaauccuagcagaucuugaggaagaaaacaggaaucugcaagcagaauaugaccgucuaaagcagcagcacgaacauaaaggccuguccccacugccguccccuccugaaaugaugcccaccucuccccagaguccccgggaugcugagcucauugcugaggccaagcuacugcgucaacacaaaggccgccuggaagccaggaugcaaauccuggaagaccacaauaaacagcuggagucacaguuacacaggcuaaggcagcugcuggagcaaccccaggcagaggccaaagugaauggcacaacgguguccucuccuucuaccucucuacagagguccgacagcagucagccuaugcugcuccgagugguuggcagucaaacuucggacuccaugggugaggaagaucuucucaguccuccccaggacacaagcacaggguuagaggaggugauggagcaacucaacaacuccuucccuaguucaagaggaagaaauaccccuggaaagccaaugagagaggacacaauguaggaagucuuuuccacauggcagaugauuugggcagagcgauggaguccuuaguaucagucaugacagaugaagaaggagcagaauaaauguuuuacaacuccugauucccgcaugguuuuuauaauauucauacaacaaagaggauuagacaguaagaguuuacaagaaauaaaucuauauuuuugugaaggguagugguauuauacuguagauuucaguaguuucuaagucuguuauuguuuuguuaacaauggcagguuuuacacgucuaugcaauuguacaaaaaaguuauaagaaaacuacauguaaaaucuugauagcuaaauaacuugccauuucuuuauauggaacgcauuuuggguuguuuaaaaauuuauaacaguuauaaagaaagauuguaaacuaaagugugcuuuauaaaaaaaaguuguuuauaaaaaccccuaaaaacaaaacaaacacacacacacacacauacacacacacacacaaaacuuugaggcagcgcauuguuuugcauccuuuuggcgugauauccauaugaaauucauggcuuuuucuuuuuuugcauauuaaagauaagacuuccucuaccaccacaccaaaugacuacuacacacugcucauuugagaacugucagcugaguggggcaggcuugaguuuucauuucauauaucuauaugucuauaaguauauaaauacuauaguuauauagauaaagagauacgaauuucuauagacugacuuuuuccauuuuuuaaauguucaugucacauccuaauagaaagaaauuacuucuagucagucauccaggcuuaccugcuuggucuagaauggauuuuucccggagccggaagccaggaggaaacuacaccacacuaaaacauugucuacagcuccagauguuucucauuuuaaacaacuuuccacugacaacgaaaguaaaguaaaguauuggauuuuuuuaaagggaacaugugaaugaauacacaggacuuauuauaucagagugaguaaucgguugguugguugauugauugauugauugauacauucagcuuccugcugcuagcaaugccacgauuuagauuuaaugaugcuucaguggaaaucaaucagaagguauucugaccuugugaacaucagaagguauuuuuuaacucccaagcaguagcaggacgaugauagggcuggagggcuauggauucccagcccaucccugugaaggaguaggccacucuuuaagugaaggauuggaugauuguucauaauacauaaaguucucuguaauuacaacuaaauuauuaugcccucuucucacagucaaaaggaacugggugguuugguuuuuguugcuuuuuuagauuuauugucccaugugggaugaguuuuuaaaugccacaagacauaauuuaaaauaaauaaacuuugggaaaagguguaagacaguagccccaucacauuugugauacugacagguaucaacccagaagcccaugaacuguguuuccauccuuugcauuucucugcgaguaguuccacacagguuuguaaguaaguaagaaagaaggcaaauugauucaaauguuacaaaaaaacccuucuugguggauuagacagguuaaauauauaaacaaacaaacaaaaauugcucaaaaaagaggagaaaagcucaagaggaaaagcuaaggacugguaggaaaaagcuuuacucuuucaugccauuuuauuucuuuuugauuuuuaaaucauucauucaauagauaccaccgugugaccuauaauuuugcaaaucuguuaccucugacaucaaguguaauuagcuuuuggagagugggcugacaucaaguguaauuagcuuuuggagaguggguuuuguccauuauuaauaauuaauuaauuaacaucaaacacggcuucucaugcuauuucuaccucacuuugguuuugggguguuccugauaauugugcacaccugaguucacagcuucaccacuuguccauugcguuauuuucuuuuuccuuuauaauucuuucuuuuuccuucauaauuuucaaaagaaaacccaaagcucuaagguaacaaauuaccaaauuacaugaagauuugguuuuugucuugcauuuuuuuccuuuaugugacgcuggaccuuuucuuuacccaaggauuuuuaaaacucagauuuaaaacaagggguuacuuuacauccuacuaagaaguuuaaguaaguaaguuucauucuaaaaucagagguaaauagagugcauaaauaauuuuguuuuaaucuuuuuguuuuucuuuuagacacauuagcucuggagugagucugucauaauauuugaacaaaaauugagagcuuuauugcugcauuuuaagcauaauuaauuuggacauuauuucguguuguguucuuuauaaccaccgaguauuaaacuguaaaucauaauguaacugaagcauaaacaucacauggcauguuuugucauuguuuucagguacugaguucuuacuugaguaucauaauauauuguguuuuaacaccaacacuguaacauuuacgaauuauuuuuuuaaacuucaguuuuacugcauuuucacaacauaucagacuucaccaaauauaugccuuacuauuguauuauaguacugcuuuacuguguaucucaauaaagcacgcaguuauguuac

SEQ ID NO: 64 - RNA, 9696 nt, Homo sapiens
uuccccagcagcugcugcucgcucagcucacaagccaaggccaggggacagggcggcagcgacuccucuggcucccgagaaguggauccggucgcggccacuacgaugccgggagccgccgggguccuccuccuucugcugcucuccggaggccucgggggcguacaggcgcagcggccgcagcagcagcggcagucacaggcacaucagcaaagagguuuauucccugcuguccugaaucuugcuucuaaugcucuuaucacgaccaaugcaacauguggagaaaaaggaccugaaauguacugcaaauugguagaacaugucccugggcagccugugaggaacccgcagugucgaaucugcaaucaaaacagcagcaauccaaaccagagacacccgauuacaaaugcuauugauggaaagaacacuugguggcagagucccaguauuaagaauggaaucgaauaccauuaugugacaauuacccuggauuuacagcagguguuccagaucgcguaugugauugugaaggcagcuaacuccccccggccuggaaacuggauuuuggaacgcucucuugaugauguugaauacaagcccuggcaguaucaugcugugacagacacggagugccuaacgcuuuacaauauuuauccccgcacugggccaccgucauaugccaaagaugaugaggucaucugcacuucauuuuacuccaagauacaccccuuagaaaauggagagauucacaucucuuuaaucaaugggagaccaagugccgaugauccuucuccagaacugcuagaauuuaccuccgcucgcuauauucgccugagauuucagaggauccgcacacugaaugcugacuugaugauguuugcucacaaagacccaagagaaauugaccccauugucaccagaagauauuacuacucggucaaggauauuucaguuggagggaugugcaucugcuauggucaugccagggcuuguccacuugauccagcgacaaauaaaucucgcugugagugugagcauaacacauguggcgauagcugugaucagugcuguccaggauuccaucagaaacccuggagagcuggaacuuuucuaacuaaaacugaaugugaagcaugcaauugucauggaaaagcugaagaaugcuauuaugaugaaaauguugccagaagaaaucugaguuugaauauacguggaaaguacauuggagggggugucugcauuaauuguacccaaaacacugcugguauaaacugcgagacauguacugauggcuucuucagacccaaagggguaucuccaaauuauccaaggccaugccagccaugucauugcgauccaauugguuccuuaaaugaagucugugucaaggaugagaaacaugcucgacgagguuuggcaccuggauccugucauugcaaaacugguuuuggaggugugagcugugaucggugugccaggggcuacacuggcuacccggacugcaaagccuguaacugcaguggguuagggagcaaaaaugaggauccuuguuuuggccccuguaucugcaaggaaaauguugaaggaggagacuguagucguugcaaauccggcuucuucaauuugcaagaggauaauuggaaaggcugcgaugaguguuucuguucagggguuucaaacagaugucagaguuccuacuggaccuauggcaaaauacaagauaugaguggcugguaucugacugaccuuccuggccgcauucgaguggcuccccagcaggacgacuuggacucaccucagcagaucagcaucaguaacgcggaggcccggcaagcccugccgcacagcuacuacuggagcgcgccggcucccuaucugggaaacaaacucccagcaguaggaggacaguugacauuuaccauaucauaugaccuugaagaagaggaagaagauacagaacguguucuccagcuuaugauuaucuuagaggguaaugacuugagcaucagcacagcccaagaugagguguaccugcacccaucugaagaacauacuaauguauuguuacuuaaagaagaaucauuuaccauacauggcacacauuuuccaguccguagaaaggaauuuaugacagugcuugcgaauuugaagagaguccuccuacaaaucacauacagcuuugggauggaugccaucuucagguugagcucuguuaaccuugaauccgcugucuccuauccuacugauggaagcauugcagcagcuguagaagugugucagugcccaccaggguauacuggcuccucuugugaaucuuguuggccuaggcacaggcgaguuaacggcacuauuuuugguggcaucugugagccaugucagugcuuuggucaugcggaguccugugaugacgucacuggagaaugccugaacuguaaggaucacacagguggcccauauugugauaaaugucuuccugguuucuauggcgagccuacuaaaggaaccucugaagacugucaacccugugccuguccacucaauaucccauccaauaacuuuagcccaacgugccauuuagaccggagucuuggauugaucugugauggaugcccugucggguacacaggaccacgcugugagaggugugcagaaggcuauuuuggacaacccucuguaccuggaggaucaugucagccaugccaaugcaaugacaaccuugacuucuccaucccuggcagcugugacagcuugucuggcuccugucugauauguaaaccagguacaacaggccgguacugugagcucugugcugauggauauuuuggagaugcaguugaugcgaagaacugucagcccugucgcuguaaugccgguggcucuuucucugagguuugccacagucaaacuggacagugugagugcagagccaacguucagggucagagaugugacaaaugcaaggcugggaccuuuggccuacaaucagcaaggggcuguguucccugcaacugcaauucuuuugggucuaagucauucgacugugaagagaguggacaauguuggugccaaccuggagucacagggaagaaaugugaccgcugugcccacggcuauuucaacuuccaagaaggaggcugcacagcuugugaauguucucaucuggguaauaauugugacccaaagacugggcgaugcauuugcccucccaauaccauuggagagaaauguucuaaaugugcacccaauaccuggggccacagcauuaccacugguuguaaggcuuguaacugcagcacagugggauccuuggauuuccaaugcaauguaaauacaggccaaugcaacugucauccaaaauucucuggugcaaaauguacagagugcagucgaggucacuggaacuacccucgcugcaaucucugugacugcuuccucccugggacagaugccacaaccugugauucagagacuaaaaaaugcuccuguagugaucaaacugggcagugcacuuguaaggugaauguggaaggcauccacugugacagaugccggccuggcaaauucggacucgaugccaagaauccacuuggcugcagcagcugcuauugcuucggcacuacuacccagugcucugaagcaaaaggacugauccggacgugggugacucugaaggcugagcagaccauucuaccccugguagaugaggcucugcagcacacgaccaccaagggcauuguuuuucaacauccagagauuguugcccacauggaccugaugagagaagaucuccauuuggaaccuuuuuauuggaaacuuccagaacaauuugaaggaaagaaguugauggccuaugggggcaaacucaaguaugcaaucuauuucgaggcucgggaagaaacagguuucucuacauauaauccucaagugaucauucgaggugggacaccuacucaugcuagaauuaucgucaggcauauggcugcuccucugauuggccaauugacaaggcaugaaauugaaaugacagagaaagaauggaaauauuauggggaugauccucgaguccauagaacugugacccgagaagacuucuuggauauacuauaugauauucauuacauucuuaucaaagcuacuuauggaaauuucaugcgacaaagcaggauuucugaaaucucaauggagguagcugaacaaggacguggaacaacaaugacuccuccagcugacuugauugaaaaaugugauuguccccugggcuauucuggccuguccugugaggcaugcuugccgggauuuuaucgacugcguucucaaccagguggccgcaccccuggaccaacccugggcaccuguguuccaugucaauguaauggacacagcagccugugugacccugaaacaucgauaugccagaauugucaacaucacacugcuggugacuucugugaacgaugugcucuuggauacuauggaauugucaagggauugccaaaugacugucagcaaugugccugcccucugauuucuuccaguaacaauuucagccccucuugugucgcagaaggacuugacgacuaccgcugcacggcuuguccacggggauaugaaggccaguacugugaaaggugugccccuggcuauacuggcaguccaggcaacccuggaggcuccugccaagaaugugagugugaucccuauggcucacugccugugcccugugacccugucacaggauucugcacgugccgaccuggagccacgggaaggaagugugacggcugcaagcacuggcaugcacgcgagggcugggaguguguuuuuuguggagaugagugcacuggccuucuucucggugacuuggcucgccuggagcagauggucaugagcaucaaccucacugguccgcugccugcgccauauaaaaugcuguauggucuugaaaauaugacucaggagcuaaagcacuugcugucaccucagcgggccccagagaggcuuauucagcuggcagagggcaaucugaauacacucgugaccgaaaugaacgagcugcugaccagggcuaccaaagugacagcagauggcgagcagaccggacaggaugcugagaggaccaacacaagagcaaagucccugggagaauucauuaaggagcuugcccgggaugcagaagcuguaaaugaaaaagcuauaaaacuaaaugaaacucuaggaacucgagacgaggccuuugagagaaauuuggaagggcuucagaaagagauugaccagaugauuaaagaacugaggaggaaaaaucuagagacacaaaaggaaauugcugaagaugaguugguagcugcagaagcccuucugaaaaaagugaagaagcuguuuggagagucccggggggaaaaugaagaaauggagaaggaucuccgggaaaaacuggcugacuacaaaaacaaaguugaugaugcuugggaccuuuugagagaagccacagauaaaaucagagaagcuaaucgccuauuugcaguaaaucagaaaaacaugacugcauuggagaaaaagaaggaggcuguugaaagcggcaaacgacaaauugagaacacuuuaaaagagggcaaugacauacucgaugaagccaaccgucuugcagaugaaaucaacuccaucauagacuauguugaagacauccaaacuaaauugccaccuaugucugaggagcuuaaugauaaaauagaugaccucucccaagaaauaaaggacaggaagcuugcugagaaggugucccaggcugagagccacgcagcucaguugaaugacucaucugcuguccuugauggaauccuugaugaggcuaaaaacaucuccuucaaugccacugcagccuucaaagcuuacagcaauauuaaggacuauauugaugaagcugagaaaguugccaaagaagccaaagaucuugcacaugaagcuacaaaacuggcaacagguccucgggguuuauuaaaggaagaugccaaaggcugucuucagaaaagcuucaggauucuuaacgaagccaagaaguuagcaaaugauguaaaagaaaaugaagaccaucuaaauggcuuaaaaaccaggauagaaaaugcugaugcuagaaauggggaucucuugagaacuuugaaugacacuuugggaaaguuaucagcuauuccaaaugauacagcugcuaaacugcaagcuguuaaggacaaagccagacaagccaacgacacagcuaaagauguacuggcacagauuacagagcuccaccagaaccucgauggccugaagaagaauuacaauaaacuagcagacagcgucgccaaaacgaaugcugugguuaaagauccuuccaagaacaaaaucauugccgaugcagaugccacugucaaaaauuuagaacaggaagcugaccggcuaauagauaaacucaaacccaucaaggaacuugaggauaaccuaaagaaaaacaucucugagauaaaggaauugauaaaccaagcucggaaacaagccaauucuaucaaaguaucugugucuucaggaggugacugcauucgaacauacaaaccagaaaucaagaaaggaaguuacaauaauauuguugucaacguaaagacagcuguugcugauaaccuccucuuuuaucuuggaagugccaaauuuauugacuuucuggcuauagaaaugcguaaaggcaaagucagcuuccucugggauguuggaucuggaguuggacguguagaguacccagauuugacuauugaugacucauauugguaccguaucguagcaucaagaacugggagaaauggaacuauuucugugagagcccuggauggacccaaagccagcauugugcccagcacacaccauucgacgucuccuccaggguacacgauucuagauguggaugcaaaugcaaugcuguuuguugguggccugacugggaaauuaaagaaggcugaugcuguacgugugauuacauucacuggcugcaugggagaaacauacuuugacaacaaaccuauagguuuguggaauuuccgagaaaaagaaggugacugcaaaggaugcacugucaguccucagguggaagauagugaggggacuauucaauuugauggagaagguuaugcauuggucagccgucccauucgcugguaccccaacaucuccacugucauguucaaguucagaacauuuucuucgagugcucuucugauguaucuugccacacgagaccugagagauuucaugaguguggagcucacugaugggcacauaaaagucaguuacgaucugggcucaggaauggcuuccguugucagcaaucaaaaccauaaugaugggaaauggaaaucauucacucugucaagaauucaaaaacaagccaauauaucaauuguagauauagauacuaaucaggaggagaauauagcaacuucgucuucuggaaacaacuuuggucuugacuugaaagcagaugacaaaauauauuuugguggccugccaacgcugagaaacuugaggccagaaguaaaucugaagaaauauuccggcugccucaaagauauugaaauuucaagaacuccguacaauauacucaguagucccgauuauguugguguuaccaaaggauguucccuggagaauguuuacacaguuagcuuuccuaagccugguuuuguggagcucuccccugugccaauugauguaggaacagaaaucaaccugucauucagcaccaagaaugaguccggcaucauucuuuugggaaguggagggacaccagcaccaccuaggagaaaacgaaggcagacuggacaggccuauuauguaauacuccucaacaggggccgucuggaagugcaucucuccacaggggcacgaacaaugaggaaaauugugaucagaccagagccgaaucuguuucaugauggaagagaacauuccguucauguagagcgaacuagaggcaucuuuacaguucaaguggaugaaaacagaagauacaugcaaaaccugacaguugaacagccuaucgaaguuaaaaagcuuuucguugggggugcuccaccugaauuucaaccuuccccacucagaaauauuccuccuuuugaaggcugcauauggaaucuuguuauuaacucuguccccauggacuuugcaaggccuguguccuucaaaaaugcugacauuggucgcugugcccaucagaaacuccgugaagaugaagauggagcagcuccagcugaaauaguuauccagccugagccaguucccaccccagccuuuccuacgcccaccccaguucugacacaugguccuugugcugcagaaucagaaccagcucuuuugauagggagcaagcaguucgggcuuucaagaaacagucacauugcaauugcauuugaugacaccaaaguuaaaaaccgucucacaauugaguuggaaguaagaaccgaagcugaauccggcuugcuuuuuuacauggcucgcaucaaucaugcugauuuugcaacaguucagcugagaaauggauugcccuacuucagcuaugacuuggggaguggggacacccacaccaugauccccaccaaaaucaaugauggccaguggcacaagauuaagauaaugagaaguaagcaagaaggaauucuuuauguagauggggcuuccaacagaaccaucagucccaaaaaagccgacauccuggaugucgugggaaugcuguauguugguggguuacccaucaacuacacuacccgaagaauugguccagugaccuauagcauugauggcugcgucaggaaucuccacauggcagaggccccugccgaucuggaacaacccaccuccagcuuccauguugggacauguuuugcaaaugcucagaggggaacauauuuugacggaaccgguuuugccaaagcaguugguggauucaaagugggauuggaccuucuuguagaauuugaauuccgcacaacuacaacgacuggaguucuucuggggaucaguagucaaaaaauggauggaauggguauugaaaugauugaugaaaaguugauguuucauguggacaauggugcgggcagauucacugcugucuaugaugcugggguuccagggcauuugugugauggacaauggcauaaagucacugccaacaagaucaaacaccgcauugagcucacagucgaugggaaccagguggaagcccaaagcccaaacccagcaucuacaucagcugacacaaaugacccuguguuuguuggaggcuucccagaugaccucaagcaguuuggccuaacaaccaguauuccguuccgagguugcaucagaucccugaagcucaccaaaggcacaggcaagccacuggagguuaauuuugccaaggcccuggaacugaggggcguucaaccuguaucaugcccagccaacuaauaaaaauaaguguaaccccaggaagagucugucaaaacaaguauaucaaguaaaacaaacaaauauauuuuaccuauauauguuaauuaaacuaauuugugcauguacauagaauucuuucuguauucagauggugcuaauucagacuccagacugaauuuuaauucaaguucuuucucaagucuauaaauaauauuaaacugauuauuucauucuaaaaaaaaaaaaaaaaaa

SEQ ID NO: 65 - RNA, 1370 nt, Homo sapiens
cacggccggucugugccggcugcucccgcgguuaggucccgccccgcgcagcgcgcgcagccugcggagccagcggccgugacgcgacaacgauucggcugugacgcgacaacgauucggcugugacgcgagcgcggccgcucccgaugcgcucgugccgcccccgccgugcuccucggcagccguugcucggccgguuuugguaggcccgggccgccgccaggccuccgccugagcccgcacccgccauggacaacuacgcagaucuuucggauaccgagcugaccaccuugcugcgccgguacaacaucccgcacgggccuguaguaggaucaacucguaggcuuuacgagaagaagaucuucgaguacgagacccagaggcggcggcucucgccccccagcucguccgccgccuccucuuauagcuucucugacuugaauucgacuagaggggaugcagauauguaugaucuucccaagaaagaggacgcuuuacucuaccagagcaagggcuacaaugacgacuacuaugaagagagcuacuucaccaccaggacuuauggggagcccgagucugccggcccguccagggcuguccgccagucagugacuucauucccagaugcugacgcuuuccaucaccaggugcaugaugacgaucuuuugucuucuucugaagaggagugcaaggauagggaacgccccauguacggccgggacagugccuaccagagcaucacgcacuaccgcccuguuucagccuccaggagcucccuggaccuguccuauuauccuacuuccuccuccaccucuuuuauguccuccucaucaucuuccucuucauggcucacccgccgugccauccggccugaaaaccgugcuccuggggcugggcugggccaggaucgccaggucccgcucuggggccagcugcugcuuuuccuggucuuugugaucguccucuucuucauuuaccacuucaugcaggcugaagaaggcaaccccuucuagagggagccaugagggucugggcuucagagcuaggucuuuggggaaguccuggcugacugccuuagcagugggggugggggugggggcaggggcaggggcuuuauguguuuuugcuuggggggcgcugggccuagcccagaguagugcuugcucccccugccuugucccaccagggaggcagcagacucaggcccuccaugguccucuuugucauuuuguugacaugcauuccuccuuuugucaucuuguuggggggaggggauuaaccaaaggccacccugacuuuguuuuuguggacacacaauaaaagccccguuuauuuguaaaaaaaaaaaaaaaaaaaaaaaaaaaaaaaaa

SEQ ID NO: 66 - RNA, 6911 nt, Homo sapiens
ucgaccgcccagccaggugcaaaaugccgugucauugggagacuccgcagccggagcauuagauuacagcucgacggagcucgggaagggcggcggggguggaagaugagcagaagccccuguucucggaacgccggcugacaagcggggugagcgcaggcggggcggggacccagccuagcccacuggagcagccggggguggcccguuccccuuuaagagcaacugcucuaagccaggagccagagauucgagccggccucgcccagccagcccucuccagcgaggggacccacaagcggcgccucggcccucccgaccuuuccgagcccucuuugcgcccugggcgcacggggcccuacacgcgccaagcaugcugagggucuucauccucuaugccgagaacguccacacacccgacaccgacaucagcgaugccuacugcuccgcgguguuugcaggggugaagaagagaaccaaagucaucaagaacagcgugaacccuguauggaaugagggauuugaaugggaccucaagggcaucccccuggaccagggcucugagcuucaugugguggucaaagaccaugagacgauggggaggaacagguuccugggggaagccaaggucccacuccgagagguccucgccaccccuagucuguccgccagcuucaaugccccccugcuggacaccaagaagcagcccacaggggccucgcugguccugcagguguccuacacaccgcugccuggagcugugccccuguucccgcccccuacuccucuggagcccuccccgacucugccugaccuggauguaguggcagacacaggaggagaggaagacacagaggaccagggacucacuggagaugaggcggagccauuccuggaucaaagcggaggcccgggggcucccaccaccccaaggaaacuaccuucacguccuccgccccacuaccccgggaucaaaagaaagcgaagugcgccuacaucuagaaagcugcugucagacaaaccgcaggauuuccagaucaggguccaggugaucgaggggcgccagcugccgggggugaacaucaagccuguggucaagguuaccgcugcagggcagaccaagcggacgcggauccacaagggaaacagcccacucuucaaugagacucuuuucuucaacuuguuugacucuccuggggagcuguuugaugagcccaucuuuaucacggugguagacucucguucucucaggacagaugcucuccucggggaguuccggauggacgugggcaccauuuacagagagccccggcacgccuaucucaggaaguggcugcugcucucagacccugaugacuucucugcuggggccagaggcuaccugaaaacaagccuuugugugcuggggccuggggacgaagcgccucuggagagaaaagaccccucugaagacaaggaggacauugaaagcaaccugcuccggcccacaggcguagcccugcgaggagcccacuucugccugaaggucuuccgggccgaggacuugccgcagauggacgaugccgugauggacaacgugaaacagaucuuuggcuucgagaguaacaagaagaacuugguggaccccuuuguggaggucagcuuugcggggaaaaugcugugcagcaagaucuuggagaagacggccaacccucaguggaaccagaacaucacacugccugccauguuucccuccaugugcgaaaaaaugaggauucguaucauagacugggaccgccugacucacaaugacaucguggcuaccaccuaccugaguaugucgaaaaucucugccccuggaggagaaauagaagaggagccugcaggugcugucaagccuucgaaagccucagacuuggaugacuaccugggcuuccuccccacuuuugggcccugcuacaucaaccucuauggcagucccagagaguucacaggcuucccagaccccuacacagagcucaacacaggcaagggggaagguguggcuuaucguggccggcuucugcucucccuggagaccaagcugguggagcacagugaacagaagguggaggaccuuccugcggaugacauccuccggguggagaaguaccuuaggaggcgcaaguacucccuguuugcggccuucuacucagccaccaugcugcaggauguggaugaugccauccaguuugaggucagcaucgggaacuacgggaacaaguucgacaugaccugccugccgcuggccuccaccacucaguacagccgugcagucuuugacgggugccacuacuacuaccuacccugggguaacgugaaaccugugguggugcugucauccuacugggaggacaucagccauagaaucgagacucagaaccagcugcuugggauugcugaccggcuggaagcuggccuggagcagguccaccuggcccugaaggcgcagugcuccacggaggacguggacucgcugguggcucagcugacggaugagcucaucgcaggcugcagccagccucugggugacauccaugagacacccucugccacccaccuggaccaguaccuguaccagcugcgcacccaucaccugagccaaaucacugaggcugcccuggcccugaagcucggccacagugagcucccugcagcucuggagcaggcggaggacuggcuccugcgucugcgugcccuggcagaggagccccagaacagccugccggacaucgucaucuggaugcugcagggagacaagcguguggcauaccagcgggugcccgcccaccaaguccucuucucccggcggggugccaacuacuguggcaagaauugugggaagcuacagacaaucuuucugaaauauccgauggagaaggugccuggcgcccggaugccagugcagauacgggucaagcugugguuugggcucucuguggaugagaaggaguucaaccaguuugcugaggggaagcugucugucuuugcugaaaccuaugagaacgagacuaaguuggcccuuguugggaacuggggcacaacgggccucaccuaccccaaguuuucugacgucacgggcaagaucaagcuacccaaggacagcuuccgccccucggccggcuggaccugggcuggagauugguucguguguccggagaagacucugcuccaugacauggacgccggucaccugagcuucguggaagagguguuugagaaccagacccggcuucccggaggccaguggaucuacaugagugacaacuacaccgaugugaacggggagaaggugcuucccaaggaugacauugagugcccacugggcuggaagugggaagaugaggaaugguccacagaccucaaccgggcugucgaugagcaaggcugggaguauagcaucaccauccccccggagcggaagccgaagcacugggucccugcugagaagauguacuacacacaccgacggcggcgcugggugcgccugcgcaggagggaucucagccaaauggaagcacugaaaaggcacaggcaggcggaggcggagggcgagggcugggaguacgccucucuuuuuggcuggaaguuccaccucgaguaccgcaagacagaugccuuccgccgccgccgcuggcgccgucgcauggagccacuggagaagacggggccugcagcuguguuugcccuugagggggcccugggcggcgugauggaugacaagagugaagauuccauguccgucuccaccuugagcuucggugugaacagacccacgauuuccugcauauucgacuaugggaaccgcuaccaucuacgcugcuacauguaccaggcccgggaccuggcugcgauggacaaggacucuuuuucugaucccuaugccaucgucuccuuccugcaccagagccagaagacggugguggugaagaacacccuuaaccccaccugggaccagacgcucaucuucuacgagaucgagaucuuuggcgagccggccacaguugcugagcaaccgcccagcauugugguggagcuguacgaccaugacacuuauggugcagacgaguuuaugggucgcugcaucugucaaccgagucuggaacggaugccacggcuggccugguucccacugacgaggggcagccagccgucgggggagcugcuggccucuuuugagcucauccagagagagaagccggccauccaccauauuccugguuuugaggugcaggagacaucaaggauccuggaugagucugaggacacagaccugcccuacccaccaccccagagggaggccaacaucuacaugguuccucagaacaucaagccagcgcuccagcguaccgccaucgagauccuggcauggggccugcggaacaugaagaguuaccagcuggccaacaucuccucccccagccucgugguagagugugggggccagacggugcaguccugugucaucaggaaccuccggaagaaccccaacuuugacaucugcacccucuucauggaagugaugcugcccagggaggagcucuacugcccccccaucaccgucaaggucaucgauaaccgccaguuuggccgccggccuguggugggccaguguaccauccgcucccuggagagcuuccugugugaccccuacucggcggagaguccauccccacaggguggcccagacgaugugagccuacucaguccuggggaagacgugcucaucgacauugaugacaaggagccccucauccccauccaggaggaagaguucaucgauugguggagcaaauucuuugccuccauaggggagagggaaaagugcggcuccuaccuggagaaggauuuugacacccugaaggucuaugacacacagcuggagaauguggaggccuuugagggccugucugacuuuuguaacaccuucaagcuguaccggggcaagacgcaggaggagacagaagauccaucugugauuggugaauuuaagggccucuucaaaauuuauccccucccagaagacccagccauccccaugcccccaagacaguuccaccagcuggccgcccagggaccccaggagugcuugguccguaucuacauuguccgagcauuuggccugcagcccaaggaccccaauggaaagugugauccuuacaucaagaucuccauagggaagaaaucagugagugaccaggauaacuacauccccugcacgcuggagcccguauuuggaaagauguucgagcugaccugcacucugccucuggagaaggaccuaaagaucacucucuaugacuaugaccuccucuccaaggacgaaaagaucggugagacggucgucgaccuggagaacaggcugcuguccaaguuuggggcucgcuguggacucccacagaccuacugugucucuggaccgaaccaguggcgggaccagcuccgccccucccagcuccuccaccucuucugccagcagcauagagucaaggcaccuguguaccggacagaccguguaauguuucaggauaaagaauauuccauugaagagauagaggcuggcaggaucccaaacccacaccugggcccaguggaggagcgucuggcucugcaugugcuucagcagcagggccuggucccggagcacguggagucacggccccucuacagcccccugcagccagacaucgagcaggggaagcugcagaugugggucgaccuauuuccgaaggcccuggggcggccuggaccucccuucaacaucaccccacggagagccagaagguuuuuccugcguuguauuaucuggaauaccagagaugugauccuggaugaccugagccucacgggggagaagaugagcgacauuuaugugaaagguuggaugauuggcuuugaagaacacaagcaaaagacagacgugcauuaucguucccugggaggugaaggcaacuucaacuggagguucauuuuccccuucgacuaccugccagcugagcaagucuguaccauugccaagaaggaugccuucuggaggcuggacaagacugagagcaaaaucccagcacgagugguguuccagaucugggacaaugacaaguucuccuuugaugauuuucugggcucccugcagcucgaucucaaccgcaugcccaagccagccaagacagccaagaagugcuccuuggaccagcuggaugaugcuuuccacccagaaugguuugugucccuuuuugagcagaaaacagugaagggcugguggcccuguguagcagaagagggugagaagaaaauacuggcgggcaagcuggaaaugaccuuggagauuguagcagagagugagcaugaggagcggccugcuggccagggccgggaugagcccaacaugaacccuaagcuugaggacccaaggcgccccgacaccuccuuccugugguuuaccuccccauacaagaccaugaaguucauccuguggcggcguuuccggugggccaucauccucuucaucauccucuucauccugcugcuguuccuggccaucuucaucuacgccuucccgaacuaugcugccaugaagcuggugaagcccuucagcugaggacucuccugcccuguagaaggggccgugggguccccuccagcaugggacuggccugccuccuccgcccagcucggcgagcuccuccagaccuccuaggccugauuguccugccagggugggcagacagacagauggaccggcccacacucccagaguugcuaacauggagcucugagaucaccccacuuccaucauuuccuucucccccaacccaacgcuuuuuuggaucagcucagacauauuucaguauaaaacaguuggaaccacaaaaaaaaaaaaaaaaaaaaaaaaa

SEQ ID NO: 67 - RNA, 5066 nt, Homo sapiens
  misc_feature (594)..(693): n is a, c, g, or u
  misc_feature (1130)..(1229): n is a, c, g, or u
  misc_feature (3750)..(3849): n is a, c, g, or u
  misc_feature (4480)..(4607): n is a, c, g, or u
aggagcaagccgagagccagccggccggcgcacuccgacuccgagcagucucuguccuucgacccgagccccgcgcccuuuccgggaccccugccccgcgggcagcgcugccaaccugccggccauggagaccccgucccagcggcgcgccacccgcagcggggcgcaggccagcuccacuccgcugucgcccacccgcaucacccggcugcaggagaaggaggaccugcaggagcucaaugaucgcuuggcggucuacaucgaccgugugcgcucgcuggaaacggagaacgcagggcugcgccuucgcaucaccgagucugaagagguggucagccgcgagguguccggcaucaaggccgccuacgaggccgagcucggggaugcccgcaagacccuugacucaguagccaaggagcgcgcccgccugcagcuggagcugagcaaagugcgugaggaguuuaaggagcugaaagcgcggugaguucgcccagguggcugcgugccuggcggggaguggagagggcggcgggccggcgccccuggccggccgcaggaagggagugagagggccuggaggccgauaacuuugcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncugguaauugcaggcauagcagcgccagcccccauggcugaccuccugggagccuggcacugucuaggcacacagacuccuucucuuaaaucuacucuccccucucuucuuuagcaauaccaagaaggagggugaccugauagcugcucaggcucggcugaaggaccuggaggcucugcugaacuccaaggaggccgcacugagcacugcucucagugagaagcgcacgcuggagggcgagcugcaugaucugcggggccagguggccaaggugaggccacccugcagggcccacccauggccccaccuaacacauguacacucacucuucuaccuaggcccucccccauguggugccuggucugaccugucaccugauuucagagccauucaccuguccuagagucauuuuacccacugaggucacaucuuauccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngucacccaggcuggagugcaguagugcgaucucggcucacugcaaccuccaccuccuggauucaagcgauucuugugccucagccuccugaguagcugggacuacaggcgugugccaccaucaugccuggcuacuuuuuuguauuagauauauauuuucucucuuagcacaguaccuaccaagagugagugaguagauguccugaccccugcaggcauccaaggcccuccuucccuggaccuguuuccacaugugugaaggggugcacaggcagcagcccaccucucagcuuccuuccaguucuuguguucugugaccccuuuuccucaucucugccugcuuccucacagcuugaggcagcccuaggugaggccaagaagcaacuucaggaugagaugcugcggcggguggaugcugagaacaggcugcagaccaugaaggaggaacuggacuuccagaagaacaucuacagugagguggggacugugcuuugcaagccagagggcuggggcugggugaugacagacuugggcugggcuaggggggaccagcugugugcagagcucgccuuccugagucccuugcccuaguggacagggaguuggggguggccagcacucagcucccagguuaaaguggggcugguaguggcucauggaguagggcugggcagggagccccgccccugggucuuggccucccaggaacuaauucugauuuugguuucuguguccuuccuccaacccuuccaggagcugcgugagaccaagcgccgucaugagacccgacugguggagauugacaaugggaagcagcgugaguuugagagccggcuggcggaugcgcugcaggaacugcgggcccagcaugaggaccagguggagcaguauaagaaggagcuggagaagacuuauucugccaaggugcuugcucucgauugguucccucacugccucugcccuuggcagcccuacccuuacccacgcugggcuaugccuucuggggaucaggcagaugguggcagggagcucaggguggcccaggaccuggggcuguagcagugaugcccaacucaggccugugccuccaccccucccagucaccacaguccuaacccuuuguccuccccuccagcuggacaaugccaggcagucugcugagaggaacagcaaccuggugggggcugcccacgaggagcugcagcagucgcgcauccgcaucgacagccucucugcccagcucagccagcuccagaagcaggugauaccccaccucaccccucucuccaggggccuagagucugggccggaugcaggcuggaagcccaggguugggggugggggugggggugggagguuccugaggaggagagggaugaaaaguguccccacaaccacagagaagggucgcaggauguggagucagauggccugugugcuguuucuguacacucuuaccucaccuucacuucucagggcuuugguuuucccauucgaaaauggaggcuguucuuaaucucccuaacucagaguugccacaggacucugcaaugugagguguuaaaagcaucaguauuuuucuaguuggcugugcuauuugugacaggagaaaaagucuagccucagaacgagagguuucaguuagacaaggggaaggacuucccaguugccagccaagacuauguuuagagcuugugauguucagagcuggcucugaugagggcucuggggaagcucugauugcagauccuggagagaguagccaggugucuccuacaccgacccacgucccuccuuccccauacuuagggcccuugggagcucaccaaacccucccaccccccuucagcuggcagccaaggaggcgaagcuucgagaccuggaggacucacuggcccgugagcgggacaccagccggcggcugcuggcggaaaaggagcgggagauggccgagaugcgggcaaggaugcagcagcagcuggacgaguaccaggagcuucuggacaucaagcuggcccuggacauggagauccacgccuaccgcaagcucuuggagggcgaggaggagaggugggcuggggagacgucggggaggugcuggcaguguccucuggccggcaacuggccuugacuagacccccacuuggucucccucuccccaggcuacgccugucccccagcccuaccucgcagcgcagccguggccgugcuuccucucacucaucccagacacagggugggggcagcgucaccaaaaagcgcaaacuggaguccacugagagccgcagcagcuucucacagcacgcacgcacuagcgggcgcguggccguggaggagguggaugaggagggcaaguuuguccggcugcgcaacaaguccaaugagguaggcuccugcucagggucuaaggggauacagcugcaucagggagagaguggcaagacagaaggauggcauguggagagaggaacauccuugcccucagaggguggaccagggugagccuguauaucuccuccacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcugcguauguguccacagaucauggcuauuauccccgggggaagggcagugacagggguguguguagauggaaggagaggccucaauugcaggcaggcagagggcugggccuuugagcaagauacacccaagagccugggugagccuccccgaccuuccucuucccuaucuucccggcaggaccaguccaugggcaauuggcagaucaagcgccagaauggagaugaucccuugcugacuuaccgguucccaccaaaguucacccugaaggcugggcagguggugacggugaguggcagggcgcuugggacucuggggaggccuuggguggcgaugggagcgcugggguaaguguccuuuucuccucuccagaucugggcugcaggagcuggggccacccacagccccccuaccgaccugguguggaaggcacagaacaccuggggcugcgggaacagccugcguacggcucucaucaacuccacuggggaaguaaguaggccugggccuggcugcuugcuggacgaggcucccccugauggccaacaucggagccagcugcccccaacccaaguuugccaauucagggccccuuucuagagcucucuguugcaggcuccagacuucucnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnugaguuccuuagcuccaucaccacagaggacagaguaagcagcaggccggacaaagggcaggccacaagaaaaguugcagguggucacugggguagacaugcuguacaacccuucccuggcccugacccuuggaccugguuccauguccccaccaggaaguggccauacgcaagcuggugcgcucagugacugugguugaggacgacgaggaugaggauggagaugaccugcuccaucaccaccaugugagugguagccgccgcugaggccgagccugcacuggggccacccagccaggccugggggcagccucuccccagccuccccgugccaaaaaucuuuucauuaaagaauguuuuggaacuuuacucgcuggccuggccuuucuucucucuccucccuauaccuugaacagggaacccaggugucugggugcccuacucugguaaggaagggag